(19) United States
(12) Patent Application Publication
(10) Pub. No.: US 2005/0261290 A1
(43) Pub. Date: Nov. 24, 2005

(54) Novel compounds of proline and morpholine derivatives

(76) Inventors: Hengmiao Cheng, San Diego, CA (US); Stephan James Cripps, San Diego, CA (US); Klaus Ruprecht Dress, San Diego, CA (US); Jacqui Elizabeth Hoffman, San Diego, CA (US); Buwen Huang, San Diego, CA (US); Stanley William Kupchinsky, San Diego, CA (US); Phuong Thi Quy Le, San Diego, CA (US); Sajiv Krishnan Nair, Vista, CA (US); Timothy James Parrott, Carlsbad, CA (US); Christopher Ronald Smith, San Diego, CA (US); Yong Wang, San Diego, CA (US); Yi Yang, San Diego, CA (US)

Correspondence Address: AGOURON PHARMACEUTICALS, INC., 10777 SCIENCE CENTER DRIVE, SAN DIEGO, CA, 92121, US

(21) Appl. No.: 11/122,599

(22) Filed: May 4, 2005

Related U.S. Application Data
(60) Provisional application No. 60/569,362, filed on May 6, 2004.

(51) Int. Cl. A61K 31/5377; A61K 31/445; A61K 31/4025; A61K 31/401; C07D 413/02; C07D 43/02

(52) U.S. Cl. 514/235.2; 514/319; 514/422; 514/423; 544/128; 546/203; 548/518; 548/528

(57) ABSTRACT

The present invention relates to compounds with the formulas (I), (II), and (III), or a pharmaceutically acceptable salt thereof: 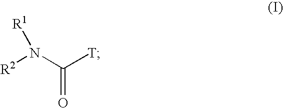
wherein T is a (4 to 10)-membered heterocyclyl selected from the group consisting of and wherein R¹, R² and R³ are as defined in the specification. The invention also relates to pharmaceutical compositions comprising the compounds of formulas (I), (II), and (III) and methods of treating a condition that is mediated by the modulation of the 11-β-hsd-1 enzyme, the method comprising administering to a mammal an effective amount of a compound of formulas (I), (II), and (III).

This application claims the benefit of U.S. Application Ser. No. 60/569,326 filed May 6, 2004, hereby incorporated by reference in its entirety for all purposes.

### FIELD OF INVENTION

The present invention relates to novel compounds, to pharmaceutical compositions comprising the compounds, as well as to the use of the compounds in medicine and for the preparation of a medicament which acts on the human 11-β-hydroxysteroid dehydrogenase type 1 enzyme (11-β-hsd-1).

### BACKGROUND OF THE INVENTION

It has been known for more than half a century that glucocorticoids have a central role in diabetes. For example, the removal of the pituitary or the adrenal gland from a diabetic animal alleviates the most severe symptoms of diabetes and lowers the concentration of glucose in the blood (Long, C. D. and F. D. W. Leukins (1936) J. Exp. Med. 63: 465-490; Houssay, B. A. (1942) Endocrinology 30: 884-892). Additionally, it is also well established that glucocorticoids enable the effect of glucagon on the liver.

The role of 11-β-hsd-1 as an important regulator of local glucocorticoid effects and thus of hepatic glucose production is well substantiated (see e.g. Jamieson, et al. (2000) J. Endocrinol. 165: p. 685-692). The hepatic insulin sensitivity was improved in healthy human volunteers treated with the non-specific 11-β-hsd-1 inhibitor carbenoxolone (Walker, B. R., et al. (1995) J. Clin. Endocrinol. Metab. 80: 3155-3159). Furthermore, the expected mechanism has been established by different experiments with mice and rats. These studies showed that the mRNA levels and activities of two key enzymes in hepatic glucose production were reduced, namely the rate-limiting enzyme in gluconeogenesis, phosphoenolpyruvate carboxykinase (PEPCK), and glucose-6-phosphatase (G6 Pase) catalyzing the last common step of gluconeogenesis and glycogenolysis. Finally, the blood glucose level and hepatic glucose production was reduced in mice having the 11-β-hsd-1 gene knocked-out. Data from this model also confirms that inhibition of 11-β-hsd-1 will not cause hypoglycemia, as predicted, since the basal levels of PEPCK and G6 Pase are regulated independently of glucocorticoids (Kotelevtsev, Y., et al., (1997) Proc. Natl. Acad. Sci. USA 94:14924-14929).

Abdominal obesity is closely associated with glucose intolerance, hyperinsulinemia, hypertriglyceridemia, and other factors of the so-called Metabolic Syndrome (e.g. raised blood pressure, decreased levels of HDL and increased levels of VLDL) (Montague & O'Rahilly, Diabetes 49: 883-888, 2000). Obesity is an important factor in Metabolic Syndrome as well as in the majority (>80%) of type 2 diabetic, and omental fat appears to be of central importance. Inhibition of the enzyme in pre-adipocytes (stromal cells) has been shown to decrease the rate of differentiation into adipocytes. This is predicted to result in diminished expansion (possibly reduction) of the omental fat depot, i.e. reduced central obesity (Bujalska, I. J., Kumar, S., and Stewart, P. M. (1997) Lancet 349: 1210-1213).

The morpholine and proline derivative compounds of the present invention are 11 β-hsd-1 inhibitors, and are therefore believed to be useful in the treatment of diabetes, obesity, glaucoma, osteoporosis, cognitive disorders, immune disorders, depression, hypertension, and metabolic diseases.

### SUMMARY OF THE INVENTION

The invention relates to a compound of formula (I): 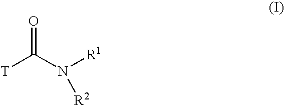
wherein;
R¹ is independently selected from the group consisting of (C₁-C₆)alkyl, —(CR⁴R⁵)ₜ(C₃-C₁₂)cycloalkyl, —(CR⁴R⁵)ₜ(C₆-C₁₂)aryl, and —(CR⁴R⁵)ₜ(4 to 10)-membered heterocyclyl;
k is independently selected from 1 or 2;
j is independently selected from the group consisting of 0, 1, and 2;
t, u, p, q and v are each independently selected from the group consisting of 0, 1, 2, 3, 4, and 5;
T is a (4 to 10)-membered heterocyclyl containing at least one nitrogen atom, wherein said nitrogen atom is optionally substituted by at least one R³ group;
R² is selected from H or (C₁-C₆)alkyl;
   each R³ group is independently selected from the group consisting of —CF₃, —CHF₂, —CH₂F, trifluoromethoxy, (C₁-C₆)alkoxy, (C₁-C₆)alkyl, (C₂-C₆)alkenyl, (C₂-C₆)alkynyl, —(C═O)—R⁴, —(C═O)—O—R⁴, —(CR⁴R⁵)ₜ(C₆-C₁₂)aryl, —(CR⁴R⁵)ₜ(C₃-C₁₂)cycloalkyl, —(CR⁴R⁵)ₜ(4 to 10)-membered heterocyclyl, —(CR⁴R⁵)ₜ—(C═O)(CR⁴R⁵)ₜ(C₆-C₁₂)aryl, and —(CR⁴R⁵)ₜ—(C═O)(CR⁴R⁵)ₜ(4 to 10)-membered heterocyclyl;
   each R⁴ and R⁵ group is independently selected from H or (C₁-C₆)alkyl;
   any nitrogen atom of any (4 to 10)-membered heterocyclyl of the foregoing R³ group is optionally substituted with a substituent independently selected from the group consisting of (C₁-C₆)alkyl, —(SO)ₖ—R⁴, —(C═O)—O—R⁴, and —(C═O)—R⁴;
   each carbon atom of T, R¹, R² and R³ is optionally substituted by 1 to 4 R⁶ groups;
   each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(C═O)—R⁷, —(C═O)—O—R⁷, —O—R⁷, —O—(C═O)—R⁷, —O—(C═O)—NR⁷R⁸, —NR⁸—((C═O)—R⁹) —(C═O)NR⁸R⁹, —NR⁸R⁹, —NR⁸—(OR⁹), —NR⁸—((C═O)—O—R⁹), —S(O)ₖ—NR⁸R⁹, —S(O)ₖ—R⁸, —O—S(O)ₖ—R⁸, —NR⁸—S(O)ₖ—R⁹, —(CR¹⁰R¹¹)ᵥ(C₆-C₁₂)aryl, —(CR¹⁰R¹¹)ᵥ(C₃-C₁₂)cycloalkyl, —(CR¹⁰R¹¹)_{q}(4 to 10)-membered heterocyclyl, —(CR¹⁰R¹¹)_{q}(C═O)(CR¹⁰R¹¹)ᵥ(C₆-C₁₂)aryl, —(CR¹⁰R¹¹)ᵥ(C═O)(CR¹⁰R¹¹)_{q}(C₃-C₁₂)cycloalkyl, —(CR¹⁰R¹¹)_{q}(C═O)(CR¹⁰R¹¹)ᵥ(4 to 10)-membered heterocyclyl, —(CR¹⁰R¹¹)ᵥO(CR¹⁰R¹¹)_{q}(C₆-C₁₂)aryl, —(CR¹⁰R¹¹)O(CR¹⁰R¹¹)_{q}(C₃-C₁₀)cycloalkyl, —(CR¹⁰R¹¹)ᵥO(CR¹⁰R¹¹)_{q}(4 to 10)-membered heterocyclyl, —(CR¹⁰R¹¹)_{q}S(O)ⱼ(CR¹⁰R¹¹)ᵥ(C₆-C₁₂)aryl, —(CR¹⁰R¹¹)_{q}S(O)ⱼ(CR¹⁰R¹¹)ᵥ(C₃-C₁₂)cycloalkyl, and —(CR¹⁰R¹¹)_{q}S(O)ⱼ (CR¹⁰R¹¹)ᵥ(4 to 10)-membered heterocyclyl;
   any 1 or 2 carbon atoms of any (4 to 10)-membered heterocyclyl moiety of the foregoing R⁶ groups are optionally substituted with an oxo group;
   any carbon atom of any (C₁-C₆)alkyl, any (C₆-C₁₂)aryl, any (C₃-C₁₀)cycloalkyl, or any (4 to 10)-membered heterocyclyl of the foregoing R⁶ groups are optionally substituted with 1 to 3 substituents independently selected from the group consisting of halo, cyano, nitro, —CF₃, —CFH₂, —CF₂H, trifluoromethoxy, azido, —O—R¹², —(C═O)—R¹², —(C═O)—O—R¹², —O—(C═O)—R¹³, —NR¹³(C═O)R¹⁴, —(C═O)NR¹⁴R¹⁵, —NR¹⁴R¹⁵, —N¹⁴—(O¹⁵), (C₁-C₆)alkyl, (C₂-C₆)alkenyl, (C₂-C₆)alkynyl, —(CR¹⁶R¹⁷)ᵤ(C₆-C₁₂)aryl, —(CR¹⁶R¹⁷)ᵤ(C₃-C₁₂)cycloalkyl, and —(CR¹⁶R¹⁷)ᵤ(4 to 10)-membered heterocyclyl;
   each R⁷, R⁸, R⁹, R¹⁰, R¹¹, R¹², R¹³, R¹⁴, R¹⁵, R¹⁶ and R¹⁷ group is independently selected from the group consisting of H, (C₁-C₆)alkyl, —(C═O)NH(R¹⁸), —(CR¹⁸R¹⁹)ₚ(C₆-C₁₂)aryl, —(CR¹⁸R¹⁹)ₚ(C₃-C₁₂)cycloalkyl, and —(CR¹⁸R¹⁹)ₚ(4 to 10)-membered heterocyclyl;
   any 1 or 2 carbon atoms of the (4 to 10)-membered heterocyclyl of said each R⁷, R⁸, R⁹,
R¹⁰, R¹¹, R¹², R¹³, R¹⁴, R¹⁵, R¹⁶ and R¹⁷ group is optionally substituted with an oxo group;
   any carbon atoms of any (C₁-C₆)alkyl, any (C₆-C₁₂)aryl, any (C₃-C₁₂)cycloalkyl or any (4 to 10)-membered heterocyclyl of the foregoing R⁷, R⁸, R⁹, R¹⁰, R¹¹, R¹², R¹³, R¹⁴, R¹⁵, R¹⁶ and R¹⁷ groups are optionally substituted with 1 to 3 substituents independently selected from the group consisting of halo, cyano, nitro, —NR²⁰R²¹, —CF₃, —CHF₂, —CH₂F, hydroxy, trifluoromethoxy, (C₁-C₆)alkyl, (C₂-C₆)alkenyl, (C₂-C₆)alkynyl, and (C₁-C₆)alkoxy;
   each R¹⁶, R¹⁹, R²⁰, and R²¹ group is independently selected from H or (C₁-C₆)alkyl;
   and wherein any of the above mentioned substituents comprising a —CH₃ (methyl), —CH₂ (methylene), or —CH (methine) group which is not attached to a halo, —SO or —SO₂ group, or to a N, O or S atom optionally bears on said group a substituent independently selected from hydroxy, halo,
   —(C₁-C₆)alkyl-(C(C₁-C₆)alkoxy, —NH₂, —NH((C₁-C₆)(alkyl)) and —N((C₁-C₆)(alkyl))₂;
      or a pharmaceutically acceptable salt or solvate thereof.

An embodiment of the invention relates to a compound according to formula (I), wherein T is a (5 to 7)-membered heterocyclyl containing at least one nitrogen atom.

Another embodiment of the invention relates to a compound according to formula (I), wherein R² is H or methyl.

Yet another embodiment of the invention relates to a compound according to formula (I), wherein R¹ is independently selected from the group consisting of adamantyl, benzyl, cyclohexyl, 2,3-dihydro-1H-inden-2-yl, —CH₂-pyridinyl, naphthalenyl, —CH₂CH₂-morpholinyl, azabicyclo(2.2.1.)heptyl, bicyclo(2.2.1.)heptyl, cycloheptyl, —CH₂-cyclopentyl, pentacyclo(4.2.0.0^{2,5}0^{3,8}.0^{4,7})octyl, tetrahydronaphthalenyl, and naphthyridinyl; wherein each carbon atom is optionally substituted by 1 to 4 R⁶ groups, each R⁶ group is independently selected from the group consisting of halo, cyano, —CF₃, trifluoromethoxy, hydroxy, (C₁-C₆)alkoxy, (C₁-C₆)alkyl, —O—R⁷, —(C═O)—R⁷, —(C═O)—O—R⁷, —O—(C═O)—NR⁷R⁸, —NR⁸⁹, —NR⁸—((C═O)—R⁹), —NR⁸—((C═O)—O—R⁹), —NR⁸—(S(O)ₖ—R⁹), and —(C═O)—NR⁸R⁹.

In still yet another embodiment, the invention relates to a compound according to formula (I), wherein T independently selected from the group consisting 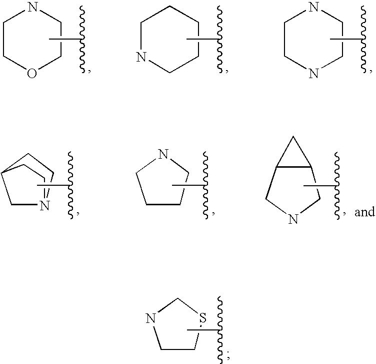
wherein said nitrogen atom is optionally substituted by at least one R³ group, wherein each said R³ group is independently selected from the group consisting of (C₁-C₆)alkyl, —(CR⁴R⁵)ₜ(C₆-C₁₂)aryl,
   —(CR⁴R⁵)ₜ(C₃-C₁₂)cycloalkyl, —CF₃, (C₁-C₆)alkoxy, —(C═O)—O—R⁴, and —(CR⁴R⁵)ₜ(4 to 10)-membered heterocyclyl.

An embodiment of the invention relates to a compound of formula (II): 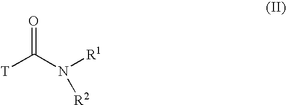
wherein;
R¹ is independently selected from the group consisting of —(CR⁴R⁵)ₜ(C₃-C₁₂)cycloalkyl, —(CR⁴R⁵)ₜ(C₆-C₁₂)aryl, and —(CR⁴R⁵)ₜ(4 to 10)-membered heterocyclyl;
k is independently selected from 1 or 2;
j is independently selected from the group consisting of 0, 1, and 2;
t, u, p, q and v are each independently selected from the group consisting of 0, 1, 2, 3, 4, and 5;
T is a (5 to 7)-membered heterocyclyl containing at least one nitrogen atom, wherein said nitrogen atom is optionally substituted by at least one R³ group;
R² is selected from H or methyl;
each R³ is independently selected from the group consisting of (C₁-C₆)alkyl, —(CR⁴R⁵)ₜ(C₆-C₁₂)aryl, —(CR⁴R⁵)ₜ(C₃-C₁₂)cycloalkyl, —(CR⁴R⁵)ₜ(4 to 10)-membered heterocyclyl, —CF₃, (C₁-C₆)alkoxy, and
   —(C═O)—O—R⁴;
each R⁴ and R⁵ group is independently selected from H or (C₁-C₆)alkyl;
any nitrogen atom of any (4 to 10)-membered heterocyclyl of the foregoing R³ group is optionally substituted with a substituent independently selected from the group consisting of (C₁-C₆)alkyl, —(SO)ₖ—R⁴, —(C═O)—O—R⁴, —(C═O)—R⁴;
each carbon atom of T, R¹, R² and R³ is optionally substituted by 1 to 3 R⁶ groups;
each R⁶ group is independently selected from the group consisting of halo, cyano, —CF₃, trifluoromethoxy, hydroxy, (C₁-C₆)alkoxy, (C₁-C₆)alkyl, —O—R⁷, —(C═O)—R⁷, —(C═O)—O—R⁷, —O—(C═O)—NR⁷R⁸, —NR⁸R⁹, —NR⁸—((C═O)R⁹), —NR⁸—((C═O)—O—R⁹), —NR⁸—(S(O)ₖ—R⁹), —(C═O)—NR⁸R⁹;
any 1 or 2 carbon atoms of any (4 to 10)-membered heterocyclyl moiety of the foregoing R⁶ groups are optionally substituted with an oxo group;
any carbon atom of any (C₁-C₆)alkyl of the foregoing R⁶ groups are optionally substituted with 1 to 3 substituents independently selected from the group consisting of halo, cyano, —CF₃, —O—R¹⁰, (C₁-C₆)alkyl, NR¹⁰R¹¹, and —(C═O)—NR¹¹R¹²;
each R⁷, R⁸, R⁹, R¹⁰, R¹¹, and R¹² group is independently selected from H, —(C₁-C₆)alkyl;
any carbon atoms of any (C₁-C₆)alkyl of the foregoing R⁷, R⁸, R⁹, R¹⁰, R¹¹, and R¹² groups are optionally substituted with 1 to 3 substituents independently selected from halo, cyano, nitro, —NR¹³R¹⁴,
   —CF₃, —CHF₂, —CH₂F, trifluoromethoxy, (C₁-C₆)alkyl, (C₂-C₆)alkenyl, (C₂-C₆)alkynyl, hydroxy, and (C₁-C₆)alkoxy;
each R¹³ and R¹⁴ group is independently selected from H or (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from hydroxy, halo,
   —(C₁-C₆)alkyl, —(C₁-C₆)alkoxy, —NH₂, —NH((C₁-C₆)(alkyl)) and —N((C₁-C₆)(alkyl))₂;
or a pharmaceutically acceptable salt or solvate thereof.

Another embodiment of the invention relates to the compound according to formula (II), wherein T independently selected from the group consisting of 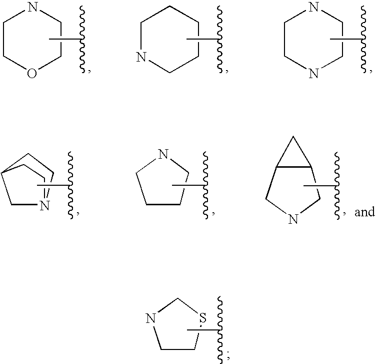
wherein said nitrogen atom is optionally substituted by at least one R³ group, wherein each said R³ group is independently selected from the group consisting of (C₁-C₆)alkyl, —(CR⁴R⁵)ₜ(C₆-C₁₂)aryl, —CF₃, (C₁-C₆)alkoxy, —(C═O)—O—R⁴, —(CR⁴R⁵)ₜ(C₃-C₁₂)cycloalkyl, and —(CR⁴R⁵)ₜ(4 to 10)-membered heterocyclyl.

In yet another embodiment, the invention relates to the compound according to formula (II), wherein R² is H or methyl.

An embodiment of the invention relates to a compound according to formula (II), wherein R¹ is independently selected from the group consisting of adamantyl, benzyl, cyclohexyl, 2,3-dihydro-1H-inden-2-yl, —CH₂-pyridinyl, naphthalenyl, —CH₂CH₂-morpholinyl, azabicyclo(2.2.1.)heptyl, bicyclo(2.2.1.)heptyl, cycloheptyl, —CH₂-cyclopentyl, pentacyclo(4.2.0.^{2,5}.0^{3,8}.0^{4,7})octyl, tetrahydronaphthalenyl, and naphthyridinyl;
wherein each carbon atom is optionally substituted by 1 to 4 R⁶ groups, each R⁶ group is independently selected from the group consisting of halo, cyano, —CF₃, trifluoromethoxy, hydroxy, (C₁-C₆)alkoxy, (C₁-C₆)alkyl, —O—R⁷, —(C═O)—R⁷, —(C═O)—O—R⁷, —O—(C═O)—NR⁷R⁸, —NR⁸R⁹, —NR⁸—((C═O)—R⁹), —NR⁸—((C═O)—O—R⁹), —NR⁸—(S(O)ₖ—R⁹), and —(C═O)—NR⁸R⁹.

In another embodiment, the invention relates to a compound of formula (III): 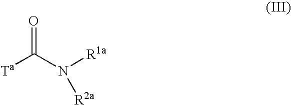
wherein;
R^{1a} is independently selected from the group consisting of adamantyl, bicyclo(2.2.1.)heptyl, and cyclohexyl;
R^{2a} is H;
T^{a} is a (5 or 6)-membered heterocyclyl containing at least one nitrogen atom, independently selected from the group consisting of pyrrolidinyl, morpholinyl, and piperidinyl;
wherein said nitrogen atom is optionally substituted by at least one R^{3a} group;
each R^{3a} is independently selected from the group consisting of methyl, ethyl, propyl, and benzyl;
each carbon atom of R^{1a} and R^{3a} is optionally substituted by 1 to 4 R^{6a} groups;
each R^{6a} group is independently selected from the group consisting of —N(CH₃)(CH₃), —NH₂, —N(CH₃)(CH₂C₆H₅), —N(H)(CH₃), pyrrolidinyl, -piperidinyl-((C═O)CH₃), -piperidinyl-(CH₃), cyclohexyl, cyclopentyl, -piperidinyl-(SO₂)CH₃, hydroxy, and cyano.

An embodiment of the invention relates to a compound of formula

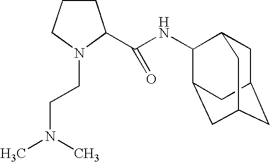

Another embodiment of the invention relates to a compound of formula

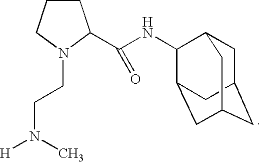

Yet another embodiment of the invention relates to a compound of formula

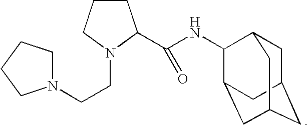

Yet another embodiment of the invention relates to a compound of formula

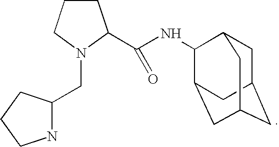

Another embodiment of the invention relates to a compound of formula

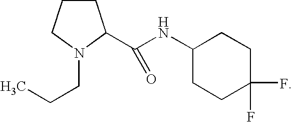

Another embodiment of the invention relates to a compound of formula

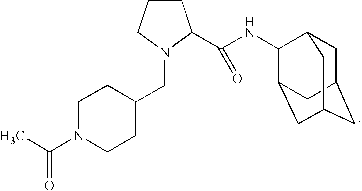

Yet another embodiment of the invention relates to a compound of formula

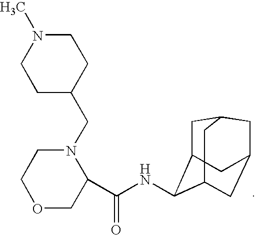

An embodiment of the invention relates to a compound of formula

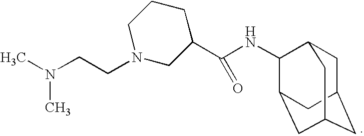

Another embodiment of the invention relates to a compound of formula

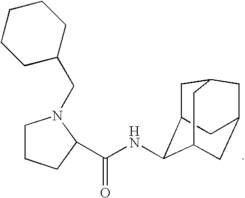

An embodiment of the invention relates to a compound of formula

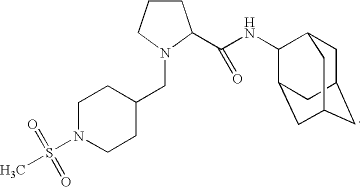

Another embodiment of the invention relates to a compound of formula

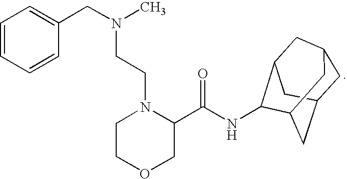

Yet another embodiment of the invention relates to a compound of formula

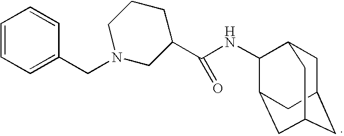

Yet another embodiment of the invention relates to a compound of formula

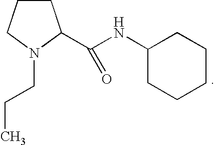

Another embodiment of the invention relates to a compound of formula

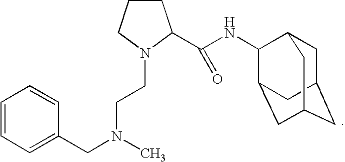

Yet another embodiment of the invention relates to a compound of formula

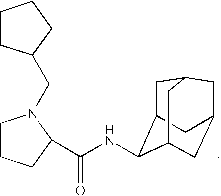

Another embodiment of the invention relates to a compound of formula

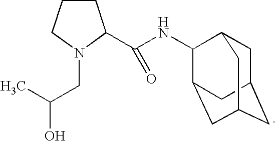

An embodiment of the invention relates to a compound of formula

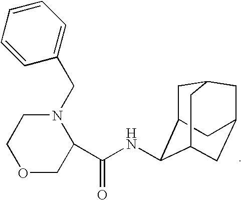

Another embodiment of the invention relates to a compound of formula

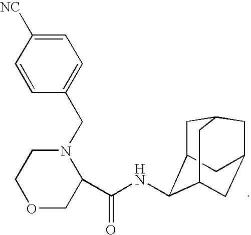

Yet another embodiment of the invention relates to a compound of formula

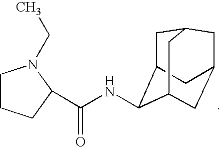

Yet another embodiment of the invention relates to a compound of formula

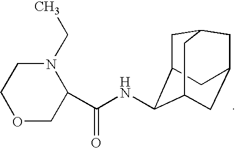

An embodiment of the invention relates to a compound of formula

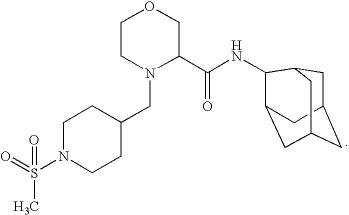

Another embodiment of the invention relates to a compound of formula

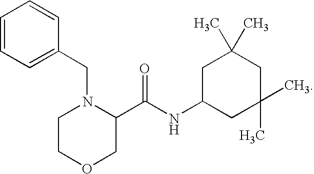

Yet another embodiment of the invention relates to a compound of formula

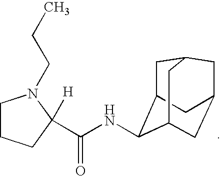

An embodiment of the invention relates to a compound of formula

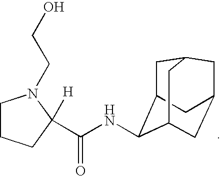

An embodiment of the invention relates to a compound of formula

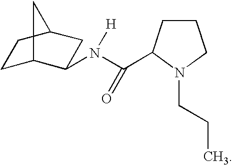

Another embodiment of the invention relates to a compound of formula

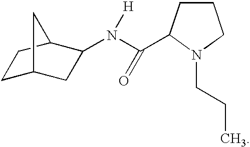

In yet another embodiment of the invention relates to a compound of formula

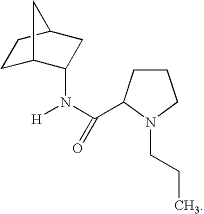

In yet another embodiment of the invention relates to a compound of formula

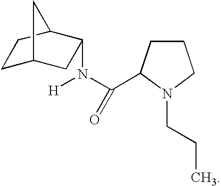

Another embodiment of the invention relates to a compound of formula

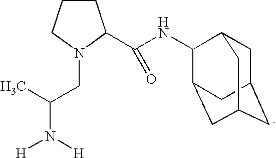

An embodiment of the invention relates to a compound of formula

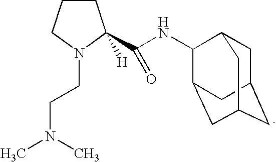

Another embodiment of the invention relates to a compound of formula

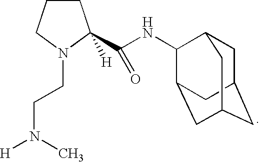

Yet another embodiment of the invention relates to a compound of formula

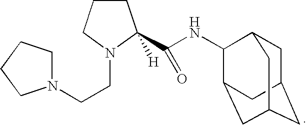

Yet another embodiment of the invention relates to a compound of formula

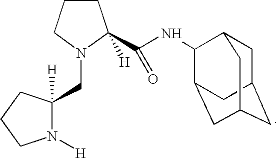

An embodiment of the invention relates to a compound of formula

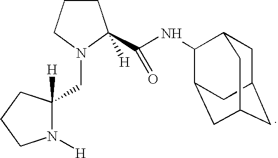

Another embodiment of the invention relates to a compound of formula

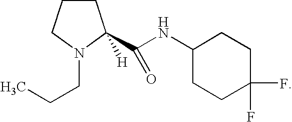

Another embodiment of the invention relates to a compound of formula

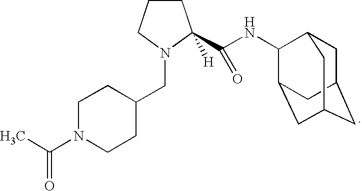

Yet another embodiment of the invention relates to a compound of formula

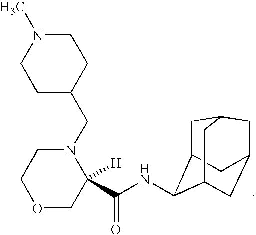

An embodiment of the invention relates to a compound of formula

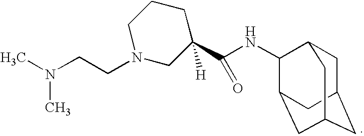

Another embodiment of the invention relates to a compound of formula

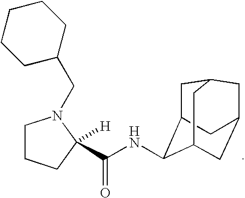

An embodiment of the invention relates to a compound of formula

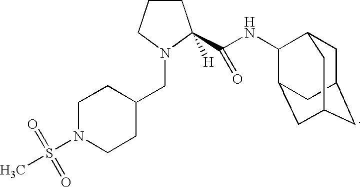

Another embodiment of the invention relates to a compound of formula

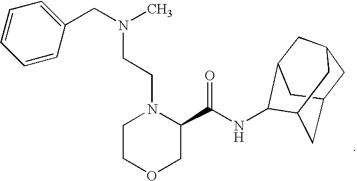

Yet another embodiment of the invention relates to a compound of formula

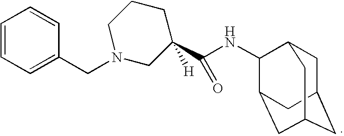

Yet another embodiment of the invention relates to a compound of formula

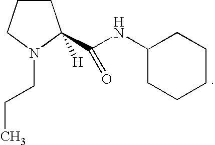

An embodiment of the invention relates to a compound of formula

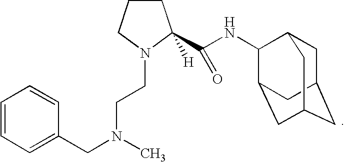

Another embodiment of the invention relates to a compound of formula

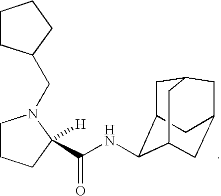

Yet another embodiment of the invention relates to a compound of formula

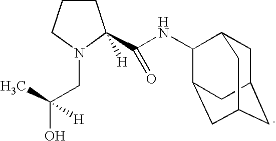

Another embodiment of the invention relates to a compound of formula

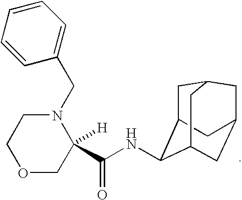

An embodiment of the invention relates to a compound of formula

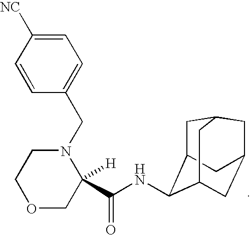

Another embodiment of the invention relates to a compound of formula

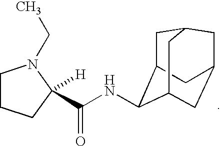

Yet another embodiment of the invention relates to a compound of formula

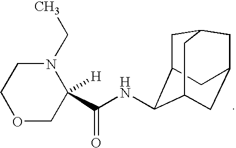

Yet another embodiment of the invention relates to a compound of formula

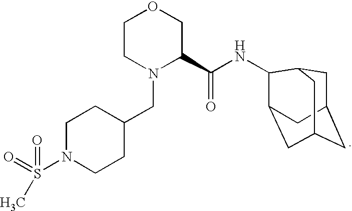

An embodiment of the invention relates to a compound of formula

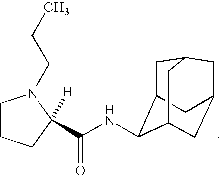

Another embodiment of the invention relates to a compound of formula

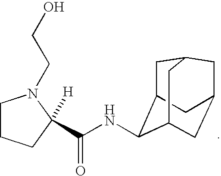

An embodiment of the invention relates to a compound of formula

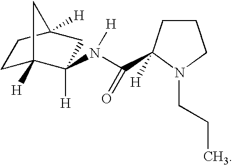

Another embodiment of the invention relates to a compound of formula

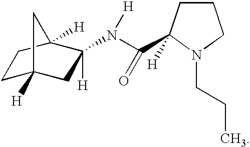

In yet another embodiment of the invention relates to a compound of formula

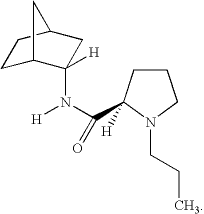

Another embodiment of the invention relates to a compound of formula

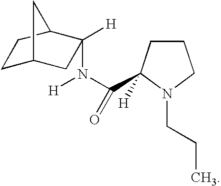

An embodiment of the invention relates to a compound of formula

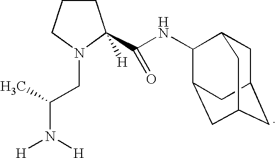

An embodiment of the invention relates to a pharmaceutical composition comprising an effective amount of a compound of formula (I), or a pharmaceutically acceptable salt or solvate thereof, and a pharmaceutically acceptable carrier.

Another embodiment of the invention relates to a method of treating a condition that is mediated by the modulation of the 11-β-hsd-1 enzyme, the method comprising administering to a mammal an effective amount of a compound according to formula (I), (II), or (III), or a pharmaceutically acceptable salt or solvate thereof.

In yet another embodiment, the invention relates to a method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, viral diseases, ophthalmic disorders, inflammatory disorders, or diseases in which the liver is a target organ, the method comprising administering to a mammal an effective amount of a compound according to formula (I), (II), or (III), or a pharmaceutically acceptable salt or solvate thereof.

In yet another embodiment, the invention relates to a method of treating glaucoma, the method comprising administering to a mammal an effective amount of a compound according to formula (I), (II), or (III), or a pharmaceutically acceptable salt or solvate thereof.

An embodiment of the invention relates to the method of treating glaucoma, comprising administering to a mammal an effective amount of a compound according to formula (I), (II), or (III), or a pharmaceutically acceptable salt or solvate thereof, in combination with lantanoprost.

Another embodiment of the invention relates to the method of treating glaucoma, comprising administering to a mammal an effective amount of a compound according to formula (I), (II), or (III), or a pharmaceutically acceptable salt or solvate thereof, in combination with a carbonic anhydrase inhibitor.

In yet another embodiment, the invention relates to the method of treating diabetes, comprising administering to a mammal an effective amount of a compound according to formula (I), (II), or (III), or a pharmaceutically acceptable salt or solvate thereof, in combination with a PPAR agonist.

The invention relates to a method of preparing a compound of formula (D): 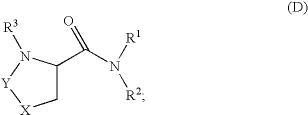
wherein;
R¹ is independently selected from the group consisting of (C₁-C₆)alkyl, —(CR⁴R⁵)ₜ(C₃-C₁₂)cycloalkyl, —(CR⁴R⁵)ₜ(C₆-C₁₂)aryl, and —(CR⁴R⁵)ₜ(4 to 10)-membered heterocyclyl;
t is independently selected from the group consisting of 0, 1, 2, 3, 4, and 5;
R² is selected from H or (C₁-C₆)alkyl;
R³ is independently selected from the group consisting of —CF₃, —CHF₂, —CH₂F, trifluoromethoxy, (C₁-C₆)alkoxy, (C₁-C₆)alkyl, (C₂-C₆)alkenyl, (C₂-C₆)alkynyl, —(C═O)—R⁴, —(C═O)—O—R⁴, —(CR⁴R⁵)ₜ(C₆-C₁₂)aryl, —(CR⁴R⁵)ₜ(C₃-C₁₂)cycloalkyl, —(CR⁴R⁵)ₜ(4 to 10)-membered heterocyclyl, —(CR⁴R⁵)ₜ—(C═O)(CR⁴R⁵)ₜ(C₆-C₁₂)aryl, and —(CR⁴R⁵)ₜ—(C═O)(CR⁴R⁵)ₜ(4 to 10)-membered heterocyclyl;
each R⁴ and R⁵ group is independently selected from H or (C₁-C₆)alkyl;
X is independently selected from the group consisting of —CR⁴R⁵, —O—, —S—, and —NR⁴—;
Y is —CR⁴R⁵;
comprising the steps of:
(a₁) treating a compound of formula (C): 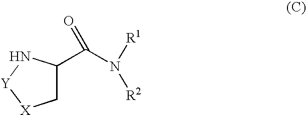
with R³-LV in a solvent in the presence of a base;
wherein;
LV is a suitable leaving group; and
X, Y, R¹, R², and R³ are as defined above.

Another embodiment of the invention relates to the method, wherein in step (a₁) LV is independently selected from the group consisting of Cl, Br, and methanesulfonate.

Another embodiment of the invention relates to the method, wherein the solvent in step (a₁) is selected from dichloromethane or N,N-dimethylformamide.

In yet another embodiment, the method, wherein the base in step (a₁) is independently selected from the group consisting of K₂CO₃, NaHCO₃, and (C₂H₅)₃N.

In yet another embodiment, the method, wherein step (a₁) proceeds at a temperature from about 20 degrees Celsius to about the boiling point of the solvent.

An embodiment of the invention relates to a method of preparing a compound of formula (D): 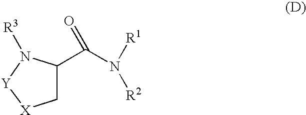
wherein;
R¹ is independently selected from the group consisting of (C₁-C₆)alkyl, —(CR⁴R⁵)ₜ(C₃-C₁₂)cycloalkyl, —(CR⁴R⁵)ₜ(C₆-C₁₂)aryl, and —(CR⁴R⁵)ₜ(4 to 10)-membered heterocyclyl;
t is independently selected from the group consisting of 0, 1, 2, 3, 4, and 5;
R² is selected from H or (C₁-C₆)alkyl;
R³ is independently selected from the group consisting of —CF₃, —CHF₂, —CH₂F, trifluoromethoxy, (C₁-C₆)alkoxy, (C₁-C₆)alkyl, (C₂-C₆)alkenyl, (C₂-C₆)alkynyl, —(C═O)—R⁴, —(C═O)—O—R⁴, —(CR⁴R⁵)ₜ(C₆-C₁₂)aryl, —(CR⁴R⁵)ₜ(C₃-C₁₂)cycloalkyl, —(CR⁴R⁵)ₜ(4 to 10)-membered heterocyclyl, —(CR⁴R⁵)—(C═O)(CR⁴R⁵)ₜ(C₆-C₁₂)aryl, and —(CR⁴R⁵), —(C═O)(CR⁴R⁵)ₜ(4 to 10)-membered heterocyclyl;
each R⁴ and R⁵ group is independently selected from H or (C₁-C₆)alkyl;
X is independently selected from the group consisting of —CR⁴R⁵, —O—, —S—, and —NR⁴—;
Y is —CR⁴R⁵;
comprising the steps of:
(a₂) treating a compound of formula (C): 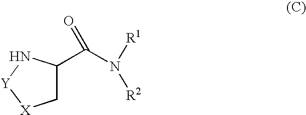
by reductive amination with an aldehyde or ketone in a solvent in the presence of an acid and a reducing agent;
wherein;
X, Y, R¹, and R² are defined above.

Another embodiment of the invention relates to the method, wherein the solvent in step (a₂) is independently selected from the group consisting of THF, MeOH, and CH₂Cl₂.

In yet another embodiment, the invention relates to the method, wherein the ketone in step (a₂) is acetone.

In yet another embodiment, the invention relatest to the method, wherein the aldehyde in step (a₂) is selected from formaldehyde or cyclopentanecarboxaldehyde.

An embodiment of the invention relates to the method, wherein the acid in step (a₂) is acetic acid.

Another embodiment of the invention relates to the method, wherein the reducing agent in step (a₂) is NaBCNH₃ or NaB(OAc)₃H.

In yet another embodiment, the invention relates to the method, wherein step (a₂) proceeds at a temperature range from about 20 degrees Celsius to about 60 degrees Celsius.

An embodiment of the invention relates to the method, further comprising the steps of preparing said compound of formula (C) comprising:
(b) treating a compound of formula (B) 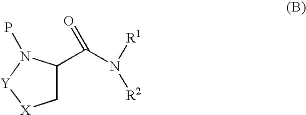
to produce said compound of formula (C) with a suitable deprotecting agent; wherein;
P is a protecting group; and
X, Y, R¹, and R² are defined as above.

Another embodiment of the invention relates to the method to produce said compound of formula (C), wherein the protecting group of step (b) is selected from t-butoxycarbonyl or benzyloxycarbonyl.

In yet another embodiment, the method of preparing, wherein the deprotecting agent is an acid.

Another embodiment of the invention relates to the method of preparing, wherein the acid is trifluoroacetic acid.

In yet another embodiment, the invention relates to the method of preparing, further comprising the steps of preparing said compound of formula (B) comprising:
(c) treating a compound of formula (A), optionally in the presence of an activating agent: 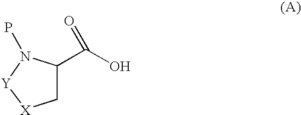
with an amine to produce said compound of formula (B);
wherein;
P, X and Y are as defined above.

In another embodiment, the invention relates to the method of preparing, wherein the amine is selected from the group consisting of 2-adamantanamine-hydrochloride salt, 2-adamantanamine, and benzyl amine.

In yet another embodiment, the method of preparing, wherein said activating agent is independently selected from the group consisting of O-(7-azabenzotriazol-1-yl)-N,N,N′,N′-tetramethyluronium hexafluorophosphate, 1-hydroxybenzotriazole, and 1-(3-dimethylaminopropyl)-3-ethylcarbodiimide hydrochloride.

### Definitions

As used herein, the terms “comprising” and “including” are used in their open, non-limiting sense.

The term “alkyl,” as used herein, unless otherwise indicated, includes saturated monovalent hydrocarbon radicals having straight or branched moieties.

The term “alkenyl,” as used herein, unless otherwise indicated, includes alkyl moieties having at least one carbon-carbon double bond wherein alkyl is as defined above and including E and Z isomers of said alkenyl moiety.

The term “alkynyl,” as used herein, unless otherwise indicated, includes alkyl moieties having at least one carbon-carbon triple bond wherein alkyl is as defined above.

The term “alkoxy,” as used herein, unless otherwise indicated, includes O-alkyl groups wherein alkyl is as defined above.

The term “amino,” as used herein, is intended to include the —NH₂ radical, and any substitutions of the N atom.

The terms “halogen” and “halo,” as used herein represent chlorine, fluorine, bromine or iodine.

The term “trifluoromethyl,” as used herein, is meant to represent a —CF₃ group.

The term “trifluoromethoxy,” as used herein, is meant to represent a —OCF₃ group.

The term “cyano,” as used herein, is meant to represent a —CN group.

The term “OMs,” as used herein, is intended to mean, unless otherwise indicated is intended to mean methanesulfonate.

The term “HOBt,” 1-hydroxybenzotriazole is intended to mean, unless otherwise indicated is intended to mean 1-hdroxybenzotriazole.

The term “Me,” as used herein, unless otherwise indicated, is intended to mean means methyl.

The term “MeOH,” as used herein, unless otherwise indicated, is intended to mean means methanol.

The term “Et,” as used herein, unless otherwise indicated, is intended to mean means ethyl.

The term “Et₂O,” as used herein, unless otherwise indicated, is intended to mean means diethylether.

The term “EtOH,” as used herein, unless otherwise indicated, is intended to mean means ethanol.

The term “Et₃N,” as used herein, unless otherwise indicated, is intended to mean means triethylamine.

The term “EtOAc,” as used herein, unless otherwise indicated, is ethyl acetate.

The term “AlMe₂Cl,” as used herein, unless otherwise indicated, is intended to mean dimethyl aluminum chloride.

The term “Ph,” as used herein, unless otherwise indicated, is intended to mean phenyl.

The term “Ac,” as used herein, unless otherwise indicated, is intended to mean means acetyl.

The term “TFA,” as used herein, unless otherwise indicated, is intended to mean trifluoroacetic acid.

The term “TEA,” as used herein, unless otherwise indicated, is intended to mean triethanolamine.

The term “HATU,” as used herein, unless otherwise indicated, is intended to mean N,N,N′,N′-tetramethyluronium hexafluorophosphate.

The term “DIPEA,” as used herein, unless otherwise indicated, is intended to mean diisopropyl ethyl amine.

The term “DCE,” as used herein, unless otherwise indicated, is intended to mean 1,2-dichloro ethane.

The term “THF,” as used herein, unless otherwise indicated, is intended to mean tetrahydrofuran.

The term “BHT,” as used herein, unless otherwise indicated, is intended to mean butylated hydroxy toluene.

The term “Boc,” as used herein, unless otherwise indicated, is intended to mean t-butoxycarbonyl.

The term “(Boc)₂O,” as used herein, unless otherwise indicated, is intended to mean di-tert-butyl dicarbonate.

The term “CBZ,” as used herein, unless otherwise indicated is intended to mean benzyloxycarbonyl.

The term NMM,” as used herein, unless otherwise indicated is intended to mean N-methyl-morpholine.

The term “MTBE,” as used herein, unless otherwise indicated is intended to mean tert-butyl methyl ether.

The term “DMAP,” as used herein, unless otherwise indicated is intended to mean 4-(dimethylamino)pyridine.

The term “EDC,” as used herein, unless otherwise indicated is intended to mean 1-(3-dimethylaminopropyl)-3-ethylcarbodiimide hydrochloride.

The term “TIOH,” as used herein, unless otherwise indicated, is intended to mean thallium(1)hydroxide.

The term “TIOEt,” as used herein, unless otherwise indicated, is intended to mean thallium(1)ethoxide.

The term “PCy₃,” as used herein, is intended to mean tricyclohexylphosphine.

The term “Pd₂(dba)₃,” as used herein, unless otherwise indicated, is intended to mean tris(dibenzylideneacetone)dipalladium(0).

The term “Pd(OAc)₂,” as used herein, unless otherwise indicated, is intended to mean palladium(II) acetate.

The term “Pd(PPh₃)₂Cl₂,” as used herein, unless otherwise indicated, is intended to mean dichlorobis(triphenylphosphine)palladium(II).

The term “Pd(PPh₃)₄” as used herein, unless otherwise indicated, is intended to mean tetrakis(triphenylphophine)palladium(0).

The term “Pd(dppf)Cl₂,” as used herein, is intended to mean (1,1′-bis(diphenylphosphino)ferrocene)dichloropalladium(II), complex with dichloromethane (1:1).

The term “Pd/C,” as used herein, unless otherwise indicated, is intended to mean palladium on carbon.

The term “PyBOP,” as used herein, unless otherwise indicated, is intended to mean benzotriazol-1-yl-oxytripyrrolidinophosphonium hexafluorophosphate.

The term “DIEA,” as used herein unless otherwise indicated, is intended to mean N,N-diisopropylethylamine.

The term “G6P,” as used herein, unless otherwise indicated, is intended to mean glucose-6-phosphate.

The term “NIDDM, as used herein, unless otherwise indicated, is intended to mean non insulin dependent diabetes mellitus.

The term “NAHMDS,” as used herein unless otherwise indicated, is intended to mean sodium bis(trimethylsilyl)amide.

The term “NADPH,” as used herein, unless otherwise indicated, is intended to mean nicotinamide adenine dinucleotide phosphate, reduced form.

The term “CDCl₃ or CHLORFORM-D,” as used herein, is intended to mean deuterochloroform.

The term “CD₃OD,” as used herein, is intended to mean deuteromethanol.

The term “CD₃CN,” as used herein, is intended to mean deuteroacetonitrile.

The term “DEAD,” as used herein, is intended to mean diethyl azodicarboxylate.

The term “DIAD,” as used herein, is intended to mean diisopropyl azodicarboxylate.

The term “TsCH₂NC,” as used herein, is intended to mean tosylmethyl isocyanide.

The term “CISO₃H,” as used herein, is intended to mean chlorosulfonic acid.

The term “DMSO-d₆” or “DMSO-D₆,” as used herein, is intended to mean deuterodimethyl sulfoxide.

The term “DME,” as used herein, is intended to mean 1,2-dimethoxyethane.

The term “DMF,” as used herein, is intended to mean N,N-dimethylformamide.

The term “DMSO,” as used herein, is intended to mean, unless otherwise indicated dimethylsulfoxide.

The term “DI,” as used herein, is intended to mean deionized.

The term “KOAc,” as used herein, is intended to mean potassium acetate.

The term “neat,” as used herein, is meant to represent an absence of solvent.

The term “mmol,” as used herein, is intended to mean millimole.

The term “eqv,” as used herein, is intended to mean equivalent.

The term “mL,” as used herein, is intended to mean milliliter.

The term “U,” as used herein, is intended to mean units.

The term “mm,” as used herein, is intended to mean millimeter.

The term “g,” as used herein, is intended to mean gram.

The term “kg,” as used herein, is intended to mean kilogram.

The term “h,” as used herein, is intended to mean hour.

The term “min,” as used herein, is intended to mean minute.

The term “μL,” as used herein, is intended to mean microliter.

The term “μM,” as used herein, is intended to mean micromolar.

The term “μm,” as used herein, is intended to mean micrometer.

The term “M,” as used herein, is intended to mean molar.

The term “N,” as used herein, is intended to mean normal.

The term “nm,” as used herein, is intended to mean nanometer.

The term “nM,” as used herein, is intended to mean nanoMolar.

The term “amu,” as used herein, is intended to mean atomic mass unit.

The term “OC,” as used herein, is intended to mean Celsius.

The term “m/z,” as used herein, is intended to mean, unless otherwise indicated, mass/charge ratio.

The term “wt/wt,” as used herein, is intended to mean weight/weight.

The term “v/v,” as used herein, is intended to mean volume/volume.

The term “mL/min,” as used herein, is intended to mean milliliter/minute.

The term “UV,” as used herein, is intended to mean ultraviolet.

The term “APCI-MS,” as used herein, is intended to mean atmospheric pressure chemical ionization mass spectroscopy.

The term “HPLC,” as used herein, is intended to mean high performance liquid chromatograph.

The term “LC,” as used herein, is intended to mean liquid chromatograph.

The term “LCMS,” as used herein, is intended to mean liquid chromatography mass spectroscopy.

The term “SFC,” as used herein, is intended to mean supercritical fluid chromatography.

The term “sat,” as used herein, is intended to mean saturated.

The term “aq,” as used herein, is intended to mean aqueous.

The term “ELSD,” as used herein, is intended to mean evaporative light scattering detection.

The term “MS,” as used herein, is intended to mean mass spectroscopy.

The term “HRMS (ESI),” as used herein, is intended to mean high resolution mass spectrometry (electrospray ionization).

The term “Anal.,” as used herein, is intended to mean analytical.

The term “Calcd,” as used herein, is intended to mean calculated.

The term “NA,” as used herein, unless otherwise indicated, is intended to mean not available.

The term “RT,” as used herein, unless otherwise indicated, is intended to mean room temperature.

The term “Celite®,” as used herein, unless otherwise indicated, is intended to mean a white solid diatomite filter agent commercially available from World Minerals located in Los Angeles, Calif. USA.

In the formulas of (I), (II), and (III), where terms such as —(CR⁴R⁵)ₜ or —(CR¹⁰R¹¹)ᵥ, for example, are used, R⁴, R⁵, R¹⁰ and R¹¹ may vary with each iteration of t or v above 1. For instance, where t or v is 2 the terms —(CR⁴R⁵)ₜ or —(CR¹⁰R¹¹), may equal —CH₂CH₂—, or —CH(CH₃)C(CH₂CH₃)(CH₂CH₂CH₃)—, or any number of similar moieties falling within the scope of the definitions of R⁴, R⁵, R¹⁰ and R¹¹.

The term “Kᵢ,” as used herein, is intended to mean values of enzyme inhibition constant.

The term “Kᵢ” app, as used herein, is intended to mean K apparent.

The term “IC₅₀,” as used herein, is intended to mean concentrations required for at least 50% enzyme inhibition.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-10 carbon atoms, such as cyclopropyl, cyclobutyl, cyclopentyl, cyclohexyl, cycloheptyl, and adamantyl. Illustrative examples of cycloalkyl are derived from, but not limited to, the following:

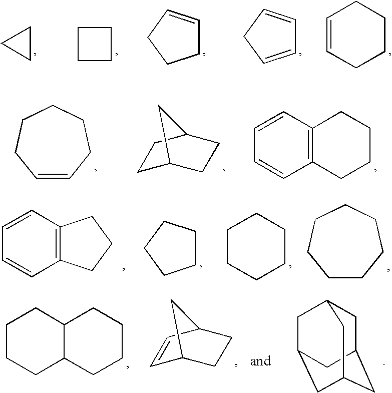

The term “aryl”, as used herein, unless otherwise indicated, includes an organic radical derived from an aromatic hydrocarbon by removal of one hydrogen, such as phenyl or naphthyl.

The term “(4 to 10)-membered heterocyclyl”, as used herein, unless otherwise indicated, includes aromatic and non-aromatic heterocyclic groups containing one to four heteroatoms each selected from O, S and N, wherein each heterocyclic group has from 4-10 atoms, respectively, in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3 membered heterocyclic group is aziridine, an example of a 4 membered heterocyclic group is azetidinyl (derived from azetidine). An example of a 5 membered heterocyclic group is thiazolyl, an example of a 7 membered ring is azepinyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached). The 4 to 10 membered heterocyclic may be optionally substituted on any ring carbon, sulfur, or nitrogen atom(s) by one to two oxo, per ring. An example of a heterocyclic group wherein the ring atoms are substituted with oxo moieties is 1,1-dioxo-thiomorpholinyl. Other Illustrative examples of 4 to 10 membered heterocyclic are derived from, but not limited to, the following:

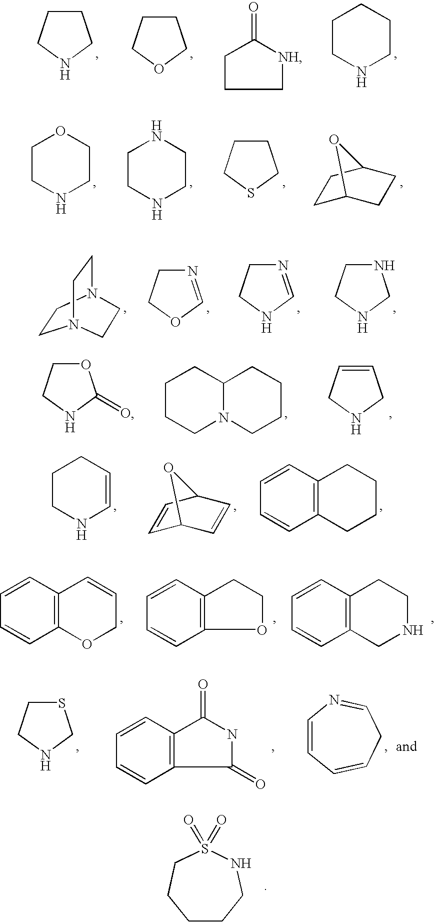

Unless otherwise indicated, the term “oxo” refers to ═O.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO (dimethylsulfoxide), ethyl acetate, acetic acid, or ethanolamine.

The compounds of the present invention may have asymmetric carbon atoms. The carbon-carbon bonds of the compounds of the present invention may be depicted herein using a solid line [symbol] a solid wedge [symbol] [symbol] wavy line, or a dotted wedge [symbol] The use of a solid line to depict bonds to asymmetric carbon atoms is meant to indicate that all possible stereoisomers at that carbon atom are included. The use of either a solid or dotted wedge to depict bonds to asymmetric carbon atoms is meant to indicate that only the stereoisomer shown is meant to be included. The use of a wavy line to depict bonds to asymmetric carbon atoms is meant to indicate the diastereomer is present. It is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Solutions of individual stereoisomeric compounds of the present invention may rotate plane-polarized light. The use of either a “(+)” or “(−)” symbol in the name of a compound of the invention indicates that a solution of a particular stereoisomer rotates plane-polarized light in the (+) or (−) direction, as measured using techniques known to those of ordinary skill in the art.

Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

Alternatively, individual stereoisomeric compounds of the present invention may be prepared in enantiomerically enriched form by asymmetric synthesis. Asymmetric synthesis may be performed using techniques known to those of skill in the art, such as the use of asymmetric starting materials that are commercially available or readily prepared using methods known to those of ordinary skill in the art, the use of asymmetric auxiliaries that may be removed at the completion of the synthesis, or the resolution of intermediate compounds using enzymatic methods. The choice of such a method will depend on factors that include, but are not limited to, the availability of starting materials, the relative efficiency of a method, and whether such methods are useful for the compounds of the invention containing particular functional groups. Such choices are within the knowledge of one of ordinary skill in the art.

When the compounds of the present invention contain asymmetric carbon atoms, the derivative salts, prodrugs and solvates may exist as single stereoisomers, racemates, and/or mixtures of enantiomers and/or diastereomers. All such single stereoisomers, racemates, and mixtures thereof are intended to be within the scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

If a derivative used in the method of the invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of derivatives, prodrugs, salts, or solvates that are solids, it is understood by those skilled in the art that the derivatives, prodrugs, salts, and solvates used in the method of the invention, may exist in different polymorph or crystal forms, all of which are intended to be within the scope of the present invention and specified formulas. In addition, the derivative, salts, prodrugs and solvates used in the method of the invention may exist as tautomers, all of which are intended to be within the broad scope of the present invention.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

Certain compounds of formulas (I), (II), and (III) may have asymmetric centers and therefore exist in different enantiomeric forms. All optical isomers and stereoisomers of the compounds of formulas (I), (II), and (III), and mixtures thereof, are considered to be within the scope of the invention. With respect to the compounds of formulas (I), (II), and (III), the invention includes the use of a racemate, one or more enantiomeric forms, one or more diastereomeric forms, or mixtures thereof. The compounds of formulas (I), (II), and (III) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in formulas (I), (II), and (III), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts or solvates of said compounds which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formulas (I), (II), and (III) of this invention thereof can generally be prepared by carrying out the procedures disclosed in the Schemes and/or in the Examples below, by substituting a readily available isotopically labeled reagent for a non-isotopically labeled reagent.

Other aspects, advantages, and features of the invention will become apparent from the detailed description below.

The phrase “pharmaceutically acceptable salt(s)”, as used herein, unless otherwise indicated, includes salts of acidic or basic groups which may be present in the compounds of formulas (I), (II), and (III). The compounds of formulas (I), (II), and (III) that are basic in nature are capable of forming a wide variety of salts with various inorganic and organic acids. The acids that may be used to prepare pharmaceutically acceptable acid addition salts of such basic compounds of formulas (I), (II), and (III) are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethosuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicyate, stearate, subacetate, succinate, tannate, tartrate, teoclate, tosylate, triethiodode, and valerate salts.

The term “diseases in which the liver is a target organ”, as used herein, unless otherwise indicated, means diabetes, hepatitis, liver cancer, liver fibrosis, and malaria.

The term “Metabolic syndrome”, as used herein, unless otherwise indicated means psoriasis, diabetes mellitus, wound healing, inflammation, neurodegenerative diseases, galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “modulate” or “modulating”, as used herein, refers to the ability of a modulator for a member of the steroid/thyroid superfamily to either directly (by binding to the receptor as a ligand) or indirectly (as a precursor for a ligand or an inducer which promotes production of ligand from a precursor) induce expression of gene(s) maintained under hormone expression control, or to repress expression of gene(s) maintained under such control.

The term “obesity” or “obese”, as used herein, refers generally to individuals who are at least about 20-30% over the average weight for his/her age, sex and height. Technically, “obese” is defined, for males, as individuals whose body mass index is greater than 27.8 kg/m², and for females, as individuals whose body mass index is greater than 27.3 kg/m². Those of skill in the art readily recognize that the invention method is not limited to those who fall within the above criteria. Indeed, the method of the invention can also be advantageously practiced by individuals who fall outside of these traditional criteria, for example, by those who may be prone to obesity.

The term “inflammatory disorders”, as used herein, refers to disorders such as rheumatoid arthritis, ankylosing spondylitis, psoriatic arthritis, psoriasis, chondrocalcinosis, gout, inflammatory bowel disease, ulcerative colitis, Crohn's disease, fibromyalgia, and cachexia.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The phrase “amount . . . effective to lower blood glucose levels”, as used herein, refers to levels of compound sufficient to provide circulating concentrations high enough to accomplish the desired effect. Such a concentration typically falls in the range of about 10 nM up to 2 μM; with concentrations in the range of about 100 nM up to 500 nM being one example. As noted previously, since the activity of different compounds which fall within the definition of formulas (I), (II), and (III), where terms such as as set forth above may vary considerably, and since individual subjects may present a wide variation in severity of symptoms, it is up to the practitioner to determine a subject's response to treatment and vary the dosages accordingly.

The phrase “insulin resistance”, as used herein, refers to the reduced sensitivity to the actions of insulin in the whole body or individual tissues, such as skeletal muscle tissue, myocardial tissue, fat tissue or liver tissue. Insulin resistance occurs in many individuals with or without diabetes mellitus.

The phrase “insulin resistance syndrome”, as used herein, refers to the cluster of manifestations that include insulin resistance, hyperinsulinemia, non insulin dependent diabetes mellitus (NIDDM), arterial hypertension, central (visceral) obesity, and dyslipidemia.

Certain compounds of formulas (I), (II), and (III) may have asymmetric centers and therefore exist in different enantiomeric forms. All optical isomers and stereoisomers of the compounds of formulas (I), (II), and (III), and mixtures thereof, are considered to be within the scope of the invention. With respect to the compounds of formulas (I), (II), and (III), the invention includes the use of a racemate, one or more enantiomeric forms, one or more diastereomeric forms, or mixtures thereof. The compounds of formulas (I), (II), and (III) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in formulas (I), (II), and (III), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, 3H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts or solvates of said compounds which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly useful for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be more useful in some circumstances. Isotopically labeled compounds of formulas (I), (II), and (III) of this invention thereof can generally be prepared by carrying out the procedures found in the Schemes and/or in the Examples below, by substituting a readily available isotopically labelled reagent for a non-isotopically labelled reagent.

Other aspects, advantages, and features of the invention will become apparent from the detailed description below.

### DETAILED DESCRIPTION AND EMBODIMENTS OF THE INVENTION

The following reaction Schemes illustrate the preparation of the compounds of the present invention. Unless otherwise indicated, R¹-R²¹, R^{1a}-R^{3a}, and T in the reaction schemes and the discussion that follows are as defined above.

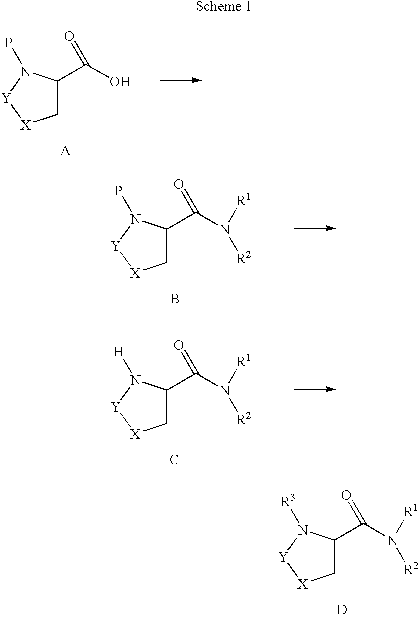

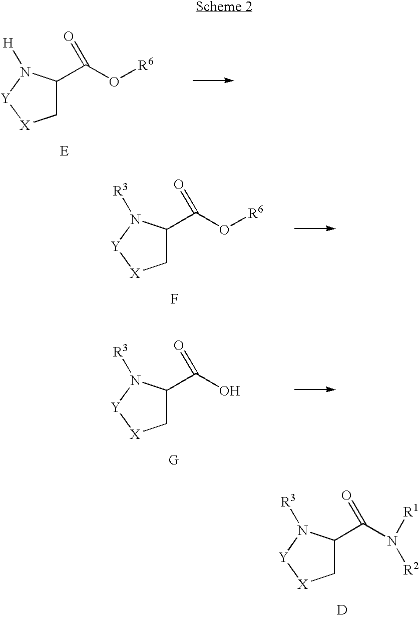

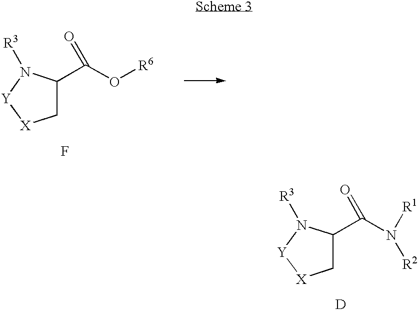

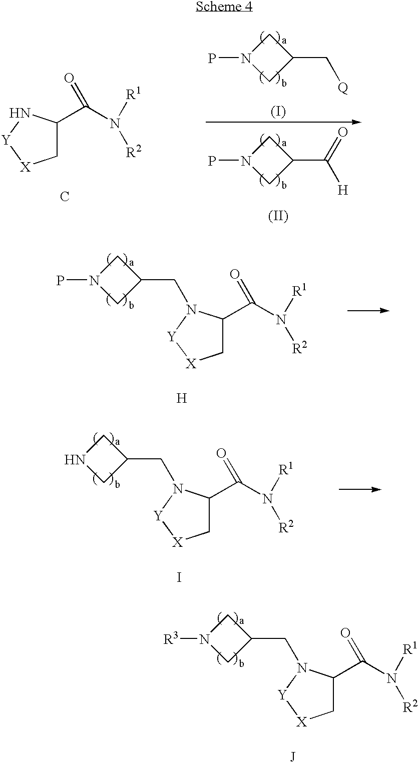

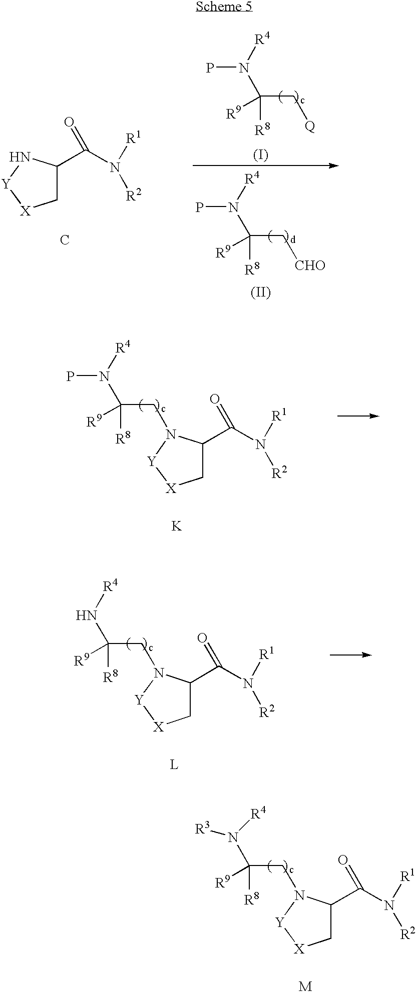

Referring to Scheme 1 above, the compound of formula D may be prepared by reacting a compound of formula C with R³LV wherein LV is a leaving group such as Cl, Br, I, OMs, etc. in a suitable solvent (e.g. dichloromethane or DMF) advantageously, in the presence of a base (e.g. K₂CO₃, NaHCO₃, Et₃N), from room temperature to the boiling point of the solvent, typically from about 20 degrees Celsius to about 100 degrees Celsius. Alternatively, the compound of formula D can also be prepared by reductive amination of compound of formula C with suitable aldehyde such as, acetone, or a suitable ketone, such as formaldehyde or cyclopentanecarboxaldehyde, in a suitable solvent such as THF, MeOH, CH₂Cl₂, in the presence of an acid such as acetic acid, and a reducing agent such as NaBCNH₃ or NaB(OAc)₃H at a temperature ranging from room temperature to 60 degree Celsius. Alternatively, the compound of formula D can also be prepared by reacting the compound of formula C with acyl halide such as acetyl chloride in a suitable solvent such as THF or CH₂Cl₂, in the presence of an amine such as triethylamine or pyridine at a temperature ranging from −78 degree Celsius to 60 degree Celsius. Alternatively, the compound of formula D can also be prepared by reacting the compound of formula C with sulfonyl halide such as methanesulfonyl chloride in a suitable solvent such as THF or CH₂Cl₂, in the presence of an amine such as triethylamine or pyridine at a temperature ranging from −78 degree Celsius to 60 degree Celsius. Compound of formula C can be prepared by removing the protecting group P in the compound of formula B. The compound of formula B can be prepared by coupling the compound of formula A with an amine, such as R¹R²NH, following standard amide bond formation methods by a method known to those skilled in the art. Compound formula A is an acid wherein P is a protecting functional group such as BOC or CBZ; R¹ is independently alkyl, cycloalkyl, aryl, or (4 to 10)-membered heterocyclyl, etc. and R² is independently H and alkyl; X is independently —CR⁴R⁵, —O—, —S—, —NR⁴—, etc; and Y is —(CR⁴R⁵), wherein t is 1, 2, or 3.

Referring to Scheme 2 above, the compound of formula D can be prepared by coupling the compound of formula G with R¹R²NH following standard amide bond formation methods by a method known to those skilled in the art. Compound of formula G may be prepared by treatment of compound of formula F with a base such as NaOH, KOH, LiOH in a suitable solvent such as MeOH and water at a temperature ranging from room temperature to 60 degree Celsius. Compound of formula F may be prepared by reacting a compound of formula E with R³LV wherein LV is a leaving group such as Cl, Br, I, OMs, etc in a suitable solvent (e.g. dichloromethane or DMF) advantageously, in the presence of a base (e.g. K₂CO₃, NaHCO₃, Et₃N), from room temperature to the boiling point of the solvent, typically from about 20 degrees Celsius to about 100 degrees Celsius. Alternatively, the compound of formula F can also be prepared by reductive amination of compound of formula E with an aldehyde or ketone in a suitable solvent such as THF, MeOH, CH₂Cl₂, in the presence of an acid such as acetic acid, and a reducing agent such as NaBCNH₃ or NaB(OAc)₃H at a temperature ranging from room temperature to 60 degree Celsius. Compound E is an amine wherein R⁶ is a protecting functional group such as Me; R¹ is independently alkyl, cycloalkyl, aryl, or (4-10)-membered heterocyclyl, etc. and R² is independently H and alkyl; X is independently —CR⁴R⁵, —O—, —S—, —NR⁴—, etc; and Y is —(CR⁴R⁵)ₜ wherein t is 1, 2, or 3.

Referring to Scheme 3 above, the compound of formula D can be prepared by treatment of the compound of formula F with R¹R²NH in a suitable solvent at a suitable temperature or in a suitable solvent in the presence of a Lewis acid such as AlCl₃.

Referring to Scheme 4 above, the compound of formula J, wherein a is an interger of 0, 1, 2, or 3, and b is an interger of 1, 2, or 3, may be prepared by reacting a compound of formula I with R³LV wherein LV is a leaving group such as Cl, Br, I, OMs, etc. in a suitable solvent (e.g. dichloromethane or DMF) advantageously, in the presence of a base (e.g. K₂CO₃, NaHCO₃, Et₃N), from room temperature to the boiling point of the solvent, typically from about 20 degrees Celsius to about 100 degrees Celsius. Alternatively, the compound of formula J can also be prepared by reductive amination of compound of formula C with an aldehyde or ketone in a suitable solvent such as THF, MeOH, CH₂Cl₂, in the presence of an acid such as acetic acid, and a reducing agent such as NaBCNH₃ or NaB(OAc)₃H at a temperature ranging from a temperature of about 20° C. to about 60 degree Celsius. Alternatively, the compound of formula J can also be prepared by reacting compound of formula I with acyl halide such as acetyl chloride in a suitable solvent such as THF or CH₂Cl₂, in the presence of an amine such as triethylamine or pyridine at a temperature ranging from −78 degree Celsius to 60 degree Celsius. Alternatively, the compound of formula J can also be prepared by reacting compound of formula I with sulfonyl halide such as methanesulfonyl chloride in a suitable solvent such as THF or CH₂Cl₂, in the presence of an amine such as triethylamine or pyridine at a temperature ranging from −78 degree Celsius to 60 degree Celsius. Compound of formula I can be prepared by removing the protecting group P in the compound of formula H. The compound of formula H can be may be prepared by SN2 displacement with the reagent I in a suitable solvent (e.g. dichloromethane or DMF) advantageously, in the presence of a base (e.g. K₂CO₃, NaHCO₃, Et₃N), from room temperature to the boiling point of the solvent, typically from about 20 degrees Celsius to about 100 degrees Celsius. Alternatively, the compound of formula H can also be prepared by reductive amination of compound of formula C with reagent II in a suitable solvent such as THF, MeOH, CH₂Cl₂, in the presence of an acid such as acetic acid, and a reducing agent such as NaBCNH₃ or NaB(OAc)₃H at a temperature ranging from room temperature to 60 degree Celsius.

Referring to Scheme 5 above, the compound of formula M, wherein c is an interger of 1, 2, or 3, may be prepared by reacting a compound of formula L with R³LV wherein LV is a leaving group such as Cl, Br, I, OMs, etc. in a suitable solvent (e.g. dichloromethane or DMF) advantageously, in the presence of a base (e.g. K₂CO₃, NaHCO₃, Et₃N), from room temperature to the boiling point of the solvent, typically from about 20 degrees Celsius to about 100 degrees Celsius. Alternatively, the compound of formula M can also be prepared by reductive amination of compound of formula L with an aldehyde or ketone in a suitable solvent such as THF, MeOH, CH₂Cl₂, in the presence of an acid such as acetic acid, and a reducing agent such as NaBCNH₃ or NaB(OAc)₃H at a temperature ranging from room temperature to 60 degree Celsius. Alternatively, the compound of formula M can also be prepared by reacting compound of formula L with acyl halide such as acetyl chloride in a suitable solvent such as THF or CH₂Cl₂, in the presence of an amine such as triethylamine or pyridine at a temperature ranging from −78 degree Celsius to 60 degree Celsius. Alternatively, the compound of formula M can also be prepared by reacting compound of formula L with sulfonyl halide such as methanesulfonyl chloride in a suitable solvent such as THF or CH₂Cl₂, in the presence of an amine such as triethylamine or pyridine at a temperature ranging from −78 degree Celsius to 60 degree Celsius. Compound of formula L can be prepared by removing the protecting group P in the compound of formula K. The compound of formula K can be may be prepared by SN2 displacement with the reagent I in a suitable solvent (e.g. dichloromethane or DMF) advantageously, in the presence of a base (e.g. K₂CO₃, NaHCO₃, Et₃N), from room temperature to the boiling point of the solvent, typically from about 20 degrees Celsius to about 100 degrees Celsius. Alternatively, the compound of formula K can also be prepared by reductive amination of compound of formula C with reagent II, wherein d is an interger of 0, 1 or 2, in a suitable solvent such as THF, MeOH, CH₂Cl₂, in the presence of an acid such as acetic acid, and a reducing agent such as NaBCNH₃ or NaB(OAc)₃H at a temperature ranging from room temperature to 60 degree Celsius.

The compounds of the present invention may have asymmetric carbon atoms, and may therefore be made from starting materials that are sterospecific. Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formulas (I), (II), and (III) that are basic in nature are capable of forming a wide variety of different salts with various inorganic and organic acids. Although such salts must be pharmaceutically acceptable for administration to animals, it is often desirable in practice to initially isolate the compound of formulas (I), (II), and (III) from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formulas (I), (II), and (III) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formulas (I), (II), and (III). Such non-toxic base salts include those derived from such pharmacologically acceptable cations as sodium, potassium, calcium, and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention may be modulators of 11-β-hsd-1. The compounds of the present invention may modulate processes mediated by 11-β-hsd-1, which refer to biological, physiological, endocrinological, and other bodily processes which are mediated by receptor or receptor combinations which are responsive to the 11-β-hsd-1 inhibitors described herein (e.g., diabetes, hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g.) brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts. Modulation of such processes can be accomplished in vitro or in vivo. In vivo modulation can be carried out in a wide range of subjects, such as, for example, humans, rodents, sheep, pigs, cows, and the like.

The compounds according to the present invention may be used in several indications which involve modulations of 11-β-hsd-1 enzyme. Thus, the compounds according to the present invention may be used against dementia (see WO97/07789), osteoporosis (see Canalis, E., 1996, “Mechanisms of glucocorticoid action in bone: implications to glucocorticoid-induced osteoporosis,” Journal of Clinical Endocrinology and Metabolism, 81, 3441-3447) and may also be used disorders in the immune system (see Franchimont, et al, “Inhibition of Th1 immune response by glucocorticoids: dexamethasone selectively inhibits IL-12-induced Stat 4 phosphorylation in T lymphocytes”, The Journal of Immunology 2000, Feb. 15, vol 164 (4), pages 1768-74) and also in the above listed indications.

Inhibition of 11-β-hsd-1 in mature adipocytes is expected to attenuate secretion of the plasminogen activator inhibitor 1 (PAI-1) an independent cardiovascular risk factor (Halleux, C. M., et al. (1999) J. Clin. Endocrinol. Metab. 84: 4097-4105). Furthermore, there is a clear correlation between glucocorticoid “activity” and cardiovascular risk factor suggesting that a reduction of the glucocorticoid effects would be beneficial (Walker, B. R., et al., (1998), Hypertension 31: 891-895; Fraser, R., et al., (1999), Hypertension, 33: 1364-1368).

Adrenalectomy attenuates the effect of fasting to increase both food intake and hypothalamic neuropeptide Y expression. This supports the role of glucocorticoids in promoting food intake and suggests that inhibition of 11-β-hsd-1 in the brain might increase satiety and therefore reduce food intake (Woods, S. C., et al., (1998), Science, 280:1378-1383).

### Possible Beneficial Effect on the Pancreas

Inhibition of 11-β-hsd-1 in isolated murine pancreatic β-cells improves the glucose-stimulated insulin secretion (Davani, B., et al. (2000) J. Biol. Chem., Nov. 10, 2000; 275(45): 34841-4). Glucocorticoids were previously known to reduce pancreatic insulin release in vivo (Billaudel, B. and B. C. J. Sutter, (1979), Horm. Metab. Res. 11: 555-560). Thus, inhibition of 11-βhsd-1 is predicted to yield other beneficial effects for diabetes treatment, besides effects on liver and fat.

Stress and glucocorticoids influence cognitive function (de Quervain, D. J.-F., B. Roozendaal, and J. L. McGaugh, (1998), Nature, 394: 787-790). The enzyme 11-β-hsd-1 controls the level of glucocorticoid action in the brain and thus contributes to neurotoxicity (Rajan, V., Edwards, C. R. W. and Seckl, J. R., (1996) Neuroscience 16: 65-70; Seckl, J. R., Front. Neuroendocrinol., (2000), 18: 49-99). Unpublished results indicate significant memory improvement in rats treated with a non-specific 11-β-hsd-1 inhibitor. Based the above and on the known effects of glucocorticoids in the brain, it may also be suggested that inhibiting 11β-hsd-1 in the brain may result in reduced anxiety (Tronche, F., et al., (1999), Nature Genetics 23: 99-103). Thus, taken together, the hypothesis is that inhibition of 11-β-hsd-1 in the human brain would prevent reactivation of cortisone into cortisol and protect against deleterious glucocorticoid-mediated effects on neuronal survival and other aspects of neuronal function, including cognitive impairment, depression, and increased appetite (previous section).

The general perception is that glucocorticoids suppress the immune system. But in fact there is a dynamic interaction between the immune system and the HPA (hypothalamo-pituitary-adrenal) axis (Rook, G. A. W., (1999), Baillier's Clin. Endocrinol. Metab., 13: 576-581). The balance between the cell-mediated response and humoral responses is modulated by glucocorticoids. A high glucocorticoid activity, such as at a state of stress, is associated with a humoral response. Thus, inhibition of the enzyme 11-β-hsd-1 has been suggested as a means of shifting the response towards a cell-based reaction.

In certain disease states, including tuberculosis, lepra and psoriasis the immune reaction is normaly biased towards a humoral response when in fact the appropriate response would be cell based. Temporal inhibition of 11-β-hsd-1, local or systemic, might be used to push the immune system into the appropriate response (Mason, D., (1991), Immunology Today, 12: 57-60; Rook, et al., supra).

Recent data suggests that the levels of the glucocorticoid target receptors and the 11-β-hsd-1 enzymes determine the susceptibility to glaucoma (Stokes, J., et al., (2000) Invest. Ophthalmol., 41:1629-1638). Further, inhibition of 11-β-hsd-1 was recently presented as a novel approach to lower the intraocular pressure (Walker, E. A., et al, poster P3-698 at the Endocrine Society Meeting Jun. 12-15, 1999, San Diego). Ingestion of carbenoxolone, a non-specific inhibitor of 11-β-hsd-1, was shown to reduce the intraocular pressure by 20% in normal subjects. In the eye, expression of 11-β-hsd-1 is confined to basal cells of the corneal epithelium and the non-pigmented epithelialium of the cornea (the site of aqueous production), to ciliary muscle and to the sphincter and dilator muscles of the iris. In contrast, the distant isoenzyme 11 beta-hydroxysteroid dehydrogenase type 2 is highly expressed in the non-pigmented ciliary epithelium and corneal endothelium. None of the enzymes is found at the trabecular meshwork, the site of drainage. Thus, 11-β-hsd-1 is suggested to have a role in aqueous production, rather than drainage, but it is presently unknown if this is by interfering with activation of the glucocorticoid or the mineralocorticoid receptor, or both.

Glucocorticoids have an essential role in skeletal development and function but are detrimental in excess. Glucocorticoid-induced bone loss is derived, at least in part, via inhibition of bone formation, which includes suppression of osteoblast proliferation and collagen synthesis (Kim, C. H., Cheng, S. L., and Kim, G. S., (1999) J. Endocrinol., 162: 371-379). The negative effect on bone nodule formation could be blocked by the non-specific inhibitor carbenoxolone suggesting an important role of 11-β-hsd-1 in the glucocorticoid effect (Bellows, C. G., Ciaccia, A. and. Heersche, J. N. M, (1998), Bone 23: 119-125). Other data suggest a role of 11-β-hsd-1 in providing sufficiently high levels of active glucocorticoid in osteoclasts, and thus in augmenting bone resorption (Cooper, M. S., et al., (2000), Bone, 27:375-381). Taken together, these different data suggest that inhibition of 11-β-hsd-1 may have beneficial effects against osteoporosis by more than one mechanism working in parallel.

Bile acids inhibit 11β-hydroxysteroid dehydrogenase type 2. This results in a shift in the overall body balance in favor of cortisol over cortisone, as shown by studying the ratio of the urinary metabolites (Quattropani, C., Vogt, B., Odermatt, A., Dick, B. Frey, B. M., Frey, F. J., November 2001, J Clin Invest., 108(9):1299-305. “Reduced activity of 11beta-hydroxysteroid dehydrogenase in patients with cholestasis”). Reducing the activity of 11-β-hsd-1 in the liver by a selective inhibitor is predicted to reverse this imbalance, and acutely counter the symptoms such as hypertension, while awaiting surgical treatment removing the biliary obstruction.

The compounds of the present invention may also be useful in the treatment of other metabolic disorders associated with impaired glucose utilization and insulin resistance include major late-stage complications of NIDDM, such as diabetic angiopathy, atherosclerosis, diabetic nephropathy, diabetic neuropathy, and diabetic ocular complications such as retinopathy, cataract formation and glaucoma, and many other conditions linked to NIDDM, including dyslipidemia glucocorticoid induced insulin resistance, dyslipidemia, polycysitic ovarian syndrome, obesity, hyperglycemia, hyperlipidemia, hypercholesteremia, hypertriglyceridemia, hyperinsulinemia, and hypertension. Brief definitions of these conditions are available in any medical dictionary, for instance, Stedman's Medical Dictionary (10^{th} Ed.).

Assay

The inhibition constant, Ki, was measured in a buffer containing 100 mM triethanolamine, 200 mM NaCl, 0.02% n-dodecyl β-maltoside, 5% glycerol, 5 mM β-mercaptoethanol, 1% DMSO, pH 8.0. In a typical assay, the activity of human 11b-hsd-1 is measured on a Corning 96-well plate for a total volume of 300 uUwell in the presence and absence of inhibitor. In each well, varying amounts of compounds are incubated with a fixed amount of 11b-hsd-1 (4 nM) and NADPH (500 μM) for 30 to 40 min at room temperature in the assay buffer. The enzyme concentration was determined by titration using reversible tight-binding inhibitors. The activity remaining after the pre-incubation period is measured by adding a fixed concentration of 3H— cortisone (200 nM) and the regeneration system constituted with 2 mM glucose-6-phosphate, 1 U/mL glucose-6-phosphate dehydrogenase and 6 mM MgCl₂. The final concentration of cortisone in the assay buffer is lower than the Kₘ value (328 nM). In each well, the enzyme activity is quenched by mixing an aliquot of the assay buffer with an equal volume of DMSO in a second 96-well plate. 15 uL of these final samples are loaded on a C-18A column, Varian Polaris (3 um, 50×4.6 mm) connected to an Agilent 1100 HPLC with 96-well plate autosampler and a 3-ram detector from 1N/US System. 3H-Cortisone and 3H-cortisol are separated on the column using an isocratic mixture of 38%-62% methanol-water. The area of 3H-cortisol is calculated and plotted versus time to determine a linear velocity. A Kᵢ value was then determined using the following equation from J. F. Morrison (1969):

$$\frac{{v}_{i}}{{v}_{o}} = 1 - ( \frac{( I + E + {K}_{i} ) - \sqrt{{( I + E + {K}_{i} )}^{2} - 4 \cdot I \cdot E}}{2 \cdot I} )$$

Where vᵢ, and vₒ are the rates of cortisol formation in the presence and in the absence of inhibitor, respectively, I is the inhibitor concentration and E is the 11 b-hsd-1 concentration in the assay buffer. All the concentrations reported are the final concentrations in the assay buffer See also Morrison, J. F., “Kinetics of the reversible inhibition of enzyme-catalysed reactions by tight-binding inhibitors,” Biochim Biophys Acta., 1969; 185: 269-86.

[1,2-3H]-cortisone was purchased from American Radiolabeled Chemicals Inc. NADPH, Glucose-6-Phosphate (G6P), and Glucose-6-Phosphate dehydrogenase was purchased from Sigma.

Pharmaceutical Compositions/Formulations, Dosaging and Modes of Administration

Methods of preparing various pharmaceutical compositions with a specific amount of active compound are known, or will be apparent, to those skilled in this art. In addition, those of ordinary skill in the art are familiar with formulation and administration techniques. Such topics would be discussed, e.g. in Goodman and Gilman's The Pharmaceutical Basis of Therapeutics, current ed., Pergamon Press; and Remington's Pharmaceutical Sciences, current ed., Mack Publishing, Co., Easton, Pa. These techniques can be employed in appropriate aspects and embodiments of the methods and compositions described herein. The following examples are provided for illustrative purposes only and are not meant to serve as limitations of the present invention.

The compounds of formulas (I), (II), and (III) may be provided in suitable topical, oral and parenteral pharmaceutical formulations for use in the treatment of 11-β-hsd-1 mediated diseases. The compounds of the present invention may be administered orally as tablets or capsules, as oily or aqueous suspensions, lozenges, troches, powders, granules, emulsions, syrups or elixirs. The compositions for oral use may include one or more agents for flavoring, sweetening, coloring and preserving in order to produce pharmaceutically elegant and palatable preparations. Tablets may contain pharmaceutically acceptable excipients as an aid in the manufacture of such tablets. As is conventional in the art these tablets may be coated with a pharmaceutically acceptable enteric coating, such as glyceryl monostearate or glyceryl distearate, to delay disintegration and absorption in the gastrointestinal tract to provide a sustained action over a longer period.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain active ingredients in admixture with excipients suitable for the manufacture of an aqueous suspension. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occurring phosphatide such as lecithin, a condensation product of ethylene oxide and a long chain fatty acid, for example polyoxyethylene stearate, a condensation product of ethylene oxide and a long chain aliphatic alcohol such as heptadecaethylenoxycetanol, a condensation product of ethylene oxide and a partial ester derived from a fatty acid and hexitol such as polyoxyethylene sorbitol monooleate or a fatty acid hexitol anhydrides such as polyoxyethylene sorbitan monooleate.

The pharmaceutical compositions may be in the form of a sterile injectable aqueous or oleagenous suspension. This suspension may be formulated according to known methods using those suitable dispersing or wetting agents and suspending agents that have been mentioned above. The sterile injectable preparation may also be formulated as a suspension in a non toxic perenterally-acceptable diluent or solvent, for example as a solution in 1,3-butanediol. Among the acceptable vehicles and solvents that may be employed are water, Ringers solution and isotonic sodium chloride solution. For this purpose any bland fixed oil may be employed including synthetic mono- or diglycerides. In addition fatty acids such as oleic acid find use in the preparation of injectables.

The compounds of formulas (I), (II), and (III) may also be administered in the form of suppositories for rectal administration of the drug. These compositions can be prepared by mixing the drug with a suitable non-irritating excipient that is solid at about 25 Celcius but liquid at rectal temperature and will therefore melt in the rectum to release the drug. Such materials include cocoa butter and other glycerides.

For topical use preparations, for example, creams, ointments, jellies solutions, or suspensions, containing the compounds of the present invention are employed.

The compounds of formulas (I), (II), and (III) may also be administered in the form of liposome delivery systems such as small unilamellar vesicles, large unilamellar vesicles and multimellar vesicles. Liposomes can be formed from a variety of phospholipides, such as cholesterol, stearylamine or phosphatidylcholines.

Dosage levels of the compounds of the present invention are of the order of about 0.5 mg/kg body weight to about 100 mg/kg body weight. An exemplary dosage rate is between about 30 mg/kg body weight to about 100 mg/kg body weight. It will be understood, however, that the specific dose level for any particular patient will depend upon a number of factors including the activity of the particular compound being administered, the age, body weight, general health, sex, diet, time of administration, route of administration, rate of excretion, drug combination and the severity of the particular disease undergoing therapy. To enhance the therapeutic activity of the present compounds they may be administered concomitantly with other orally active antidiabetic compounds such as the sulfonylureas, for example, tolbutamide and the like.

For administration to the eye, a compound of the present invention is delivered in a pharmaceutically acceptable ophthalmic vehicle such that the compound is maintained in contact with the ocular surface for a sufficient time period to allow the compound to penetrate the cornea and/or sclera and internal regions of the eye, including, for example, the anterior chamber, posterior chamber, vitreous body, aqueous humor, vitreous humor, cornea, iris/ciliary's, lens, choroid/retina and sclera. The pharmaceutically acceptable ophthalmic vehicle may be an ointment, vegetable oil, or an encapsulating material. A compound of the invention may also be injected directly into the vitreous humor or aqueous humor.

Further, a compound may be also be administered by well known, acceptable methods, such as subtenon and/or subconjunctival injections. As is well known in the ophthalmic art, the macula is comprised primarily of retinal cones and is the region of maximum visual acuity in the retina. A Tenon's capsule or Tenon's membrane is disposed on the sclera. A conjunctiva covers a short area of the globe of the eye posterior to the limbus (the bulbar conjunctiva) and folds up (the upper cul-de-sac) or down (the lower cul-de-sac) to cover the inner areas of the upper eyelid and lower eyelid, respectively. The conjunctiva is disposed on top of Tenon's capsule. The sclera and Tenon's capsule define the exterior surface of the globe of the eye. For treatment of age related macular degeneration (ARMD), choroid neovascularization, retinopathies (such as diabetic retinopathy, retinopathy of prematurity), retinitis, uveitis, cystoid macular edema (CME), glaucoma, and other diseases or conditions of the posterior segment of the eye, it is preferable to dispose a depot of a specific quantity of an ophthalmically acceptable pharmaceutically active agent directly on the outer surface of the sclera and below Tenon's capsule. In addition, in cases of ARMD and CME it is most preferable to dispose the depot directly on the outer surface of the sclera, below Tenon's capsule, and generally above the macula.

The compounds may be formulated as a depot preparation. Such long-acting formulations may be administered by implantation (for example, subcutaneously or intramuscularly) intramuscular injection or by the above mentioned subtenon or intravitreal injection. Alternatively, the active ingredient may be in powder form for constitution with a suitable vehicle, e.g., sterile pyrogen-free water, before use.

Within particularly preferred embodiments of the invention, the compounds may be prepared for topical administration in saline (combined with any of the preservatives and antimicrobial agents commonly used in ocular preparations), and administered in eyedrop form. The solution or suspension may be prepared in its pure form and administered several times daily. Alternatively, the present compositions, prepared as described above, may also be administered directly to the cornea.

Within preferred embodiments, the composition is prepared with a muco-adhesive polymer which binds to cornea. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The preparation of preferred compounds of the present invention is described in detail in the following examples, but the artisan will recognize that the chemical reactions described may be readily adapted to prepare a number of other compounds of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or known in the art will be recognized as having applicability for preparing other compounds of the invention.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### EXAMPLES

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

The structures of the compounds are confirmed by either elemental analysis or NMR, where peaks assigned to the characteristic protons in the titled compound are presented where appropriate. ¹H NMR shift (δ_{H}) are given in parts per million (ppm) down field from an internal reference standard.

The invention will now be described in reference to the following EXAMPLES. These EXAMPLES are not to be regarded as limiting the scope of the present invention, but shall only serve in an illustrative manner.

Analysis and Purification Procedures for Final Products related to Methods a through R

The crude reaction mixtures were analyzed by HPLC. Prior to purification, samples were filtered through Whatman® GF/F Unifilter (#7700-7210), commercially available from Whatman® of Clifton, N.J. USA. Purification of samples was performed by reverse phase HPLC. Fractions were collected in 23 mL prepared tubes and centrifugal evaporated to dryness. Dried product was weighed and dissolved in DMSO. Products were then analyzed and submitted for screening.

NMR data was acquired on a Bruker DRX 300 NMR Spectrometer® using a broadband decoupling scheme to decouple the protons from the carbons. The Bruker DRX 300 NMR Spectrometer® is commercially available from Buker Biospin Corporation of Billercia, Mass.

Analytical LCMS Method (Pre-Purification)

Column: Peeke Scientific® HI-Q C-18, 50×4.6 mm, commercially available from Peeke Scientific® of Redwood City, Calif., 5 μm, Eluent A: Water with 0.05% TFA, Eluent B: Acetonitrile with 0.05% TFA, Gradient: linear gradient of 0-100% B in 3.0 min, then 100% B for 0.5 min, then 100-0% B in 0.25 min, hold 100% A for 0.75 min, Flow: 2.25 mL/min, Column Temperature: 25° C., Injection Amount: 15 μL of a 286 μM crude solution in methanol/DMSO/water 90/5/5, UV Detection: 260 and 210 nm, Mass Spectrometry: APCI, positive mode, mass scan range 111.6-1000 amu.

Preparative LC Method (Gilson)

Column: Peeke Scientific® HI-Q C18, 50 mm×20 mm, 5 lm, Eluent A: 0.05% TFA in Water, Eluent B: 0.05% TFA in Acetonitrile, Pre-inject Equilibration: 0.50 min, Post-inject Hold: 0.16 min, Gradient: 0-100% B in 2.55 min, then ramp 100% back to 0% in 0.09 min, Flow: 50.0 mL/min, Column Temp: Ambient, Injection Amount: 1200 μL of filtered crude reaction mixture in DMSO, Detection: UV at 210 nm or 260 nm.

Analytical LCMS Purification

Purification Conditions included a Waters® Bondapak column C18, 37-55 micron (particle size), 47×300 mm (column size) having a flow rate of 75 mL/min, a detector of UV 220 nm, where Buffer A is: 0.1% HOAc in H₂O and Buffer B is: 0.1% HOAc in CH₃CN. The Waters® Bondapak column C18 is commercially available from Varian, Inc. of Palo Alto, Calif., USA.

The column was equilibrated in Buffer A for 20 min. The sample was dissolved in 10 mL of DMSO, filtered, and injected onto the column. The gradient was held at 100% in Buffer A for 5 min and then increased linearly to 90% Buffer A/10% Buffer B in 20 min and then held at 10% Buffer B for another 25 min. The desired product came out at about 26 min during the isocratic hold of the gradient. The fractions were checked, pooled, and lyophilized to afford a syrup.

Analytical LCMS Method (Post-Purification)

Column: Peeke Scientific® HI-Q C-18, 50×4.6 mm, 5 μm, Eluent A: Water with 0.05% TFA, Eluent B: Acetonitrile with 0.05% TFA, Gradient: linear gradient of 0-100% B in 1.75 min, then 100% B for 0.35 min, then 100-50% B for 0.5 min, Flow: 3.00 mL/min, Column Temperature: 25° C., Injection Amount: 15 μL of a 300 μM solution in methanol/DMSO 99/1, UV Detection: 260 nm, Mass Spectrometry: APCI, positive mode, mass scan range 100-1000 amu, ELSD: gain=9, temp 40° C., nitrogen pressure 3.5 bar.

### Method A

### Example 1

### (R)-4-Ethyl-morpholine-3-carboxylic acid adamantan-2-ylamide

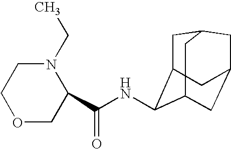

(R)-morpholine-3-carboxylic acid adamantan-2-ylamide trifluoroacetic acid salt (74 mg) was dissolved in DMF (1 mL), followed by the addition of Et₃N (60.1 μL) and Etl (32 μL), and the reaction solution was stirred at about 20° C. for 7 hours. Etl (64 μL) and DMF (1 mL) were added, and the reaction solution was stirred at a temperature of about 20° C. The reaction mixture was diluted with 2:1 of EtOAc:benzene (50 mL), washed with saturated with NaHCO₃ (10 mL), brine (twice with 10 mL). The organic layer was dried over MgSO₄ and concentrated in vacuo. The product was pumped under high vacuum overnight. The product was then converted to its HCl salt by dissolving in MeOH (2 mL), followed by the addition of 1 M HCl in ether (0.5 mL) to afford (R)-4-ethyl-morpholine-3-carboxylic acid adamantan-2-ylamide hydrochloride salt (55 mg, 86%).

### Prep (1a): (R)-4-Boc-morpholine-3-carboxylic acid adamantan-2-ylamide

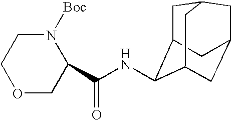

N-Boc-R-morpholinic acid (500 mg, 2.16 mmol), 2-adamantanamine-hydrochloride salt (188 mg, 2.59 mmol), HATU (986 mg, 2.59 mmol) were placed in a round bottom flask and dried under high vacuum for 2 hours. DMF (10 mL) and CH₂Cl₂ (10 mL) were added to dissolve reagents, followed by the addition of triethylamine (1.21 mL, 8.64 mmol), the resultant reaction mixture was stirred at about 20° C. overnight. The reaction solution was taken into 100 mL of 2:1 EtOAc:benzene, and washed with saturated NaHCO₃ (twice with 15 mL), brine (15 mL), 0.2 N HCl solution (twice with 15 mL), and brine (twice with 15 mL). The organic layer was dried over MgSO₄, and concentrated in vacua. The product was purified by flash chromatography eluting with 20% EtOAc in CH₂Cl₂ to afford (R)-4-Boc-morpholine-3-carboxylic acid adamantan-2-ylamide (289 mg, 37%; LCMS: 365.2).

### Prep (1b): (R)-morpholine-3-carboxylic acid adamantan-2-ylamide trifluoroacetic acid salt

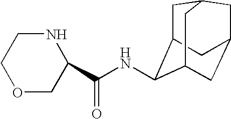

(R)-4-Boc-morpholine-3-carboxylic acid adamantan-2-ylamide (289 mg) was dissolved in neat trifluoroacetic acid (5 mL) and stirred at about 20° C. for 1 hour. The reaction solution was then concentrated in vacua. The resultant gummy solid was tritiated with anhydrous diethyl ether to afford (R)-morpholine-3-carboxylic acid adamantan-2-ylamide trifluoroacetic acid salt (300 mg, 100%; LCMS: 265.1).

### Example 3

### N-benzyl-1-(cyclohexylmethyl)-D-prolinamide

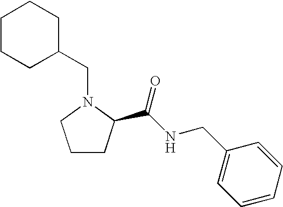

To a solution of N-benzyl-D-prolinamide (133 mg, 0.314 mmol) in DMF (3.5 mL) was added TEA (137 μL, 0.979 mmol) and cyclohexylmethyl bromide (75 μL, 0.54 mmol). The resultant solution was stirred at about 20° C. for 2.5 hours. Additional TEA (0.20 mL, 1.4 mmol) and cyclohexylmethyl bromide (0.10 mL, 0.72 mmol) were added and the resultant solution was heated to 100° C. and stirred overnight. The reaction mixture was cooled to about 20° C. and concentrated in vacua. The residue was purified by flash chromatography eluting with hexanes/EtOAc (20-50%) to afford the title compound (39 mg, 42% yield).

### Prep (3a): tert-butyl-(2R)-2-[(benzylamino)carbonyl]pyrrolidine-1-carboxylate

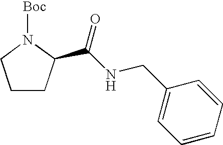

N-(tert-butoxycarbonyl)-D-proline (500 mg, 2.32 mmol) was placed in a round bottom flask. DMAP (14 mg, 0.12 mmol) in 2.3 mL CH₂Cl₂, HOBt (345 mg, 2.55 mmol) in 6.0 mL CH₂Cl₂, benzyl amine (380 μL, 3.48 mmol), EDC (489 mg, 2.55 mmol) in 6.0 mL CH₂Cl₂, and NMM (510 μL, 4.64 mmol) were added, respectively, to the flask. The resultant mixture was stirred at about 20° C. overnight. The reaction mixture was concentrated in vacuo and the residue was partitioned between EtOAc (400 mL) and 0.5 N HCl (40 mL). The organic layer was separated and washed with 0.5 N HCl(40 mL), brine (40 mL), saturated NaHCO₃ (twice with 40 mL), brine (40 mL), dried (MgSO₄), filtered, and concentrated in vacuo. The residue was purified by flash chromatography eluting with hexanes/EtOAc (20-45%) to afford the title compound (630 mg, 89% yield). ¹H NMR (400 MHz, DMSO-D6) δ ppm 1.23-1.31 (6H, m) 1.40 (3H, s) 1.72-1.84 (3H, m) 2.04-2.16 (1H, m) 3.24-3.33 (2H, m) 3.36-3.44 (1H, m) 4.04-4.12 (1H, m) 4.12-4.23 (1H, m) 4.29-4.37 (1H, m) 7.27 (5H, td, J=14.84, 7.96 Hz) 8.37 (1H, s); LCMS (M+1): 305.

### Prep (3b): N-benzyl-D-prolinamide

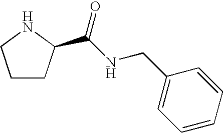

To a solution of tert-butyl-(2R)-2-[(benzylamino)carbonyl]pyrrolidine-1-carboxylate (560 mg, 1.84 mmol) in CH₂Cl₂ (9 mL), cooled to a temperature of about 0° C. to about 5° C., was added TFA (9 mL). After 2 hours, the solution was concentrated in vacuo. The residue was azeotroped with toluene (twice with 10 mL) then placed under high vacuum overnight to afford the title compound as the TFA salt (776 mg). ¹H NMR (400 MHz, CHLOROFORM-D) δ ppm 1.95 (3H, s) 2.34 (1H, d, J=6.82 Hz) 3.31 (2H, s) 4.32-4.42 (2H, m) 4.60 (1H, s) 7.15-7.24 (3H, m) 7.26-7.32 (2H, m) 7.58 (1H, s) 8.08 (1H, t, J=4.93 Hz) 10.72 (1H, s); LCMS (M+1): 305.

### Example 5

### N-benzyl-1-(cyclohexylmethyl)-L-prolinamide

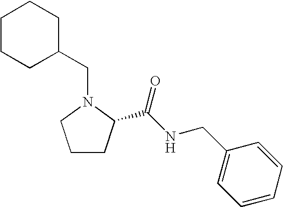

To a solution of N-benzyl-L-prolinamide (156 mg, 0.490 mmol) in DMF (4.0 mL) was added TEA (237 μL, 1.96 mmol) and cyclohexylmethyl bromide (136 μL, 0.979 mmol). The resultant solution was heated to about 100° C. for 6 hours. The reaction mixture was cooled to a temperature of about 20° C. overnight then diluted with 2:1 EtOAc/benxene (200 mL). The organic solution was washed with 0.5 N HCl (twice with 40 mL), brine (40 mL), saturated NaHCO₃ (twice with 40 mL), brine (40 mL), dried (MgSO₄), filtered, and concentrated in vacuo to afford 31 mg product. The combined aqueous layers were concentrated in vacuo. The residue was partitioned between EtOAc (200 mL) and H₂O (20 mL). The organic layer was separated and the aqueous layer was extracted with EtOAc (200 mL). The organic extracts were combined, dried (MgSO₄), filtered, and concentrated in vacuo to afford 51 mg crude product. These two batches of crude product were combined and purified by flash chromatography twice eluting with hexanes/EtOAc (20-50%) to afford the title compound (48 mg, 33% yield).

### Prep (5a): tert-butyl-(2S)-2-[(benzylamino)carbonyl]pyrrolidine-1-carboxylate

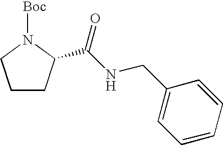

N-(tert-butoxycarbonyl)-L-proline (500 mg, 2.32 mmol) was placed in a round bottom flask. DMAP (14 mg, 0.12 mmol) in 2.3 mL CH₂Cl₂, HOBt (345 mg, 2.55 mmol) in 6.0 mL CH₂Cl₂, benzyl amine (380 μL, 3.48 mmol), EDC (489 mg, 2.55 mmol) in 6.0 mL CH₂Cl₂, and NMM (510 μL, 4.64 mmol) were added, respectively, to the flask. The resultant mixture was stirred at a temperature of about 20° C. overnight. The reaction mixture was concentrated in vacuo and the residue was partitioned between EtOAc (400 mL) and 0.5 N HCl (40 mL). The organic layer was separated and washed with 0.5 N HCl (40 mL), brine (40 mL), saturated NaHCO₃ (twice with 40 mL), brine (40 mL), dried (MgSO₄), filtered, and concentrated in vacuo. The residue was purified by flash chromatography eluting with hexanes/EtOAc (20-50%) to afford the title compound (647 mg, 92% yield). ¹H NMR (400 MHz, DMSO-D6) δ ppm 1.23-1.31 (6H, m) 1.40 (3H, s) 1.72-1.84 (3H, m) 2.04-2.16 (1H, m) 3.24-3.33 (2H, m) 3.36-3.44 (1H, m) 4.04-4.12 (1H, m) 4.12-4.23 (1H, m) 4.29-4.37 (1H, m) 7.27 (5H, td, J=14.84, 7.96 Hz) 8.37 (1H, s); LCMS (M+1): 305.
Prep (5b): N-benzyl-L-prolinamide

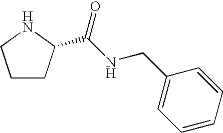

To a solution of tert-butyl-(2S)-2-[(benzylamino)carbonyl]pyrrolidine-1-carboxylate (580 mg, 1.91 mmol) in CH₂Cl₂ (9 mL), cooled to a temperature of about 0° C. to about 5° C., was added TFA (9 mL). After 2 hours, the solution was concentrated in vacuo. The residue was azeotroped with toluene (twice with 10 mL) then placed under high vacuum overnight to afford the title compound as the TFA salt (721 mg). ¹H NMR (400 MHz, CHLOROFORM-D) δ ppm 1.95 (3H, s) 2.34 (1H, d, J=6.82 Hz) 3.31 (2H, s) 4.32-4.42 (2H, m) 4.60 (1H, s) 7.15-7.24 (3H, m) 7.26-7.32 (2H, m) 7.58 (1H, s) 8.08 (1H, t, J=4.93 Hz) 10.72 (1H, s); LCMS (M+1): 305.

### Example 6

### N-2-adamantyl-1-ethyl-D-prolinamide

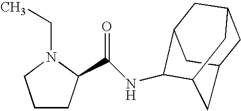

Ethyl iodide (108 g) was added to a slurry of N-2-adamantyl-D-prolinamide hydrochloride (40 g, 140 mmol) and triethylamine (150 mL, 1120 mmol) in DMA (300 mL) at 7° C. The reaction mixture was allowed to stir overnight in an ice-water bath. The reaction mixture was filtered and the solids were washed with ethyl acetate (1 L). The combined filtrates were diluted with MTBE (600 mL) and washed with saturated NaHCO₃ solution (once with 500 mL) and brine (once with 500 mL). The solvents were removed to get an amber colored oil. The crude compound was purified by chromatography (silica gel, 500 g), eluted with 1.5% 2N NH₃ in methol in CH₂Cl₂. The pure amine fractions, after evaporation, were dissolved in ethanol (100 mL) and cooled to a temperature of about 5° C. A hydrogen chloride solution (prepared from acetyl chloride (50 mL) and methanol (150 mL)) was added to the ethanol solution of the free amine. The solvents were removed after ten minutes and the resulting grey colored solids were treated with ethyl acetate (800 mL). The precipitated solids were filtered and dried at a temperature of about 20° C. under vacuum to afford the title compound (36.1 g).

### Prep (6a): tert-butyl-(2R)-2-[(2-adamantylamino)carbonyl]pyrrolidin-1-carboxylate

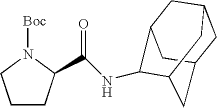

N-(tert-butoxycarbonyl)-D-proline (43.6 g, 202 mmol) was added to a slurry of 2-adamantylamine hydrochloride (38.3 g, 204 mmol), DMF (500 mL) and triethylamine (40.0 g, 395 mmol). The resulting very thick suspension was stirred vigorously and cooled to a temperature of about 11° C. The coupling reagent PyBOP (120.0 g, 230 mmol) in DMF (100 mL) was added while maintaining the temperature below 16° C. and the heterogeneous reaction mixture was left in an ice-water bath overnight. The reaction mixture was partitioned between water (3L) and ethyl acetate:MTBE (at a ratio of 1:1 with 4 L). The water layer was back-extracted with ethyl acetate:MTBE (at a ratio of 1:1 twice with 1 L). The combined organic layers were washed with brine (twice with 1 L) and dried over MgSO₄. The solvents were removed by evaporation and the product was purified by chromatography (silica gel 500 g; eluted with hexane:ethyl acetate 3:1).

Yield: 62.9 g. ¹H NMR (400 MHz, DMSO-D6) δ ppm 1.28-1.40 (9H, m) 1.48 (2H, d, J=12.38 Hz) 1.65-1.72 (4H, m) 1.72-1.83 (11H, m) 1.93-2.01 (1H, m) 2.02-2.13 (1H, m) 3.22-3.29 (1H, m) 3.75-3.85 (1H, m) 4.17-4.25 (1H, m) 7.62 (1H, d, J=7.58 Hz); LCMS (M+1): 349.
Prep (6b): N-2-adamantyl-D-prolinamide

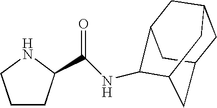

tert-Butyl-(2R)-2-[(2-adamantylamino)carbonyl]pyrrolidine-1-carboxylate (62.9 g, 180 mmol) in CH₂Cl₂ (400 mL) was cooled to a temperature of about 8° C. and a solution of hydrogen chloride (20.0 g, 540 mmol) in diethyl ether (700 mL) was added. The resultant clear solution was stirred at temperature of about 20° C. for 2 days. The precipitated solid was filtered, washed with CH₂Cl₂: Et₂O (at a ratio of 1:1 with 150 mL) and dried at 40° C. to give the desired product as a white solid (46.2 g). ¹H NMR (400 MHz, CHLOROFORM-D) δ ppm 1.51 (2H, d, J=12.63 Hz) 1.69 (2H, s) 1.74-2.01 (13H, m) 2.26-2.35 (1H, m) 3.22 (2H, ddd, J=17.62, 11.43, 6.06 Hz) 3.87 (1H, d, J=6.82 Hz) 4.19-4.27 (1H, m) 8.29-8.37 (1H, m) 8.47 (1H, s) 9.36 (1H, s); LCMS (M+1): 249.

### Example 9

### N-1-adamantyl-1-(cyclohexylmethyl)-D-prolinamide

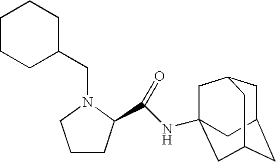

To a solution of N-1-adamantyl-D-prolinamide (300 mg, 0.828 mmol) in DMF (2 mL) was added TEA (577 μL, 4.14 mmol) followed by cyclohexylmethyl bromide (229 μL, 1.66 mmol). The resultant solution was subjected to microwave conditions for 20 minutes at 100° C. The reaction mixture was diluted with MTBE (200 mL). The organic solution was washed with saturated NaHCO₃ (three times with 20 mL), brine (20 mL), dried (MgSO₄), filtered, and concentrated in vacuo. To a solution of the residue in MeOH (5 mL), cooled to a temperature of about 0° C. to about 5° C. was added HCl (1 M in diethyl ether, 3 mL). The resultant solution was stirred for 30 minutes then concentrated in vacuo. The residue was triturated with diethyl ether to afford the title compound as the HCl salt (95 mg, 31% yield).

### Prep (9a): tert-butyl-(2R)-2-[(1-adamantylamino)carbonyl]pyrrolidine-1-carboxylate

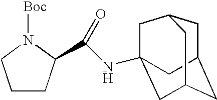

N-(tert-butoxycarbonyl)-D-proline (1.00 g, 5.65 mmol), EDC (982 mg, 5.12 mmol), HOBt (692 mg, 5.12 mmol), DMAP (28 mg, 0.23 mmol), and 1-adamanyl amine (1.06 g, 6.98 mmol) were charged into a round bottom flask. CH₂Cl₂ (25 mL) was added to dissolve the reagents followed by NMM (1.02 mL, 9.3 mL). The resultant solution was stirred at temperature of about 20° C. overnight. The solution was concentrated in vacuo and the residue was partitioned between EtOAc (400 mL) and 0.5 N HCl (40 mL). The organic layer was separated and washed with 0.5 N HCl (40 mL), brine (40 mL), saturated NaHCO₃ (twice with 40 mL), brine (40 mL), dried (MgSO₄), filtered, and concentrated in vacuo. The residue was purified by flash chromatography eluting with hexanes/EtOAc (5-50%) to afford the title compound (1.7 g, 105% yield). ¹H NMR (400 MHz, DMSO-D6) δ ppm 1.32-1.39 (10H, m) 1.56-1.64 (6H, m) 1.66-1.80 (3H, m) 1.87-1.94 (6H, s) 1.96-2.07 (4H, m) 3.20-3.28 (1H, m) 3.94-4.05 (1H, m) 7.21 (1H, s); LCMS (M+1): 349.

### Prep (9b): N-1-adamantyl-D-prolinamide

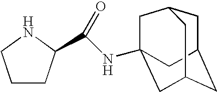

To a solution of tert-butyl-(2R)-2-[(1-adamantylamino)carbonyl]pyrrolidine-1-carboxylate, (1.64 g 4.71 mmol) in CH₂Cl₂ (5 mL) was added TFA (5 mL). The resultant solution was stirred at a temperature of about 20° C. for 3 hours. The reaction mixture was concentrated in vacua. The residue was azeotroped with toluene then triturated with diethyl ether to afford the title compound as the TFA salt (2.25 g). ¹H NMR (400 MHz, CHLOROFORM-D) δ ppm 1.60-1.70 (6H, m) 1.94-2.01 (8H, m) 2.05 (3H, s) 2.34-2.45 (1H, m) 3.38 (2H, t, J=6.44 Hz) 4.52 (1H, dd, J=7.83, 5.81 Hz) 7.35 (1H, s); LCMS (M+1): 249.

### Method B

### Example 11

### (3R)-N-cyclohexyl-4-(cyclohexylmethyl)-N-methylmorpholine-3-carboxamide

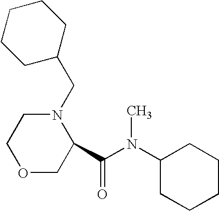

(R)-4-Boc-Morpholine-3-carboxylic acid (508.7 mg, 2.2 mmol) was reacted with N-Methylcyclohexylamine (249 mg) in a 1:1 ratio at a temperature of about 20° C. overnight in the presence of 1.2 eqv of HATU (O-(7-Azabenzotriazol-1-yl)-N,N,N′,N′-tetramethyluronium hexafluorophosphate) and 1.2 eqv of TEA (Trimethylamine) using NMP (4-Methylmorpholine) as the solvent. The reaction was worked up using EtOAc and H₂O. The EtOAc layer was dried with Na₂SO₄, concentrated, and purified by normal phase (using Biotage column) using EtOAc and Hexane. The intermediate was deprotected using 1:1 TFA:Methylene chloride overnight. The solvent was evaporated and the crude product was washed three times with n-Heptane. The crude material was then reacted with 1 eqv (296.1 mg) of cyclohexanecarboxaldehyde in the presence of 2.4 eqv of NaHB(OAc)₃ with CH₃CN as solvent and allowed to stir overnight. The reaction was then concentrated to dryness and worked up using EtOAc and H₂O. The EtOAc layer was dried using Na₂SO₄, concentrated, and purified using reverse phase (with 0.1% HOAc in H₂O and CH₃CN as buffer/solvent). The purified product was a syrup (638.8 mg, 90% yield).

### Example 28

### (4R)-N-2-adamantyl-1-cyclopentylmethyl-4-hydroxy-D-prolinamide

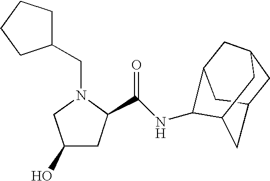

To a solution of (4R)-N-2-adamantyl-4-hydroxy-D-prolinamide (100 mg, 0.264 mmol), cooled to a temperature of about 0° C. to about 5° C. in MeOH (5 mL) was added cyclopentylaldehyde (52 mg, 0.529 mmol) followed by NaCNBH₃ (18 mg, 0.29 mmol). The solution was stirred for 30 minutes at a temperature of about 0° C. to about 5° C., then at a temperature of about 20° C. overnight. The reaction mixture was concentrated in vacuo and the residue was dissolved in EtOAc (100 mL). The organic solution was washed with saturated NaHCO₃ (twice with 15 mL), brine (15 mL), dried (MgSO₄), filtered and concentrated in vacuo. The product was purified by flash chromatography eluting with CH₂Cl₂/MeOH (0-7%) to afford the title compound as a foamy solid (81 mg, 88%).

### Prep (28a): tert-butyl-(2R,4R)-2-[(2-adamantylamino)carbonyl]4-hydroxypyrrolidine-1-carboxylate

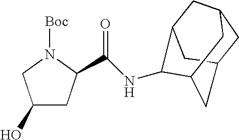

To a solution of (4R)-1-(tert-butoxycarbonyl)-4-hydroxy-D-proline (2.5 g, 10.8 mmol) in DMF (50 mL) was added 2-adamantyl amine hydrochloride (2.13 g, 11.4 mmol). To the mixture was added HATU (4.32, 11.4 mmol) followed by triethylamine (4.52 mL, 32.4 mmol). The reaction mixture was stirred overnight at a temperature of about 20° C. and filtered. The mother liquor was diluted with 2:1 EtOAc:benzene (750 mL) and washed with 0.5 N HCl (twice with 70 mL), brine (70 mL), saturated NaHCO₃ (twice with 70 mL), brine (70 mL), dried (MgSO₄), filtered, and concentrated in vacuo. The product was purified by flash chromotagraphy eluting with hexanes/EtOAc (25%) followed by a second column eluting with CHCl₃/MeOH (2%) to afford the title compound (4.04 g, 103%). ¹H NMR (400 MHz, MeOD) δ ppm 1.39-1.48 (m, 9H) 1.63 (d, J=12.88 Hz, 2H) 1.78 (s, 2H) 1.80-1.91 (m, 8H) 1.92-2.02 (m, 3H) 2.28-2.50 (m, 1H) 3.50 (d, J=3.79 Hz, 2H) 3.95 (s, 1H) 4.26 (s, 1H) 4.32 (td, J=5.31, 2.53 Hz, 1H).

### Prep (28b): (4R)—N-2-adamantyl hydroxy-D-prolinamide

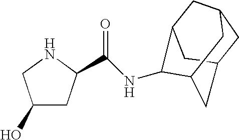

To a solution of tert-butyl-(2R,4R)-2-[(2-adamantylamino)carbonyl]-4-hydroxypyrrolidine-1-carboxylate (4.04 g, 11.1 mmol), cooled to a temperature of about 0° C. to about 5° C. in CH₂Cl₂ (25 mL) was added trifluoroacetic acid (25 mL, 395 mmol). The resultant solution was warmed to a temperature of about 20° C. and stirred overnight. The reaction mixture was concentrated, azeotroped with toluene (three times), then triturated with diethyl ether to afford the title compound as a white solid (3.37 g, 80%). ¹H NMR (400 MHz, MeOD) δ ppm 1.66 (d, J=12.88 Hz, 2H) 1.80 (s, 2H) 1.82-2.03 (m, 10H) 2.04-2.10 (m, 1H) 2.63 (ddd, J=14.02, 10.11, 4.93 Hz, 1H) 3.33-3.40 (m, 2H) 4.02 (s, 1H) 4.34 (dd, J=10.23, 4.93 Hz, 1H) 4.50 (tt, J=4.52, 2.31 Hz, 1H).

Method C

### Example 18

### N-2-adamantyl-1-acetyl-D-prolinamide

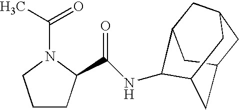

To solution of N-2-adamantyl-D-prolinamide (250 mg, 1.00 mmol) in THF (4 mL) was added triethylamine (702 μL, 5.03 mmol), followed by acetyl chloride (358 mL, 5.03 mmol). The exotherm was controlled using an ice-water bath. The reaction mixture turned from a colorless solution to cloudy orange mixture. After 1 hour, the mixture was diluted with EtOAc (100 mL), washed with 0.5 N HCl (10 mL), brine (10 mL), saturated NaHCO₃ (10 mL), brine (10 mL), dried (MgSO₄), filtered and concentrated in vacuo. The product was purified by flask chromatography eluting with hexanes/EtOAc (5-60%), followed by a second column eluting with CHCl₃/MeOH (0-4%) to afford the title compound (96 mg, 33%).

### Method D

### Example 47

### (4R)-N-2-adamantyl-4-hydroxy-1-[(1-methylpiperidin-4-yl)methyl]-D prolinamide

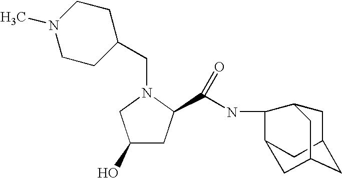

To a solution containing (4R)-N-2-adamantyl-4-hydroxy-1-(piperidin-4-ylmethyl)-D-prolinamide (200 mg, 0.42 mmol) in anhydrous THF (2.0 mL), CHCl₃ (3.5 mL), DMAC (0.5 mL), molecular sieves was added formaldehyde 37% solution (0.313 mL) and formic acid (0.15 mL) at a temperature of about 20° C. After stirring at 70° C. for 16 hours, the reaction solvents were removed under reduced pressure. The resulting residue was diluted with EtOAc and washed with saturated NaHCO₃. The aqueous layer was extracted with EtOAc. The combined organic extracts were dried with K₂CO₃ and filtered. The solvents were removed under reduced pressure and the resulting residue was purified using high performance flash chromatography eluted with 10% 7N NH₃ in MeOH in EtOAc to give desired product (90 mg, 57%).

### Prep (47a): tert-butyl 4-({(2R,4R)-2-[(2-adamantylamino)carbonyl]-4-hydroxypyrrolidin-1-yl}methyl)piperidine-1-carboxylate

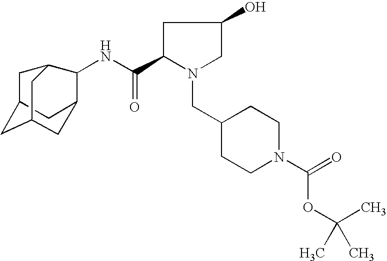

A solution of (4R)-N-2-adamantyl-4-hydroxy-D-prolinamide.TFA salt (100 mg, 1.06 mmol), molecular sieves, and 1-Boc-4-piperidinecarboxaldehyde (451 mg, 2.11 mmol) in methanol (4.5 mL) was stirred at a temperature of about 20° C. for 10 minutes. Then to this solution, sodiumcyanoborohydride (199.3 mg, 3.17 mmol) was added. After stirring the mixture for 16 hours the reaction mixture was quenched with water and the solvent was removed under reduced pressure. The reaction residue was diluted with EtOAc and water. The layers were separated. After being dried with K₂CO₃ and filtered, the organic solvents were removed under reduced pressure and the resulting residue was purified using high performance flash chromatography eluted with 40% acetone in hexane to give desired product (430 mg, 88%).

### Prep (47b): (4R)-2-adamantyl-4-hydroxy-1-(piperidin-4-ylmethyl)-D-prolinamide

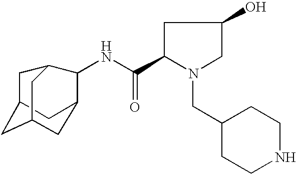

To tert-butyl 4-({(2R,4R)-2-[(2-adamantylamino)carbonyl]-4-hydroxypyrrolidin-1-yl}methyl)piperidine-1-carboxylate (420 mg, 0.91 mmol) in CH₂Cl₂ (10 mL), TFA (1.5 mL) was added at a temperature of about 20° C. After stirring at a temperature of about 20° C. for 16 hours, the reaction mixture was concentrated under reduced pressure. The resulting residue was triturated with EtOAc to give the desired product as a white solid 400 mg.

### Example 42

### (4R)-N-cyclohexyl-4-hydroxy-1-[(1-methylpiperidin-4-yl)methyl]-D prolinamide

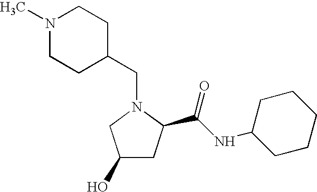

To a solution of (4R)-N-cyclohexyl-4-hydroxy-1-(piperidin-4-ylmethyl)-D-prolinamide (225 mg, 0.555 mmol) in 5:1 THF:chloroform, formic acid (170 μL, 4.44 mmol) and formaldehyde (37% in water, 330 μL, 4.44 mmol) were added. The resulting solution was refluxed for 4 hours then cooled to a temperature of about 20° C., diluted with ethyl acetate (125 mL), washed with saturated sodium carbonate (20 mL), brine (20 mL), dried (MgSO₄), filtered and concentrated in vacuo. The residue was purified by flash chromatography eluting with ethyl acetate/7 N methanolic ammonia (10%) to afford the title compound (75 mg, 42% over two steps).

### Prep (42a): tert-butyl(2R,4R)-2-[(cyclohexylamino)carbonyl]-4-hydroxypyrrolidine-1-carboxylate

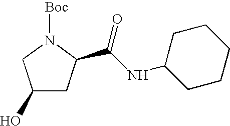

To a solution of (4R)-1-(tert-butoxycarbonyl)-4-hydroxy-D-proline (2.00 g, 8.66 mmol) in DMF (40 mL) was added cyclohexylamine (1.04 mL, 9.09 mmol), HATU (3.46 g, 9.09 mmol), then triethylamine (2.41 mL, 17.3 mmol). The resulting solution was stirred at a temperature of about 20° C. overnight then diluted with 2:1 ethyl acetate:benzene (400 mL). The organic solution was washed with 0.5 N HCl (twice with 50 mL), brine (40 mL), saturated NaHCO₃ (twice with 40 mL), brine (50 mL), dried (MgSO₄), filtered, and concentrated in vacuo. The crude product was purified by flash chromatography eluting with hexanes/acetone (15-45%) to afford the title compound as a white solid (2.329, 86%). ¹H NMR (400 MHz, MeOD) δ ppm 1.18-1.30 (m, 3H) 1.31-1.39 (m, 2H) 1.43 (s, 9H) 1.58-1.67 (m, J=11.12 Hz, 1H) 1.71-1.78 (m, J=11.12 Hz, 2H) 1.81-1.93 (m, 3H) 2.33-2.45 (m, 1H) 3.41-3.46 (m, 1H) 3.51-3.56 (m, 1H) 3.60-3.68 (m, 1H) 4.12-4.19 (m, 1H) 4.27 (ddd, J=7.58, 4.93, 2.91 Hz, 1H). LC-MS (APCl+) m/z 213.2 (M+H)⁺; t_{R}=^{2.967} min.

### Prep (42b): (4R)-N-cyclohexyl-4-hydroxy-D-prolinamide

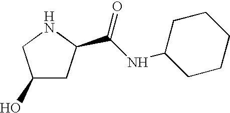

To a solution of tert-butyl(2R,4R)-2-[(cyclohexylamino)carbonyl]-4-hydroxypyrrolidine-1-carboxylate (2.27 g, 7.27 mmol) in dichloromethane (20 mL), cooled to a temperature of about 0° C. to about 5° C., was added trifluoroacetic acid (20 mL, 260 mmol). The resulting solution was stirred at a temperature of about 20° C. overnight then concentrated. The residue was azeotroped with toluene (three times with 30 mL) then triturated with diethyl ether to afford the title compound as the trifluoroacetate salt (2.35 g, 99%). ¹H NMR (400 MHz, MeOD) δ ppm 1.18-1.29 (m, 3H) 1.31-1.42 (m, 2H) 1.61-1.68 (m, 1H) 1.72-1.80 (m, 2H) 1.85-1.92 (m, J=10.86 Hz, 2H) 2.04-2.10 (m, J=13.93, 4.45, 2.18, 2.18 Hz, 1H) 2.52-2.60 (m, 1H) 3.33-3.36 (m, J=1.77 Hz, 1H) 3.63-3.73 (m, 2H) 4.22 (dd, J₁=10.11, 4.80 Hz, 1H) 4.49 (tt, J=4.42, 2.27 Hz, 1H). LC-MS (APCl+) m/z213.2 (M+H)⁺; t_{R}=0.804 min.

### Prep (42c): tert-butyl-4-({(2R,4R)-2-[(cyclohexylamino)carbonyl]hydroxypyrrolidin-1-yl}methyl)piperidine-1-carboxylate

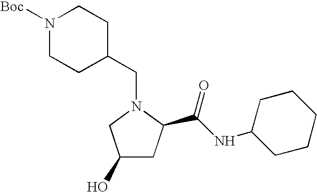

To a solution of (4R)-N-cyclohexyl-4-hydroxy-D-prolinamide (250 mg, 0.766 mmol) in methanol (10 mL) was added 1-Boc-4-piperidinecarboxaldehyde (180 mg, 0.843 mmol) followed by NaCNBH₃ (53 mg, 0.843 mmol). The resulting solution was stirred at a temperature of about 20° C. overnight then concentrated in vacuo. The residue was dissolved in ethyl acetate (200 mL), washed with saturated NaHCO₃ (twice with 20 mL), brine (20 mL), dried (MgSO₄), filtered and concentrated in vacuo. The crude product was purified by flash chromatography eluting with hexanes/ethyl acetate (25-55%) then dichloromethane/methanol (10%) to afford the title compound as a white solid (227 mg, 72%). ¹H NMR (400 MHz, MeOD) δ ppm 0.98-1.08 (m, 2H) 1.19-1.31 (m, 3H) 1.32-1.39 (m, 2H) 1.39-1.46 (s, 9H) 1.59-1.67 (m, J=3.54 Hz, 2H) 1.67-1.78 (m, 4H) 1.80-1.88 (m, J=10.86 Hz, 2H) 1.98-2.05 (m, J=11.37 Hz, 1H) 2.30-2.39 (m, 2H) 2.39-2.47 (m, 2H) 2.75 (s, 2H) 2.94 (dd, J=10.61, 4.80 Hz, 1H) 3.14 (d, J=9.85 Hz, 1H) 3.57-3.68 (m, 1H) 4.06 (t, J=13.64 Hz, 2H) 4.24-4.32 (m, J=3.92, 3.92 Hz, 1H). LC-MS (APCl+) m/z 410.3 (M+H)⁺; t_{R}=3.021 min.

### Prep (42d): (4R)-N-cyclohexyl-4-hydroxy-1-(piperidin-4-ylmethyl)-D-prolinamide

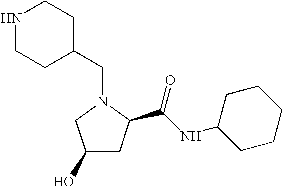

To a solution of tert-butyl 4-({(2R,4R)-2-[(cyclohexylamino)carbonyl]-4-hydroxypyrrolidin-1-yl)methyl)piperidine-1-carboxylate (227 mg, 0.555 mmol) in dichloromethane (5.0 mL), cooled a temperature of about 0° C. to about 5° C., was added trifluoroacetic acid (1.5 mL, 19 mmol). The resulting solution was stirred at a temperature of about 20° C. for 30 minutes then concentrated in vacuo. The residue was azeotroped with toluene three times, then diethyl ether twice to afford the title compound as the trifluoroacetate salt, which was used without further purification.

¹H NMR (400 MHz, MeOD) δ ppm 1.22-1.33 (m, 4H) 1.34-1.39 (m, 2H) 1.44-1.55 (m, 2H) 1.62-1.69 (m, 1H) 1.73-1.82 (m, 2H) 1.85-1.92 (m, 2H) 1.99-2.07 (m, 1H) 2.09-2.15 (m, 2H) 2.18-2.26 (m, 1H) 2.70-2.79 (m, 1H) 2.97-3.06 (m, 2H) 3.18 (dd, J=6.57, 3.28 Hz, 2H) 3.40-3.47 (m, J=13.64 Hz, 2H) 3.65-3.73 (m, J=10.61, 10.61, 4.29 Hz, 1H) 3.77 (d, J=11.62 Hz, 1H) 4.25 (dd, J=10.23, 4.93 Hz, 1H) 4.53 (ddd, J=4.23, 1.96, 1.64 Hz, 1H). LC-MS (APCl+) m/z 310.3 (M+H).

### Method E

### Example 44

### (3R)-N-2-adamantyl-4-[2-(dimethylamino)ethyl]morpholine-3-carboxamide

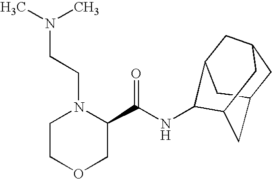

To a solution of (3R)-N-2-adamantyl-4-(2-aminoethyl)morpholine-3-carboxamide (137 mg, 0.334 mmol) in DMF (1.4 mL) and THF (2.0 mL) was added formic acid (103 μL, 2.67 mmol), formaldehyde (37% in water, 236 μL, 2.67 mmol) and 3 Å molecular sieves. The resulting mixture was refluxed for 1 hour, cooled to a temperature of about 20° C., filtered, and concentrated in vacuo. The residue was purified by flash chromatography dichloromethane/7 N methanolic ammonia (0-7.5%) to afford the title compound (55 mg, 49%), which was converted to the hydrochloride salt (67 mg).

### Prep (44a): tert-butyl(2-((3R)-3-[(2-adamantylamino)carbonyl]morpholin-4-yl}ethyl)carbamate

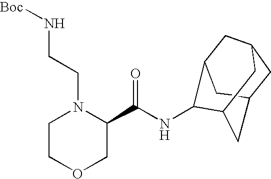

To a solution of (3R)-N-2-adamantylmorpholine-3-carboxamide (200 mg, 0.529 mmol) and tert-butyl(2-oxoethyl)carbamate (93 mg, 1.72 mmol) in methanol (6 mL) was added 3 Å molecular sieves (800 mg) followed by NaCNBH₃ (37 mg, 0.528 mmol) in two portions 5 minutes apart. The resulting mixture was stirred at a temperature of about 20° C. for 6 hours. Additional tert-butyl(2-oxoethyl)carbamate (1 eqv) and NaCNBH₃ (1 eqv) was added and the reaction mixture was stirred at a temperature of about 20° C. for 2.5 days then heated to 50° C. and stirred for 7 hours. Additional tert-butyl(2-oxoethyl)carbamate (0.5 eqv), NaBCNH₃ (0.5 eqv), and molecular sieves (400 mg) were added and the mixture was stirred for 50° C. overnight. The reaction mixture was cooled to a temperature of about 20° C. and filtered through Celite®. The mother liquor was concentrated and the residue was partitioned between ethyl acetate (100 mL) and saturated NaHCO₃ (15 mL). The organic layer was separated and washed with brine (15 mL), dried (MgSO₄), filtered, and concentrated in vacuo. The crude product was purified by flash chromatography eluting with dichloromethane/acetone (0-30%) to afford the title compound (126 mg, 63%). ¹H NMR (400 MHz, MeOD) δ ppm 1.43 (s, 9H) 1.62-1.71 (m, J=10.86, 10.86 Hz, 2H) 1.79 (s, 2H) 1.82-1.89 (m, 6H) 1.90-1.96 (m, 4H) 2.24-2.33 (m, 2H) 2.64 (dt, J=12.63, 7.58 Hz, 1H) 2.99-3.08 (m, 2H) 3.20 (dd, J=7.83, 5.31 Hz, 2H) 3.51-3.54 (m, 1H) 3.62 (td, J=11.05, 2.40 Hz, 1H) 3.79-3.86 (m, 2H) 3.95 (s, 1H); LC-MS (APCl+) m/Z 408.3 (M+H); t_{R}^{=3.630} min.

### Prep (44b): (3R)-N-2-adamantyl-4-(2-aminoethyl)morpholine-3-carboxamide

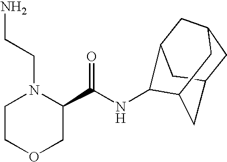

To a solution of tert-butyl(2-{(3R)-3-[(2-adamantylamino)carbonyl]morpholin-4-yl)ethyl)carbamate (136 mg, 0.334 mmol) in dichloromethane (3 mL), cooled to a temperature of about 0° C. to about 5° C., was added HCl (4 N in dioxane, 833 μL, 3.34 mmol). The solution was warmed to a temperature of about 20° C. and after 3 hours the solids were filtered to give the title compound as the hydrochloride salt (137 mg, 100%). ¹H NMR (400 MHz, MeOD) δ ppm 1.63-1.70 (m, 2H) 1.80 (s, 3H) 1.83-1.88 (m, 3H) 1.89-1.93 (m, J=5.31, 2.27 Hz, 3H) 1.93-1.97 (m, 2H) 2.01 (d, J=13.14 Hz, 1H) 3.35-3.44 (m, 4H) 3.64-3.75 (m, 3H) 3.82-3.90 (m, 1H) 4.03-4.11 (m, 2H) 4.20-4.26 (m, 2H) 8.55 (d, J=6.82 Hz, 1H). LC-MS (APCl+) m/z 308.3 (M+H); t_{R}=2.323 min.

### Method F

### Example 45

### N-2-adamantyl-4-amino-1-(cyclopentylmethyl)-D-prolinamide

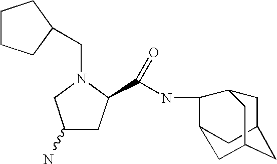

A suspension of N-2-adamantyl-1-(cyclopentylmethyl)-4-(hydroxyimino)-D-prolinamide (40 mg, 0.11 mmol) in methanol (1 mL), concentrated aqueous ammonia (0.02 mL), and R^{a}/Ni was shaken with hydrogen. After two hours, the reaction mixture was filtered through a Celite® pad. The filtered cake was washed with methanol (three times with 3 mL). The solvents were removed under reduced pressure and the resulting residue was using reversed phase Kromasil® C18, 0.05% TFA in water and acetonitrile to provide the titled product as a TFA salt (7.4 mg).

### Prep (45a): N-2-adamantyl-1-(cyclopentylmethyl)-4-oxo-D-prolinamide

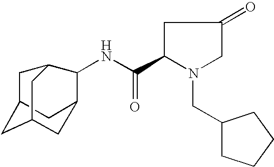

To a solution of oxalyl chloride (0.35 mL, 3.98 mmol) in methylene chloride (4 mL) was added DMSO (1.41 mL, 19.9 mmol) at −78° C. drop-wise. After stirring for 25 minutes, to the reaction mixture, a solution of (4R)-2-adamantyl-1-(cyclopentylmethyl)-4-hydroxy-D-prolinamide (230 mg, 0.664 mmol) in methylene chloride (2.5 mL) was added drop-wise. After stirring the reaction at −78° C. for 25 minutes, the reaction mixture was quenched with TEA (0.5 mL, 4.74 mmol). After stirring at a temperature of about 20° C. for 25 minutes, the reaction suspension was diluted with CH₂Cl₂ (40 mL) and water (15 mL). The aqueous layer was extracted with CH₂Cl₂ (twice with 15 mL). After dried with MgSO₄ and filtered, the organic solvents were removed under reduced pressure and the resulting residue was purified using high performance flash chromatography eluted with 50% acetone in hexane to give desired product (100 mg, 44%).

### Prep (45b): N-2-adamantyl-1-(cyclopentylmethyl)-4-(hydroxyimino)-D-prolinamide

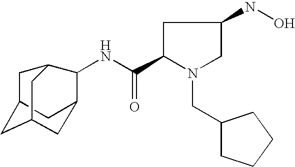

To a solution of hydroxylamine.HCl (40.3 mg, 0.58 mmol) in a mixture of water (0.1 mL) and methanol (1.0 mL) was added drop-wise a solution of N-2-adamantyl-1-(cyclopentylmethyl)-4-oxo-D-prolinamide (100 mg, 0.29 mmol) in methanol (1.0 mL) and K₂CO₃ (44.5 mg, 0.32 mmol). After stirring at a temperature of about 20° C. for 30 minutes, water (0.1 mL) was added. After stirring at a temperature of about 20° C. over night, the reaction mixture was concentrated under reduce pressure. To the resulting residue, water (1.0 mL) was added and the suspension was stirred at a temperature of about 20° C. for 20 minutes. The solid was filtered and purified using high performance flash chromatography eluted with 50% acetone in hexane to give desired product (40 mg, 38%).

### Method G

### Example 87

### 1-(2-Hydroxy-2-methyl-propyl)-pyrrolidine-2-carboxylic acid cyclohexylamide

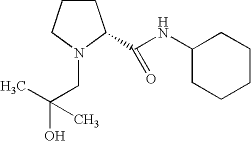

A mixture of pyrrolidine-2-carboxylic acid cyclohexylamide (500 mg, 1.63 mmol), 1,2-epoxy-2-methylpropane (commercially available from Aldrich®, 2.5 eqv, 0.36 mL, 4.1 mmol) and triethylamine (3 eqv, 0.68 mL, 4.9 mmol) in methanol was stirred at a temperature of about 20° C. for 18 hours. After such time the mixture was concentrated in vacuo and portioned between dichloromethane (80 mL) and saturated aqueous sodium hydrogen carbonate (80 mL). The organic phase was dried (magnesium sulfate) and purified via flash column chromatography (SiO₂, dichiromethane:methanol 100:0-97:3) to return named compound as a clear colorless oil (341 mg, 1.27 mmol, 78% yield).

### Prep (87a): Pyrrolidine-2-carboxylic acid cyclohexylamide

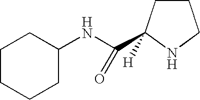

To a solution of Boc-D-proline (commercially available from Aldrich®, 5 g, 23.3 mmol), triethylamine (35.0 mmol, 4.5 mL), O-benzotriazol-1-yl-N,N,N′N′-tetramethyluronium hexafluorophosphate (27.9 mmol, 10.6 g) in dimethylformamide (130 mL) was added cyclohexylamine (commercially available from Aldrich®, 27.9 mmol, 3.2 mL) at a temperature of about 20° C. Mixture stirred for 18 hours at a temperature of about 20° C., then concentrated in vacuo. The residue was taken up in ethyl acetate (300 mL) and washed with sodium hydroxide (0.1M, 200 mL), water (200 mL), and brine (100 mL) and dried over sodium sulfate and concentrated in vacuo. The residue was taken up in dichloromethane (100 mL) to which trifluoroacetic acid was added and the mixture stirred for 18 hours at a temperature of about 20° C. After such time the mixture was concentrated in vacuo to yield the title compound as a pale yellow oil in quantitative yield. APCl+197 [M+H]+100%.

### Method H

### Example 88

### 1-(2-Methoxy-2-methyl-propyl)-pyrrolidine-2-carboxylic acid cyclohexylamide

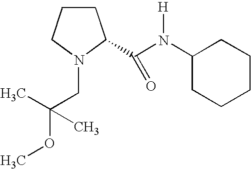

To a solution of 1-(2-Hydroxy-2-methyl-propyl)-pyrrolidine-2-carboxylic acid cyclohexylamide (298 mg, 1.1 mmol) and iodomethane (2.0 mmol, 0.12 mL) in tetrahydrofuran (15 mL) at a temperature of about 0° C. was added sodium hydride (60% dispersion in oil, 89 mg, 2.2 mmol). After 2 hours the mixture was allowed to warm to a temperature of about 20° C. After further 3 hours mixture was concentrated in vacuo, portioned between dichloromethane (50 mL) and aqueous sodium hydrogen carbonate (50 mL). The organic phase was dried over magnesium sulfate and purified via flash column chromatography (SiO₂, dichloromethane/methanol 0-3%) to yield the title compound as a white solid (75 mg, 24% yield).

### Method I

### Example 110

### 1-[2-(Benzyl-methyl-amino)ethyl]-pyrrolidine-2-carboxylic acid adamantan-2-ylamide

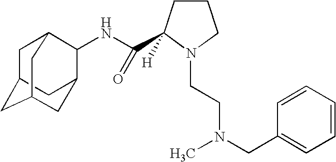

To a solution of 2-(Benzyl-methyl-amino)-ethanol (commercially available from Aldrich®, 1.0 g, 6.0 mmol), triethylamine (1.5 eqv, 0.7 mL, 9.0 mmol) in dichloromethane at 0° C. was added methanesulfonyl chloride (1.5 eqv, 0.7 mL, 9.0 mmol). After 45 minutes the mixture was poured on to cold water (10 mL) and extracted with dichloromethane (three times with 50 mL). Combined organic extracts were washed with saturated sodium chloride (50 mL) and dried over magnesium sulfate, filtered and concentrated in vacuo. The reside was taken up in acetonitrile (20 mL) to which triethylamine (2 eqv, 12 mmol, 1.6 mL) and N-2-adamantyl-D-prolinamide (1 eqv, 2.17 g, 6 mmol) was added. The mixture was stirred for 18 hours at a temperature of about 20° C. and purified via flash column chromatography (SiO₂, Ethyl acetate/methanol 0-6%) to return title compound as a clear colorless oil (1.75 g, 4.4 mmol, 74% yield).

### Method J

### Example 111

### 142-Methylamino-ethyl)-pyrrolidine-2-carboxylic acid adamantan-2-ylamide

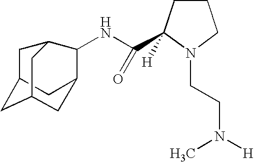

1-[2-(Benzyl-methyl-amino)-ethyl]-pyrrolidine-2-carboxylic acid adamantan-2-ylamide (0.5 g, 1.64 mmol) was dissolved in acetic acid (10 mL) to which 10% palladium on carbon (0.13 g) was added. The mixture was stirred for 18 hours under an atmosphere of hydrogen gas. After such time the mixture was filtered through a pad of Celite®, which was then washed with methanol (three times with 20 mL). The filtrate was then concentrated to 20 mL and poured onto crushed ice and made basic via the addition of ammonium hydroxide (30 mL) and extracted with dichloromethane (five time with 20 mL). The combined organic extracts were washed with brine (50 mL), dried over magnesium sulfate, filtered and concentrated in vacuo to return desired product as a foam (452 mg, 60% yield).

### Method K

### Example 121

### Piperidine-3-carboxylic acid adamantan-2-ylamide

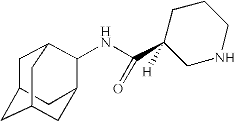

3-(Adamantan-2-ylcarbamoyl)-piperidine-1-carboxylic acid tert-butyl ester (3 g, 8.3 mmol) was taken up in dichloromethane (33 mL) to which trifluoroacetic acid (10 mL) was added and the mixture stirred for 18 hours at a temperature of about 20° C. After such time the mixture was concentrated in vacuo to return the named compound as a white solid in 92% yield.

### Prep (121a): 3-(Adamantan-2-ylcarbamoyl)-piperidine-1-carboxylic acid tert-butyl ester

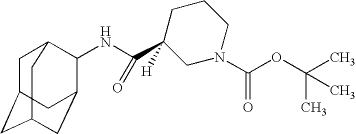

To a solution of N-Boc-(S)-nipeicotic acid (CNH Tachnologies, 5 g, 21.8 mmol), triethylamine (2.4 eqv, 52.3 mmol, 7.3 mL), O-benzotriazol-1-yl-N,N,N′N′-tetramethyluronium hexafluorophosphate (1.2 eqv, 26.2 mmol, 9.95 g) in dimethylformamide (87 mL) was added 2-aminoadamantane hydrochloride (commercially available from Aldrich®, 1.2 eqv, 26.2 mmol, 4.9 g) at a temperature of about 20° C. The mixture stirred for 18 hours at a temperature of about 20° C., then concentrated in vacuo. The residue was taken up in ethyl acetate (300 mL) and washed with saturated sodium hydrogen carbonate (200 mL) and brine (100 mL) and dried over sodium sulfate and concentrated in vacuo. The residue was purified via flash column chromatography (SiO₂, dichloromethane) to return title compound as an off white solid (3.32 g, 9.2 mmol, 44% yield).APCl+363 [M+H]+100%.

### Method L

### Example 129

### 1-(2-Acetylamino-ethyl)pyrrolidine-2-carboxylic acid adamantan-2-ylamide

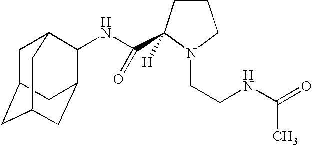

To a solution of 1-(2-Amino-ethyl)-pyrrolidine-2-carboxylic acid adamantan-2-ylamide hydrochloride (100 mg, 0.31 mmol) and triethylamine (0.14 mL, 1 mmol) in dichloromethane (20 mL) was added acetyl chloride (0.026 mL, 0.37 mmol). The mixture was stirred at a temperature of about 20° C. for 18 hours. After such time the mixture was washed with aqueous sodium hydrogen carbonate (20 mL), dried over magnesium sulfate and purified via flash column chromatography (SiO₂, dichloromethane/methanol 0-10% to yield the title compound as a white foam (63 mg, 0.19 mmol, 51% yield).

### Prep (129a): 1-(2-Amino-ethyl)-pyrrolidine-2-carboxylic acid adamantan-2-ylamide hydrochloride

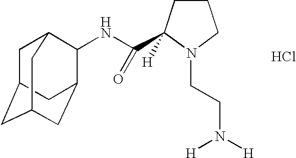

To a solution of {2-[2-(Adamantan-2-ylcarbamoyl)-pyrrolidin-1-yl]-ethyl}-carbamic acid tert-butyl ester (1.5 g, 3.8 mmol) in dichloromethane (30 mL) was added 4N hydrochloric acid in 1,4-dioxane (20 mL). Stirred for 4 hours at a temperature of about 20° C. After such time diethyl ether (50 mL) added and stirred for a further 1 hour. White precipitate formed and was filtered and washed with diethyl ether (twice with 15 mL) and dried to yield the title compound as a white solid (800 mg, 2.4 mmol, 64% yield). APCl+292 [M+H]⁺100%.

### Prep (129b): (2-[2-(Adamantan-2-ylcarbamoyl)-pyrrolidin-1-yl]-ethyl}-carbamic acid tert-butyl ester

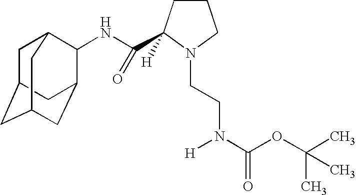

To a solution of N-2-adamantyl-D-prolinamide (1.5 g, 4.2 mmol), N-Boc-2-aminoacetaldehyde (commercially available from Aldrich®, 1 g, 6.3 mmol) in methanol 20 mL was added 3 Å molecular sieves (500 mg) followed by sodium cycanoborohydride (6.3 mmol, 390 mg) at a temperature of about 20° C. The mixture was heated to 50° C. for 6 hours. After such time the mixture was filtered through a pad of Celite® concentrated in vacuo and the residue portioned between dichloromethane (200 mL) and saturated aqueous sodium hydrogen carbonate (150 mL). The organic phase was dried over magnesium sulfate and purified via flash column chromatography (SiO₂, dichloromethane/methanol 0-5%) to yield the title compound as a white foam (1.5 g, 3.8 mmol, 91% yield). APCl+392 [M+H]+100%.

### Method M

### Example 130

### 1-(2-Methanesulfonylamino-ethyl)pyrrolidine-2-carboxylic acid adamantan-2-ylamide

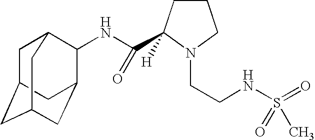

To a solution of 1-(2-Amino-ethyl)-pyrrolidine-2-carboxylic acid adamantan-2-ylamide hydrochloride (100 mg, 0.31 mmol) and triethylamine (0.14 mL, 1 mmol) in dichloromethane (20 mL) was added methanesulfonyl chloride (0.029 mL, 0.37 mmol). The mixture was stirred at a temperature of about 20° C. for 18 hours. After such time the mixture was washed with aqueous sodium hydrogen carbonate (20 mL), dried over magnesium sulfate and purified via flash column chromatography (SiO₂, dichloromethane/methanol 0-10% to yield the title compound a white foam (71 mg, 0.19 mmol, 51% yield).

### Method N

### Example 172

### N-2-adamantyl-1-(2-piperidin-1-ylethyl)-D-prolinamide

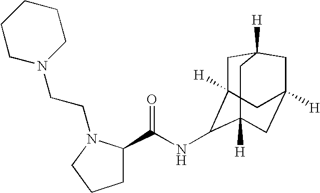

To a solution of 2-piperidin-1-ylethanol (129 mg, 1 mmol in 4 mL anhydrous dichloroethane), the following reagents were added in the following order: triethylamine (0.42 mL, 3 mmol), DMAP (0.08 mL, 0.1 mmol, 0.25 M, in dichloroethane), and methanesulfonyl chloride (228 mg, 2 mmol, in 4 mL dichloroethane). After the reaction mixture was stirred at a temperature of about 20° C. for 3 hours, the solvent was removed in vacuo, and the residue was subject to the next step without further purification. To the above residue dissolved in 4 mL anhydrous DMF, the following reagents were added in the following order: NaI (300 mg, 2 mmol), diisopropylethylamine (0.35 mL, 2 mmol), and N-2-adamantyl-D-prolinamide (248 mg, 1 mmol, in 4 mL anhydrous DMF). The reaction mixture was stirred and heated to a temperature of about 100° C. for 16 hours. After removing the solvent, the residue was dissolved in 20 mL ethyl acetate, and extracted with 1 M aqueous potassium carbonate (once with 10 mL), and then brine (once with 10 mL). The organic phase was dried over sodium sulfate, concentrated to dryness. The residue was subjected to flash chromatography on silica gel with 5% 7N NH₃-MeOH in ethyl acetate to yeild 91 mg of the title compound (26% overall).

### Method O

### Example 157

### N-2-adamantyl-1-[(2RS)-2-(dimethylamino)propyl]-D-prolinamide

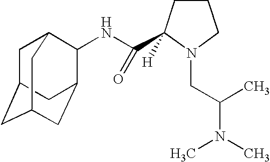

To an ice cold solution of N-2-adamantyl-1-[(2S)-2-hydroxypropyl]-D-prolinamide, (306 mg, 1 mmol) and triethylamine (1.5 mmol, 0.21 mL) in dichloromethane (5 mL) was added methansulfonyl chloride (1.5 mmol, 0.116 mL). After stirring for 15 minutes at 0° C. the reaction mixture was poured onto ice-cold water (15 mL) and extracted with dichloromethane (three times with 80 mL). The combined organic extracts were washed with brine, dried over magnesium sulfate, filtered and concentrated in vacuo. This reside was taken up in acetonitrile (5 mL) to which triethylamine (3 mmol, 0.42 mL) and dimethylamine hydrochloride (2 mmol, 163 mg) were added. After 18 hours stirring at a temperature of about 20° C., the mixture was concentrated in vacuo, the residue was taken up in dichloromethane and washed with sodium hydrogen carbonate, dried (magnesium sulfate) and purified via flash column chromatography (SiO₂, Ethyl acetate: 7N NH₃/MeOH 0-10%) to yield the title compound, a clear colorless oil (125 mg, 0.38 mmol, 38% yield) as a 1:1 diastereoisomeric mixture.

### Method P

### Example 167

### (2R)-N-2-adamantyl-1-(cyclopentylmethyl)-4-methylpiperazine-2-carboxamide

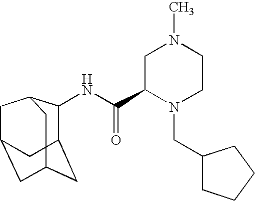

In a round bottom flask, (2R)-N-2-adamantyl-1-(cyclopentylmethyl)piperazine-2-carboxamide (0.20 g, 0.58 mmol) in CHCl₃ (10 mL) was dissolved, then formaldehyde (0.17 mL, 2.32 mmol at 37% in water) and formic acid (0.088 mL, 2.32 mmol) were added and then stirred for 12 hours at a temperature of about 20° C. Next, Na(OAc)₃BH₄ (0.49 g, 2.32 mmol) was added over 5 minutes and then the mixture was stirred for 3 hours. The reaction solution was diluted with EtOAc (50 mL) and partitioned between NaHCO₃ (twice with 30 mL). The organic layer was dried over Na₂SO₄ and concentrated. The residue was purified through silica (100 mL) eluting with hexane:EtOAc (1:1). The purified fractions were collected and concentrated. The residue was dissolved in Et₂O (10 mL) and 1 N HCl in Et₂O was added to generate a precipitate. The product was then dried on high vacuum for 12 hours to afford (2R)-N-2-adamantyi-1-(cyclopentylmethy)-4-methylpiperazine-2-carboxamide as white solid (0.089 g, 37.6%).

### Prep (167a): (2R)-4-(tert-butoxycarbonyl)-1-(cyclopentylmethyl)piperazine-2-carboxylic acid

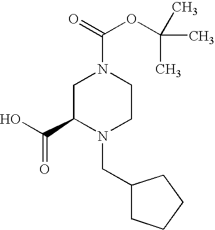

In a round bottom flask, (2R)-4-(tert-butoxycarbonyl)piperazine-2-carboxylic acid (1.50 g, 6.52 mmol) in THF (20 mL) was dissolved, then cyclopentanecarbaldehyde (0.70 mL, 7.62 mmol) with acetic acid (1.20 mL) was added and then stirred for 0.5 hours. Next, NaBH(OAc)₃ (2.07 g, 9.77 mmol) was added over 5 minutes and then stirred for 12 hours. The mixture was filtered though a cellose filter. The mother liquid was concentrated and placed on the high vacuum to afford (2R)-4-(tert-butoxycarbonyl)-1-(cyclopentylmethyl)piperazine-2-carboxylic acid as a white solid (1.98 g, 97.4%). ¹H NMR (400 MHz, DMSO-d₆) δ ppm: 3.48-3.40 (m, 1H), 3.36-3.25 (m, 2H), 3.12-3.00 (m, 2H), 2.28-2.24 (m, 1H), 2.17 (bs, 1H), 2.08-2.08-2.01 (m, 1H), 1.69-1.59 (m, 2H), 1.55-1.44 (m, 4H), 1.38 (s, 9H), 1.35-1.20 (m, 2H), 1.14-1.06 (m, 1H). LCMS (ESI): m/z: 313.2.

### Prep (167b): tert-butyl(3R)-3-[(2-adamantylamino)carbonyl]-4-(cyclopentylmethyl)piperazine-1-carboxylate

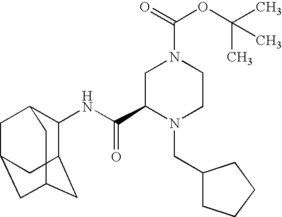

In a flask, (2R)-4-(tert-butoxycarbonyl)-1-(cyclopentylmethyl)piperazine-2-carboxylic acid (1.72 g, 5.46 mmol) was dissolved in DMF (10 mL), then adamantan-2-amine hydrochloride (1.22 g, 1.93 mmol) was added. Next, DIEA (1.93 mL, 11.84 mmol) and HATU (2.45 g, 6.53 mmol) was added and then stirred for 12 hours. The mixture was diluted with EtOAc (50 mL) and partitioned with NaHCO₃ (twice with 30 mL). The organic layer was dried over Na₂SO₄ and concentrated. The residue was purified through silica (100 mL) eluting with hexane/EtOAc (1:1). The purified fractions were colleted and concentrated. The residue was placed on high vacuum for 12 hours to afford tert-butyl(3R)-3-[(2-adamantylamino)carbonyl]-4-(cyclopentylmethyl) as a white foam (0.65 g, 26.8%). ¹H NMR (400 MHz, CDCl₃) o ppm: 7.21 (bs, 1H), 4.04 (d, J=8.08 Hz, 1H), 3.88 (bs, 1H), 3.12-3.03 (m, 2H), 2.82-2.79 (m, 1H), 2.47 (t, J=11.87 Hz, 1H), 2.27-2.07 (m, 3H), 1.91-1.75 (m, 24H), 1.45 (s, 9H). LCMS (ESI): m/z[M+H]: 446.2. Prep (167c): (2R)-N-2-adamantyl-1-(cyclopentylmethyl)piperazine-2-carboxamide

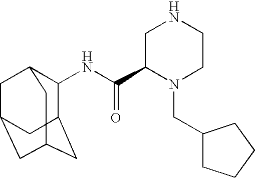

In a flask, tert-butyl(3R)-3-[(2-adamantylamino)carbonyl]-4-(cyclopentylmethyl) (0.40 g, 0.89 mmol) was dissolved in CH₂Cl₂ (10 mL) then TFA (10 mL) was added and then stirred for 2 hours. Toluene (10 mL) was added to the mixture and then concentrated. The residue was placed in a vacuum over for 12 hours at a temperature of 40° C. to afford (2R-N-2-adamantyl-1-(cyclopentylmethyl)piperazine-2-carboxamide as a white foam (0.29 g, 96.1%). ¹H NMR (400 MHz, CDCl₃) δ ppm: 7.83 (d, J=7.83 Hz, 1H), 4.75 (dd, J=10.10, 3.80 Hz, 1H), 4.08-3.79 (m, 5H), 3.72-3.62 (m, 2H), 3.15 (d, J=7.33 Hz, 1H), 2.28 (qn, J=7.83 Hz, 1H), 1.98-1.60 (m, 24H), 1.33-1.14 (m, 1H). LCMS (ACPl): m/z [M+H]: 346.2.

### Method Q

### Example 170

### N-2-Adamantyl-1′-{2-[(tert-butoxycarbonyl)amino]-2-methylpropyl}-D-prolinamide

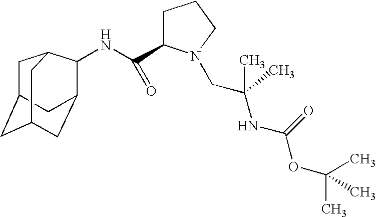

N-2-Adamantyl-D-prolinamide hydrochloride (780 mg, 2.74 mmol, 1.23 eqv) was added in one portion to a suspension of tert-butyl(1,1-dimethyl-2-oxoethyl)carbamate (418 mg, 2.23 mmol, 1 eqv) and sodium cyanoborohydride (590 mg, 8.9 mmol, 4.0 eqv) in methanol (15 mL) at 0° C. The reaction mixture was warmed to a temperataure of about 24° C. after 5 minutes. After 24 hours, methanol was removed in vacuo (at a pressure of about 25 mm Hg). The resulting residue was diluted with saturated aqueous ammonium chloride (30 mL) and extracted with dichloromethane (twice with 15 mL). The organic extracts were combined and washed with saturated aqueous sodium chloride (20 mL), dried over sodium sulfate, filtered, and concentrated. Purification using Biotage (0→5% methanol in dichloromethane followed by 5→10% methanol in dichloromethane with 1% ammonium hydroxide) yielded the named product as a clear colorless oil (82 mg, 9%).

### Method R

### Example 171

### N-2-adamantyl-1-(2-amino-2-methylpropyl)-D-prolinamide

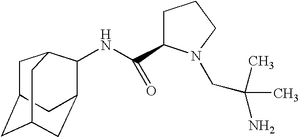

Trifluoroacetic acid (1 mL) was added dropwise to a solution of N-2-adamantyl-1-{2-[(tert-butoxycarbonyl)amino]-2-methylpropyl}-D-prolinamide (82 mg, 0.20 mmol, 1 eqv) in dichloromethane (3 mL) at a temperature of about 24° C. After 1 h, the reaction mixture was concentrated in vacuo (at a pressure of about 25 mm Hg). The resulting residue was purified using a Biotage (0→5.5% methanol in dichloromethane with 1% ammonium hydroxide) to yield the named product (58 mg, 93%).

Analysis and Purification Procedures for Final Products related to Methods S through T

The crude reaction mixtures were analyzed by HPLC using Analytical Method 1 (LC/MS/UV). Prior to purification, all samples were filtered through Whatman® GF/F Unifilter (#7700-7210). Purification of samples was performed by reverse phase HPLC using three different methods (see below). HPLC fractions were collected in 23 mL pre-tared tubes and centrifugal evaporated to dryness. Dried product was weighed and dissolved in DMSO. Products were then analyzed using Analytical Method 2 (LC/MS/UV/ELSD) and submitted for screening.

Analytical LCMS Method 1 (Pre-purification)

Column: Peeke Scientific HI-Q C-18, 50×4.6 mm, 5 μm, Eluent A: Water with 0.05% TFA, Eluent B: Acetonitrile with 0.05% TFA, Gradient: linear gradient of 0-100% B in 3.0 min, then 100% B for 0.5 min, then 100-0% B in 0.25 min, hold 100% A for 0.75 min, Flow: 2.25 mL/min, Column Temperature: 25° C., Injection Amount: 15 μL of a 286 μM crude solution in methanol/DMSO/water 90/5/5, UV Detection: 260 and 210 nm, Mass Spectrometry: APCI, positive mode, mass scan range 111.6-1000 amu.

Analytical LCMS Method 2 (Post-Purification)

Column: Peeke Scientific HI-Q C-18, 50×4.6 mm, 5 μm, Eluent A: Water with 0.05% TFA, Eluent B: Acetonitrile with 0.05% TFA, Gradient: linear gradient of 0-100% B in 1.75 min, then 100% B for 0.35 min, then 100-50% B for 0.5 min, Flow: 3.00 mL/min, Column Temperature: 25° C., Injection Amount: 15 μL of a 300 mM solution in methano/DMSO 99/1, UV Detection: 260 nm, Mass Spectrometry: APCI, positive mode, mass scan range 100-1000 amu, ELSD: gain=9, temp 40° C., nitrogen pressure 3.5 bar.

Preparative LC Method 1 (Gilson)

Column: Peeke Scientific HI-Q C18, 50 mm×20 mm, 5 mm, Eluent A: 0.05% TFA in Water, Eluent B: 0.05% TFA in Acetonitrile, Pre-inject Equilibration: 0.50 min, Post-inject Hold: 0.16 min, Gradient: 0-100% B in 2.55 min, then ramp 100% back to 0% in 0.09 min, Flow: 50.0 mL/min, Column Temp: Ambient, Injection Amount: 1200 μL of filtered crude reaction mixture in DMSO, Detection: UV at 210 nm or 260 nm.

Preparative LC Method 2 (Dionex)

Column: Peeke Scientific® HI-Q C18, 50 mm×20 mm, 5 μm, Eluent A: 0.05% TFA in Water, Eluent B: 0.05% TFA in Acetonitrile, Pre-inject Equilibration: 1.53 min, Post-inject Hold: 0.01 min, Gradient: 0-100% B in 5.1 min, hold 100% B for 1.5 min, then ramp 100% back to 0% B in 0.25 min, Flow: 25.0 mL/min, Column Temp: Ambient, Injection Amount: 1200 FL of filtered crude reaction mixture in DMSO, Detection: UV at 220, 240, 260 and 280 nm, collection triggered at 220 nm.

Preparative LC Method 3 (Waters)

Column: Peeke Scientific® HI-Q C18, 50 mm×20 mm, 5 μm, Eluent A: 0.05% TFA in Water, Eluent B: 0.05% TFA in Acetonitrile, Pre-inject Equilibration: 1.0 min, Post-inject Hold: 1.00 min, Gradient: Hold 5% B for 1.0 min, then ramp 5%-90% B over 2.55 min, hold 90% B for 0.2 min, then ramp 90% back to 5% B in 0.10 min, Flow: 50.0 mL/min, Column Temp: Ambient, Injection Amount: 1200 μL of filtered crude reaction mixture in DMSO, Detection: ESI-MS positive mode, 120-1000 amu.

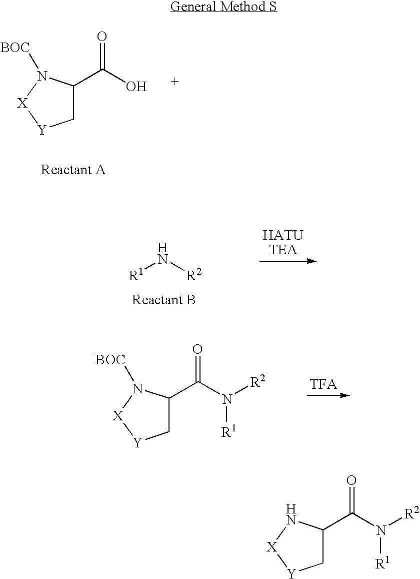

The Boc-protected amino acid (Reactant A, 400 μL, 0.1 mmol, 1.00 eq, 0.25 M in anhydrous DMF), the amine (Reactant B, 400 mL, 0.1 mmol, 1.00 eqv, 0.25 M in anhydrous DMF), HATU (200 μL, 0.103 mmol, 1.03 eqv, 0.52 M in anhydrous DMF), and TEA (42 μL, 0.3 mmol, 3.0 eqv) were added to a well of a 2 mL deep-well plate. The plate was sealed with a Teflon/Silicone-lied plate vice and heated in an oven at 60° C. for 16 h. The solvent was evaporated and TFA (250 μL, 3.2 mmol, 32 eqv) was added to the residue. The plate was sealed with the Teflon/Silicone-lied plate vice and vortexed at temperature of about 20° C. for 5 hours. The TFA was evaporated and the residue was dissolved in a mixture of EtOAc/EtOH/30% aq. ammonia (2:2:1). The plate was sealed with the plate vice and vortexed until the residue was dissolved. The solvent was evaporated and the residue was dissolved in DMSO (1.325 mL) containing 0.01% BHT to yield a 0.714 M solution. The solution was injected into an automated HPLC system for purification. The solvent of the product containing fraction was evaporated, the residue dissolved in DMSO, analyzed, and submitted for screening.

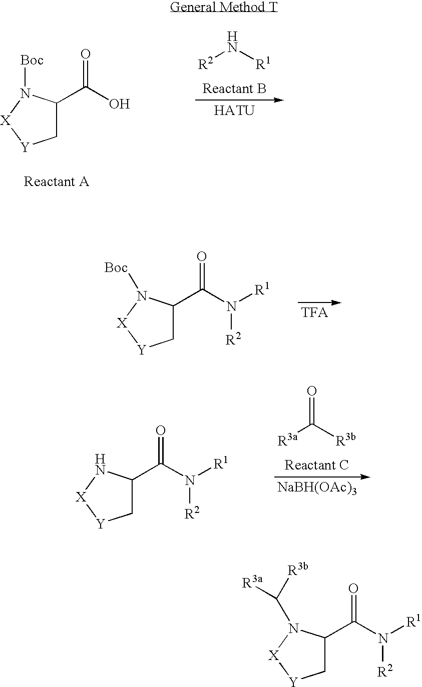

The Boc protected amino acid (Reactant A, 320 μL, 80 μmol, 1.00 eq, 0.25 M in anhydrous DMF), TEA (80 μL, 160 μmol, 2.00 eq, 2 M solution in anhydrous DMF), the amine (Reactant B, 320 μL, 80 μmol, 1.00 eqv, 0.25 M solution in anhydrous DMF), and HATU (320 μL, 80 μmol, 1.00 eqv, 0.25 M in anhydrous DMF) are added to a 13×100 mm test tube. The test tube was sealed and vortexed at a temperature of about 20° C. overnight (over 20 hours). The solvent was evaporated, the residue was dissolved in DCE (1600 μL) and the resulting solution was washed with 5% aq. NaHCO₃ (1050 μL) and water (1050 μL). The aq. phase was re-extraced with DCE (1050 μL) and the organic phases were combined. The solvent was evaporated. TFA (425 μL, 1.7 mmol, 21 eq, 4 M in DCE) was added and the reaction was vortexed for at least 24 h at a temperature of about 20° C. The solvent and excess TFA was evaporated. DMF (105 μL) and DI PEA (105 μL) were added and the test tube was vortexed for 1 h at a temperature of about 20° C. The aldehyde (Reactant C, 320 μL, 80 μmol, 1.00 eq, 0.25 M in DCE) and NaBH(OAc)₃ (1050 μL, 263 μmol, 3.28 eq, 0.25 M suspension in DCE) were added. The test tube was sealed and vortexed for over 20 hours at a temperature of about 20° C. The reaction mixture was washed with NH₃ (1350 μL, 10% in water), the aq. NH₃ was re-extracted with DCE (1050 μL), the organic phases were combined, and the solvent evaporated. The solvent was evaporated and the residue was dissolved in DMSO containing 0.01% BHT to yield a 0.0575 M solution. The solution was injected into an automated HPLC system for purification. The solvent of the product containing fraction was evaporated, the residue dissolved in DMSO, analyzed, and submitted for screening.

### Synthesis Procedures for Non-Commercial Starting Materials

### Synthesis of endo and exo-2-[tert-butoxycarbonyl)-2-azabicyclo[2.2.1]heptane-3-carboxylic acid

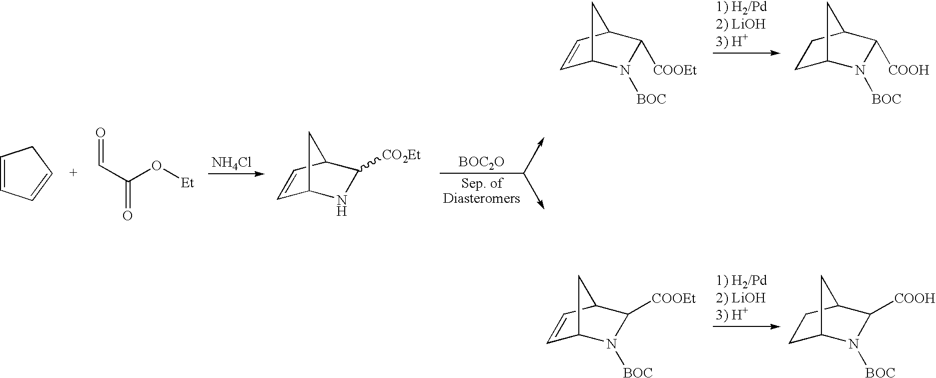

Freshly distilled cyclopentadiene (1 atm, 41° C., 40 cm Vigrox column, 16.5 g), saturated aq. ammonia chloride (800 mL) and ethyl glyoxylate (75 mL, 50% in toluene) were vigorously stirred overnight at a temperature of about 20° C. The acidic mixture was extracted twice with hexanes/ether 3:1 and then treated with 50% NaOH until a pH of 9 to 11 was reached. The now basic mixture was extracted with ether (3 times) and the combined extracts were dried over MgSO₄, filtered, and concentrated to yield a yellow oil (38 g) that was used directly in the next step. The crude intermediate was dissolved in THF (200 mL) and TEA (15 mL). In portions, (BOC)₂O (55 g) was added. The reaction was exotherm and developed CO₂. The mixture was stirred overnight at a temperature of about 20° C. The solvent was evaporated, the residue was dissolved in hexanes/EtOAc 1:1 and washed with water (twice). The organic phase was dried over MgSO₄, filtered, and concentrated. The endo and exo isomers were separated by column chromatography using 15 to 25% EtOAc in hexanes. The mixed fractions were repurified by column chromatography to give 36.3 g of the endo and 12.0 g of the exo product.

12.0 g of the exo product was dissolved in 200 mL EtOAc and 0.5 g 10% Pd/C was added. The mixture was hydrogenated using a Parr hydrogenator. After 13 fillings of the flask, the hydrogenation was complete. The mixture was filtered, the filter washed with EtOAc, and the filtrate concentrated. The crude ester was dissolved in 25 mL THF and 25 mL MeOH and a solution of 3.5 g LiOH monohydrate in 50 mL water was added. The mixture was stirred for 24 h at a temperature of about 20° C. After evaporation, acidification to pH 4, and extraction with ether the exo acid was obtained with a contamination of 10% of the endo product. The exo acid was isolated in pure form by recrystallization from ether/hexanes (6.7 g, 62%). H-NMR (300 MHz, CDCl₃) δ=4.1 (s, 1H), 3.8 (s, 1H), 2.9 (br s, 1H), 1.8-1.6 (m, 4H), 1.4 (s, 9H), 1.3 (br, 2H).

The endo product was obtained in a way similar to the one used for the synthesis of the exo product. H-NMR (300 MHz, CDCl₃) δ=7.75 (br, 1H) 4.35 (s, 1H) 4.20 (s, 1H) 2.80 (s, 1H) 1.80 (br, 2H) 1.70-1.40 (m, 4H) 1.40 (s, 9H).

### General Reaction Scheme for the Synthesis of (2S,4S)-4-(4-aroxy)-pyrrolidine-1,2-dicarboxylic acid 1-tert-butyl esters

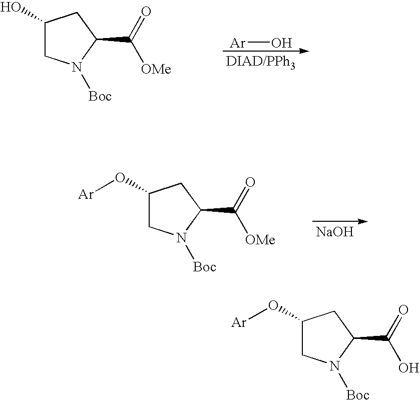

### Prep-1: (2S,4S)-4-(4-Fluoro-phenoxy)-pyrrolidine-1,2-dicarboxylic acid 1-tert-butyl ester

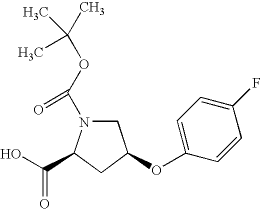

### 1-(tert-Butyl) 2-methyl(2S,4S)-4-(4-fluorophenoxy)tetrahydro-1H-1,2-pyrroledicarboxylate

1-(tert-Butyl)2-methyl(2S,4R)-4-hydroxytetrahydro-1H1,2-pyrroledicarboxylate (39.78 g, 0.162 mol), triphenylphosphine (46.74 g, 0.178 mol) and 4-fluorophenol (20.0 g, 0.178 mol) were dissolved in THF (200 mL). After all components were dissolved a solution of DIAD (39.31 g, 0.186 mol) in THF (50 mL) was added drop wise under cooling. The mixture was kept to stir for 15 h. Then THF was evaporated. Ether (250 mL) and hexane (200 mL) were added to the reaction mixture. The precipitate formed was filtered and the solvent was evaporated to furnish 72.32 g of product as viscous oil.

### 4-(4-Fluoro-phenoxy)-pyrrolidine-1,2-dicarboxylic acid 1-tert-butyl ester

Crude 1-(tert-butyl) 2-methyl(2S,4S)-4-(4-fluorophenoxy)tetrahydro-1H1,2-pyrroledicarboxylate (72.32 g, 0.162 mol) was dissolved in 300 mL of methanol. NaOH solution (16.2 g, 0.405 mol in 50 mL of water) was added to the mixture. Then the mixture was stirred at a temperature of about 20° C. for 10 h. Methanol was evaporated and the residue was treated with 400 mL of water. The precipitate was filtered and the filtrate was extracted with dichloromethane (twice with 200 mL), acidified with 20% solution of citric acid to pH 5 and the product was extracted with dichloromethane (three times with 150 mL). The organic extracts were dried (Na₂SO₄) and the solvent was evaporated. The residue was dissolved in 200 mL of ether and 200 mL of hexane to furnish after crystallization 24.3 g of 4-(4-fluoro-phenoxy)-pyrrolidine-1,2-dicarboxylic acid 1-tert-butyl ester as colorless crystals. Additional 5.1 g of this compound was obtained from the mother solution. The total yield of was 53% (29.4 g). Satisfactory C, H, N-analysis was obtained. LCMS: 1.68 min, 324 m/z. H-NMR (400 MHz, CDCl₃) δ=7.00-7.89 (m, 2H), 7.80-7.69 (m, 2H), 4.85 (d, 1H), 4.60-4.43 (m, 1H), 3.79-3.63 (m, 2H), 2.76-2.73 (M, 1H), 2.50 (br, 1H), 2.30 (br, 1H), 1.45 (s, 9H).

The compounds in Table 1 were prepared in a similar way.

TABLE 1

| Preparation | Structure and Name | H-NMR | LCMS |
|---|---|---|---|
| Prep-2 | 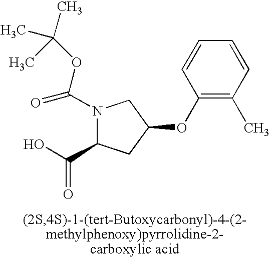 | H-NMR (400 MHz, CDCl₃) δ =7.18-7.08 (m, 2H), 6.90-6.82 (m, 1H), 6.71 (d2, 1H), 4.92 (s, 1H), 4.55 (br s, 1H), 3.70-3.65 (m, 2H), 2.80 (br, 1H), 2.60-2.35 (m, 1H), 2.12 (s, 3H), 1.45 (s, 9H). | LCMS: 6.82 min, 320.9 m/z |
| Prep-3 | 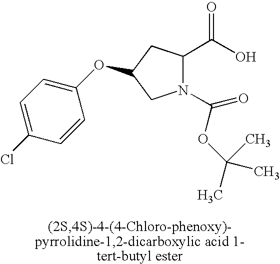 | H-NMR (300 MHz, CDCl₃) δ =9.48 (br, 1H) 7.26-7.14 (m, 2H) 6.82-6.74 (m, 2H) 5.00 (s, 1H) 4.88-4.55 (m, 1H) 3.74 (br, 2H) 2.68-2.29 (m, 2H) 1.48 (s, 9H). | LCMS: 4.96 min, 242.0 m/z. |
| Prep-4 | 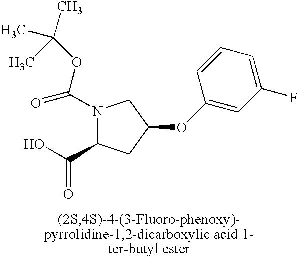 | H-NMR (400 MHz, CDCl₃) δ =7.25-7.15 (m, 1H), 6.68-6.51 (m, 3H), 5.11 (s, 1H), 4.52 (br s, 1H), 3.80-3.65 (m, 2H), 2.85-2.75 (br m, 1H), 2.55 (br, 1H), 2.40 (br, 1H), 1.49 (s, 9H). | LCMS: 1.699 min, 324.0 m/z. |
| Prep-5 | 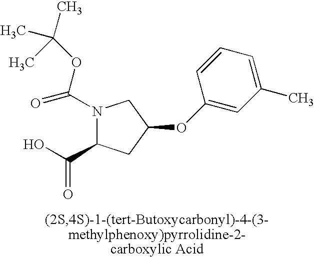 | H-NMR (400 MHz, CDCl₃) δ =7.25 (s, 1H) 7.15 (br, 1H) 6.75 (d, 1H) 6.65 (br, 2H) 4.90 (s, 1H) 4.60-4.45 (m, 1H) 3.80-3.65 (m, 2H) 2.75 (br, 1H) 2.52 (br, 1H) 2.30 (s, 3H) 1.49 (s, 9H). | LCMS: 1.78 min, 221.9 m/z. |
| Prep-6 | 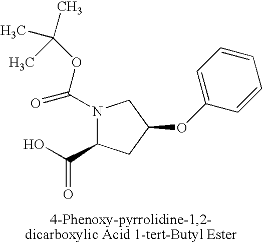 | H-NMR (400 MHz, CDCl₃) δ =7.25 (br, 2H) 6.95 (br, 1H) 6.80 (br, 2H) 4.95 (s, 1H) 4.60-4.45 (m, 1H) 3.80-3.65 (m, 2H) 2.75-2.70 (m, 1H) 2.52 (br, 1H) 2.40 (br, 1H) 1.49 (s, 9H). | LCMS: 1.685 min, 330.2 m/z |
| Prep-7 | 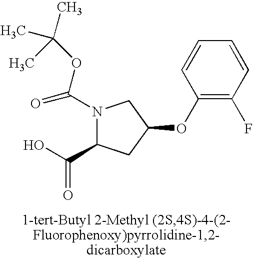 | H-NMR (400 MHz, CDCl₃) δ =12.53 (br, 1H) 7.10 (t, 1H) 7.15 (d, 1H) 6.95-6.90 (m, 1H) 5.05 (br, 1H) 4.35-4.25 (m, 1H) 3.80-3.70 (m, 1H) 3.45 (d, 1H) 2.70-2.55 (m, 1H) 2.25-2.20 (d, 1H) 1.45 (s, 4H). | LCMS: 1.70 min, 3.24.0 m/z. |
| Prep-8 | 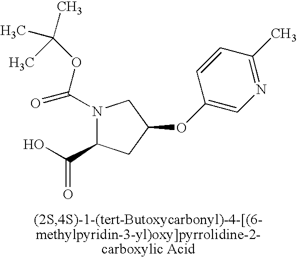 | H-NMR (400 MHz, DMSO-d₆) δ = 12.55 (br, 1H) 8.05 (s, 1H) 7.25 (d, 1H) 7.15 (d, 1H) 5.05 (br, 1H) 4.28 (t, 1H) 3.70 (dt, 1H) 3.40-3.25 (m, 1H) 2.65-2.55 (m, 1H) 2.40 (s, 3H) 2.15 (d, 1H) 1.40 (s, 4H) 1.35 (s, 5H) | LCMS: 1.151 min, 323.3 m/ |
| Prep-9 | 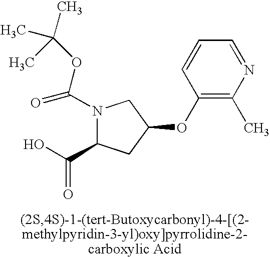 | H-NMR (400 MHz, DMSO-d₆) δ = 12.55 (br, 1H) 8.00 (d, 1H) 7.30 (d, 1H) 7.15 (t, 1H) 5.05-5.00 (m, 1 H) 4.41 (dd, 1H) 3.75 (dt, 1H) 3.40 (t, 1H) 2.70-2.55 (m, 1H) 2.30 (s, 3H) 2.25-2.20 (m, 1H) 1.40 (s, 4H) 1.39 (s, 5H). | LCMS: 1.170 min, 323.1 m/z |
| Prep-10 | 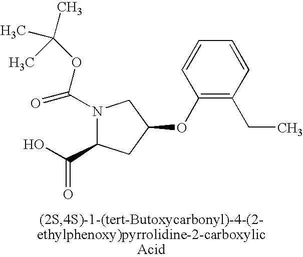 | H-NMR (400 MHz, CDCl₃) δ =7.15 (br, 2H) 6.90 (br, 1H) 6.73 (d, 1H) 4.90 (br, 1H) 4.51 (dd, 1H) 3.85-3.60 (m, 1H) 2.80 (d, 1H) 2.60-2.40 (m, 3H) 1.50 (s, 9H) 1.15 (br, 3H). | LCMS: 1.773 min, 334.0 m/z. |
| Prep-11 | 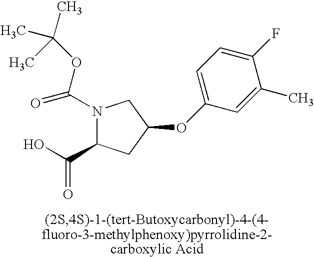 | H-NMR (400 MHz, CDCl₃) δ =6.85 (br, 1H) 6.60 (br, 2H) 4.85 (s, 1H) 4.50 (brd, 1H) 3.70 (brd, 1H) 2.75 (brd, 1H) 2.50 (br, 1H) 2.35 (br, 1H) 2.15 (s, 3H) 1.49 (s, 6H) 1.48 (s, 3H). | LCMS: 1.375 min, 338.0 m/z. |
| Prep-12 | 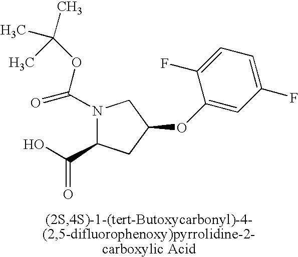 | H-NMR (400 MHz, CDCl₃) δ =7.00 (br, 1H) 6.75-6.55 (m, 2H) 4.95 (s, 1H) 4.60-4.45 (m, 1H) 3.85-3.66 (m, 2H) 2.85-2.75 (m, 1H) 2.65-2.50 (m, 1H) 2.55-2.45 (m, 1H) 1.55 (s, 6H) 1.50 (s, 3H) | LCMS: 6.53 min, 341.9 m/z. |
| Prep-13 | 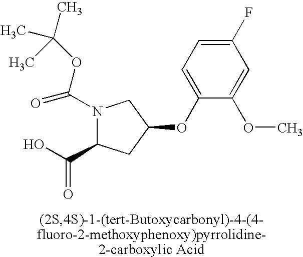 | H-NMR (400 MHz, CDCl₃) δ =6.90 (br, 1H) 6.55-6.50 (m, 2H) 4.75 (br, 1H) 4.55-4.40 (m, 1H) 3.80 (s, 3H) 3.75-3.65 (m, 1H) 3.55-3.35 (m, 2H) 2.75 (br, 1H) 2.55 (br, 1H) 2.30 (br, 1H) 1.50 (s, 9H). | LCMS: 1.701 min, 354.0 m/z. |
| Prep-14 | 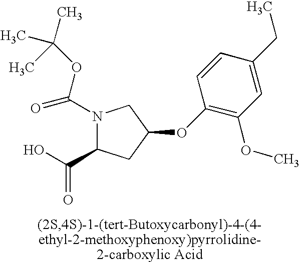 | H-NMR (400 MHz, CDCl₃) δ =12.50 (br, 1H) 6.85 (s, 1H) 6.80 (t, 1H) 6.65 (d, 1H) 4.85 (s, 1H) 4.25 (t, 1H) 3.75 (s, 3H) 3.65 (dt, 1H) 3.30 (m, 1H) 2.55-2.45 (q, 2H) 2.10 (d, 1H) 1.40 (s, 4H) 1.35 (s, 5H) 1.10 (t, 3H). | LCMS: 1.848 min, 364.0 m/z. |
| Prep-15 | 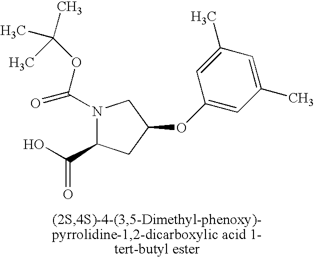 | H-NMR (400 MHz, CDCl₃) δ =6.65 (s, 1H) 6.45 (s, 2H) 4.90 (s, 1H) 4.45-4.35 (m, 1H) 3.75-3.60 (m, 2H) 2.75 (m, 1H) 2.50 (br, 1H) 2.40 (br, 1H) 2.25 (s, 6H) 1.50 (s, 9H). | LCMS: 1.858 min, 334.0 m/z. |
| Prep-16 | 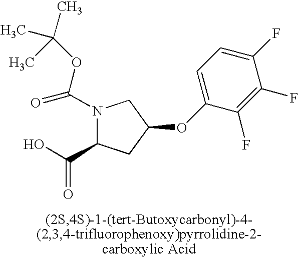 | H-NMR (400 MHz, DMSO-d₆) δ =12.60 (br, 1H) 7.25 (dd, 1H) 7.00 (br, 1H) 5.05 (br, 1H) 4.25 (t, 1H) 3.75 (dt, 1H) 3.40 (d, 1H), 2.65-2.50 (m, 1H) 2.25-2.15 (m, 1H) 1.40 (s, 4H) 1.35 (s, 5H). | LCMS: 1.753 min, 359.9 m/z. |
| Prep-17 | 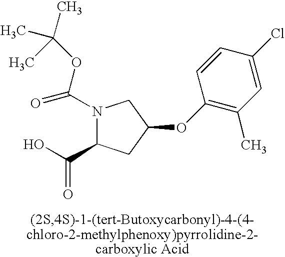 | H-NMR (400 MHz, CDCl₃) δ =7.10-7.00 (m, 2H) 6.60 (d, 1H) 4.85 (s, 1H) 4.60-4.45 (m, 1H) 3.70-3.60 (m, 2H) 2.78-2.70 (m, 1H) 2.55 (br, 1H) 2.40 (br, 1H) 2.05 (s, 3H) 1.50 (s, 6H) 1.45 (s, 3H). | LCMS: 1.888 min, 354.0 m/z. |
| Prep-18 | 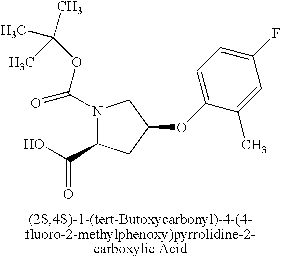 | H-NMR (400 MHz, DMSO-d₆) δ = 12.55 (br, 1H) 7.05 (d, 1H) 6.95 (t, 1H) 6.90-6.80 (m, 1H) 5.00-4.90 (m, 1H) 4.30 (dd, 1H) 3.70 (dt, 1H) 3.38 (d, 1H) 2.60-2.50 (m, 1H) 2.25-2.15 (m, 1H) 2.05 (s, 3H) 1.40 (s, 5H) 1.35 (s, 4H). | LCMS: 1.888 min, 354.0 m/z. |
| Prep-19 | 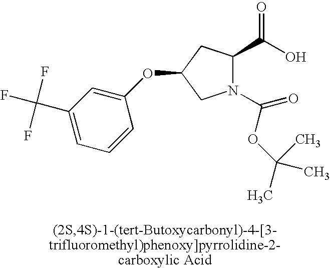 | H-NMR (400 MHz, DMSO-d₆) δ = 12.55 (br, 1H) 7.55 (t, 1H) 7.30 (d, 1H) 7.20 (d, 1H) 7.15 (s, 1H) 5.15-5.05 (m, 1H) 4.30 (t, 1H) 3.70 (dt, 1H) 3.40 (dd, 1H) 2.65-2.55 (m, 1H) 2.20-2.10 (m, 1H) 1.40 (s, 5H) 1.35 (s, 4H). | LCMS: 1.846 min, 373.9 m/z. |
| Prep-20 | 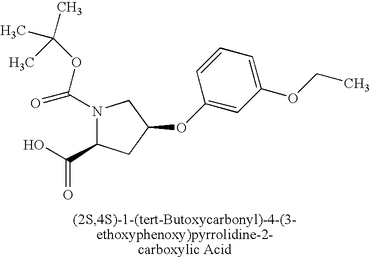 | H-NMR (400 MHz, DMSO-d₆) δ = 12.45 (br, 1H) 7.15 (t, 1H) 6.50 (d, 1H) 6.45 (d, 1H) 6.35 (brd, 1H) 5.00 (brs, 1H) 4.25 (t, 1H) 4.00 (q, 2H) 3.70 (dt, 1H) 3.40-3.00 (m, 1H) 2.60-2.50 (m, 1H) 2.10 (d, 1H) 1.40 (s, 3H) 1.35 (s, 6H) 1.25 (t, 3.H). | LCMS: 1.771 min, 350.0 m/z. |
| Prep-21 | 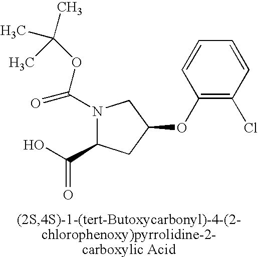 | H-NMR (400 MHz, DMSO-d₆) δ = 12.50 (br, 1H) 7.40 (d, 1H) 7.28 (t, 1H) 7.10 (d, 1H) 6.95 (t, 1H) 5.05 (br, 1H) 4.30 (brt, 1H) 3.85-3.75 (m, 1H) 3.40 (d, 1H) 2.70-2.50 (m, 1H) 2.20 (d, 1H) 1.40 (s, 4H) 1.35 (s, 5H). | LCMS: 1.735 min, 339.9 m/z |
| Prep-22 | 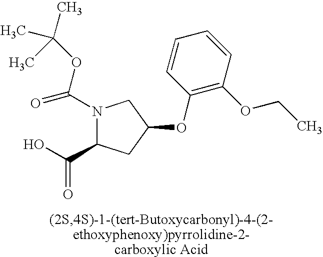 | H-NMR (400 MHz, DMSO-d₆) δ = 12.50 (br, 1H) 7.05-6.80 (m, 4H) 4.90 (s, 1H) 4.25 (t, 1H) 3.95 (q, 2H) 3.65 (dt, 1H) 3.35 (d, 1H) 2.65-2.55 (m, 1H) 2.15 (dd, 1H) 1.40 (s, 4H) 1.35 (s, 5H) 1.25 (t, 3H). | LCMS: 1.755 min, 350.0 m/z |
| Prep-23 | 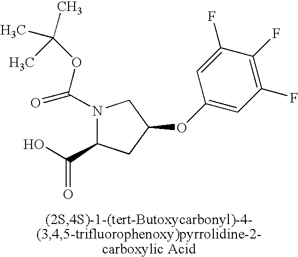 | H-NMR (400 MHz, DMSO-d₆) δ = 12.55 (br, 1H) 6.95-6.85 (m, 2H) 5.00 (br, 1H) 4.35 (t, 1H) 3.70 (dt, 1H) 3.40-3.35 (m, 1H) 2.65-2.50 (m, 1H) 2.15-2.05 (m 1H) 1.40 (s, 4H) 1.35 (s, 5H). | LCMS: 9.142 min, 359.9 m/z. |
| Prep-24 | 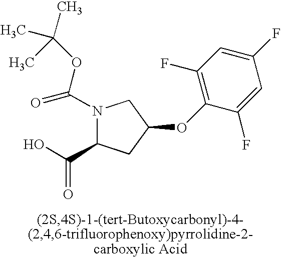 | H-NMR (400 MHz, DMSO-d₆) δ = 12.50 (br, 1H) 7.20 (t, 2H) 4.80 (s, 1H) 4.25 (t, 1H) 3.70 (dt, 1H) 3.50 (d, 1H) 3.30 (br, 1H) 2.60-2.50 (m, 1H) 2.25-2.15 (m, 1H) 1.40 (s, 4H) 1.35 (s, 5H). | LCMS: 1.743 min, 359.8 m/z |
| Prep-25 | 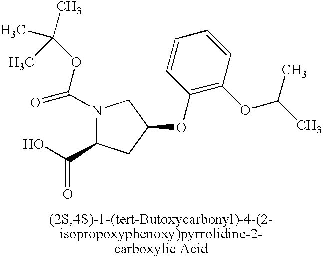 | H-NMR (400 MHz, DMSO-d₆) δ = 12.50 (br, 1H) 7.05-6.85 (m, 4H) 5.10 (s, 1H) 4.45 (quin, 1H) 4.20 (brt, 1H) 3.70 (brdt, 1H) 3.35 (brd, 1H) 2.60-2.50 (m, 1H) 2.20-2.15 (m, 1H) 1.40 (s, 3H) 1.35 (s, 6H) 1.15 (d, 6H). | LCMS: 1.862 min, 364.0 m/z. |
| Prep-26 | 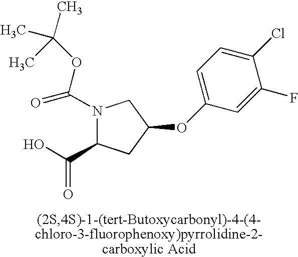 | H-NMR (400 MHz, DMSO-d₆) δ = 12.55 (s, 1H) 7.45 (t, 1H) 7.00 (brd, 1H) 6.70 (brd, 1H) 5.05 (br, 1H) 4.25 (t, 1H) 3.70 (dt, 1H) 3.40-3.35 (m, 1H) 2.65-2.55 (m, 1H) 2.15 (brd, 1H) 1.40 (s, 4H) 1.35 (s, 5H). | LCMS: 9.346 min, 357.9 m/z. |

### General Reaction Scheme for the Synthesis of trans-1-(tert-Butoxycarbonyl)-3-alkyl-pyrrolidine-2-carboxylic Acids and trans-1-(tert-Butoxycarbonyl)-3-aryl-pyrrolidine-2-carboxylic Acids

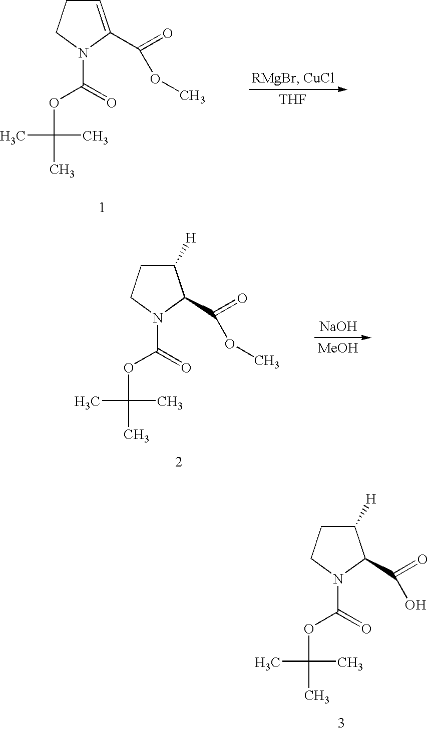

### Prep-27: trans-1-(tert-Butoxycarbonyl)-3-isopropylpyrrolidine-2-carboxylic Acid

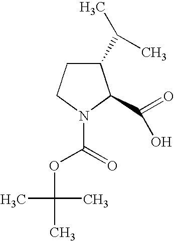

### trans-1-tert-Butyl 2-Methyl-3-isopropylpyrrolidine-1,2-dicarboxylate (2)

1 M Solution of ePrMgBr in THF (800 mL, 0.8 mol) was added at 60° C. to a suspension of CuCl (39.6 g, 0.4 mol) in absolute THF (300 mL). After the addition was completed, the reaction mixture was heated to −30° C. and left to stand at this temperature for 60 min. Then the reaction mixture was cooled again to −80° C., and 1-tert-butyl 2-methyl 4,5-dihydro-1H-pyrrole-1,2-dicarboxylate (compound of formula 1; 45.4 g, 0.2 mol) was added over a period of 1 h at this temperature. After 1 h, the mixture was quenched at −70° C. with citric acid (200 g) and water (400 mL). The organic layer was separated, and the aqueous one was extracted with ether (twice with 200 mL). The combined organic extracts were dried over anhydrous Na₂SO₄ and evaporated. The obtained liquid residue was dissolved in ether (400 mL) and passed through a layer of SiO₂ (six times with 12 cm), eluting with ether to give 63.7 g of 2 (R_{f} 0.48).

### trans1-(tert-Butoxycarbonyl)-3-butylpyrrolidine-2-carboxylic Acid (3)

NaOH (20 g, 0.5 mol) and water (70 mL) were added to a solution of ester having the formula of compound 2 (63.7 g) in THF (200 mL) and methanol (200 mL). After the addition was completed, the reaction mixture was stirred at a temperature of about 20° C. for 16 h, then evaporated to 100 mL and quenched by the addition of water (400 mL). The mixture was then washed with toluene (300 mL), and the aqueous layer was separated and acidified with citric acid (60 g). The product was extracted with dichloromethane (twice with 200 mL), and the combined organic extract was dried over Na₂SO₄ and evaporated. The liquid residue was recrystallized from hexane (200 mL) to give a compound of formula 3 as white crystals in 64.3% (33.1 g) yield. Satisfactory C, H, N-analysis was obtained. LCMS: 1.285 min, 256.1 m/z. H-NMR (400 MHz, DMSO) δ=12.45 (br, 1H) 3.78 (dd, 1H) 3.45-3.35 (m, 1H) 3.30-3.15 (m, 1H) 2.05-1.85 (m, 2H) 1.70-1.63 (m, 2H) 1.40 (s, 4H) 1.35 (s, 4H) 0.88 (d, 3H) 0.80 (d, 3H).

The compounds in Table 2 were prepared in a similar way.

TABLE 2

| Preparation | Structure and Name | H-NMR | LCMS |
|---|---|---|---|
| Prep-28 | 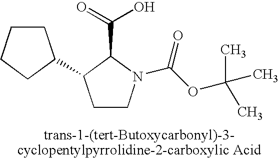 | H-NMR (300 MHz, DMSO-d6) δ = 3.82 (d, 1H) 3.45-3,25 (m, 2H) 2.15-2.03 (m, 1H) 2.05-1.45 (m, 8H) 1.37 (s, 9H) 1.30-1.05 (m, 2H). | LCMS: 5.28 min, 184.2 m/z. |
| Prep-29 | 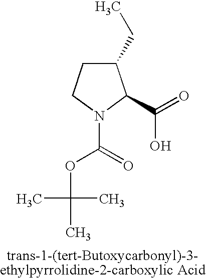 | H-NMR (400 MHz, DMSO-d6) δ = 12.45 (br, 1H) 3.70 (dd, 1,H) 3.45-3.35 (m, 1H) 3.30-3.15 (m, 1H) 2.10-1.95 (m, 2H) 1.60-1.45 (m, 2H) 1.35 (s, 3H) 1.30 (s, 6H) 0.90 (t, 3H) | LCMS: 1.617 min, 242.1 m/z. |
| Prep-30 | 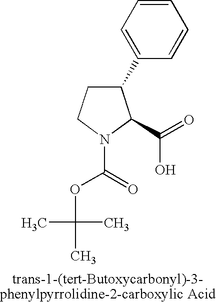 | H-NMR (400 MHz, DMSO-d6) δ = 12.50 (br, 1H) 7.40-7.15 (m, 5H) 4.05 (dd, 1H) 3.60-3.50 (m, 1H) 3.45-3.30 (m, 2H) 2.25-2.10 (m, 1H) 1.95-1.90 (m, 1H) 1.45 (s, 5H) 1.30 (s, 4H). | LCMS: 1.330 min, 290.1 m/z. |
| Prep-31 | 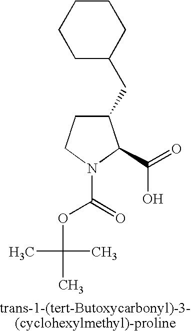 | H-NMR (400 MHz, DMSO-d6) δ = 12.40 (br, 1H) 3.65 (dd, 1H) 3.45-3.35 (m, 1H) 3.25-3.15 (m, 1H) 2.55-2.15 (m, 1H) 2.00-1.90 (m, 1H) 1.75-1.55 (m, 2H) 1.45-1.05 (m, 7H) 1.40 (s, 4H) 1.35 (s, 5H) 0.90-0.75 (m, 2H). | LCMS: 1.815 min, 310.1 m/z. |
| Prep-32 | 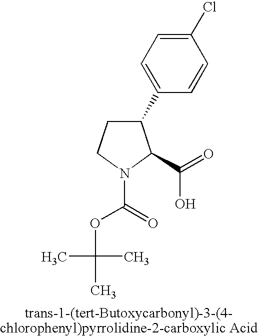 | H-NMR (400 MHz, DMSO-d6) δ = 12.55 (br, 1H) 7.40-7.25 (m, 4H) 4.00 (dd, 1H) 3.60-3.50 (m, 1H) 3.40-3.30 (m, 2H) 2.20 (m, 1H) 2.00 (m, 1H) 1.40 (s, 3H) 1.35 (s, 6H). | LCMS: 2.801 min, 323.7 m/z. |
| Prep-33 | 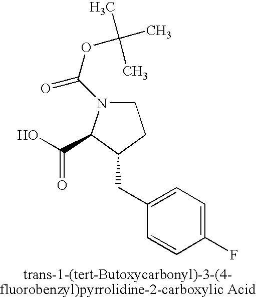 | H-NMR (400 MHz, DMSO-d6) δ = 12.50 (br, 1H) 7.25 (dd, 2H) 7.11 (t, 2H) 3.80 (dd, 1H) 3.45-3.35 (m, 1H) 3.30-3.20 (m, 1H) 2.85-2.75 (m, 1H) 2.70-2.60 (m, 1H) 2.45-2.35 (m, 1H) 1.85-1.75 (m, 1H) 1.60-1.50 (m, 1H) 1.40 (s, 3H) 1.35 (s, 6H) | LCMS: 2.786 min, 321.8 m/z. |
| Prep-34 | 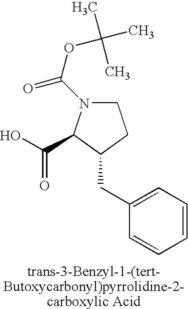 | H-NMR (400 MHz, DMSO-d6) δ = 12.50 (s, 1H) 7.30 (t, 2H) 7.23-718 (m, 3H) 3.80 (dd, 1H) 3.45-3.35 (m, 1H) 3.30-3.20 (m, 1H) 2.90-2.80 (m, 1H) 2.65 (t, 1H) 2.45-2.35 (m, 1H) 1.80-1.70 (m, 1H) 1.60-1.50 (m, 1H) 1.40 (s, 3H) 1.35 (s, 6H). | LCMS: 2.710 min, 303.8 m/z. |

The structure, name, physical and biological data, and Methods are further described in tabular form below in Table 3.

TABLE 3

| | | % inh | | | | |
|---|---|---|---|---|---|---|
| | Ki | @ | | | | |
| Ex- | app | 0.1 | Structure | | | MS |
| ample | (nM) | μM | IUPAC Name | Method | ¹H NMR | (m/z) |
| 1 | 2.1 | 100 | 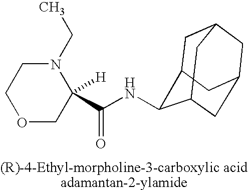 | A | (400 MHz, MeOD) δ: 3.96 (s, 1H), 3.80-3.88 (m, 2H), 3.62 (m, 1H), 3.46 (dd, J=11.24, 9.73 Hz, 1H), 2.94-3.04 (m, 2H), 2.67 (m, 1H), 2.19-2.29 (m, 2H), 1.79-1.93(m, 12H), 1.68 (d, J=12.13 Hz, 2H), 1.05-1.14 (m, 3H). | 293 |
| 2 | 2.4 | 100 | 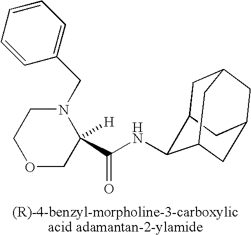 | A | (400 MHz, MeOD) δ: 7.27-7.36 (m, 5H), 4.85 (s, 2H), 3.96 (s, 1H), 3.86-3.91 (m, 1H), 3.85 9s, 1H), 3.67-3.77 (m, 1H), 3.5-3.60 (m, 2H), 3.28 (m, 2H), 3.07 9dd, J=9.35, 3.54 HZ, 1 H), 2.73-2.78 (m, 1H), 2.25 (m, 1H), 1.89 (s, 1H), 1.74-1.84 (m, 9H), 1.66 (m, 1H), 1.57 (d, J=12.13 HZ, 1H). | 355 |
| 3 | 15 | 92 | 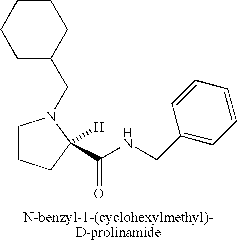 | A | (400 MHz, DMSO-D₆) δ7.95 (s, 1H), 7.29 (d, J=6.57 Hz, 2H), 7.23 (d, J=6.06 Hz, 3H), 4.15-4.20 (m, 2H), 3.05 (s, 1H), 2.84-2.93 (m, 1H), 2.13-2.31 (m, 3H), 1.98-2.09 (m, 2H), 1.52-1.87 (m, 6H), 1.03-1.34 (m, 5H), 0.69-0.80 (m, 1H), 0.54-0.65 (m, 1H). | 301 |
| 4 | 3.6 | 94 | 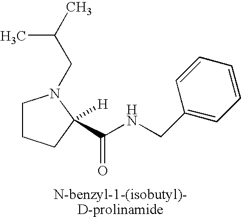 | A | (400 MHz, DMSO-D₆) δ9.55 (s, 1H), 9.10 (s, 1H), 7.23-7.35 (m, SH), 4.31-4.41 (m, 2H), 4.18-4.27 (m, 1H), 3.63-3.74 (m, 1H), 3.12-3.23 (m, 1H), 3.00 (s, 1H), 2.01 -2.12 (m,1H), 1.84-1.96 (m, 3H), 0.96 (d, J=6.57 Hz, 3 H), 0.90 (d, J=5.67 Hz, 3H). | 261 |
| 5 | NA | 29 | 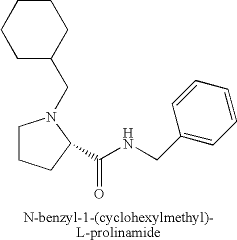 | A | (400 MHz, DMSO-D₆) δ7.95 (s, 1H), 7.29 (d, J=6.57 Hz, 2H), 7.23 (d, J=6.06 Hz, 3H), 4.15-4.20 (m, 2H), 3.05 (s, 1H), 2.84-2.93(m, 1H), 2.13-2.31 (m, 3H), 1.98-2.09 (m, 2H), 1.52-1.87 (m, 6H), 1.03-1.34 (m, 5H), 0.69-0.80 (m, 1H), 0.54-0.65 (m, 1H). | 301 |
| 6 | 1.3 | 100 | 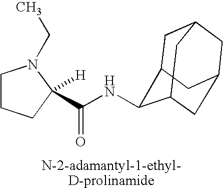 | A | (400 MHz, DMSO-D₆) δ7.42 (s, 1H), 8.78 (d, J=7.07 Hz, 1 H), 4.28 (q, J=7.83 Hz, 1H), 3.89 (d, J=7.33 Hz, 1H), 3.54-3.64 (m, 1H), 3.09-3.20 (m, 4H), 2.40-2.47 (m, 1H), 1.95-2.06 (m, 4H), 1.72-1.84 (m, 10H), 1.50 (d, J=12.38 Hz, 2H), 1.09-1.18(m, 3H). | 277 |
| 7 | 3.2 | 100 | 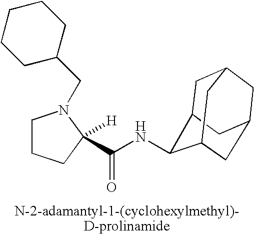 | A | (400 MHz, DMSO-D₆) δ8.98 (s, 1H), 8.51 9d, J=7.07 Hz, 1H), 4.12 (q, J=8.00 Hz, 1H), 3.88 (d, J=6.82 Hz, 1H), 3.65-3.72 (m, 1H), 3.16 (dd, J=10.86, 7.83 Hz, 1H), 2.93-3.03 9m, 2H), 2.39-2.48 (m, 2H), 1.99-2.03 (m, 1H), 1.50-1.99 (m, 21H), 1.04-1.19 (m, 3H), 0.86-0.96 (m, 2H). | 345 |
| 8 | 2.0 | 100 | 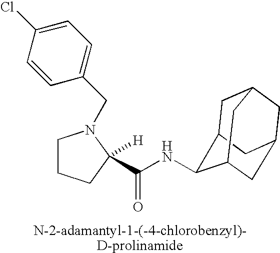 | A | (400 MHz, DMSO-D₆) δ9.64 (s, 1H), 8.42 (d, J=7.58 Hz, 1 H), 7.48-7.54 (m, 2H), 7.41 -7.47 (m, 2H), 4.39-4.47 (m, 1H), 4.34 (dd, J=11.87, 7.83 Hz, 2H), 3.56-3.67 (m, 2H), 3.22-3.42 (m, 4H), 2.00-2.11 (m, 1H), 1.63-1.94 (m, 10H), 1.24-1.47 (d, J=19.20 Hz, 2H), 0.79-0.89 (m, 1H). | 373 |
| 9 | 1.7 | 100 | 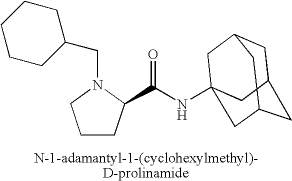 | A | (400 MHz, DMSO-D₆) δ8.91 (s, 1H), 8.42 (s, 1H), 3.91-4.04 (m, 2H), 3.61-3.72 (m, 1H), 3.14 (dt, J=18.63, 8.12 Hz, 1H), 2.96 (m, 2H), 2.36-2.47 (m, 1H), 2.02 (s, 4H), 1.71-1.98 (m, 10H), 1.50-1.69 (m, 10H), 1.10-1.22 (m, 3H), 0.84-0.96 (m, 2H). | 345 |
| 10 | 0.85 | 100 | 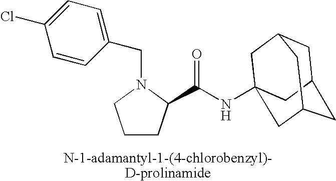 | A | (400 MHz, DMSO-D₆) δ9.50 (s, 1H), 7.87 (s, 1H), .44-7.50 (m, 4H), 4.40 (d, J=12.63 Hz, 1H), 4.24-4.32 (m, 1H), 3.93-4.03 (m, 1H), 3.55-3.64 (m, 1H), 3.21-3.31 (m, 1H), 2.32-2.42 (m, 1H), 2.00-2.10 (m, 1H), 1.95 (s, 3H), 1.70-1.86 (m, 2H), 1.68 (d, J=1.26 Hz, 6H), 1.51-1.61 (m, 6H). | 373 |
| 11 | 38.3 | 80 | 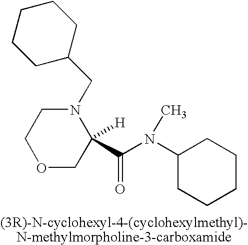 | B | ¹³C NMR (75.47 MHz, CD₃CN) δ 19.38, 24.67, 24.81, 24.97, 24.99, 25.05, 25.24, 25.30, 25.54, 25.60, 26.18, 26.60, 28.51, 28.68, 28.96, 28.99, 30.51, 30.63, 30.73, 30.92, 31.32, 34.20, 34.27, 50.46, 50.70, 52.46, 55.26, 61.31, 61.68, 65.74, 66.01, 66.79, 67.86, 168.64, 168.93 | 323 |
| 12 | 14 | 89 | 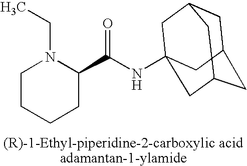 | A | (400 MHz, DMSO-D₆) δ3.66 (m, 1H), 3.45 (m, 1 H), 2.98 (m, 3H), 1.99 (m, 11H), 1.68(m, 11H), 1.39 (d, J=9.60 Hz, 1H), 1.20 (m, 3H). | 291 |
| 13 | 1 | 85 | 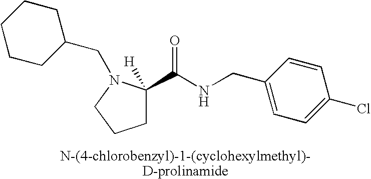 | A | (400 MHz, DMSO-D₆) δ0.84-0.95 (m, 2H), 1.04-1.15 (m, 4H), 1.57(d, J=9.35 Hz, 2H), 1.61-1.67 (m, 2H), 1.83 (d, J=2.53 Hz, 1H), 1.85-1.92 (m, 2H), 1.97-2.09 (m, 1H), 2.39-2.46 (m, 1H), 2.98 (d, J=6.57 Hz, 2H), 3.09-3.19 (m, 1H), 3.61-3.72 (m, 1H), 4.12 (t, J=8.34 Hz, 1H), 4.35 (d, J=5.81 Hz, 2H), 7.29 (d, J=8.34 Hz, 2H), 7.40 (d, J=8.34 Hz, 2H), | 335 |
| | | | | | # 9.20 (t, J=5.81 Hz, 1H). | |
| 14 | 2.4 | 100 | 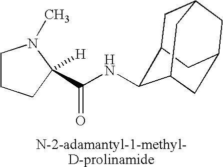 | B | (400 MHz, CHCl₃-D) δ 7.8 (s, 1H), 4.0 (d, J=8.84 Hz, 1H), 3.08-3.14 (m, 1H), 2.85 (dd, J=9.85, 5.05 Hz, 1H), 2.30-2.37 (m, 1H), 2.21 (ddd, J=18.19, 12.13, 9.09 Hz, 1H), 1.91 (s, 1H), 1.83 (s, 8H), 1.72-1.80 (m, 8H), 1.59-1.66 (m, 3H). | 263 |
| 15 | 1.2 | 100 | 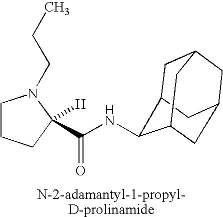 | A | (400 MHz, CHCl₃-D) δ 0.92 (t, J=7.33 Hz, 3H), 1.43-(m, 2H), 1.57 (s. 2H), 1.60-1.67 (m, 2H), 1.72-1.78 (m, 4H), 1.80-1.89 (m, 9H), 2.09-2.20 (m, 1H), 2.28 (m, 1 H), 2.37-2.44 (m, 1H), 2.57 (m, 1H), 3.00 (J=10.11, 4.55 Hz, 1H), 3.16-3.21 (m, 1H), 3.99 (d, J=8.34 Hz, 1H), 7.96 (d, J=7.58 Hz, 1 H). | 291 |
| 16 | 1 | 100 | 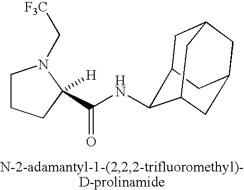 | A | (400 MHz, CHCl₃-D) δ1.61-1.68 (m, 2H), 1.70 (S, 1H), 1.72-1.77 (m, 3H), 1.77-1.81 (m, 1H), 1.81-1.92 (m, 9H), 1.95 (m, 1H), 2.16-2.27 (m, 1H), 2.60 (td, J=9.73, 6.32Hz, 1H), 3.11-3.22 (m, 1H), 3.24-3.33 (m, 1H), 3.39-3.47 (m, 2H), 4.00 (d, J=8.34 Hz, 1H), 7.96 (d, J=6.06 Hz, 1H). | 331 |
| 17 | 3.2 | 98 | 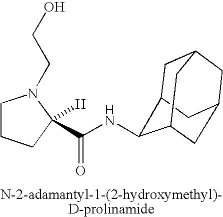 | A | (400 MHz, CHCl₃-D) δ1.54-1.66 (m, 5H), 1.73 (s, 2H), 1.75-1.78 (m, J=6.06 Hz, 1H), 1.79-1.81 (m, J=2.27 Hz, 1H), 1.83 (s, 6H), 1.88-1.92 (m, 2H), 2.20 (m, 1H), 2.34-2.45 (m, 1H), 2.63 (td, J=8.15, 3.92 Hz, 1H), 2.90 (m, J=12.82, 8.02, 4.93 Hz, 1H), 3.14 (s, 1H), 3.24-3.32 (m, 1H), 3.66-3.78 (m, 2H), 4.01 (d, J=8.59 Hz, 1H), 7.78 (d, J=5.05 Hz, 1H). | 293 |
| 18 | 16.4 | 85 | 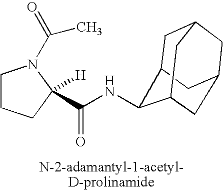 | C | (400 MHz, MeOD) δ 1.59-1.68 (m, 2H), 1.76-1.80 (m, 2H), 1.80-1.93 (m, 9H), 1.94-2.06 (m, 5H), 2.07-2.10 (m, 3H), 2.10-2.19 (m, 1H), 3.53-3.65 (m, 2H), 3.90-3.98 (m, 1H), 4.50 (td, J=7.89, 3.41 Hz, 1H). | 291 |
| 19 | 17 | 86 | 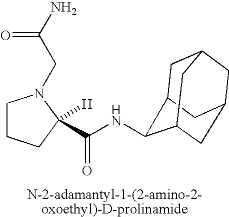 | A | (400 MHz, MeOD) δ 1.60-1.68 (m, 2H), 1.77-1.81 (m, 2H), 1.81-1.92 (m, 10H), 1.94-2.02 (m, 2H), 2.15-2.25 (m, 1H), 2.44-2.51 (m, 1H), 3.16 (d, J=16.17 Hz, 1H), 3.23-3.27 (m, 2H), 3.33-3.39 (m, 2H0, 3.94 (s, 1H). | 306 |
| 20 | 23.3 | 72 | 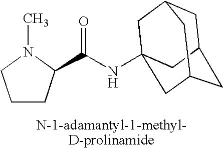 | B | (400 MHz, MeOD) δ 1.69-1.79 (m, 9H), 2.01 (d, J=2.78 Hz, 6H), 2.06 (d, J=1.77 Hz, 3H), 2.09-2.20 (m, 1H), 2.30-2.38 (m, 4H), 2.63-2.70 (m, 1H), 3.04-3.13 (m, 1H). | 263 |
| 21 | 1.2 | 100 | 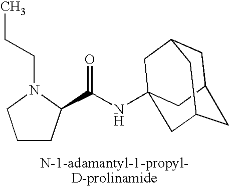 | A | (400 MHz, MeOD) δ 0.97 (s, 3H), 1.45-1.56 (m, 2H), 1.68-1.80 (m, 9H), 2.00 (d, J=3.03 Hz, 6H), 2.04-2.09 (m, 3H), 2.09-2.17 (m, 1H), 2.29 (td, J=9.22, 6.32 Hz, 1H), 2.41-2.52 (m, 2H), 2.81 (dd, J=9.98, 4.17 Hz, 1H), 3.18 (dd, J=8.72, 6.69 Hz, 1H). | 291 |
| 22 | 0.85 | 100 | 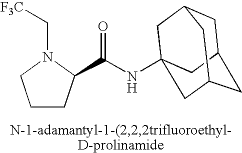 | A | (400 MHz, MeOD) δ 1.69-1.74 (m, 7H), 1.75-1.86 (m, 3H), 1.99 (d, J=2.78 Hz, 6H), 2.06 (s, 3H), 2.10-2.21 (m, 1H), 2.63 (td, J=9.35, 6.32 Hz, 1H), 3.14-3.19 (m, 1 H), 3.21-3.27 (m, 1H). | 331 |
| 23 | 2.6 | 94 | 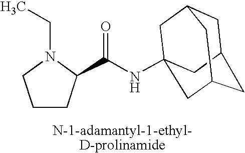 | A | (400 MHz, MeOD) δ 1.08 (t, J=7.2 Hz, 3H), 1.68-1.79 (m, 9H), 2.00 (d, J=2.78 Hz, 6H), 2.04-2.14 (m, 4H), 2.32 (td, J=9.28, 6.44 Hz, 1H), 2.47-2.55 (m, 1H), 2.57-2.64 (m, 1H), 2.82 (dd, J=9.98, 4.17 Hz, 1H), 3.19 (dd, J=8.84, 6.57 Hz). | 277 |
| 24 | 1.4 | 100 | 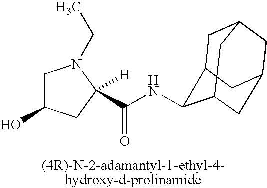 | B | (400 MHz, MeOD) δ 1.11 (t, J=7.20 Hz, 3H), 1.61-1.72 (m, 2H), 1.76-1.82 (m, 3H), 1.82-1.93 (m, 9H), 1.99(d, J=14.91 Hz, 1H), 2.42 (m, 1H), 2.48-2.58 (m, 2H), 2.66 (m, 1H), 3.02 (dd, J=10.86, 4.04Hz, 1H), 3.20 (d, J=10.11 Hz, 1H), 3.92 (s, 1H), 4.30 (t, J=4.55 Hz, 1H). | 293 |
| 25 | 1.7 | 100 | 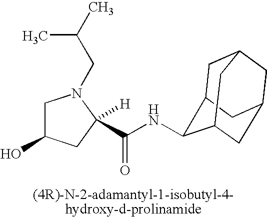 | B | (400 MHz, MeOD) δ 0.90 (d, J=6.57 Hz, 3H), 1.02 (d, J=6.67 Hz, 3H), 1.58-1.70 (m, 2h), 1.72-1.77 (m, 1H), 5H), 1.90-1.95 (m, 2H), 1.98 (s, 1H), 2.28-2.35 (m, 2H), 2.38-2.47 (m, 2H), 2.99 (dd, J=10.86, 4.29 Hz, 1H), 3.16 (d, J=9.85 Hz, 1H), 3.91 (s, 1H), 4.31 (t, J=4.55 Hz, 1H). | 321 |
| 26 | 1 | 100 | 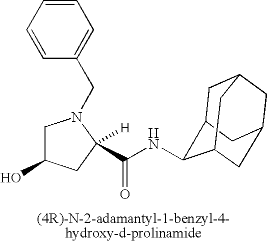 | B | (400 MHz, MeOD) δ 1.57-1.65 (m, 2H), 1.78 (m, 5H), 1.81-1.90 (m, 7H), 1.98 (d, J=13.14 Hz, 1H), 2.49 (m, 1H), 2.57 (dd, J=10.23, 3.92 Hz, 1H), 3.07 (d, J=10.11 Hz, 1H), 3.22 (dd, J=10.74, 4.17 Hz, 1H), 3.59 (d, J=13.14 Hz, 1H), 3.80-3.88 (m, 2H), 4.30 (t, J=4.55 Hz, 1H), 7.23-7.26 (m, 1H), 7.28-7.36 (m, 4H). | 355 |
| 27 | 1.1 | 100 | 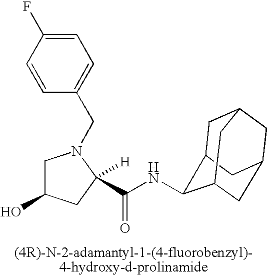 | B | (400 MHz, MeOD) δ 1.62 (t, J=11.75 Hz, 2H), 1.74-1.82 (m, 6H), 1.82-1.90 (m, 6H), 1.97 (d, J=12.63 Hz, 1h), 2.49 (m, 1H), 2.56 (dd, J=10.11, 4.04 Hz, 1H), 3.06 (d, J=10.36Hz, 1H), 3.21 (dd, J=10.61, 4.29 Hz, 1h), 3.60 (d, J=13.14 Hz, 1H), 3.82 (d, J=13.64 Hz, 2H), 4.30 (t, J=4.55 Hz, 1H), 7.38 (m, 2H). | 373 |
| 28 | 1 | 100 | 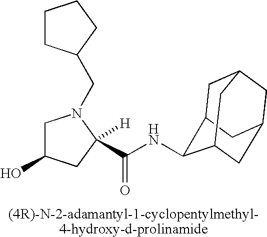 | B | (400 MHz, MeOD) δ 1.12-1.22 (m, 1H), 1.27-1.37 (m, 1H), 1.54-1.64 (m, 5H), 1.95 (m, 13H), 1.96-2.07 3.00 (dd, J=10.99, 3.92 Hz, 1H), 3.21 (d, J=10.61 Hz, 1H), 3.91 (s, 1H), 4.31 (t, J=4.42 Hz, 1H). | 347 |
| 29 | 39.2 | 69 | 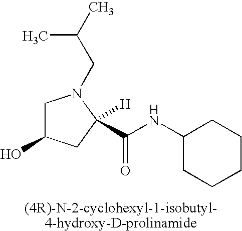 | B | (400 MHz, MeOD) δ 0.90 (d, J=6.57 Hz, 3H), 1.00 (d, J=6.57 Hz, 3H), 1.18-1.30 (m, 3H), 1.31-1.43 (m, 2H), 1.58-1.65 (m, 1H), 1.68-1.78 (m, 4H), 1.84 (d, J=12.38 Hz, 2H), 2.19-2.30 (m, 2H), 2.37-2.47 (m, 2H), 2.92 (dd, J=10.61, 4.80 Hz, 1H), 3.12 (d, J=10.11 Hz, 1 H), 3.63 (m, 1H0, 4.28 (t, J=4.29 Hz, 1H) | |
| 30 | NA | 63 | 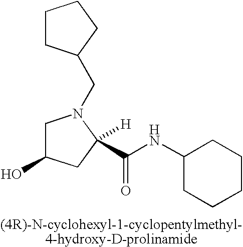 | B | (400 MHz, MeOD) δ 1.03-1.11 (m, 1H), 1.11-1.20 (m, 4H), 1.23-1.33 (m, 2H), 1.44-1.56 (m, 5H), 1.61-1.69 (m,4H), 1.77 (t, J=12.51 Hz, 3H), 1.87-1.97 (m, 1H), 2.27-2.38 (m, 4H), 2.85 (dd, J=10.48, 4.42 Hz, 1H), 3.08 (d, J=10.11 Hz, 1H), 3.54 (m, 1H), 4.19 (t, J=4.55 Hz, 1H). | 295 |
| 31 | NA | 53 | 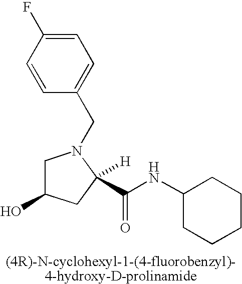 | B | (400 MHz, MeOD) δ 1.15-1.26 (m, 3H), 1.29-1.39 (m, 2H), 1.61 (d, J=12.38 Hz, 1H), 1.68-1.79 (m, 5H), 2.45-2.55 (m, 2H), 2.99 (d, J=10.36 Hz, 1H), 3.11 (dd, J=10.48, 4.9 3Hz, 1H), 3.50-3.58 (m, 2H), 3.74 (d, J=12.88 Hz, 1H), 4.26 (t, J=4.42 Hz, 1 H), 7.04 (1, J=8.72 Hz, 2H), 7.34 (dd, J=8.46, 5.68 Hz, 2H). | 321 |
| 32 | NA | 67 | 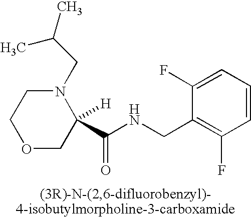 | A | (400 MHz, MeOD) δ 0.85-0.97 (m, 6H), 1.98-2.07 (m, 1H), 2.73-2.85 (m, 2H), 3.55 (m, 1H), 3.55-3.65 (m, 1H), 3.71 -3.82 (m, 2H), 3.89-4.01 (m, 2H), 4.40-4.51 (m, 2H), 6.87-6.95 (m, 2H), 7.24-7.33 (m, 1H). | 313 |
| 33 | NA | 65 | 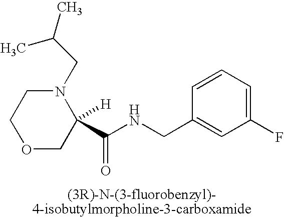 | A | (400 MHz, MoOD) δ 0.88-0.98 (m, 6H), 1.99-2.09 (m, 1H), 2.76-2.86 (m, 2H), 3.02-3.12 (m, 1H), 3.53 (d, J=12.38 Hz, 1H), 3.60-3.70 (m, 1H), 3.72-3.82 (m, 1H), 3.84-3.88 (m, 1H), 3.93 (dd, J=13.01, 3.41 Hz, 1H), 4.06 (dd, J=12.63, 3.54 Hz, 1 H), 4.29-4.38 (m, 2H), 6.90-6.98 (m, 2H), 7.02 (d, J=7.58 Hz, 1H), 7.26 (td, J=7.58, 5.94 Hz, 1H) | 295 |
| 34 | 18 | 79 | 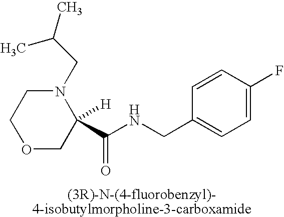 | A | (400 MHz, MeOD) δ 1.02 (d, J=6.57 Hz, 3H), 1.09 (d, J=6.57 Hz, 3H), 2.12-2.23 (m, 1H), 2.89-2.96 (m, 1H), 2.97-3.03 (m, 1H), 3.25 (td, J=12.25, 4.04 Hz, 2H), 3.66-3.73 (m, 1 H), 3.75-3.83 (m, 1 H), 3.91 (td, J=12.38, 2.27 Hz, 1H), 3.66-3.73 (m, 1H), 3.75-3.83 (m, 1 H), 3.91 (td, J=12.38, 2.27 Hz, 1H), 4.00-4.09 (m, 2H), 4.18 (dd, J=12.63, 3.54 Hz, 1H), | 295 |
| | | | | | # 4.43 (s, 2H), 7.06-7.13 (m, 2H), 7.33-7.38 (m, 2H). | |
| 35 | 22.6 | 82 | 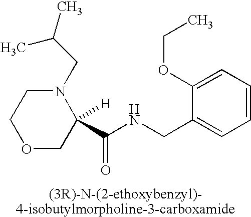 | A | (400 MHz, MeOD) δ 0.96-1.06 (m, 6H), 1.42 (t, J=6.95 Hz, 3H), 2.07-2.18 (m, 1H), 2.81-2.93 (m, 2H), 3.08-3.19 (m, 1H), 3.55-3.66 (m, 1 H), 3.73 (t, 321.4 J=11.49 Hz, 1H), 3.81-3.93 (m, 2H), 3.95-4.04 (m, 1H), 4.04-4.14 (m, 3H), 4.38-4.47 (m, 2H), 6.89-6.98 (m, 2H), 7.23 (d, J=7.33 Hz, 1H), 7.24-7.31 (m, 1H) | 321.4 |
| 36 | 21.4 | 90 | 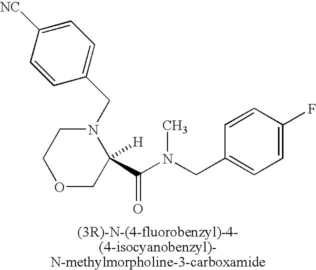 | B | (400 MHz, MeOD) δ 2.96-3.07 (m, 2H), 3.08-3.17 (m, 2H), 3.61-3.71 (m, 3H), 3.84-3.96 (m, 2H), 4.16 (dd, J=12.38, 3.28 Hz, 1H), 4.29 (d, J=13.14 Hz, 1H), 4.37 (d, J=7.07 Hz, 1H), 4.51-4.70 (m, 2H), 7.04-7.10 (m, 1H), 7.14 (t, J=8.72 Hz, 1 H), 7.31 9dt, J=8.53, 5.84 Hz, 2H), 7.65 (d, J=8.34 Hz, 2H), 7.76-7.85 (m, 2H). | 368.4 |
| 37 | 39.3 | 100 | 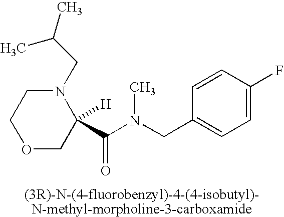 | A | (400 MHz, MeOD) δ 1.00-1.06 (m, 3H), 1.07-1.12 (m, 3H), 2.12-2.23 (m, 1H), 2.81-2.92 (m, 1H), 2.94-3.04 (m, 2H), 3.08 (s, 2H), 3.21-3.29 (m, 1H), 3.63-3.74 (m, 2H), 3.86-3.96 (m, 1H), 4.00-4.12 (m, 1H), 4.25 (DD, j=12.88, 3.54 Hz, 1 H), 4.59-4.69 (m, 3H), 7.05-7.11 (m, 2h), 7.31 (dd, J=8.34, 5.31 Hz, 2H). | 309.5 |
| 38 | 7.9 | 90.4 | 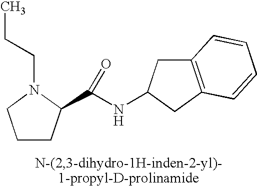 | A | (400 MHz, MeOD) δ ppm 0.76 (t, J=7.45 Hz, 3 H) 1.28-1.40 (m, 2 H) 1.68-1.80 (m, 3H) 2.08-2.18 (m, 1H) 2.22-2.30 (m, 1 H) 2.35-2.43 (m, 2 H) 2.82 (ddd, J=15.54, 10.23, 4.80 Hz, 2 H) 2.93 (dd, 273.3 J=9.85, 4.04 Hz, 1 H) 3.12 (t, J=6.95 Hz, 1 H) 3.20-3.27 (m, 2 H) 4.58 (ddd, J=11.81, 6.88, 5.05 Hz, 1 H) 7.11 -7.16 (m, 2 H) 7.21 (dt, J=8.59, 4.29 Hz, 2H) | 273.3 |
| 39 | <1 | 100 | 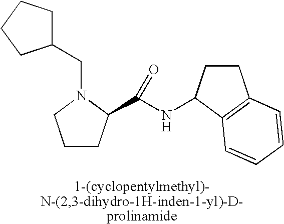 | A | (400 MHz, MeOD) δ ppm 0.87-0.97 (m, J=12.41, 7.99, 7.99, 7.99 Hz, 1 H) 0.99-1.09 (m, J=12.38, 7.96, 7.83, 7.71 Hz, 1 H) 1.35-1.41 (m, 2 H) 1.42-1.48 (m, 2 H) 1.57-1.66 (m, J=12.69, 12.69, 7.45 Hz, 3 H) 1.74 -1.85 (m, 2 H) 1.89-1.98 (m, J=15.41, 7.71, 7.71 Hz, 1 H) 2.12-2.22 (m, 1 H) 2.26-2.33 (m, 1 H) 2.40 (d, J=7.33 Hz, 2 H) 2.46-2.55 (m, | 313.3 |
| | | | | | # J=12.73, 8.04, 8.04, 4.55 Hz, 1 H) 2.84-2.93 (m, J=15.85, 7.86, 7.86 Hz, 1 H) 2.98-3.06 (m, J=15.66, 8.84, 4.55 Hz, 2 H) 3.18 (t, J=7.45 Hz, 1 H) 5.37 (t, J=7.20 Hz, 1 H) 7.15-7.26 (m, 4H) | |
| 40 | 19 | 100 | 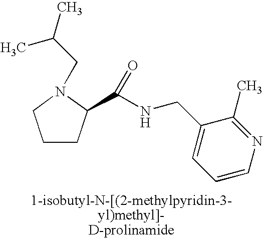 | A | (400 MHz, MeOD) δ ppm 0.76-0.84 (m, 6 H) 1.62-1.73 (m, 1 H) 1.74-1.85 (m, 3H) 2.15-2.20 (m, 1 H) 2.22-2.32 (m, 3H) 2.54 (s, 3 H) 3.02 (dd, J=9.98, 4.17 Hz, 1 H) 3.14-3.21 (m,1 H) 4.34 (d, J=15.16 Hz, 1 H) 4.54 (d, J=15.16 Hz, 1 H) 7.24 (dd, J=7.71, 4.93 Hz, 1 H) 7.66 (dd, J=7.58, 1.26 Hz, 1 H) 8.31 (dd, J=4.93, 1.64 Hz, 1 H) | 276.3 |
| 41 | <1 | 100 | 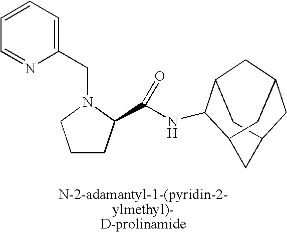 | B | (400 MHz, MeOD) δ ppm 1.57-1.67 (m, 2 H) 1.70-1.75 (m, 2 H) 1.75-1.79 (m, 3H) 1.79-1.90 (m, 10 H) 2.20-2.31 (m, 1 H) 2.51-2.58 (m, J=9.54, 9.54, 6.19 Hz, 1 H) 3.14 (t, J=7.20 Hz, 1 H) 3.34 (d, J=4.55 Hz, 1 H) 3.76-3.82 (m, 1 H) 3.84 (s, 1 H) 3.91-3.96 (m, 1 H) 7.30 (dd, j=6.95, 5.43 Hz, 1 H) 7.44 (d, J=7.83 Hz, 1 H) 7.78 (td, J=7.64, 1.64 Hz, 1 H) | 340.3 |
| | | | | | # 8.49 (d, J=4.29 Hz, 1 H) | |
| 42 | NA | 0 | 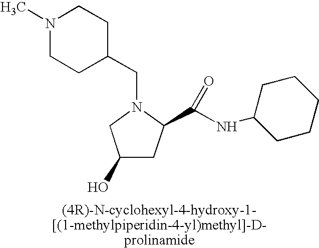 | D | (400 MHz, MeOD) δ ppm 1.08-1.20 (m, 6 H) 1.26-1.33 (m, 3 H) 1.50-1.58 (m, J=12.63, 3.79, 3.54 Hz, 1 H) 1.59-1.69 (m, 4 H) 1.73-1.80 (m, J=12.38 Hz, 2 H) 1.89-2.00 (m, 3 H) 2.18 (s, 3 H) 2.22-2.29 (m, 1 H) 2.30-2.39 (m, 2 H) 2.75-2.87 (m, 3 H) 3.03 (d, J=10.11 Hz, 1 H) 3.55 (tt, J=10.33, 3.95 Hz, 1 H) 4.18-4.22 (m, 1 H). | 324.3 |
| 43 | NA | 31.9 | 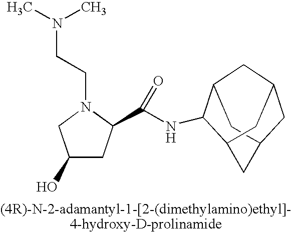 | E | (400 MHz, MeOD) δ ppm 1.49-1.61 (m, 2 H) 1.68-1.70 (m, J=1.52 Hz, 3 H) 1.72-1.83 (m, 9 H) 1.85-1.94 (m, 2 H) 2.19 (s, 6 H) 2.34-2.46 (m, 4 H) 2.70-2.76 (m, 1 H) 2.96 (dd, J=10.86, 4.29 Hz, 1 H) 3.08-3.13 (m, 1 H) 3.84 (s, 1 H) 4.18 (t, J=4.67 Hz, 1 H) | 336.3 |
| 44 | NA | 59.4 | 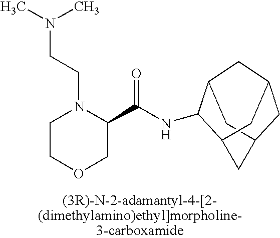 | E | (400 MHz, MeOD) δ ppm 1.63-1.70 (m, J=14.40 Hz, 2 H) 1.79 (s, 2 H) 1.82 1.87 (m, 4 H) 1.87-1.93 (m, J=6.57 Hz, 5 H) 1.94 (s, 1 H) 2.26-2.31 (m, 6 H) 2.32-2.40 (m, 2 H) 2.52-2.59 (m, 2 H) 2.74 (dt, J=12.38, 7.83 Hz, 1 H) 2.98-3.08 (m, 2 H) 3.52 (dd, J=11.24, 9.22 Hz, 1 H) 3.62 (td, J=10.99, 2.53 Hz, 1 H) 3.78-3.87 (m, 2 H) 3.97 (s, 1 H) | 336.3 |
| 45 | NA | 81 | 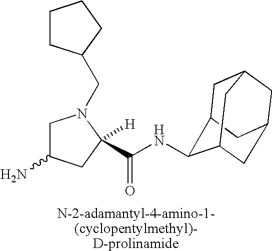 | F | 1H NMR (400 MHz, MeOD) δ ppm 1.21-1.33 (m, 3 H) 1.59-1.97 (m, 16 H) 2.08-2.18 (m, 2H) 2.43-2.72 (m, 2H) 2.74-2.83 (m, 2H) 3.03-3.16 (m, 4 H) 3.72-3-74 (m, 1 H) 3.97 (m, 1H) 4.17 (m, 1H) | 346 |
| 46 | <1 | 100 | 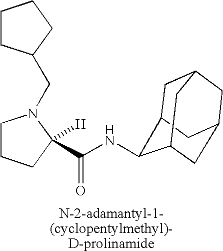 | B | (400 MHz, MeOD) δ ppm 1.23-1.34 (m, 2 H) 1.58-1.61 (m, 1 H) 1.62-1.71 (m, 5 H) 1.80 (s, 2 H) 1.84-1.94 (m, J=13.64, 11.12 Hz, 13H) 1.99-2.01 (m, J=4.29 Hz, 1 H) 2.10-2.22 (m, 2H) 2.52-2.62 (m, 1 H) 3.16-3.26 (m, 3 H) -3.76-3.85 (m, J=10.80, 6.88, 4.29 Hz, 1 H) 4.01 (s, 1 H) 4.17-4.26 (m, 1 H) | 331 |
| 47 | NA | 38 | 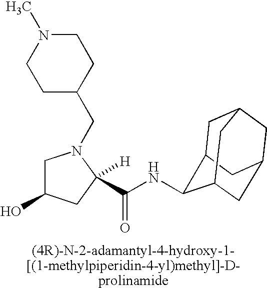 | D | 1H NMR (400 MHz, MeOD) δ ppm 1.45-1.56 (m, 1 H) 1.62-2.04 (m, 21 H) 2.26 (s, 3 H) 2.37-2.48 (m, 4 H) 2.83-2.92 (m, 2 H) 3.01 (dd, J=10.86, 4.29 Hz, 1 H) 3.16 (d, J=10.11 Hz, 1 H) 3.93 (s, 1 H) 4.32 (t, J=4.42 Hz, 1 H) | 376.5 |
| 48 | NA | 83 | 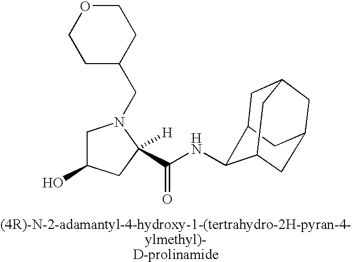 | B | 1H NMR (400 MHz, METHANOL-d₄) δ ppm 1.19-1.30 (m, 2 H) 1.62-1.97 (m, 18 H) 2.38-2.49 (m, 4 H) 3.03 (dd, J=10.74, 3.92 Hz, 1 H) 3.18 (d, J=10.11 Hz, 1 H) 3.38-3.45 (m, 2 H) 3.90-3.96 (m, 3 H) 4.33 (t, J=4.42 Hz, 1 H) | 363 |
| 49 | NA | 64 | 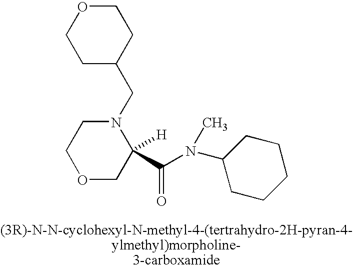 | B | 1H NMR (400 MHz, CHLOROFORM-d) δ ppm 1.03-1.84 (m, 14 H) 2.06 (m, 1 H) 2.24-2.42 (m, 2 H) 2.96-3.06 (m, 3 H) 3.30-3.41 (m, 4 H) 3.68-3.79 (m, 5 H) 3.87-3.98 (m, 3H) | 325 |
| 50 | 1.3 | 100 | 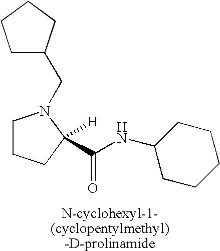 | A | 1H NMR (400 MHz, CHLOROFORM-d) δ ppm 1.09-1.21 (m, 5 H) 1.32-1.42 (m, 2 H) 1.52-2.08 (m, 15H) 2.1-2.2 (m, 1 H) 2.24-2.31 (m, 1) 2.40 (d, J=7.33 Hz, 2 H) 2.98 (dd, J=9.85, 4.04 Hz, 1 H) 3.19 (t, J=7.33 Hz, 1 H) 3.67-3.77 (m, J=19.04, 10.45, 4.23, 3.92 Hz, 1 H) | 279 |
| 51 | 1 | 100 | 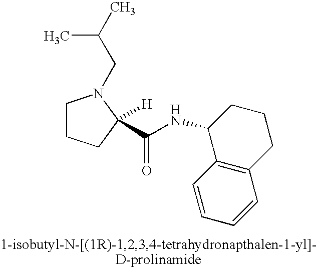 | A | 1H NMR (400 MHz, CHLOROFORM-d) δ ppm 0.57 (d, J=6.57 Hz, 3 H) 0.74 (d, J=6.57 Hz, 3 H) 1.57-1.66 (m, 2 H) 1.68-1.88 (m, 5 H) 1.98-2.06 (m, 1H) 2.14-2.25 (m, 4 H) 2.73-2.84 (m, 2 H) 3.02-3.11 (m, 2 H) 5.09-5.18 (m, 1 H) 7.06-7.10 (m, 1 H) 7.15 (ddd, J=7.01, 4.86, 1.77 Hz, 2 H) 7.21-7.25 (m, 1 H) 7.64 (d, J=8.59 Hz, 1 H) | 301 |
| 52 | 4.6 | 100 | 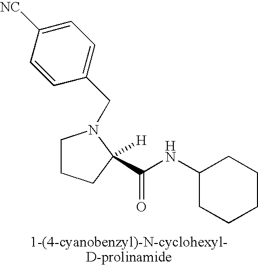 | B | 1H NMR (400 MHz, CHLOROFORM-d) δ ppm 1.08-1.20 (m, 3H) 1.32-1.43 (m, 2 H) 1.64-1.90 (m, 8 H) 2.22-2.37 (m, 2 H) 3.02 (1, J=7.07 Hz, 1 H) 3.18 (dd, J=9.60, 5.05 Hz, 1 H) 3.53 (d, J=13.39 Hz, 1 H) 3.67-3.77 (m, 1 H) 3.92 (d, J=13.64 Hz, 1 H) 7.13 (d, J=7.83 Hz, 1 H) 7.40 (d, J=7.83 Hz, 2 H) 7.64 (d, J=8.34 Hz, 2 H) | 312 |
| 53 | 4.6 | 100 | 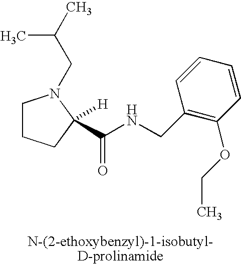 | A | 1H NMR (400 MHz, MeOD) δ ppm 1.00 (d, J=6.57 Hz, 3 H) 1.05 (d, J=6.57 Hz, 3 H) 1.42 (t, J=7.07 Hz, 3 H) 1.9-2.21 (m, 4 H) 2.47-2.57 (m, 1 H) 3.04 (ddd, J=15.92, 12.51, 7.20 Hz, 2 H) 3.20 (dt, J=11.05, 8.24 Hz, 1 H) 3.77-3.85 (m, 1 H) 4.06-4.15 (m, 3 H) 4.40-4.50 (m, 2 H) 6.90 (t, J=7.45 Hz, 1 H) 6.97 (d, J=8.34 Hz, 1 H) 7.21-7.29 (m, 2 H) | 305.5 |
| 54 | 13.2 | 94 | 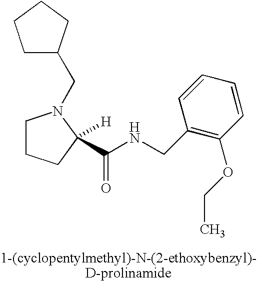 | A | 1H NMR (400 MHz, MeOD) δ ppm 1.22-1.33 (m, 2 H) 1.42 (t, J=6.95 Hz, 3H) 1.52-1.71 (m, 4 H) 1.81-1.92 (m, 2 H) 2.04-2.18 (m, 4 H) 2.47-2.57 (m, 1 H) 3.13-3.24 (m, 3 H) 3.81 (ddd, J=11.56, 7.01, 4.42 Hz, 1 H) 4.07-4.16 (m, 3 H) 4.45 (s, 2 H) 6.90 (t, J=7.45 Hz, 1 H) 6.96 (d, J=8.08 Hz, 1 H) 7.22-7.28 (m, 2H) | 331.5 |
| 55 | 3.7 | 100 | 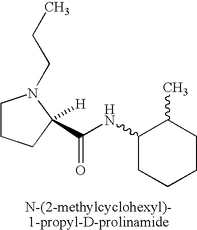 | A | 1H NMR (400 MHz, CHLOROFORM-d) δ ppm 0.84-0.95 (m, 6 H) 1.04-1.91 (m, 14 H) 2.10-2.61 (m, 4 H) 2.96-3.06 (m, 1 H) 3.13-3.22 (m, 1 H) 3.36-3.47 (m, 1 H) | 253 |
| 56 | 8 | 92 | 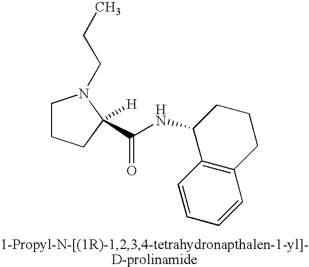 | A | 1H NMR (400 MHz, CHLOROFORM-d) δ ppm 0.85 (t, J=7.33 Hz, 3 H) 1.42-1.54 (m, 2 H) 1.78-2.0 (m, 6 H) 2.08-2.19 (m, 1 H) 2.2-2.33 (m, 2H) 2.44 (dt, J=11.62, 8.21 Hz, 1 H) 2.6-2.7 (m, 1 H) 2.75-2.86 (m, 2 H) 3.16-3.24 (m, 2 H) 5.19 -5.27 (m, 1 H) 7.19-7.23 (m, 1 H) 7.27 (ddd, J=8.97, 2.02, 1.89 Hz, 2 H) 7.32-7.35 (m, 1 H) 7.78 (d, J=6.82 Hz, 1 H) | 287 |
| 57 | 23.4 | 81 | 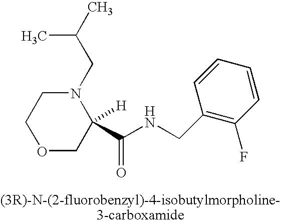 | A | 1H NMR (400 MHz, MeOD) δ ppm 0.99 (d, J=6.57 Hz, 3 H) 1.06 (d, J=6.6 Hz, 3 H) 2.09-2.19 (m, J=8.92, 6.68, 4.45, 4.45, 2.53 Hz, 1 H) 2.86-2.99 (m, 3 H) 3.22 (td, J=12.38, 4.04 Hz, 1 H) 3.67 (d, J=12.88 Hz, 1 H) 3.71-3.79 (m, 1 H) 3.87 (td, J=12.38, 2.27 Hz, 1 H) 4.01 (td, J=11.31, 3.66 Hz, 1 H) 4.14 (dd, J=12.63, 3.54 Hz, 1H) 4.43-4.51 (m, 2 H) 7.08-7.18 (m, 2 | 295 |
| | | | | | # H) 7.30-7.40 (m, 2 H) | |
| 58 | 28 | 70 | 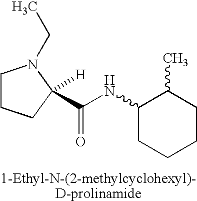 | A | 1H NMR (400 MHz, CHLOROFORM-d) δ ppm 0.87-0.93 (m, 3 H) 1.04-1.34 (m, 8 H) 1.6-1.92 (m, 7 H) 2.1-2.17 (m, 1 H) 2.31 (bs, 1 H) 2.46 (bs, 1 H) 2.68 (m, 1 H) 3.02 (bs, 1 H) 3.18 (bs, 1 H) 3.35-3.46 (m, 1 H) | 239 |
| 59 | 22 | 71 | 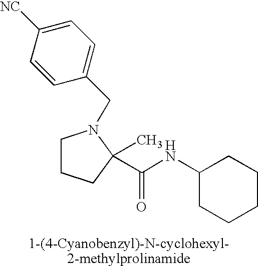 | B | 1H NMR (400 MHz, CHLOROFORM-d) δ ppm 1.09-1.27 (m, 4 H) 1.29-1.45 (m, 5 H) 1.83-1.90 (m, 7H) 2.01-2.11 (m, 1 H) 2.35-2.44 (m, 1 H) 2.91-2.99 (m, 1 H) 3.40 (d, J=13.89 Hz, 1 H) 3.69-3.79 (m, 1 H) 3.85 (d, J=13.89 Hz, 1 H) | 326 |
| 60 | 25 | 79 | 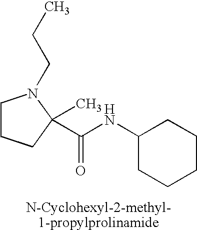 | A | 1H NMR (400 MHz, CHLOROFORM-d) δ ppm 0.86-0.95 (m, 3 H) 1.08-1.20 (m, 6 H) 1.33-1.42 (m, 3 H) 1.50-1.61 (m, 2 H) 1.64-1.76 (m, 5H) 1.84 (d, J=11.62 Hz, 2H) 1.92-2.02 (m, 1 H) 2.28-2.40 (m, 3 H) 3.12-3.21 (m, 1 H) 3.63-3.73 (m, J=18.85, 10.33, 4.17, 3.79 Hz, 1 H) 7.59 (d, J=6.06 Hz, 1 H) | 253 |
| 61 | NA | 74 | 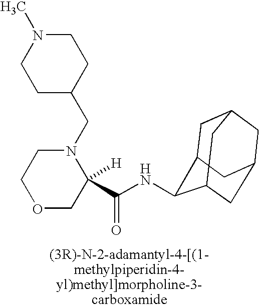 | D | (400 MHz, MeOD) δ ppm 1.52-1.57 (m, 1 H) 1.59-1.70 (m, 3 H) 1.80 (s, 3 H) 1.88 (dd, J=17.18, 2.78 Hz, 5 H) 1.93 (dd, J=9.47, 2.15 Hz, 4 H) 2.02 (d, J=14.15 Hz, 2 H) 2.29 (dt, J=12.06, 3.32 Hz, 1 H) 2.43 (d, J=14.40 Hz, 1 H) 2.88 (s, 3 H) 3.02-3.14 (m, 3 H) 3.19 (dd, J=13.14, 3.28 Hz, 1 H) 3.33-3.40 (m, 1 H) 3.52-3.61 (m, 2 H) 3.71 (t, J=12.38 Hz, 1 H) 3.80 (d, | 376 |
| | | | | | # J=12.88 Hz, 1 H) 3.85-3.96 (m, 1 H) 4.03 (d, J=5.31 Hz, 1 H) 4.09 (dd, J=13.01, 3.16 Hz, 1 H) 4.20-4.30 (m, 2 H) | |
| 62 | 1.1 | 100 | 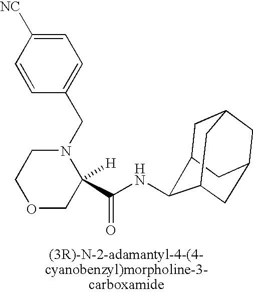 | B | (400 MHz, MeOD) δ 1.56-1.66 (M, 2H), 1.73 (s, 3H), 1.78-2.02 (m, 8H), 3.29-3.39 (m, 1 H), 3.58-3.69 (m, 2H), 3.93-4.02 (m, 2H), 4.16-4.28 (m, 3H), 4.48 (d, J=12.63 Hz, 1H), 7.61-7.70 (m, 2H), 7.81 (d, J=7.83 Hz, 2H), 8.52 (s, 1 H) | 380 |
| 63 | 1 | 100 | 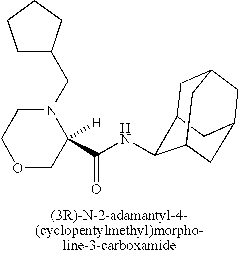 | B | (400 MHz, MeOD) δ 1.17-1.28 (m, 1H), 1.31-1.42(m, 1H), 1.59-1.71 (m, 6H), 1.80 (s, 1H), 1.84-1.96(m, 12H), 2.35 (dt, J=13.83, 7.99 Hz, 1H), 3.03-3.10 (m, 1H) 3.11-3.18 (m, 1H), 3.22-3.29 (m, 1H), 3.62-3.73 (m, 2H), 3.85 (td, J=12.63, 2.02 Hz, 1H), 4.01 (s, 1 H), 4.04 (br, 1 H), 4.06 (dd, J=13.01, 3.41 Hz, 1 H), 4.11-4.22 (m, 2H) | 347 |
| 64 | 1.3 | 100 | 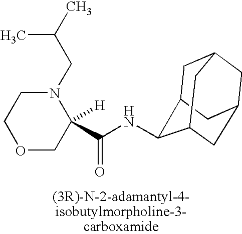 | B | (400 MHz, MeOD) δ 1.02 (d, J=6.82 Hz, 3H), 1.10 (d, J=6.57 Hz, 3H), 1.62-1.71 (m, 2H), 1.80 (s, 3H), 1.84-1.94 (m, 9H), 2.13-2.24 (m, 1H), 2.85-2.96 (m, 1H), 2.97-3.08 (m, 1H), 3.20-3.28 (m, 1 H), 3.66 (d, J=12.63 Hz, 1H), 3.72 (dd, J=12.13, 10.86 Hz, 1 H), 3.83-3.93 (m, 1 H), 3.97-4.02 (m, 1h), 4.02-4.12 (m, 2H), 4.12-4.22 (m, 2H) | 321 |
| 65 | 2.6 | 100 | 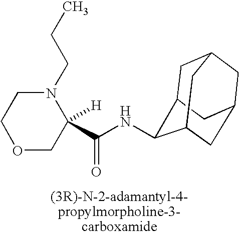 | B | (400 MHz, MeOD) δ 0.99 (t, J=7.45 Hz, 3H), 1.62-1.99 (m, 17H), 3.08 (t, J=8.21 Hz, 2H), 3.21-3.29 (m, 1H), 3.59 (d, J=12.88 Hz, 1H), 3.80 (t, J=12.00 Hz, 1H0, 4.00-4.11 (m, 2H), 4.16-4.26 (m, 2H) | 307 |
| 66 | 10 | 89 | 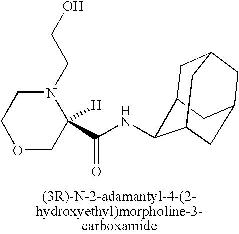 | A | (400 MHz, MeOD) δ 1.54 (d, J=12.13 Hz, 2H), 1.69-1.95 (m, 8H), 2.17-2.26 (m, 2H), 2.62-2.71 (m, 1H), 2.89-3.00 (m, 2H), 3.31-3.40 (m, 1 H), 3.44-3.56 (m, 2H), 3.66 (m, 1h), 3.71-3.82 (m, 2H), 3.85 (s, 1 H), 4.76-4.82 (m, 4H) | 309 |
| 67 | 1 | 100 | 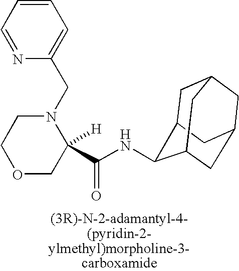 | B | (400 MHz, MeOD) δ 1.49 (d, J=12.88 Hz, 1H), 1.57 (d, J=12.88 Hz, 1H), 1.66-1.72 (m, 4H), 1.73-1.77 (m, 4H), 1.80 (d, J=14.65 Hz, 4H), 1.93 (d, J=13.14 Hz, 1H), 2.29 (td, J=11.31, 3.41 Hz, 1H), 2.66 (d, J=12.13 Hz, 1H), 3.10 (dd, J=9.35, 3.79 Hz, 1H), 3.38 (d, J=14.15 Hz, 1H), 3.45-3.55 (m, 2H), 3.68 (d, J=11.37 Hz, 1H), 3.81-3.90 (m, 3H), 7.18-7.26 (m, 1H), | 356 |
| | | | | | # 7.44 (d, J=7.83 hz, 1H0, 7.68-7.75 (m, 1h), 8.42 (d, J=5.05 Hz, 1 H) | |
| 68 | 2 | 93.5 | 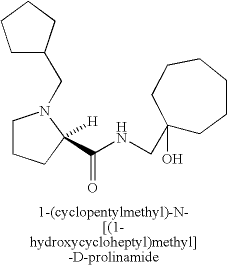 | B | (400 MHz, MeOD) δ ppm 1.23-1.34 (m, 2 H) 1.36-1.41 (m, 1 H) 1.43 (dd, J=7.33, 4.55 Hz, 1 H) 1.57-1.68 (m, 13H) 1.85-1.96 (m, 2H) 1.98-2.09 (m, 3 H) 2.11-2.23 (m, 2 H) 2.52-2.63 (m, 1 H) 3.17-3.28 (m, 5 H) 3.82 (m, 1 H) 4.21 -4.30 (m, 1 H) 8.42 (t, J=5.68 Hz, 1 H) | 323 |
| 69 | 7 | 100 | 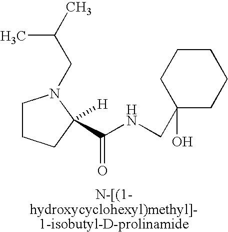 | B | (400 MHz, MeOD) δ ppm 1.04 (dd, J=14.91, 6.57 Hz, 6H) 1.27-1.38 (m, 1 H) 1.44-1.54 (m, 6 H) 1.58-1.68 (m, 2 H) 1.97-2.09 (m, 3 H) 2.13-2.24 (m, 1 H) 2.51-2.60 (m, 1 H) 2.81 (s, 2 H) 3.02-3.08 (m, 2 H) 3.18-3.26 (m, 1 H) 3.28 (d, J=1.77 Hz, 1 H) 3.81 (m, 1 H) 4.14-4.20 (m, 1 H) 8.32-8.42 (m, 1 H) | 283 |
| 70 | NA | 74 | 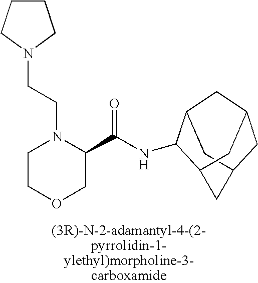 | A | (400 MHz, MeOD) δ ppm 1.64 (s, 1 H) 1.68 (s, 1 H) 1.78-1.81 (m, 2 H) 1.82-1.87 (m, 5 H) 1.87-1.91 (m, 7 H) 1.93 (s, 4 H) 1.96 (s, 1 H) 2.36 (td, J=11.18, 3.41 Hz, 1 H) 2.45 (dt, J=12.32, 6.09 Hz, 1 H) 2.72-2.81 (m, 2 H) 2.81-2.91 (m, 5.68, 5.56 Hz, 2 H) 2.97-3.03 (m, 1 H) 3.09-3.17 (m, 1 H) 3.51 (dd, J=11.24, 9.47 Hz, 1 H) 3.63 (td, J=11.05, 2.40 Hz, | 362.3 |
| | | | | | # 1 H) 3.80-3.88 (m, 2 H) 3.98 (s, 1 H) | |
| 71 | NA | NA | 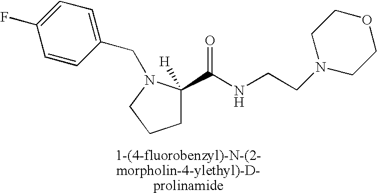 | A | (400 MHz, MeOD) δ ppm 2.00-2.12 (m, 2 H) 2.20 (ddd, J=8.21, 4.29, 4.17 Hz, 1 H) 2.55 -2.66 (m, 1 H) 3.17 (td, J=11.94, 3.16 Hz, 2 H) 3.24 (dt, J=12.88, 6.44 Hz, 2 H), 3.35-3.41 (m, 1 H) 3.51-3.62 (m, 4 H) 3.63-3.72 (m, 1 H) 3.94 (t, J=12.63 Hz, 2 H) 4.00-4.08 (m, 2 H) 4.34-4.44 (m, 2 H) 4.60 (d, J=12.88 Hz, 1 H) 7.21 (t, J=8.72 Hz, 2 H) 7.61 (dd, | 336 |
| | | | | | # J=8.46, 5.18 Hz, 2 H), 8.74 (br, 1 H) | |
| 72 | NA | NA | 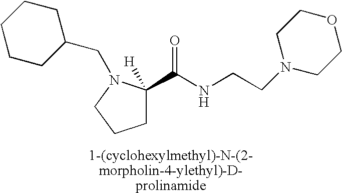 | B | (400 MHz, MeOD) δ ppm 1.00-1.11 (m, J=12.06, 11.84, 11.84, 3.16 Hz, 2 H) 1.26-1.37(m, 3H) 1.71-1.83 (m, 5 H) 1.98 (d, J=12.88 Hz, 1 H) 2.08-2.15 (m, 2 H) 2.16-2.21 (m, 1 H) 2.58 (td, J=8.40, 4.67 Hz, 1 H) 3.03 (dd, J=12.63, 5.56 Hz, 1 H) 3.15-3.26 (m, 3 H) 3.34-3.41 (m, 2 H) 3.57-3.67 (m, 4 H) 3.75-3.85 (m, 2 H) 3.92-4.00 (m, 2 H) | 324 |
| | | | | | # 4.01-4.07 (m, 2 H) 4.33 (t, J=8.21 Hz, 1 H), 8.78 (br, 1 H). | |
| 73 | NA | 83 | 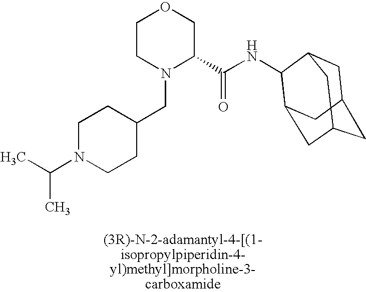 | D | (400 MHz, MeOD) δ ppm 1.07-1.14 (m, 6 H) 1.17-1.28 (m, 2 H) 1.63-1.72 (m, 4H) 1.73-1.93(m, 12 H) 1.99-2.06(m, 1 H) 2.18-2.35 (m, 5 H) 2.81 (dt, J=13.14, 6.57 Hz, 1 H) 2.90-3.02 (m, 4 H) 3.54 (dd, J=11.12, 9.09 Hz, 1 H) 3.59-3.67 (m, 1 H) 3.80-3.87 (m, 1 H) 3.97 (s, 1 H) | 404.3 |
| 74 | NA | 90 | 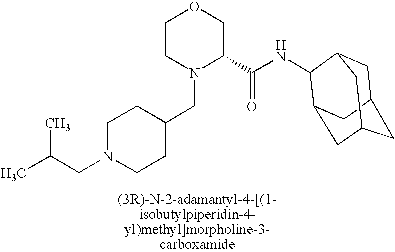 | D | (400 MHz, MeOD) δ ppm 0.91 (dd, J=6.57, 2.27 Hz, 6 H) 1.17-1.29 (m, 2 H) 1.58-1.70 (m, 4 H) 1.79-1.95 (m, 16 H) 2.10-2.21 (m, 4 H) 2.37 (dd, J=12.13, 10.11 Hz, 1 H) 2.89 (dd, J=9.22, 3.66 Hz, 3 H) 2.99 (dt, J=11.87, 2.65 Hz, 1 H) 3.52 (dd, J=11.24, 9.22 Hz, 1 H) 3.57-3.65 (m, 1 H) 3.79-3.87 (m, 2H) 3.96 (s, 1 H) | 418.3 |
| 75 | NA | 91 | 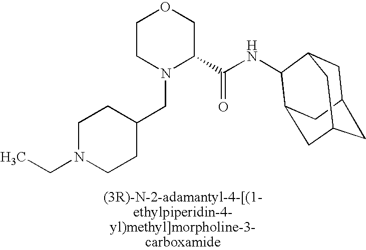 | D | (400 MHz, MeOD) δ ppm 1.13(t, J=7.20 Hz, 3 H) 1.17-1.28(m, 2H) 1.65-1.75 (m, 4 H) 1.81 1.95 (m, 12 H) 2.00-2.12 (m, 3 H) 2.16-2.26 (m, 2 H) 2.38 (dd, J=12.13, 9.85 Hz, 1 H) 2.46-2.53 (m, 2 H) 2.92 (dd, J=9.09, 3.54 Hz, 1 H) 2.98-3.06 (m, 3 H) 3.54 (dd, J=11.37, 9.09 Hz, 1 H) 3.63 (td, J=10.86, 2.53 Hz, 1 H) 3.80-3.87 (m, 2 H) 3.97 (s, 1 H) | 390.3 |
| 76 | NA | 14 | 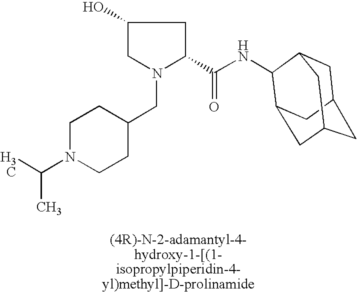 | D | (400 MHz, MeOD) δ ppm 1.08 (dd, J=6.57, 3.28 Hz, 6 H) 1.18-1.30 (m, 2 H) 1.46-1.57 (m, 1 H) 1.63-2.11 (m, 17 H) 2.15-2.25 (m, 2 H) 2.36-2.48 (m, 4 H) 2.72 (dt, J=13.14, 6.57 Hz, 1 H) 2.87-2.95 (m, 2 H) 3.01 (dd, J=10.74, 4.17 Hz, 1 H) 3.17 (d, J=10.11 Hz, 1 H) 3.93 (s, 1 H) 4.33 (t, J=4.42 Hz, 1 H) | 404.3 |
| 77 | NA | NA | 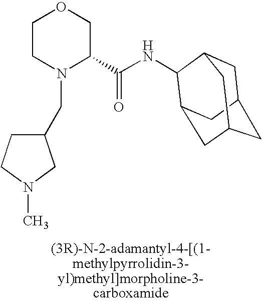 | D | (400 MHz, MeOD) δ ppm 1.62-1.73 (m, 2 H) 1.81-2.15 (m, 14 H) 2.26-2.36 (m, 6 H) 2.47 -2.57 (m, 3 H) 2.63-2.71 (m, 1 H) 2.78 2-9 (m, 1 H) 2.95-3.07 (m, 2 H) 3.56-3.66 (m, 2 H) 3.78-3.85 (m, J=11.27, 7.42, 3.54, 3.54 Hz, 2 H) 3.98 (s, 1 H) | 362.3 |
| 78 | NA | NA | 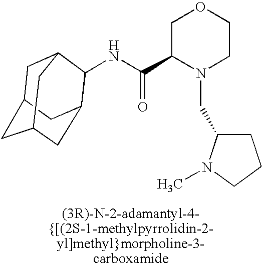 | D | (400 MHz, MeOD) δ ppm ppm 1.67 (d, J=12.88 Hz, 2 H) 1.80-2.04(m, 15H) 2.16-2.25 (m, 1 H) 2.54 (ddd, J=12.00, 9.35, 3.16 Hz, 1 H) 2.66-2.71 (m, 2 H) 2.74 (s, 3 H) 2.85-2.93 (m, 1 H) 3.00 (ddd, J=8.84, 5.94, 2.91 Hz, 1 H) 3.17 (dq, J=6.69, 6.44 Hz, 1 H) 3.25 (dd, J=8.34, 3.28 Hz, 1 H) 3.43-3.51 (m, 1 H) 3.58-3.69 (m, 2 H) 3.79 (dt, J=11.43, 3.25 Hz, 1 H) | 362.3 |
| | | | | | # 3.86 (dd, J=11.12, 3.28 Hz, 1 H) 3.99 (s, 1 H) | |
| 79 | NA | NA | 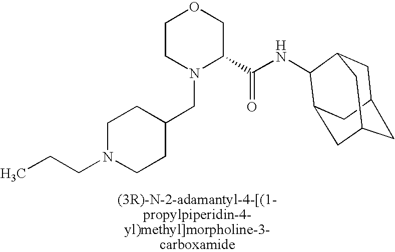 | D | (400 MHz, MeOD) δ ppm 0.92 (t, J=7.33 Hz, 3 H) 1.17-1.28 (m, 2 H) 1.49-1.60 (m, 2 H) 1.63-1.74 (m, 4 H) 1.81 (s, 2 H) 1.84-(m, 3H) 2.14-2.26 (m, 2 1.95 (m, 11 H) 1.96-2.08 H) 2.30-2.40 (m, 3 H) 2.91 (dd, J=9.09, 3.79 Hz, 1 H) 2.94-3.02 (m, J=8.87, 5.91, 2.91, 2.78 Hz, 3 H) 3.53 (dd, J=11.12, 9.09 Hz, 1 H) 3.58-3.66 (m, 1 H) 3.79-3.87 (m, 2 H) 3.97 (s, 1 H) | 404.3 |
| 80 | NA | NA | 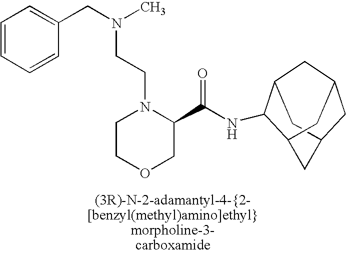 | N | (400 MHz, MeOD) δ ppm 1.59-1.67 (m, 2 H) 1.78 (s, 2 H) 1.80-1.91 (m, 10 H) 2.20-2.30 (m, 4 H) 2.33-2.41 (m, 1 H) 2.50-2.62 (m, 2 H) 2.71-2.79 (m, 1 H) 2.85-2.93 (m, 1 H) 2.98-3.04 (m, 1 H) 3.47-3.58 (m, 4 H) 3.72-3.79 (m, 1 H) 3.83 (dd, J=11.24, 3.66 Hz, 1 H) 3.93-3.98 (m, 1 H) 7.23-7.34 (m, 5 H) | 412.3 |
| 81 | NA | NA | 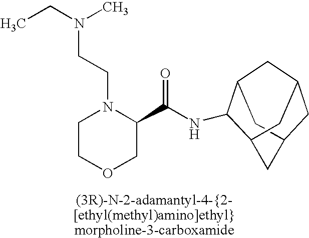 | N | (400 MHz, MeOD) δ ppm 1.43 (t, J=7.20 Hz, 3 H) 1.64-1.73 (m,2H) 1.82 (s, 3 H) 1.86-1.98 (m, 8 H) 2.01-2.08 (m, 1 H) 2.96 (s, 3 H) 3.13-3.25 (m, 1 H) 3.34-3.41 (m, 3 H) 3.41-3.53 (m, 2 H) 3.62-3.74 (m, 3 H) 3.85-3.96 (m, 1 H) 4.04-4.10 (m, 2 H) 4.11-4.18 (m, 1 H) 4.18-4.24 (m, 1 H) | 350.3 |
| 82 | NA | NA | 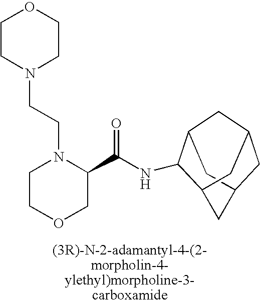 | N | (400 MHz, MeOD) δ ppm 1.64-1.71 (m, 2 H) 1.80 (s, 2 H) 1.83-1.94 (m, 10 H) 2.32-2.44 (m, 2H) 2.44-2.49 (m, 4 H) 2.50-2.59 (m, 2 H) 2.76 (ddd, J=12.00, 8.72, 6.06 Hz, 1 H) 3.01-3.08 (m, 2H) 3.53 (dd, J=11.24, 9.22 Hz, 1 H) 3.59-3.68 (m, 5 H) 3.78-3.86 (m, 2 H) 3.97 (s, 1 H) | 378 |
| 83 | NA | NA | 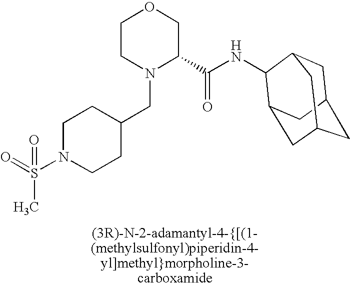 | H | (400 MHz, MeOD) δ ppm 1.29-1.44 (m, 2 H) 1.67 (t, J=10.23 Hz, 2 H) 1.81-2.07 (m, 14 H) 2.14-2.16 (m, 1H), 2.74-2.85 (m, 5 H) 3.00-3.11 (m, 2 H) 3.25-3.31 (m, 1 H) 3.69-3.78 (m, 4 H) 3.83-3.93 (m, 1 H) 4.01-4.14 (m, 3 H) 4.21 (dd, J=12.63, 3.54 Hz, 1 H) 8.50 (d, J=7.07 Hz, 1 H) | 440.3 |
| 84 | NA | 62 | 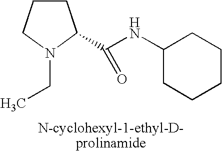 | A | (400 MHz, CDCl₃) δ: 7.37 (s, 1H), 3.64-3.85 (m, 1H), 3.17 (t, 1H), 2.98 (dd, 1H), 2.56-2.72 (m, 1 H), 2.40-2.56 (m, 1 H), 2.23-2.37 (m, 1H), 2.06-2.23 (m, 1H), 1.54-1.95 (m, 8H), 1.29-1.48 (m, 2H), 1.10-1.27 (m, 3H), 1.06 (t, 3H). | 225 |
| 85 | 9 | 94 | 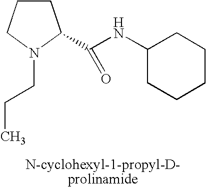 | A | (CDCl₃, 400 MHz) δ: 7.37 (1H, d), 3.74 (1H, m); 3.15 (1H, m); 2.97 (1H, m); 2.52 (1H, m); 2.41 (1H, m; 2.27 (1H, m); 2.18-2.10 (1H, m); 1.92-1.30 (12H, m); 1.24-1.08 (3H, m); 0.92 (3H, t). | 239 |
| 86 | <1 | 100 | 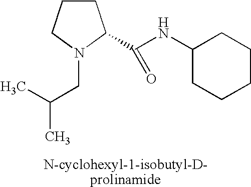 | A | (CDCl₃, 400 MHz) δ: 7.35 (s, 1H), 3.64-3.85 (m, 1 H), 3.07-3.22 (m, 1 H), 2.96 (dd, 4.80 Hz, 1 H), 2.07-2.32 (m, 5 H), 1.65-1.94 (m, 7 H), 1.29-1.48 (m, 3 H), 1.06-1.24 (m, 3 H), 0.96 (d, 3 H), 0.88 (d, 3 H). | 253 |
| 87 | NA | 54 | 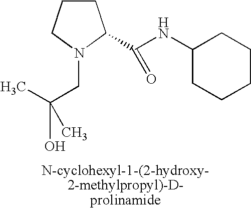 | G | (CDCl₃, 400 MHz) δ: 7.32 (1H, d); 3.75-3.65 (1H, m); 3.40-3.30 (1H, m); 3.16 (1 H, dd); 2.66 (1 H, d); 2.49 (1H, d); 2.46 (1H, dt); 2.18-2.09 (1 H, m); 1.90-1.55 (9H, m); 1.45-1.30 (2H, m), 1.25, (3H, s); 1.24 (3H, s); 1.23-1.10 (3H, m). | 268 |
| 88 | 7 | 94 | 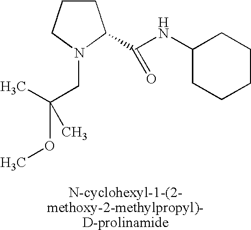 | H | (CDCl₃, 400 MHz) δ: 7.54 (1H, d); 3.75-3.65 (1H, m); 3.40-3.30 (1H, m); 3.20 (3H, s), 3.10 (1H, dd); 2.60 (1H, d); 2.53 (1H, d); 2.39 (1H, dt); 2.15-2.05 (1H, m); 1.95-1.60 (9H, m); 1.45-1.30 (2H, m), 1.18, (3H, s); 1.16 (3H, s); 1.23-1.10 (2H, m). | 283 |
| 89 | NA | 92 | 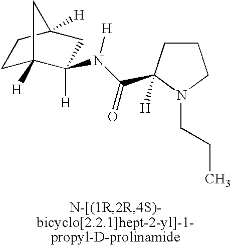 | A | (CDCl₃, 400 MHz) δ: ppm 0.65-0.77 (m, 1 H) 0.94 (t, J=7.45 Hz, 3H) 1.16-1.91 (m, 11H) 2.00-2.33 (m, 4H) 2.35-2.61 (m, 3H) 2.99 (dd, J=10.36, 4.55Hz, 1H) 3.14-3.23 (m, 1H) 4.03-4.19 (m, 1 H), 7.57 (s, 1 H) | 251 |
| 90 | NA | 100 | 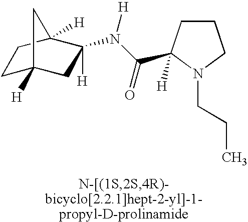 | A | (CDCl₃, 400 MHz) δ: 0.66-0.78 (m, 1H) 0.95 (t, J=7.33 Hz, 3H) 1.16-1.91 (m, 11H) 2.00-2.19 (m, 2H) 2.19-2.32 (m, 2H) 2.35-2.49 (m, 2H) 2.53-2.66 (m, 1H) 3.00 (dd, J=10.11, 4.80 Hz, 1H) 3.12-3.25 (m, 1 H) 4.01-4.18 (m, J=4.29 Hz, 1 H), 7.54 (s, 1 H) | 251 |
| 91 | NA | 100 | 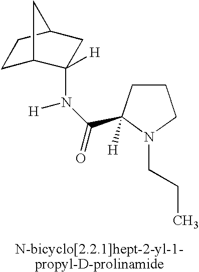 | A | (CDCl₃, 400 MHz) δ: 0.93 (t, 3H) 1.07-1.35 (m, 5H) 1.38-1.58 (m, 4H) 1.59-1.88 (m, 4H) 2.05-2.32 (m, 4H) 2.34-2.45 (m, 1H) 2.46-2.60 (m, 1 H) 2.96 (dd, 1H) 3.14 (t, 1H) 3.64-3.77 (m, 1H), 7.32 (s, 1H) | 251 |
| 92 | NA | 100 | 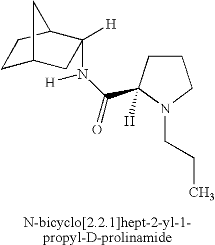 | A | (CDCl₃, 400 MHz) δ: 0.92 (t, 3H) 1.06-1.34 (m, 5H) 1.38-1.57 (m, 4H) 1.61-1.92 (m, 4H) 2.07-2.31 (m, 4H) 2.35-2.45 (m, 1H) 2.45-2.56 (m, 1 H) 2.96 (dd, 1H) 3.10-3.21 (m, 1H) 3.67-3.76 (m, 1 H), 7.33 (s, 1H) | 251 |
| 93 | 2 | 100 | 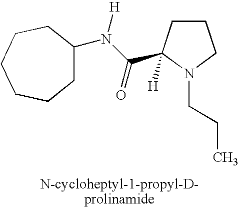 | A | (CDCl₃, 400 MHz) δ: 7.42 (s, 1H), 3.86-4.01 (m, 1 H), 3.10-3.21 (m, 1 H), 2.96 (dd, 1 H), 2.46-2.57 (m, 1 H), 2.36-2.45 (m, 1 H), 2.21-2.31 (m, 1 H), 2.08-2.20 (m, 1 H), 1.34-1.94 (m, 18 H), 0.92 (t, 3 H). | 253 |
| 94 | 11 | 86 | 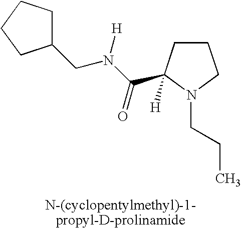 | A | (CDCl₃, 400 MHz) δ: 0.92 (t, 3H) 1.11-1.31 (m, 2H) 1.40-1.94 (m, 11H) 1.97-2.24 (m, 2H) 2.24-2.66 (m, 3H) 3.10-3.30 (m, 4H), 7.54 (s, 1H) | 239 |
| 95 | 39 | 78 | 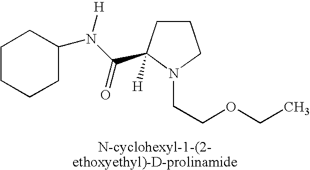 | A | (CDCl₃, 400 MHz) δ: 7.51 (1 H, d); 3.75-3.68 (1 H, m); 3.50-3.340 (3H, m); 3.24 (1H, dt), 3.06 (1H, dd); 2.83 (1H, ddd); 2.63 (1H, dt); 2.34 (1H, dt); 2.20-2.10 (1H, m); 1.90-1.80 (3H, m); 1.79-1.55 (6H, m); 1.43-1.30 (2H, m), 1.23-1.10 (6H, m). | 269 |
| 96 | 39 | 84 | 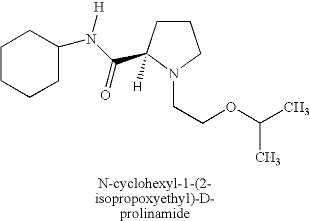 | A | (CDCl₃, 400 MHz) δ: 7.51 (1H, d); 3.80-3.70 (1H, m); 3.60-3.54 (1 H, m); 3.50-40 (2H, m), 3.22 (1H, dt); 3.07(1 H, dd), 2.82 (1H, ddd); 2.60 (1H, dt); 2.34 (1H, dt); 2.20-2.10 (1H, m); 1.90-1.80 (3H, m); 1.79-1.55 (5H, m); 1.43-1.30 (2H, m), 1.17 (6H, d), 1.23-1.10 (3H, m). | 283 |
| 97 | 7 | 100 | 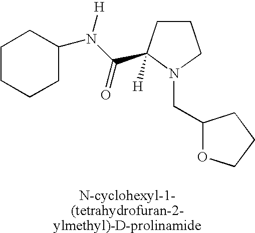 | A | (MeOD, 400 MHz) δ: 6.51 (1H, bs); 6.12 (1H, dd); 2.93-2.72 (2H, m); 2.57-2.32 (4H, m); 2.05-1.78 (2H, m), 1.21-1.08 (1H, m); 0.82-0.18 (18H, m). | 281 |
| 98 | NA | 41 | 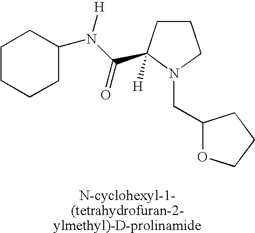 | A | (MeOD, 400 MHz) δ: 6.71 (1 H, m); 6.57 (1 H, d); 3.03 (1 H, m); 2.95 (1 H, m); 281 2.56-2.14 (5H, m), 0.94-0.16 (20H, m) | 281 |
| 99 | 8 | 91 | 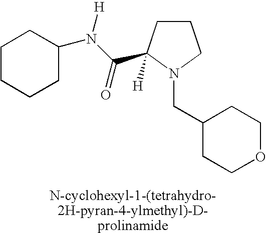 | A | (CDCl₃, 400 MHz) δ: 7.21 (1 H, d); 3.99 (2H, m); 3.80-3.70 (1 H, m); 3.39 (2H, m), 3.15 (1 H, m); 2.97 (1H, m); 2.42 (1 H, m); 2.32 (1 H, m); 2.30 (1 H, m); 2.20-2.10 (1 H, m); 1.95-1.50 (12H, m); 1.45-1.05 (6H, m). | 295 |
| 100 | 4 | 100 | 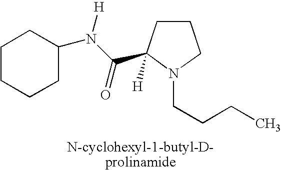 | A | (CDCl₃, 400 MHz) δ: 7.35 (1H, d), 3.74 (1H, ddd); 3.16 (1H, ddd); 2.97 (1H, dd); 2.58 (1H, dt); 2.41 (1H, ddd); 2.27 (1H, ddd); (7H, m); 0.92 (3H, t). | 253 |
| 101 | 5 | 100 | 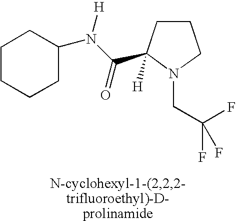 | A | (CDCl₃, 400 MHz) δ: 7.12(s, 1H), 3.61-3.87 (m, 2H), 3.38 (t, 1H), 3.18-3.30 (m, 1H) 3.02-3.18 (m, 1H), 2.46-2.63 (m, 1 H), 2.08-2.42 (m, 2H), 1.77-2.07 (m, 4H), 1.01-1.51 (m, 8H). | 279 |
| 102 | 7 | 91 | 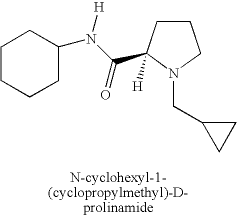 | A | (CDCl₃, 400 MHz) δ: 7.31 (s, 1H), 3.53-3.69 (m, 1H), 3.15 (t, 1H), 2.83-2.94 (m, 1H), 2.13-2.34 (m, 3H), 1.96-2.11 (m, 1H), 1.41-1.83 (m, 8H), 1.17-1.34 (m, 2H), 0.95-1.15 (m, 3H), 0.65-0.80 (m, 1 H), 0.28-0.47 (m, 2H), 0.09-0.06 (m, 2H). | 251 |
| 103 | 14 | 83 | 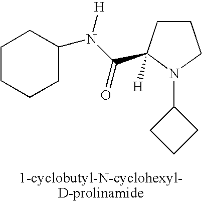 | A | (CDCl₃, 400 MHz) δ: 7.41 (s, 1H), 3.65-3.85 (m, 1H), 3.27 (t, 1H), 2.94-3.05 (m, 1H), 2.27-2.49 (m, 2H), 2.05-2.25 (m, 1H), 1.51-1.97 (m, 9H), 1.29-1.48 (m, 2H), 1.05-1.29 (m, 3H), 0.72-0.96 (m, 1 H), 0.49 (d, 2H), 0.11 (d, 2H) | 251 |
| 104 | 6 | 100 | 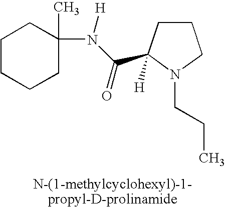 | A | (CDCl₃, 400 MHz) δ: 7.34 (1H, bs), 2.88 (1H, dd); 2.55 (1H, ddd); 2.40 (1H, ddd); 2.27 (1H, ddd); 2.19-2.09 (2H, m); 2.00-1.94 (3H, m); 1.59-1.23 (13H, m); 0.93 (3H, t). | 253 |
| 105 | 15 | 82 | 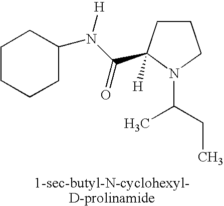 | A | (CDCl₃, 400 MHz) δ: 7.53 (s, 1H), 3.64-3.82 (m, 1H), 3.38-3.20 (m, 1 H), 2.42-2.66 (m, 2H), 2.06-1.84 (m, 3H), 1.79-2.12 (m, 4H), 1.30-1.79 (m, 8H), 1.08-1.28 (m, 3H), 1.02 (d, 1H), 0.95 (m, 2H), 0.86-0.92 (m, 3H) | 253 |
| 106 | 2 | 100 | 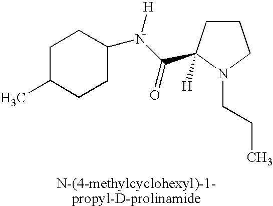 | A | (CDCl₃, 400 MHz) δ: 7.68 (s, 1H), 3.92-4.06 (m, 1H), 3.54-3.75 (m, 1 H), 3.08-3.26 (m, 2H), 2.91 -3.05 (m, 2H), 2.35-2.65 (m, 3H), 2.06-2.33 (m, 4H), 1.40-2.00 (m, 7H), 1.01-1.21 (m, 3H), 0.86-0.99 (m, 4H) | 253 |
| 107 | 2 | 100 | 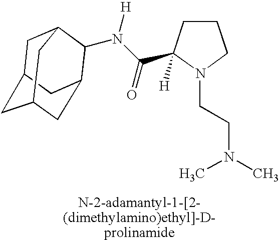 | A | (CDCl₃, 400 MHz) δ: 8.02 (m, 1 H), 3.96-4.06 (m, J=8.59 Hz, 1H), 3.19-3.29 (m, 1H), 3.09 (dd, J=10.11, 4.29 Hz, 1 H), 2.74-2.88 (m, 1H), 2.52-2.63 (m, 1H), 2.51-2.63 (m, 1H), 2.31-2.49 (m, 2H), 2.23 (s, 6H), 2.19-2.21 (m, 3H), 2.06-2.20 (m, 2H), 1.53-1.97 (m, 14H). | 320 |
| 108 | 9 | 100 | 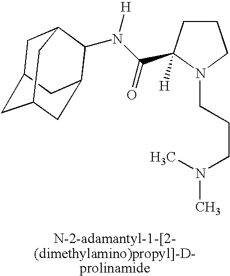 | A | (CDCl₃, 400 MHz) δ: 7.60-7.78 (m, J=8.34 Hz, 1 H), 3.95-4.04 (m, J=8.08 Hz, 1H), 3.23 (t, J=7.45 Hz, 1H), 3.04-3.14 (m, J=9.98, 4.42 Hz, 1H), 2.75-3.00 (m, 2H), 2.66 (s, 6H), 2.52-2.62 (m, 1H), 2.28-2.40 (m, 1 H), 2.10-2.23 (m, 3H), 1.55-1.98 (m, 18H). | 334 |
| 109 | NA | 77 | 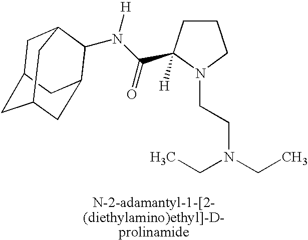 | I | (400 MHz, MeOD) δ ppm 1.35 (t, J=7.20 Hz, 6 H) 1.69-1.70 (m, 2H) 1.81-2.03 (m, 16 H) 2.24-2.34 (m,1 H) 2.50-2.57 (m, 1 H) 2.83-2.94 (m, 2 H) 3.08-3.10 (m, 1 H) 3.17-3.29 (m, 5 H) 3.38 (d, J=7.58 Hz, 1 H) 4.01 (s, 1 H) | 348 |
| 110 | NA | 100 | 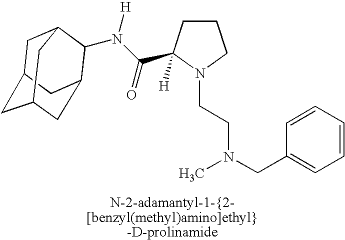 | I | (400 MHz, MeOD) δ ppm 1.61-1.69 (m, 3 H) 1.76-1.87 (m, 12 H) 1.87-1.96 (m, 2 H) 2.08-2.19 (m, 1 H) 2.19-2.26 (m, 3 H) 2.31 (td, J=9.60, 6.06 Hz, 1 H) 2.44 (ddd, J=12.32, 8.02, 4.67 Hz, 1 H) 2.56-2.63 (m, 1 H) 2.78-2.87 (m, 2 H) 3.02-3.12 (m, 2 H) 3.47-3.58 (m, 2 H) 3.90 (s, 1 H) 7.22-7.33 (m, 5 H) | 396 |
| 111 | NA | 96 | 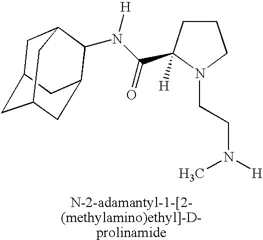 | J | (400 MHz, MeOD) δ ppm 1.65 (s, 2H) 1.80 (s, 2 H) 1.83-1.96 (m, 16H) 2.09-2.17 (m, 1H) 2.19-2.26 (m, 1H) 2.38-2.47 (m, 1H) 2.97 (s, 3H) 3.43 (dt, J=9.79, 7.36 Hz, 1H) 3.52 (ddd, J=9.85, 6.82, 5.05 Hz, 1H) 3.94 (s, 1H) 4.28 (dd, J=8.46, 3.92 Hz, 1H) | 306 |
| 112 | NA | 81 | 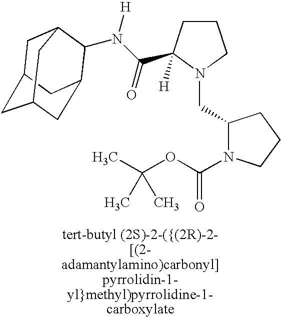 | B | (CDCl₃, 400 MHz) δ: 7.69-7.85 (m, 1 H), 3.93-4.05 (m, 1H), 3.26-3.78 (m, 6H), 3.20 (dd, J=10.11, 3.79 Hz, 1H), 2.68-2.90(m, 1H), 2.68-2.90 (m, 1H), 2.33-2.54 (m, J=12.13, 8.08 Hz, 1H), 2.03-2.26 (m, 2H), 1.53-2.03 (m, 18 H), 1.47 (s, 9H). | 432 |
| 113 | NA | 100 | 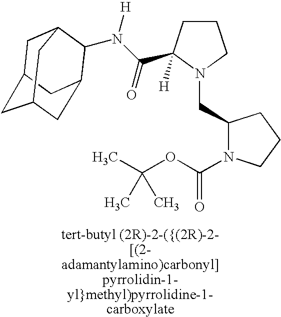 | B | (CDCl₃, 400 MHz) δ: 7.61-7.93 (m, 1H), 3.81-4.07 (m, 2H), 3.40-3.72 (m, 2H), 3.18-3.38 (m, 2H), 2.31-2.69 (m, 4H), 2.10-2.25 (m, 3H), 1.57-2.06 (m, 18H), 1.47 (s, 9H). | 432 |
| 114 | NA | 100 | 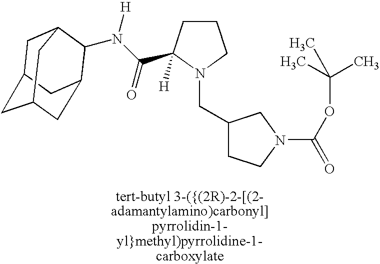 | B | (CDCl₃, 400 MHz) δ: 7.61-7.85 (m, 1 H), 3.90-4.09 (m, 1 H), 3.17-3.73 (m, 6H), 2.82-3.11 (m, 3H), 2.07-2.69 (m, 5H), 1.52-2.00 (m, 16H), 1.45 (s, 9H). | 432 |
| 115 | NA | 100 | 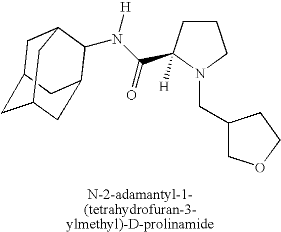 | B | (CDCl₃, 400 MHz) δ: 7.64-7.88 (m, 1H), 3.66-4.05 (m, 5H), 3.41 -3.58 (m, 1H), 3.17-3.32 (m, 1H), 2.95-3.13(m, 1H),2.56-2.69 (m, 1H), 2.27-2.57 (m, 3H), 2.26 (m, 2H), 1.59-1.97 (m, 17 H). | 333 |
| 116 | NA | 100 | 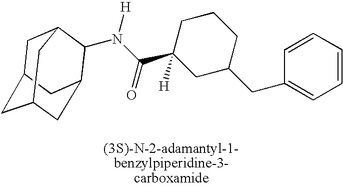 | A | (CDCl₃, 400 MHz) δ: 7.22-7.41 (m, 6H), 4.02-4.12 (m, 1H), 3.39-3.61 (m, 2H), 2.74-3.12 (m, J=28.80 Hz, 2H), 2.46-2.59 (m, 1H), 2.22-2.39 (m, 1H), 1.49-2.11 (m, 19H). | 353 |
| 117 | NA | 100 | 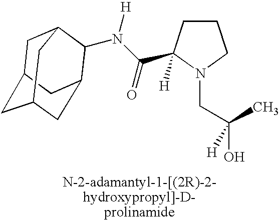 | G | (CDCl₃, 400 MHz) δ: 7.70 (1H, d); 4.03 (1H, m); 3.85 (1H, m); 3.25 (1H, ddd); 3.11 (1H, dd); 2.62-2.54 (2H, m); 2.48 (1H, dt); 2.22-2.12 (1H, m); 1.95-1.60 (18H, m); 1.20 (3H, d). | 307 |
| 118 | NA | 100 | 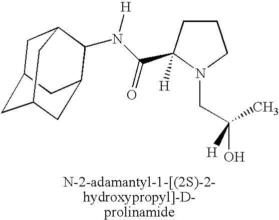 | G | (CDCl₃, 400 MHz) δ: 7.61 (1H, bs); 4.08-3.88 (2H, m); 3.39-3.06 (2H, m); 2.75-2.12 (5H, m); 2.00-1.62 (17H, m); 1.18 (3H, d). | 307 |
| 119 | NA | 100 | 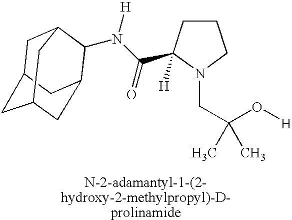 | G | (CDCl₃, 400 MHz) δ: 7.96 (s, 1 H), 3.92-4.05 (m, 1 H), 3.31-3.43 (m, 1H), 3.17 (s, 3H), 3.11-3.21 (m, 1H), 2.52-2.70 (m, 2H), 2.38-2.49 (m, 1H), 2.01-2.18 (m, 1H), 1.55-1.99 (m, 17H), 1.19 (d, J=16.17 Hz, 6H). | 335 |
| 120 | NA | 100 | 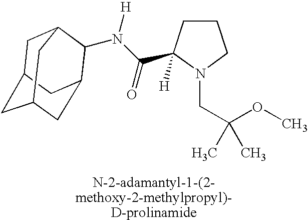 | H | (CDCl₃, 400 MHz) δ: 7.98-7.96 (m, 1H), 3.92-4.05 (m, 1H), 3.31-3.43 (m, 1H), 3.17 (s, 3H), 3.11-3.21 (m, 1H), 2.52-2.70 (m, 2H), 2.38-2.49 (m, 1H), 2.01-2.18 (m, 1H), 1.55-1.99 (m, 17H), 1.19 (d, J=16.17 Hz, 6H). | 335 |
| 121 | NA | 17 | 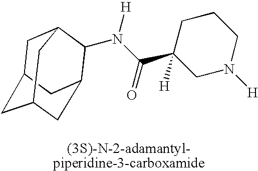 | K | (CDCl₃, 400 MHz) δ: 9.66 (s 1H), 8.94 (s, 1H), 6.35 (d, J=7.83 Hz, 1H), 3.92-4.07 (m, 1 H), 2.66-3.45 (m, 6H), 1.55-2.07 (m, 16H). | 263 |
| 122 | NA | 100 | 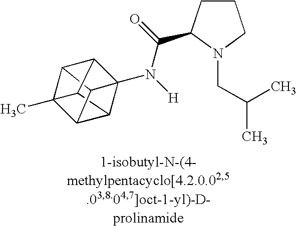 | A | (CDCl₃, 400 MHz) δ: 7.81 (s, 1H), 3.92-4.03 (m, 3H), 3.50-3.64 (m, 3H), 3.09-3.21 (m, J=2.53, 2.53 Hz, 1H), 2.97-3.01 (m, 1H), 2.48-2.67 (m, 1H), 2.37-2.48 (m, 1 H), 2.22-2.33 (m, 1H), 2.07-2.20 (m, 1H), 1.82-1.93 (m, 1H), 1.66-1.82 (m, 2H), 1.40-1.58 (m, 2H), 1.28 (s, 3H), 0.92 (t, J=7.33 Hz, 3H). | 287 |
| 123 | NA | 100 | 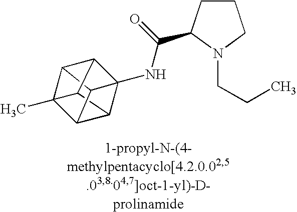 | A | (CDCl₃, 400 MHz) δ: 7.83 (s, 1H), 3.91-4.03 (m, 3H), 3.50-3.62 (m, 3H), 3.10-3.20 (m, 1H), 2.91-3.02 (m, 1H), 1.99-2.39 (m, 5H), 1.82-1.94 (m, 1H), 1.67-1.82 (m, 2H), 1.27 (s, 3H), 0.84-1.01 (m, 6H). | 273 |
| 124 | NA | 100 | 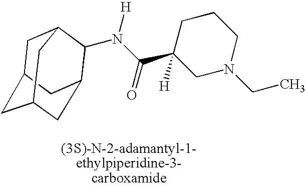 | A | (CDCl₃, 400 MHz) δ: 8.56 (s, 1H), 3.95-4.15 (m, 1H), 2.20-3.21 (m, 6H), 1.54-2.04 (m, 19 H), 1.18 (t, J=7.07 Hz, 3H). | 291 |
| 125 | NA | 81 | 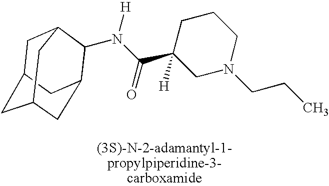 | A | (CDCl₃, 400 MHz) δ: 8.39 (s, 1H), 3.98-4.21 (m, 1H), 2.80-3.09 (m, 2H), 2.59-2.77 (m, 1H), 2.27-2.56 (m, 3H), 1.48-2.03 (m, 21 H), 0.92 (t, J=7.33 Hz, 3H). | 305 |
| 126 | NA | 71 | 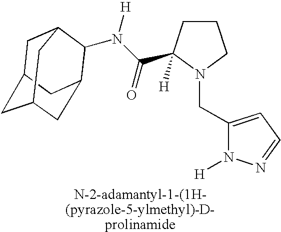 | B | (CDCl₃, 400 MHz) δ: 7.97 (d, J=8.34 Hz, 1 H), 7.53 (d, J=2.02 Hz, 1H), 6.22 (d, J=2.02 Hz, 1H), 3.86-4.06 (m, 2H), 3.69-3.80 (m, 1 H), 3.31 (dd, J=10.11, 4.55 Hz, 1H), 3.09-3.20 (m, 1H), 2.44-2.59 (m, 2H), 2.11-2.28 (m, 2H), 1.52-2.01 (m, 16H). | 329 |
| 127 | NA | 100 | 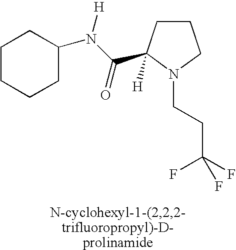 | A | (400 MHz, MeOD) δ ppm 1.19-1.40 (m, 5 H) 1.65 (ddd, J=12.76, 3.54, 3.41 Hz, 1 H) 1.75-1.83 (m, 2 H) 1.84-1.94 (m, 2H) 1.97-2.08 (m, 2H) 2.18 (td, J=7.96, 3.79 Hz, 1 H) 2.54 (td, J=8.65, 5.94 Hz, 1 H) 2.66-2.77 (m, 2 H) 3.19-3.28 (m, 1 H) 3.40-3.52 (m, 2 H) 3.72-3.79 (m, 2 H) 4.10 (dd, J=8.46, 6.95 Hz, 1 H) | 293 |
| 128 | NA | 100 | 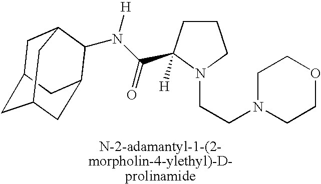 | I | (400 MHz, MeOD) δ ppm 1.68 (d, J=2.02 Hz, 2 H) 1.80 (s, 3 H) 1.85 (s, 3 H) 1.89-1.96 (m, 6 H) 1.98-2.00 (m, 1 H) 2.01-2.03 (m, 1 H) 2.05-2.15 (m, 1 H) 2.45-2.55 (m, 1 H) 2.93-3.03 (m, 6 H) 3.13 (ddd, J=13.58, 8.40, 5.56 Hz, 1 H) 3.37 (ddd, J=13.64, 8.34, 5.31 Hz, 2 H) 3.65-3.73 (m, 1 H) 3.81-3.88 (m, 4 H) 3.96-4.04 (m, 2 H) 8.25 (s, 1 H) | 362 |
| 129 | NA | 72 | 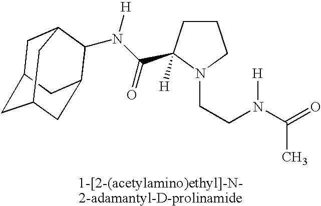 | L | (CDCl₃, 400 MHz) δ: 7.60 1H, d); 5.66 (1H, m); 4.03 (1H, m); 3.52-3.44 (1H, m); 3.34-3.24 (2H, m); 3.07 (1H, dd); 2.79 (1H, ddd); 2.61 (1 H, ddd); 3.36 (1 H, dt); 2.22-2.13 (1H, m); 1.97 (3H, s); 1.92-1.62 (17H, m) | 334 |
| 130 | NA | 56 | 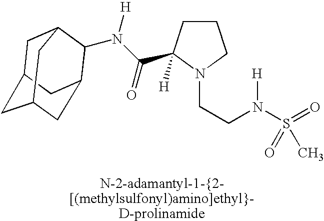 | M | (CDCl₃, 400 MHz) δ: 7.41 (1 H, d); 4.55 (1 H, m); 4.01 (1H, m); 3.28-3.20 (3H, m); 3.10 (1H, dd); 2.95 (3H, s); 2.95-2.85 (1 H, m); 2.67 (1H, ddd); 2.36 (1H, dt); 2.25-2.15 (1H, m); 1.95-1.56(1 7H, m). | 370 |
| 131 | NA | 88 | 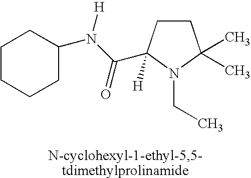 | A | (CDCl₃, 400 MHz) δ: 7.44 (s, 1H) 3.67-3.83 (m, 1H), 3.11 (d, J=9.85 Hz, 1H), 2.67-2.82 (m, 1H), 2.15-253 2.42 (m, 2H), 1.52-1.97 (m, 8H), 1.31-1.49 (m, 2H), 1.10-1.28 (m, 6H), 0.92-1.05 (m, 6H). | 253 |
| 132 | NA | 92 | 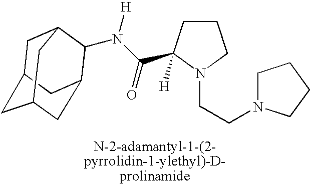 | A | (400 MHz, MeOD) δ ppm 1.65 (d, J=12.88 Hz, 2 H) 1.79 (s, 2 H) 1.83-1.94 (m, 10 H) 1.96 (d, J=6.57 Hz, 1 H) 1.99-2.02 (m, 1 H) 2.07-2.15 (m, 4 H) 2.36-2.46 (m, 1 H) 2.77-2.88 (m, 1 H) 3.16-3.26 (m, 2 H) 3.31-3.33 (m, 2 H) 3.35-3.42 (m, 4 H) 3.43-3.47 (m, 1 H) 3.51-3.58 (m, 1 H) 3.70 (t, J=7.58 Hz, 1 H) 4.00 (s, 1 H) | 346 |
| 133 | NA | NA | 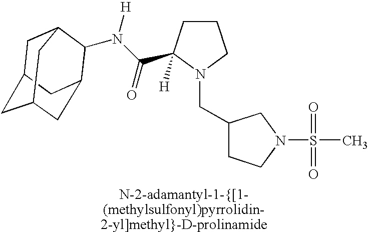 | M | (CDCl₃, 400 MHz) δ: 7.64 (dd, J=27.41, 7.96 Hz, 1 H), 3.91-4.11 (m, 1H), 3.16-3.79 (m, SH), 2.93-3.14 (m, 2H), 2.82 (d, J=5.31 Hz, 3H), 2.26-2.71 (m, 4H), 2.01-2.26 (m, 2H), 1.52-1.98 (m, 1 7H). | 410 |
| 134 | NA | 96 | 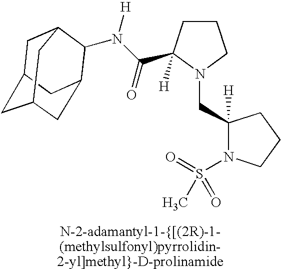 | M | (CDCl₃, 400 MHz) δ: 7.64-7.75 (m, 1 H), 3.97-4.09 (m, 1 H), 3.79-3.91 (m, 1H), 3.27-3.43 (m, 2H), 3.19-3.27 (m, 1H), 3.07-3.16 (m, 1H), 2.84 (s, 3H), 2.78-2.83 (m, 1 H), 2.59 (t, J=11.24 Hz, 1H), 2.40-2.50 (m, 1 H), 2.09-2.26 (m, 1 H), 1.54-2.07 (m, 21H). | 410 |
| 135 | NA | 100 | 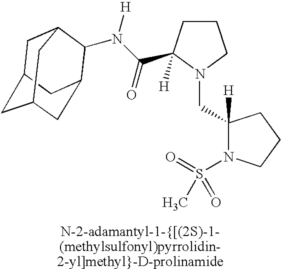 | M | (CDCl₃, 400 MHz) δ: 7.69-7.86 (m, 1 H), 3.94-4.07 (m, 1 H), 3.66-3.79 (m, 1 H), 3.27-3.43 (m, 3H), 3.12-3.23 (m, 1H), 2.90 (dd, J=12.51, 5.94 Hz, 1H), 2.80 (s, 3H), 2.38-2.56 (m, 2H), 2.06-2.25 (m, 1H), 1.56-2.04 (m, 21H) | 410 |
| 136 | NA | 100 | 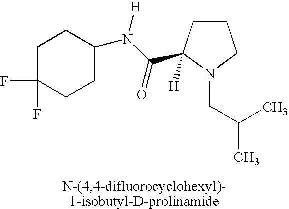 | A | (CDCl₃, 400 MHz) δ: 7.33-7.53 (m, 1 H), 3.73-3.97 (m, 1 H), 3.08-3.23 (m, 1H), 2.91-3.03 (m, 1H), 1.61-2.35 (m, 14H), 1.41-1.60 (m, 2H), 0.83-1.00 (m, 6 H) | 289 |
| 137 | NA | 87 | 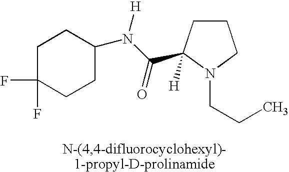 | A | (CDCl₃, 400 MHz) δ: 7.36-7.55 (m, 1 H), 3.79-3.94 (m, 1H), 3.16 (t, J=7.20 Hz, 1H), 3.00 (dd, J=10.23, 4.42 Hz, 1 H), 2.38-2.57 (m, 2H), 2.24-2.34 (m, 1H), 1.37-2.22 (m, 14H), 0.92 (t, J=7.33 Hz, 3H) | 275 |
| 138 | NA | 100 | 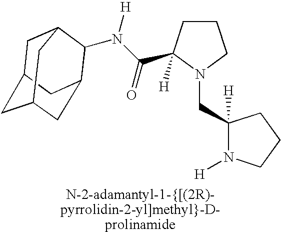 | N | (CDCl₃, 400 MHz) δ: 7.84-8.04 (m, 1H), 3.95-4.16 (m, 1 H), 2.81-3.38 (m, 5 H), 2.52-2.73 (m, 2H), 2.36-2.51 (m, 1H), 1.55-2.31 (m, 22H), 1.24-1.48 (m, 1H) | 332 |
| 139 | NA | 100 | 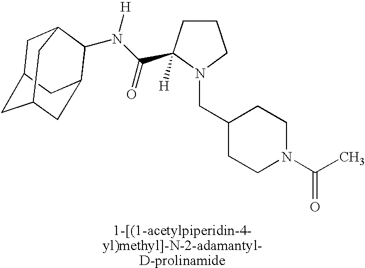 | L | (CDCl₃, 400 MHz) δ: 7.72 (d, J=8.08 Hz, 1H), 4.51-4.70 (m, 1H), 3.99 (t, J=9.85 Hz, 1H), 3.81 (d, J=13.39 Hz, 3.14-388 3.27 (m, 1H) 2.94-3.10 (m, 2H), 2.42-2.65 (m, 2H), 2.13-2.42 (m, 4H), 2.08 (d, J=2.78 Hz, 3H), 1.53 -2.04 (m, 21H) | 388 |
| 140 | NA | 100 | 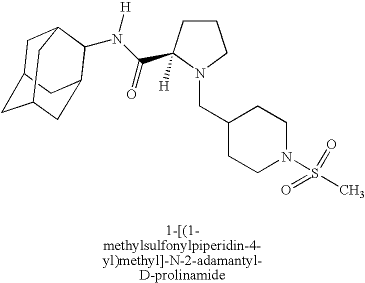 | M | (CDCl₃, 400 MHz) δ: 7.68 (d, J=8.08 Hz, 1H), 4.00 (d, J=8.08 Hz, 1 H), 3.75-3.88 (m, 2H), 3.18 (t, J=7.33 Hz, 1H), 2.98-3.07 (m, 1H), 2.77 (s, 3H), 2.59-2.71 (m, 2H), 2.47-2.56 (m, 1H), 2.34-2.44 (m, 1H), 2.11-2.34 (m, 2H), 2.00-2.10 (m, 1H), 1.50-1.98 (m, 19H), 1.19-1.40 (m, 2H) | 424 |
| 141 | NA | 76 | 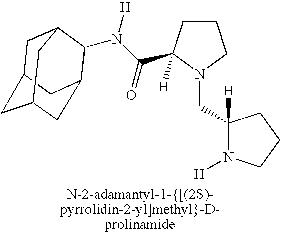 | N | (CDCl₃, 400 MHz) δ: 7.95-8.14 (m, 1H), 3.95-4.12 (m, 1H), 3.21-3.36 (m, 2H), 3.01-3.15 (m, 1H), 2.81-2.96 (m, 2H), 2.50-2.63 (m, 1H), 2.39-2.49 (m, 1H), 2.27-2.38 (m, 1H), 2.07-2.27 (m, 2H), 1.52-2.04 (m, 18H), 1.18-1.41 (m, 2H) | 332 |
| 142 | NA | 100 | 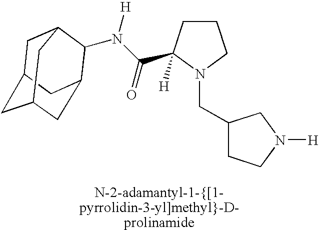 | N | (CDCl₃, 400 MHz) δ: 7.69-7.94 (m, 1H), 3.90-4.11 (m, 1H), 3.19-3.29 (m, 1H), 2.99-3.20 (m, 2H), 2.82-2.98 (m, 2H), 2.43-2.68 (m, 3H), 2.08-2.42 (m, 3H), 1.56-2.03 (m, 18H), 1.22-1.53 (m, 2H) | 332 |
| 143 | NA | 10 | 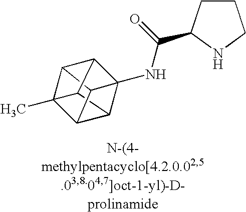 | A | (CDCl₃, 400 MHz) δ: 8.70 (s, 1H), 4.61-4.74 (m, 1H), 3.90-4.04 (m, 3H), 3.54 (t, J=4.42 Hz, 3H), 2,5 3.34-3.45 (m, 2H), 2.35-(m, 1 H), 1.96-2.14 (m, 3H), 1.27 (s, 3H) | 231 |
| 144 | NA | 87 | 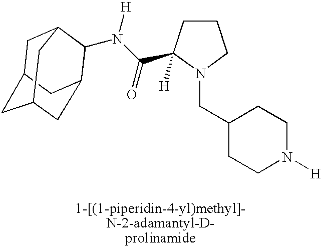 | D | (CDCl₃, 400 MHz) δ: .73-7.89 (m, 1 H), 3.93-4.05 (m, 1H), 3.18 (t, J=7.20 Hz, 1H), 3.04-3.14 (m, 2H), 3.01 (dd, J=10.11, 4.55 Hz, 1H), 2.54-2.67 (m, 2H), 2.43-2.53 (m, 1H), 2.20-2.38 (m, 2H), 2.12-2.16 (m, 3H), 1.49-2.06 (m, 18H), 1.02-1.21 (m, 2H) | 346 |
| 145 | NA | NA | 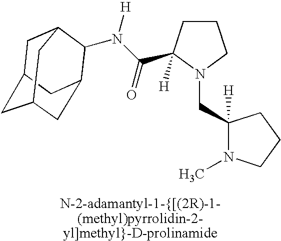 | D | (CDCl₃, 400 MHz) δ: 7.80 (d, J=8.08 Hz, 1H), 4.00 (d, J=8.34 Hz, 1H), 3.21 (t, J=7.07 Hz, 1H), 3.07-3.16 (m, 2H), 2.68-2.80 (m, 1 H), 2.50-2.62 (m, 1 H), 2.39 (s, 3H), 2.30 -2.38 (m, 3H), 2.01-2.30 (m, 3H), 1.56-1.95 (m, 19H) | 346 |
| 146 | NA | NA | 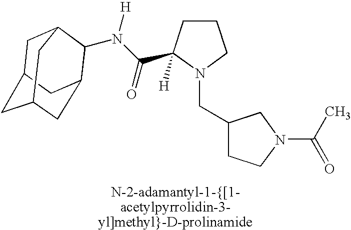 | L | (CDCl₃, 400 MHz) δ: 7.51-7.89 (m, 1H), 3.91-4.15 (m 1H), 3.31-3.77 (m, 3H), 3.18-3.30 (m, 1H), 2.98-3.17 (m, 2H), 2.42-2.71 (m, 2H), 2.26-2.42 (m, 2H), 2.07-2.25 (m, 2H), 2.03 (s, 3H), 1.47-1.96 (m, 18H) | 374 |
| 147 | NA | NA | 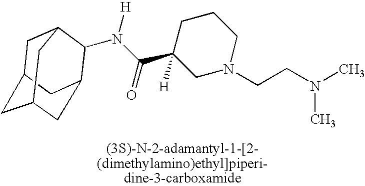 | A | (CDCl₃, 400 MHz) δ: 8.51 (s, 1 H), 4.09 (d, J=8.08 Hz, 1 H), 2.87 (d, J=37.64 Hz, 2 H), 2.77 (s, 6H), 2.44-2.61 (m, 5 H), 2.33-2.45 (m, 1 H), 2.08-2.24 (m, 1 H), 1.48-2.01 (m, 18 H). | 334 |
| 148 | NA | NA | 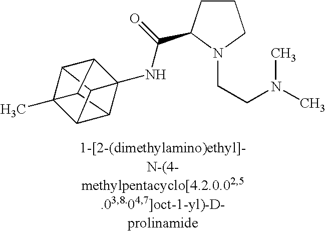 | A | (CDCl₃, 400 MHz) δ: 8.87 (s, 1H), 3.92-4.06 (m, 3 H), 3.50-3.58 (m, 3 H), 3.18-3.34 (m, 1 H), 3.07 (dd, J=9.85, 4.80 Hz, 1 H), 2.71-2.83 (m, 1 H), 2.58-2.69 (m, 1 H), 2.23-2.54 (m, 9 H), 2.05-2.23 (m, 1 H), 1.89-2.03 (m, 1 H), 1.71-1.89 (m, 2 H), 1.27 (s, 3 H). | 302 |
| 149 | NA | NA | 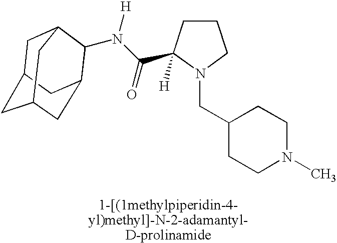 | D | (CDCl₃, 400 MHz) δ: 7.79 (d, J=8.08 Hz, 1 H), 3.99 (d, J=8.34 Hz, 1 H), 3.18 (t, J=7.45 Hz, 1 H), 3.01 (dd, J=9.98, 4.67 Hz, 1 H), 2.82-2.96 (m, 2 H), 2.41-2.55 (m, 1 H), 2.30 (s, 3H), 2.31-2.40 (m, 1 H), 2.09-2.28 (m, 3 H), 1.55-2.04 (m, 20 H), 1.39-1.54 (m, 1 H), 1.21-1.38 (m,2H). | 360 |
| 150 | NA | NA | 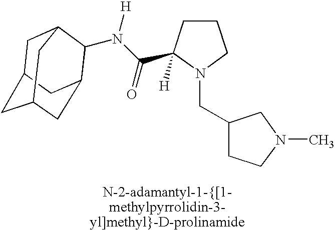 | D | (CDCl₃, 400 MHz) δ: 7.85 (d, J=8.08 Hz, 1 H), 4.00 (d, J=8.34 Hz, 1 H), 3.20 (t, J=7.33 Hz, 1 H), 3.04 (dd, J=10.11, 4.29 Hz, 1 H), 2.43-2.80 (m, 5 H), 2.39-2.35 (m, 2H), 2.33 (s, 3H), 2.34-2.42 (m, 1 H), 1.95-2.27 (m, 3 H), 1.47-1.95 (m, 17 H). | 346 |
| 151 | NA | NA | 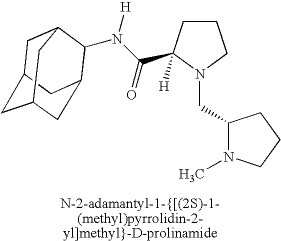 | D | (CDCl₃, 400 MHz) δ: 7.90 (d, J=6.82 Hz, 1 H), 4.02 (d, J=8.08 Hz, 1 H), 3.27 (t, J=7.07 Hz, 1 H), 2.99-3.15 (m, 2H), 2.73-2.91 (m, 1 H), 2.45 (s, 3H), 2.32 2.43 (m, 3 H), 2.08-2.32 (m, 2 H), 1.96-2.08 (m, 1 H), 1.47-1.95 (m, 20 H). | 346 |
| 152 | NA | NA | 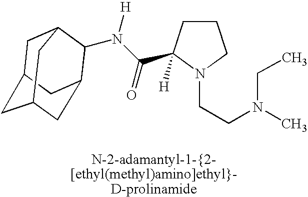 | N | (400 MHz, MeOD) δ: 1.40 (t, J=7.33 Hz, 3H) 1.64-1.72 (m, 2 H) 1.82-2.09 (m, 15 H) 2.16-2.28 (m, 2 H) 2.59-2.70 (m, 2 H) 2.94 (s, 3 H) 3.45-3.59 (m, 2 H) 3.61-3.74 (m, 2 H) 3.77-3.91 (m, 1 H) 4.05 (s, 1 H) 4.26-4.36 (m, 1 H) | 334 |
| 153 | NA | NA | 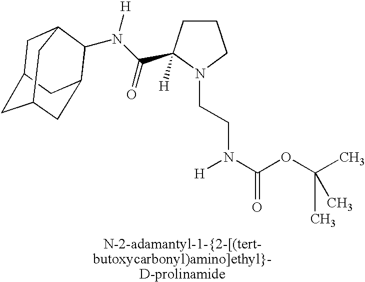 | B | (400 MHz, MeOD) δ: 1.46 (s, 9 H) 1.69-1.96 (m, 17 H) 2.11-2.24 (m, 1 H) 2.35-2.44 (m, 1 H) 2.55 (ddd, J=11.75, 5.81, 5.68 Hz, 1 H) 2.69-2.80 (m, 1 H) 3.05 (dd, J=9.85, 4.04 Hz, 1 H) 3.12-3.26 (m, 3 H) 3.55 (t, J=5.94 Hz, 1 H) 3.92 (s, 1 H) | 392 |
| 154 | NA | 96 | 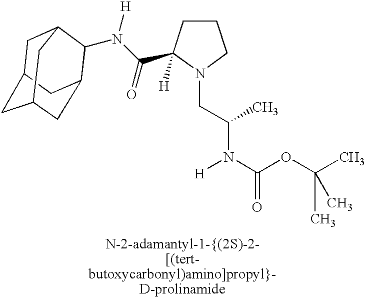 | B | (400 MHz, MeOD) δ: 1.10 (d, J=6.82 Hz, 9 H) 1.10-1.21 (m, 2H) 1.68-2.03 (m, 17H) 2.15 (dt, J=7.33, 3.66 Hz, 1 H) 2.36-2.48 (m, 2 H) 2.60 (dd, J=12.51, 9.73 Hz, 1 H) 3.08 (dd, J=9.98, 3.92 Hz, 1 H) 3.35-3.49 (m, 3 H) 3.57-3.65 (m, 1 H) 3.68-3.80 (m, 1 H) 3.91 (s, 1 H) | 406 |
| 155 | NA | NA | 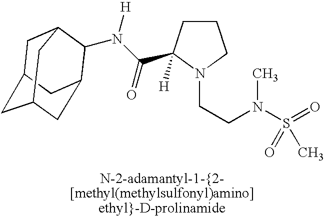 | M | (400 MHz, MeOD) δ: 1.24-1.37 (m, 1 H) 1.66 (d, J=12.63 Hz, 2 H) 1.77-2.13 (m, 15 H) 2.14-2.27 (m, 1 H) 2.55-2.67 (m, 1 H) 2.87-2.98 (m, 6 H) 3.35-3.46 (m, 1 H) 3.55-3.69 (m, 2 H) 3.94 (ddd, J=8.46, 5.18, 4.55 Hz, 1 H) 4.03 (s, 1 H) 4.35 (t, J=7.83 Hz, 1 H) 8.36-8.40 (d, J=6.57 Hz, 1 H) | 384 |
| 156 | NA | NA | 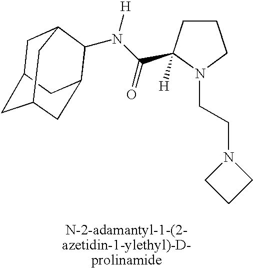 | N | (400 MHz, MeOD) δ: 1.67 (d, J=12.88 Hz, 2 H) 1.80-1.99 (m, 15 H) 2.15-2.58 (m, 2 H) 2.54 (d, J=7.33 Hz, 2 H) 2.96-3.02 (m, 4 H) 3.41-3.84 (m, 4 H) 4.01 (s, 1 H) 4.13-4.27 (m, 3 H) | 332 |
| 157 | NA | NA | 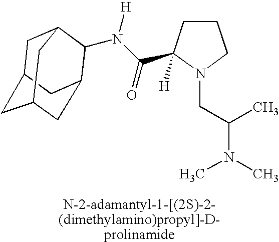 | O | (400 MHz, MeOD) δ: 1.11-1.21 (m, 3 H) 1.65 (d, J=12.13 Hz, 2 H) 1.79-2.03 (m, 16 H) 2.22-2.44 (m, 1 H) 2.78 (s, 3 H) 2.89 (s, 3 H) 3.01 -3.19 (m, 3 H) 3.19-3.28 (m, 1 H) 3.69 (s, 1 H) 3.99 (s, 1 H) 8.09 (s, 1H) | 334 |
| 158 | NA | NA | 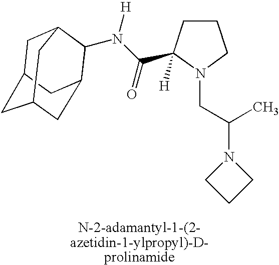 | O | (400 MHz, MeOD) δ: 1.04-1.10 (m, 2 H) 1.24-1.32 (m, 1 H) 1.62-1.70 (m, 2 H) 1.79-2.14(m, 18H) 2.44-2.54 (m, 1 H) 2.61 (ddd, J=13.64, 7.58, 7.07 Hz, 1 H) 2.67-2.72 (m, 1 H) 2.83-3.19 (m, 3 H) 3.47-3.55 (m, 1 H) 3.99 (s, 1 H) 4.09-4.17 (m, 3 H) | 346 |
| 159 | NA | NA | 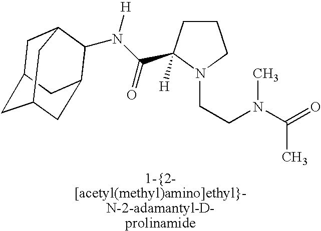 | L | (400 MHz, MeOD) δ: 1.67 (d, J=12.38 Hz, 2 H) 1.80-2.06 (m, 15 H) 2.12-2.26 (m, 5 H) 2.54-2.68 (m, 1 H) 3.05 (s, 3 H) 3.33-3.41 (m, 1 H) 3.45 (ddd, J=8.72, 5.68, 4.80 Hz, 2 H) 3.95 (td, J=8.46, 5.81 Hz, 1 H) 3.99-4.10 (m, 2H) 4.22-4.35 (m, 1 H) 8.32 (s, 1 H) | 348 |
| 160 | NA | NA | 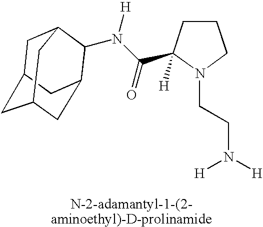 | E | (CDCl₃, 400 MHz) δ: 7.89 (1H, d); 4.02 (1H, m); 3.23 (1H, ddd); 3.08 (1H, dd); 2.87-2.70 (3H, m); 2.57-2.50 (1 H, m); 2.34 (1 H, dt); 2.24-2.13 (1 H, t); 2.10-1.60 (19H, m) | 292 |
| 161 | | 8.7 | 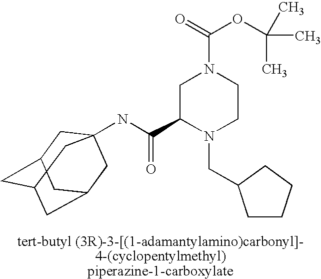 | P | (400 MHz, CDCl₃) δ: 6.53 (bs, 1 H), 4.03 (d, J = 11.60 Hz, 1H), 3.91 (bs, 1H), 304 (d, J = 11.80 Hz, 1H), 2.94 2.88 (m, 2H), 2.61 (dd, J =9.60, 3.80 Hz, 1H), 2.41 (t, J = 11.60 Hz, 1H), 2.18-2.05 (m, 7H), 1.80-1.51 (m, 16H), 1.45 (s, 9H), 1.29-1.22 (m, 2H), 1.13-1.06 (m, 1H). | 446.2 |
| 162 | NA | NA | 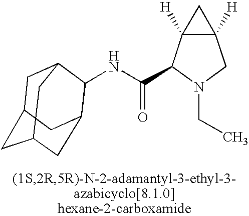 | A | (400 MHz, CDCl₃) δ: 7.49 (bs, 1 H), 4.07 (d, J = 8.84 Hz, 1H), 3.26 (d, J =8.84 Hz, 1H), 3.16 (d, J =4.30 Hz, 1H), 2.68-2.61 (m, 1H), 2.46 (dd, J = 8.85 Hz, 4.80 Hz, 1H), 2.31-2.22 (m, 1H) 2.05-1.63 (m, 15H), 1.50-1.47 (m, 1H), 1.06 (t, J = 7.07 Hz, 3H), 0.59 (q, J =4.04 Hz, 1H), 0.39 (m, 1H). | 361.2 |
| 163 | NA | 100 | 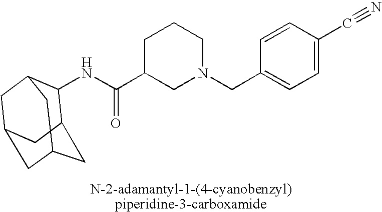 | B | (400 MHz, CDCl₃) δ 7.63 (d, J = 8.08 Hz, 2H), 7.44 (d, J = 7.60 Hz, 2H), 4.05 (d, J = 7.83 Hz, 1H), 3.72-3.51 (m, 2H), 2.87 (bs, 1H), 2.73-2.48 (m, 3H), 1.60 (m, 20H). | 378.2 |
| 164 | NA | 11 | 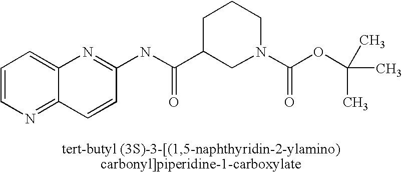 | A | (400 MHz, CDCl₃) δ 8.84 (d, J = 4.30 Hz, 1H), 8.64 (d, J = 9.35 Hz, 1H), 8.41 (d, J = 9.35 Hz, 1H), 8.12 (d, J = 8.60 Hz, 1H), 7.59 (q, J = 4.29 Hz, 1H), 4.22-4.10 (bs, 1H), 3.82 (d, J =14.20 Hz, 1 H), 3.24 (bs, 1H), 2.97 (t, J = 13.39 Hz, 1H), 2.60-2.51 (m, 1H), 2.10-2.05 (m, 1H), 1.93 (t, J = 13.64 Hz, 1H), 1.81-1.55 (m, 5H), 1.19 (s, 9H). | 357.2 |
| 165 | NA | NA | 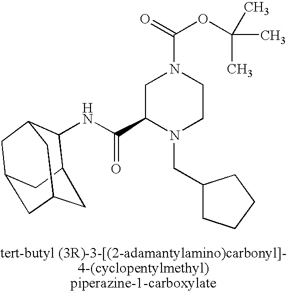 | P | (400 MHz, CDCl₃) δ 7.21 (bs, 1 H), 4.04 (d, J =8.08 Hz, 1 H), 3.88 (bs, 1 H), 3.12-3.03 (m, 2H), 2.82-2.79 (m, 1 H), 2.47 (t, J=11.87 Hz, 1 H), 2.27-2.07 (m, 3H), 1.91-1.75 (m, 24H), 1.45 (s, 9H). | 446.3 |
| 166 | NA | NA | 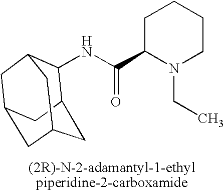 | A | (400 MHz, CDCl₃) δ 7.20 (d, J = 6.32 Hz, 1H), 4.04 (d, J = 8.84 Hz, 1H), 3.13 (d, J = 11.34 Hz, 1H), 2.71-2.60 (m, 2H), 2.17-2.09 (m, 1H), 2.01 -1 .57 (m, 23H), 1.53-1.37 (m, 2H), 1.32-1.23 (m, 1 H), 1.07 (t, J =7.08 Hz, 3H). | 291.2 |
| 167 | NA | NA | 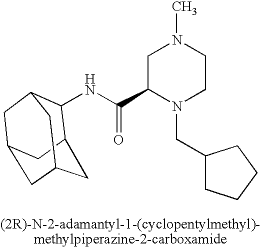 | P | (400 MHz, D2O) δ 3.74 (d, J = 9.60 Hz, 1H), 1.06 (s, 1H), 3.49 (d, J = 14.40 Hz, 1H), 3.40 (d, J = 12.89 Hz, 1H), 3.31 (d, J = 12.13 Hz, 3.13-3.05 (m, 2H), 2.64 (s, 3H), 2.63-2.60 (m, 2H), 2.00-1.89 (m, 1 H), 1.66-1.40 (m, 1 6H), 1.39-1.22 (m, 7H), 0.98-0.91 (m, 1H). | 360.4 |
| 168 | NA | NA | 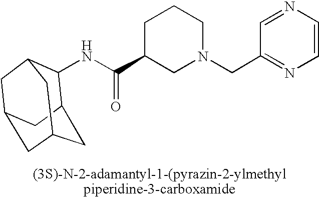 | A | (400 MHz, CDCl₃) δ 8.58 (s, 1H), 8.54 (s, 1H), 8.40 (s, 1H), 4.06 (d, J =7.33 Hz, 1H), 3.70 (s, 2H), 2.99 (bs, 1H), 2.80 (bs, 1H), 2.60-2.47 (m, 2H) 2.30 (t, j = 7.70 Hz, 1 H), 1.95-1.59 (m, 19H). | 355.2 |
| 169 | NA | NA | 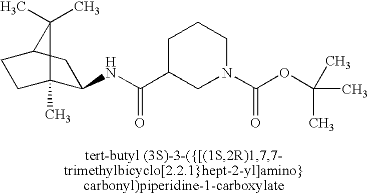 | A | (400 MHz, CDCl₃) δ 5.78 (bs, 1H), 3.93-3.88 (m, 2H), 3.76 (bs, 1 H), 2.97-2.82 (m, 1H), 2.24 (bs, 1H), 2.00-1.60 (m, 9H), 1.45 (s, 9H), 1.31-1.25 (m, 1H), 1.18-1.12 (m, 1H), 0.92 (s, 3H), 0.82 (d, J =5.59 Hz, 6H). | 265.2 (M −Boc +H) |
| 170 | NA | NA | 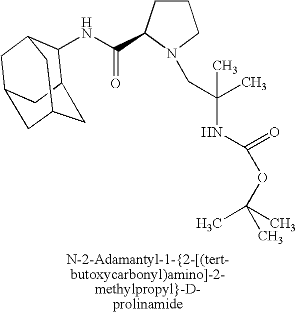 | Q | (400 MHz, CDCl₃), δ:7.80 (m, 1 H), 4.52 (s, 1 H), 4.00 (d, J = 8.3 Hz, 1 H), 3.29 (m, 1 H), 3.23 (dd, J =9.5, 3.7 Hz, 1 H), 2.71-2.91 (m, 2 H), 2.54 (m, 1 H), 2.08 (m, 1 H), 1.57-1.98 (m, 17 H), 1.42 (s, 9 H), 1.30 (s, 3 H), 1.27 (s, 3H) | 420.4 |
| 171 | NA | NA | 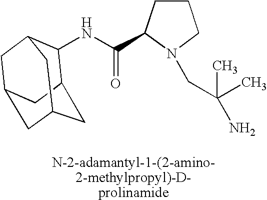 | R | (400 MHz, CDCl₃), δ:7.94 (m, 1 H), 4.02 (d, J= 8.0 Hz, 1 H), 3.35 (m, 1 H), 3.24 (dd, J = 9.3, 4.3 Hz, 1 H), 2.62 (A of AB, J_{AB} =13.1 Hz, 1 H), 2.52 (m, 1 H), 2.46 (B of AB, J_{AB} =13.4 Hz, 1 H); 2.12 (m, 1 H), 1.50-1.94 (m, 19 H), 1.15 (s, 3 H), 1.14 (s, 3H) | 320.2 |
| 172 | NA | NA | 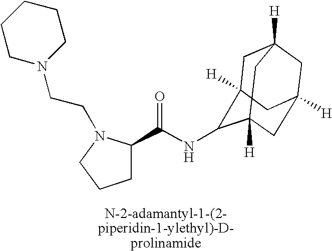 | N | (400 MHz, MeOH-d₄, ppm), δ: 3.85 (bs, 1H), 3.17 (t, 1H), 3.00 (dd, 1H), 2.80-2.70 (m, 1H), 2.62-2.54 (m, 1 H), 2.54-2.45 (m, 1H), 2.44-2.28 (m, 5H), 2.15-2.01 (m, 1H), 1.89-1.57 (m, 1 8H), 1.56-1.47 (m, 4H), 1.43-1.33 (m, 2H) | 360.3 |
| 173 | NA | NA | 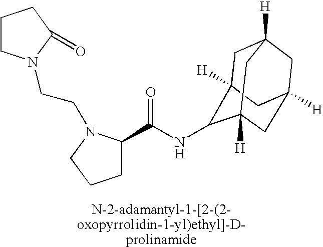 | N | (400 MHz, MeOH-d₄, ppm), δ: 3.73 (bs, 1H), 3.29 (t, 2H), 3.26-3.21 (m, 2H), 3.17-3.08 (m, 1H), 3.94-2.87 (dd, 1 H), 2.74-2.62 (m, 1H), 2.40 (qt, 1H), 2.28-2.20 (m, 1H), 2.16 (t, 2H), 2.04-1.90 (m, 1 H), 1.84 (qt, 2H), 1.76-1.42 (m, 19H) | 360.3 |
| 174 | NA | NA | 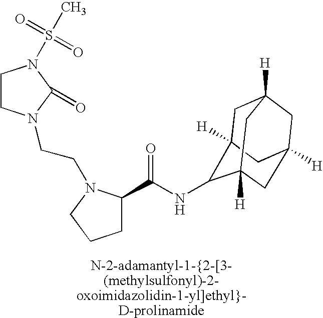 | N | (400 MHz, MeOH-d₄, ppm), δ: 3.85 (bs, 1H), 3.78-3.72 (m, 2H), 3.52-3.44 (m, 2H), 3.40-3.24 (m, 3H), 3.13 (s, 3H), 3.05-2.99 (dd, 1H), 2.88-2.77 (m, 1H), 2.57-2.47 (m, 1H), 2.40-2.31 (m, 1H), 2.17-2.05 (m, 1H), 1.89-1.56 (m, 18H) | 439.2 |
| 175 | NA | NA | 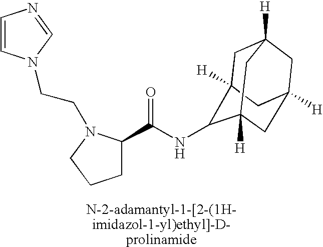 | N | (400 MHz, MeOH-d₄, ppm), δ: 7.80 (s, 1H), 7.17 (s, 1H), 6.95 (s, 1H), 4.15-4.09 (m, 1 H), 3.72 (bs, 1H), 3.25 (bs, 1H), 3.06-2.97 (m, 2H), 2.78-2.70 (m, 1H), 2.43-2.32 (m, 1H), 2.17-2.05 (m, 1H), 1.85-1.57 (m, 14H), 1.53-1.45 (m, 3H), 1.38-1.30 (m, 1H) | 343.2 |
| 176 | No Data | 100 | 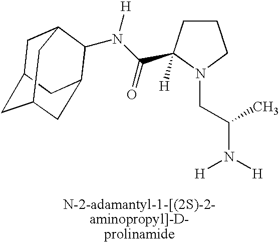 | E | (400 MHz, MeOD) δ ppm 1.24-1.35 (m, 5 H) 1.67 (d, J=12.38 Hz, 2 H) 1.81-2.04 (m, 15 H) 2.36-2.45 (m, 1 H) 2.76-2.80 (m, 1H) 2.90-2.99 (m, 2 H) 3.58 (m, 3 H) 3.68-3.78 (m, 1 H) 4.02 (s, 1 H) | 306 |
| 177 | 18 | 92.2 | 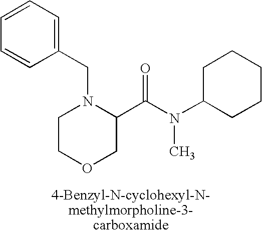 | T | NA | 317.2 |
| 178 | 1 | 100 | 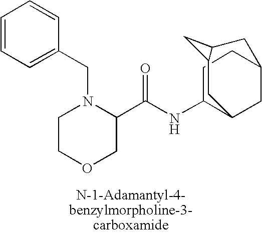 | T | NA | 355.1 |
| 179 | 2 | 100 | 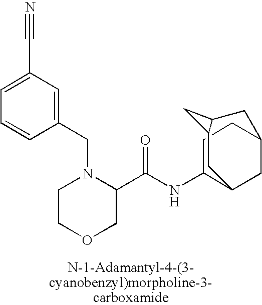 | T | NA | 380.2 |
| 180 | 32 | 72.1 | 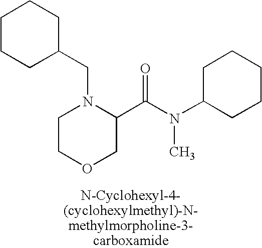 | T | NA | 323.2 |
| 181 | 1.8 | 100 | 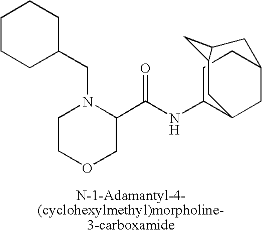 | T | NA | 361.1 |
| 182 | 1.6 | 100 | 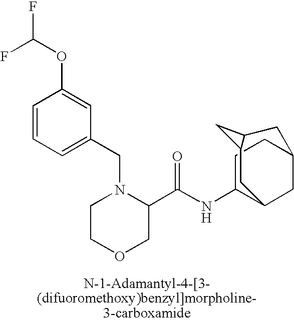 | T | NA | 421.1 |
| 183 | 2.1 | 100 | 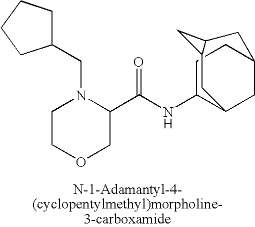 | T | NA | 347.2 |
| 184 | 3.2 | 100 | 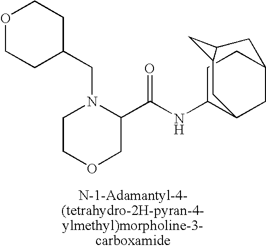 | T | NA | 363.3 |
| 185 | 14 | 91.3 | 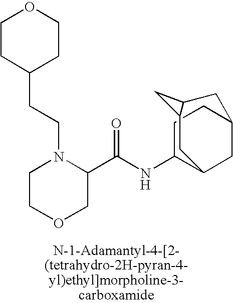 | T | NA | 377.2 |
| 186 | 1 | 100 | 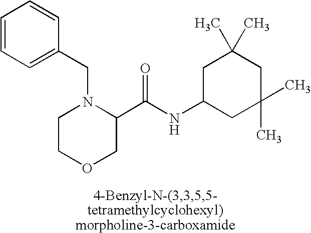 | T | NA | 359.2 |
| 187 | 3 | 100 | 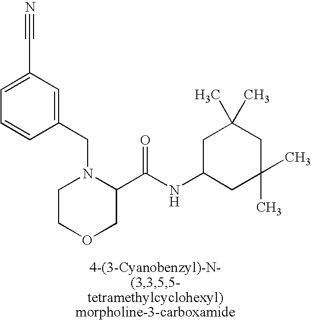 | T | NA | 384.2 |
| 188 | 1.7 | 100 | 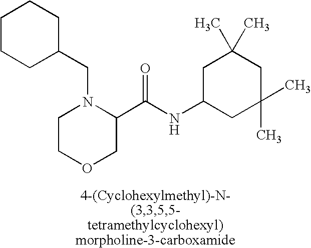 | T | NA | 365.2 |
| 189 | 1.1 | 100 | 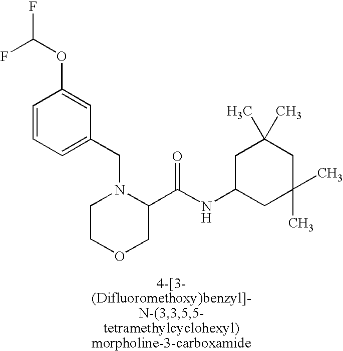 | T | NA | 425.2 |
| 190 | 1.1 | 100 | 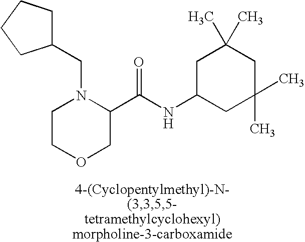 | T | NA | 351.2 |
| 191 | 28 | 83.7 | 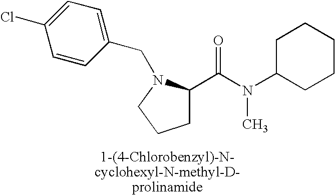 | T | NA | 335.2 |
| 192 | 9.6 | 100 | 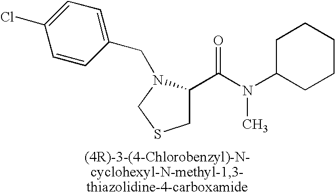 | T | NA | 353.2 |
| 193 | 14 | 100 | 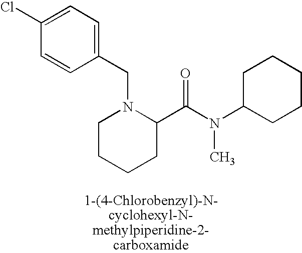 | T | NA | 349.1 |
| 194 | 25 | 86.2 | 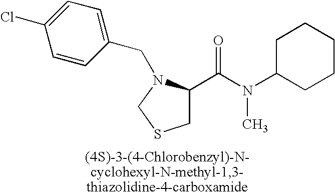 | T | NA | 353.0/ 355.1 |
| 195 | 13 | 100 | 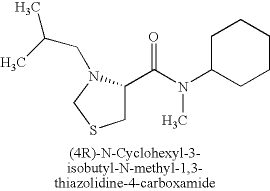 | T | NA | 285.2 |
| 196 | 27 | 85.3 | 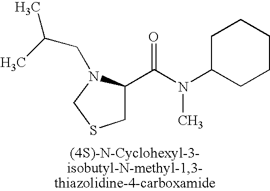 | T | NA | 285.1 |
| 197 | 50 | 73.2 | 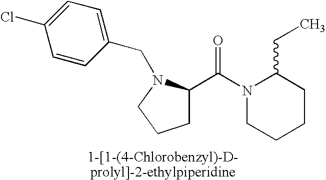 | T | NA | 335.2/ 337.2 |
| 198 | 2.5 | 100 | 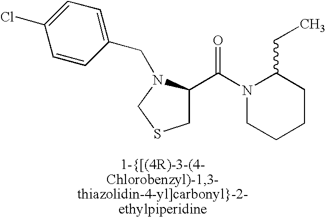 | T | NA | 353.0/ 355.1 |
| 199 | 32 | 80.2 | 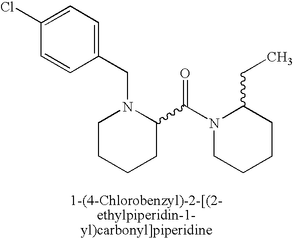 | T | NA | 349.1 |
| 200 | 36 | 91.6 | 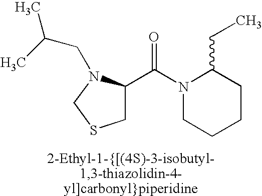 | T | NA | 285.1 |
| 201 | 5.9 | 100 | 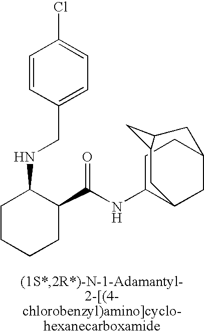 | T | NA | 401.2/ 403.1 |
| 202 | 4.8 | 100 | 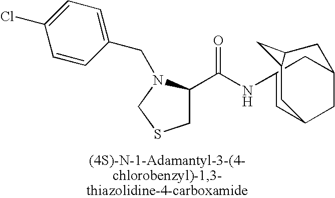 | T | NA | 391.1 |
| 203 | 1 | 100 | 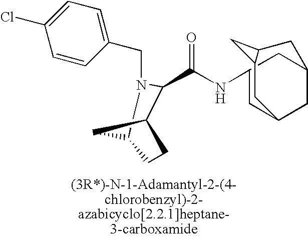 | T | NA | 399.1/ 401.2 |
| 204 | 1 | 100 | 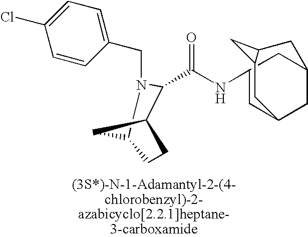 | T | NA | 399.1/ 401.2 |
| 205 | 3.17 | 100 | 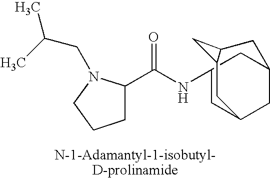 | T | NA | 305.2 |
| 206 | 5.2 | 100 | 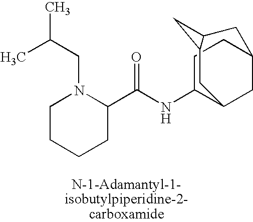 | T | NA | 319.3 |
| 207 | 11 | 92.1 | 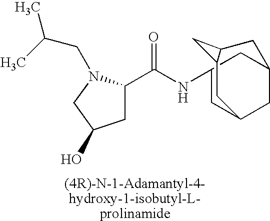 | T | NA | 321.2 |
| 208 | 1 | 100 | 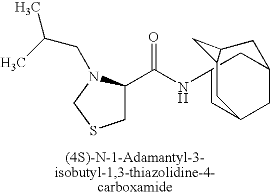 | T | NA | 323.2 |
| 209 | 1.9 | 100 | 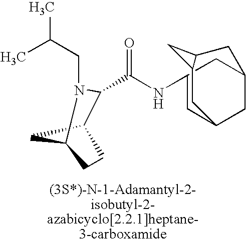 | T | NA | 331.1 |
| 210 | 1 | 100 | 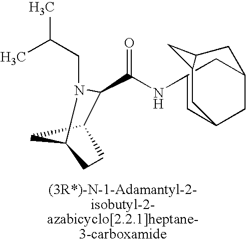 | T | NA | 331.1 |
| 211 | 32 | 80.2 | 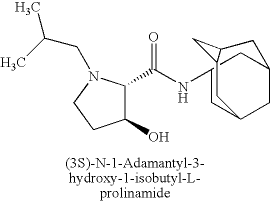 | T | NA | 321.2 |
| 212 | 85 | 77.6 | 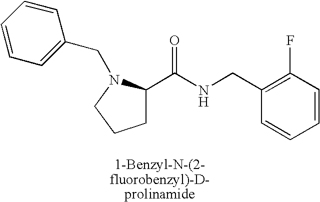 | T | NA | 313.1 |
| 213 | 120 | 78 | 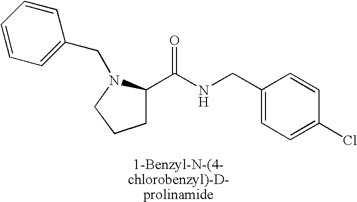 | T | NA | 329 |
| 214 | 59 | 82.7 | 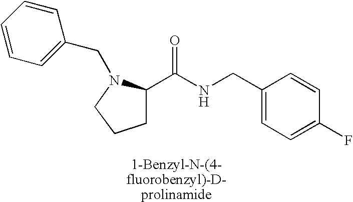 | T | NA | 313.1 |
| 215 | 1 | 100 | 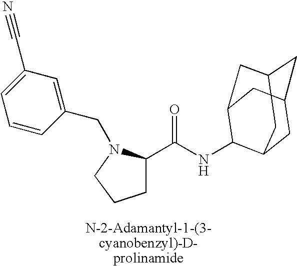 | T | NA | 364.1 |
| 216 | 1 | 96.9 | 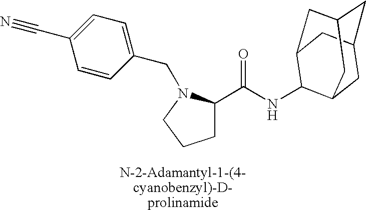 | T | NA | 364.1 |
| 217 | 171 | 84.6 | 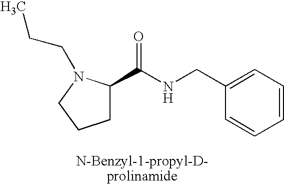 | T | NA | 247.1 |
| 218 | 2500 | 91.1 | 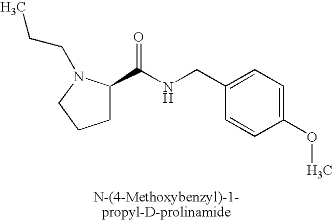 | T | NA | 277.1 |
| 219 | 168 | 86.6 | 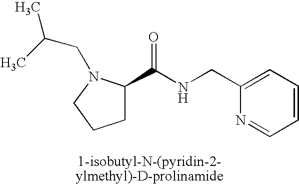 | T | NA | 262.1 |
| 220 | 5.1 | 96.7 | 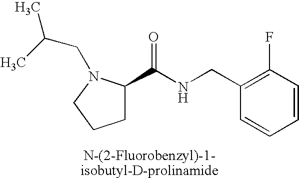 | T | NA | 279.1 |
| 221 | 5.9 | 95.7 | 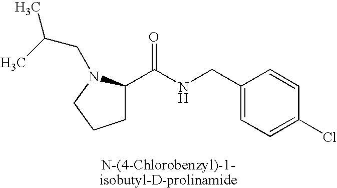 | T | NA | 297.1 |
| 222 | 6.9 | 100 | 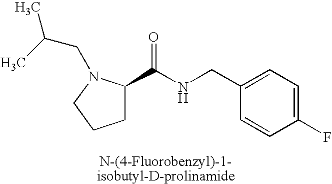 | T | NA | 279.1 |
| 223 | 26 | 86.3 | 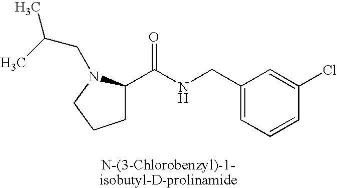 | T | NA | 295.1/ 297.1 |
| 224 | 189 | 90 | 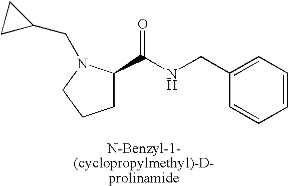 | T | NA | 259 |
| 225 | 153 | 86.2 | 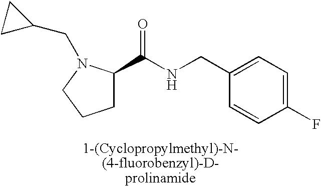 | T | NA | 277.1 |
| 226 | 22 | 82.2 | 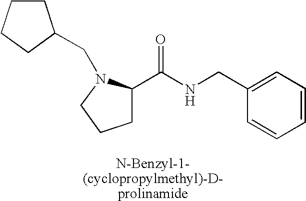 | T | NA | 287.2 |
| 227 | 36 | 80.3 | 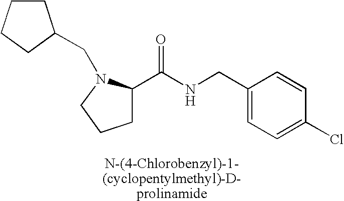 | T | NA | 321.1 |
| 228 | 54 | 89.3 | 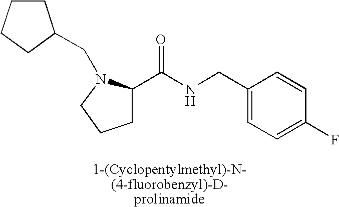 | T | NA | 305.2 |
| 229 | 48 | 73.7 | 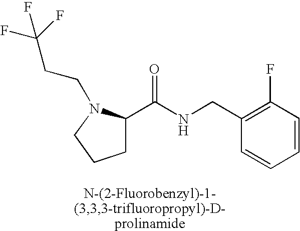 | T | NA | 319.1 |
| 230 | 1 | 100 | 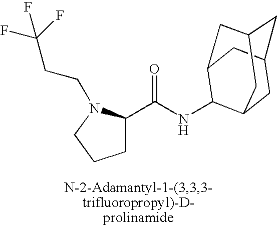 | T | NA | 345.3 |
| 231 | 1.6 | 100 | 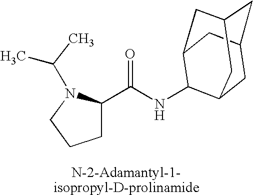 | T | NA | 291.2 |
| 232 | 47 | 71.5 | 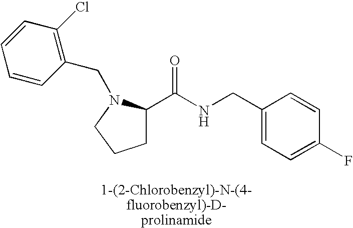 | T | NA | 347 |
| 233 | 31 | 81 | 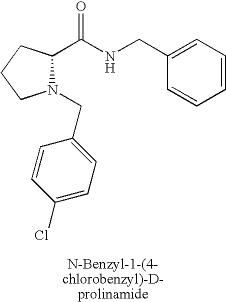 | T | NA | 347 |
| 234 | 21 | 83.5 | 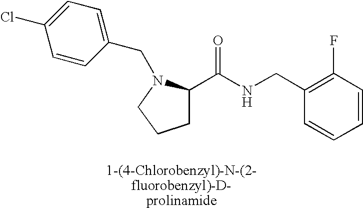 | T | NA | 329 |
| 235 | 25 | 83.8 | 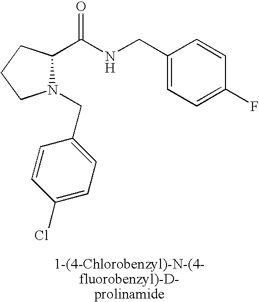 | T | NA | 347 |
| 236 | 66 | 76.8 | 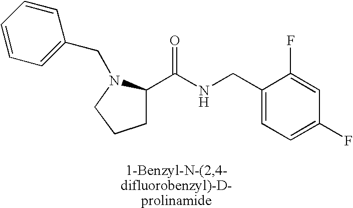 | T | NA | 331.1 |
| 237 | 10.3 | 100 | 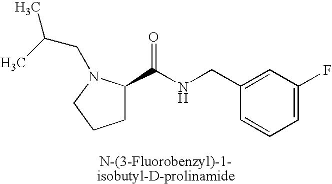 | T | NA | 279.1 |
| 238 | 16.3 | 88.4 | 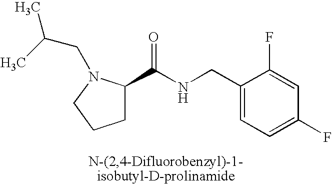 | T | NA | 297.1 |
| 239 | 5.62 | 97.6 | 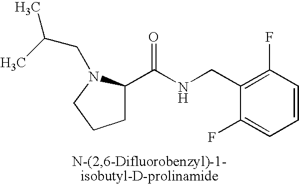 | T | NA | 297.1 |
| 240 | 44 | 76.3 | 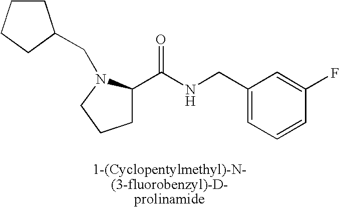 | T | NA | 305.2 |
| 241 | 32 | 81.4 | 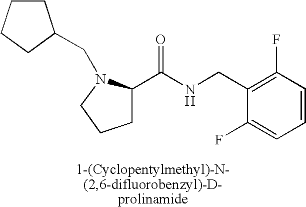 | T | NA | 323 |
| 242 | 44 | 78.9 | 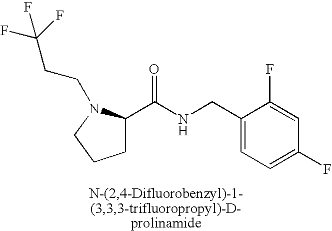 | T | NA | 337 |
| 243 | 15 | 84.9 | 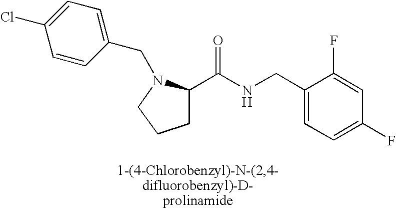 | T | NA | 365 |
| 244 | 18 | 87.1 | 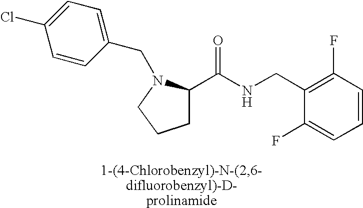 | T | NA | 365 |
| 245 | 17 | 84.6 | 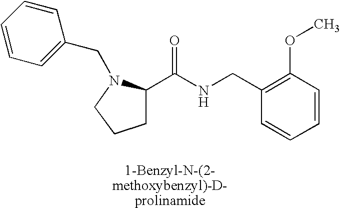 | T | NA | 325.1 |
| 246 | 16 | 87.7 | 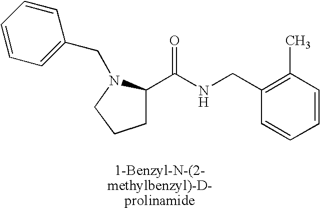 | T | NA | 309.1 |
| 247 | 14 | 88 | 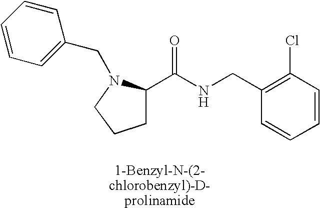 | T | NA | 329 |
| 248 | 1.3 | 100 | 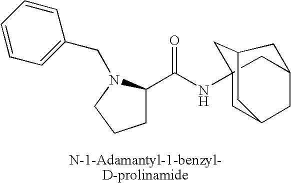 | T | NA | 339.2 |
| 249 | 1 | 100 | 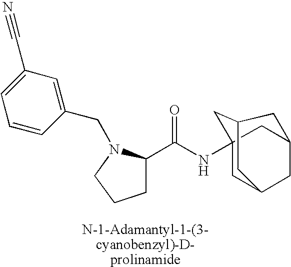 | T | NA | 364.1 |
| 250 | 5.9 | 100 | 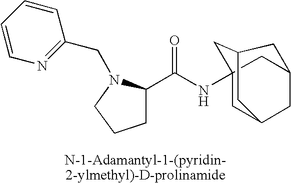 | T | NA | 340.1 |
| 251 | 47 | 83.5 | 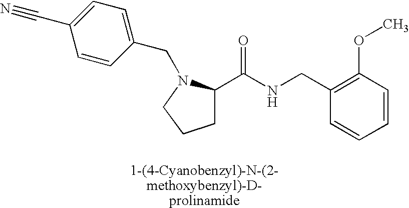 | T | NA | 350.2 |
| 252 | 52 | 82.6 | 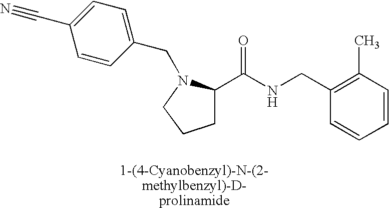 | T | NA | 334.1 |
| 253 | 24 | 100 | 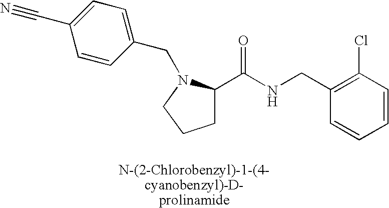 | T | NA | 354.1 |
| 254 | 1 | 100 | 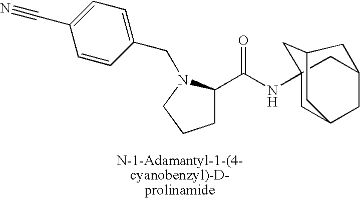 | T | NA | 364.1 |
| 255 | 8.6 | 100 | 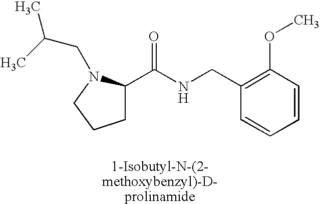 | T | NA | 291.1 |
| 256 | 7 | 100 | 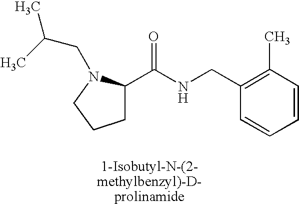 | T | NA | 275.2 |
| 257 | 2.1 | 100 | 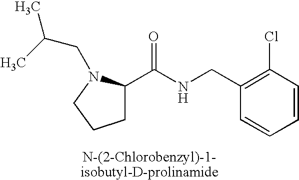 | T | NA | 295.1 |
| 258 | 24 | 90.1 | 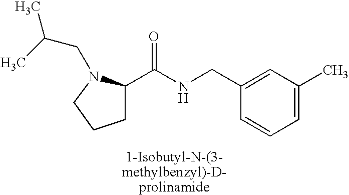 | T | NA | 275.2 |
| 259 | 11 | 100 | 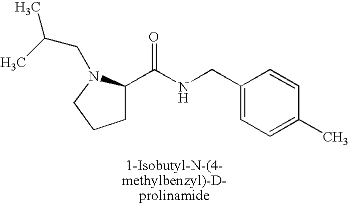 | T | NA | 275.2 |
| 260 | 16 | 89.6 | 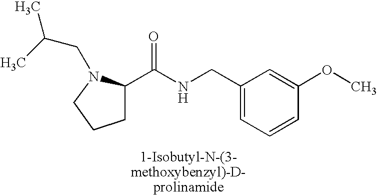 | T | NA | 291.1 |
| 261 | 25 | 100 | 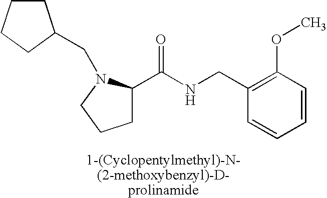 | T | NA | 317.2 |
| 262 | 60 | 76 | 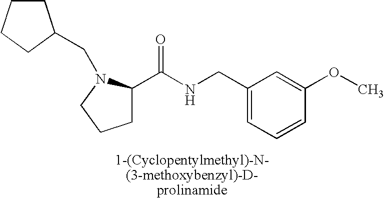 | T | NA | 317.2 |
| 263 | 62 | 77.5 | 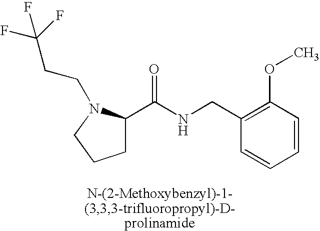 | T | NA | 331.1 |
| 264 | 10 | 86.5 | 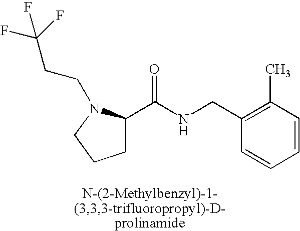 | T | NA | 315.1 |
| 265 | 4.1 | 86 | 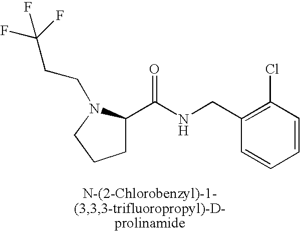 | T | NA | 335 |
| 266 | 57 | 779 | 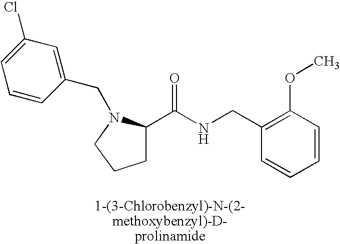 | T | NA | 359 |
| 267 | 12 | 100 | 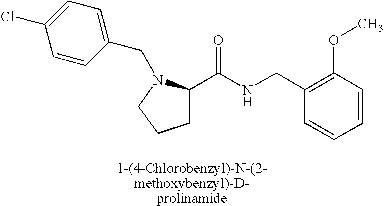 | T | NA | 359 |
| 268 | 10 | 100 | 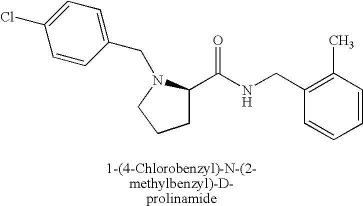 | T | NA | 343.1/ 345.1 |
| 269 | 66 | 77.8 | 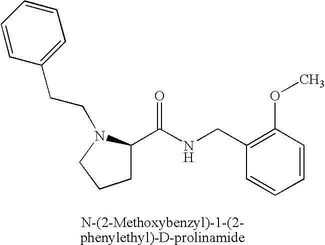 | T | NA | 339.1 |
| 270 | 35 | 84.7 | 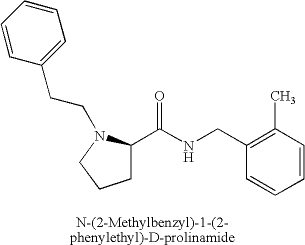 | T | NA | 323.2 |
| 271 | 26 | 83.8 | 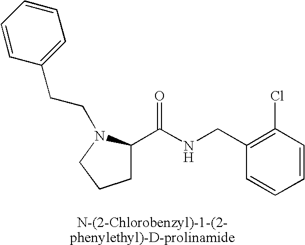 | T | NA | 343.1/ 345.1 |
| 272 | 1 | 71.7 | 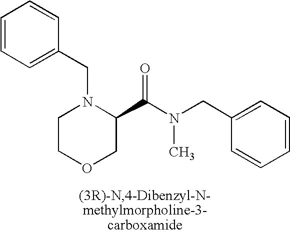 | T | NA | 325.1 |
| 273 | 4.8 | 77.2 | 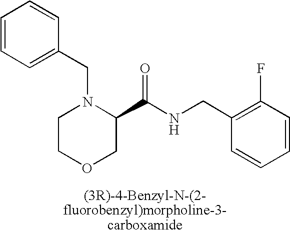 | T | NA | 380.2 |
| 274 | 2.6 | 78.4 | 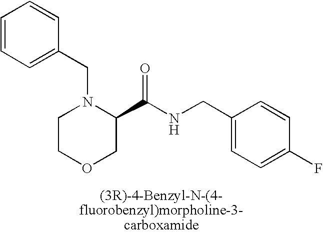 | T | NA | 329 |
| 275 | 1.5 | 100 | 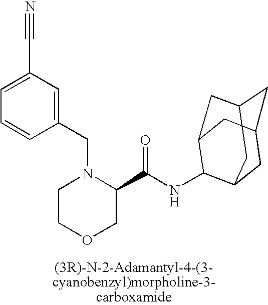 | T | NA | 380.2 |
| 276 | 2.2 | 100 | 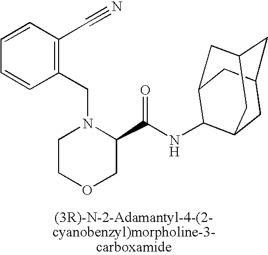 | T | NA | 354.1 |
| 277 | 80 | 73.1 | 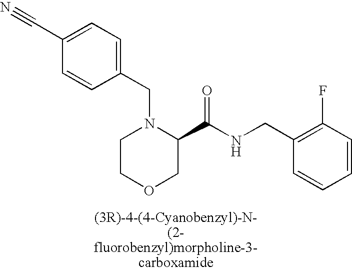 | T | NA | 356.2 |
| 278 | 49.2 | 76 | 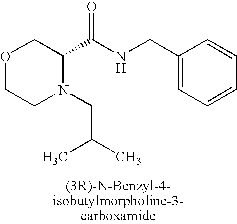 | T | NA | 277.1 |
| 279 | 3 | 100 | 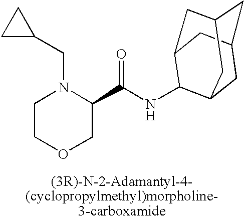 | T | NA | 319.1 |
| 280 | 49 | 81.6 | 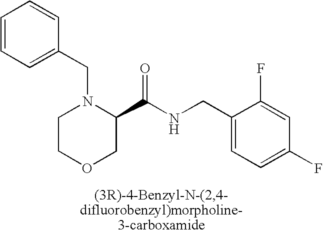 | T | NA | 347 |
| 281 | 48 | 84.9 | 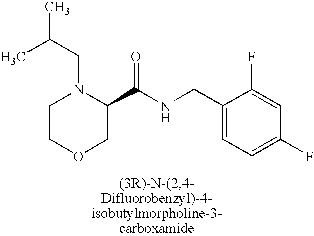 | T | NA | 313.1 |
| 282 | 2.34 | 100 | 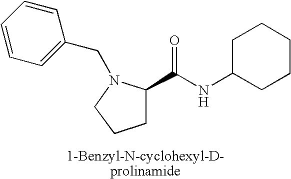 | T | NA | 287.2 |
| 283 | 5.6 | 100 | 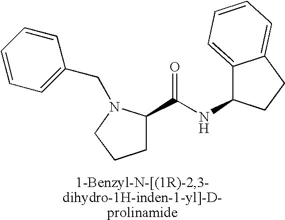 | T | NA | 321.1 |
| 284 | 11.5 | 93.1 | 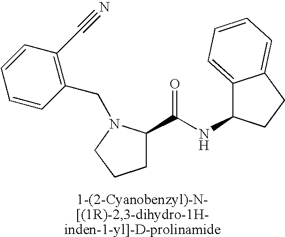 | T | NA | 346.1 |
| 285 | 7.82 | 100 | 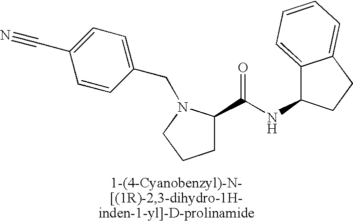 | T | NA | 346.1 |
| 286 | 5.8 | 91.4 | 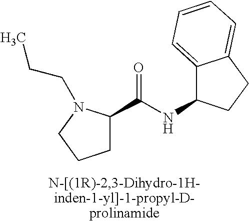 | T | NA | 273.1 |
| 287 | 3.39 | 98.7 | 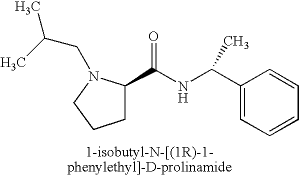 | T | NA | 275.2 |
| 288 | 5.51 | 99.1 | 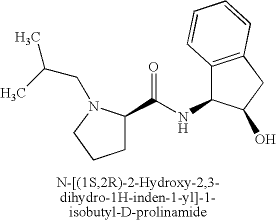 | T | NA | 303.1 |
| 289 | 1.2 | 100 | 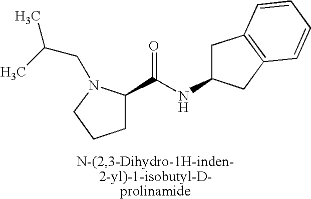 | T | NA | 287.2 |
| 290 | 6.85 | 96.2 | 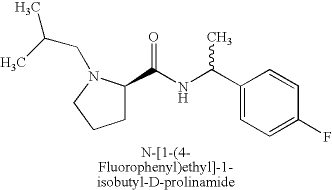 | T | NA | 293.2 |
| 291 | 1 | 100 | 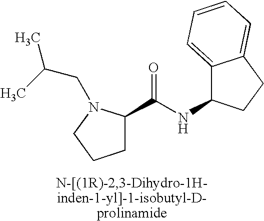 | T | NA | 287.2 |
| 292 | 9.59 | 92.1 | 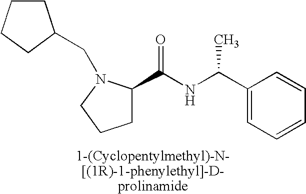 | T | NA | 301.1 |
| 293 | 22.4 | 92.6 | 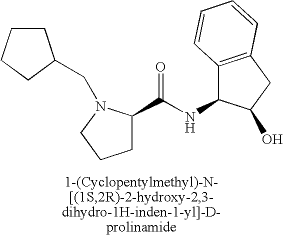 | T | NA | 329.2 |
| 294 | 8.52 | 100 | 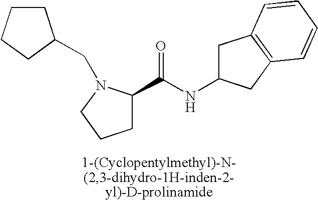 | T | NA | 313.1 |
| 295 | 4.5 | 100 | 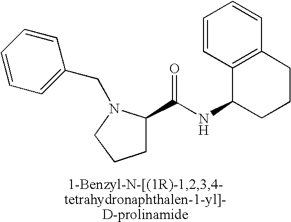 | T | NA | 335.2 |
| 296 | 11.4 | 95.7 | 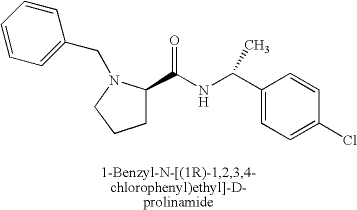 | T | NA | 345.1 |
| 297 | 3.7 | 100 | 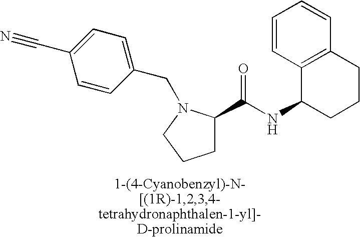 | T | NA | 360.1 |
| 298 | 1.77 | 100 | 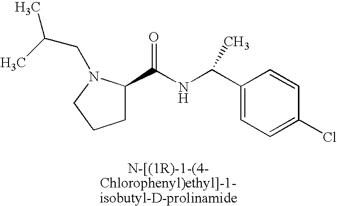 | T | NA | 309.1/ 311.2 |
| 299 | 2.23 | 100 | 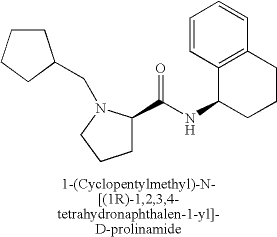 | T | NA | 327.2 |
| 300 | 5.83 | 98.4 | 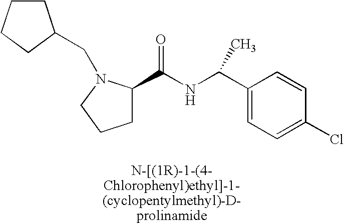 | T | NA | 435.2 |
| 301 | 11.1 | 89.2 | 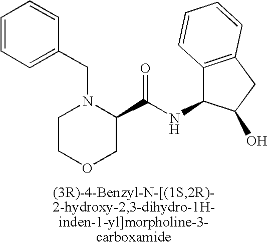 | T | NA | 353 |
| 302 | 3.2 | 99.1 | 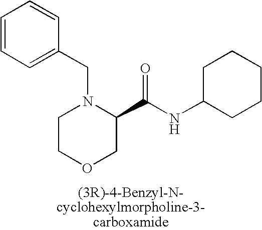 | T | NA | 303.1 |
| 303 | 4.4 | 50 | 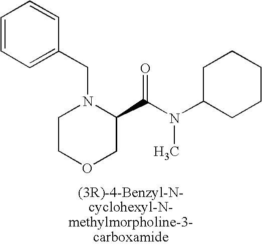 | T | NA | 317.2 |
| 304 | 3.6 | 59.4 | 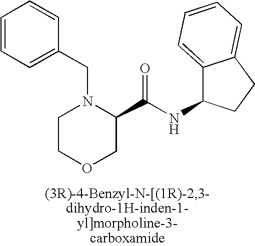 | T | NA | 337.1 |
| 305 | 3.4 | 98.7 | 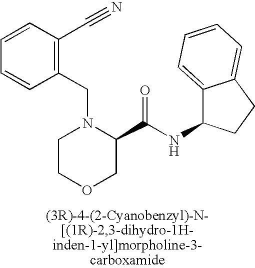 | T | NA | 362.2 |
| 306 | 4.9 | 100 | 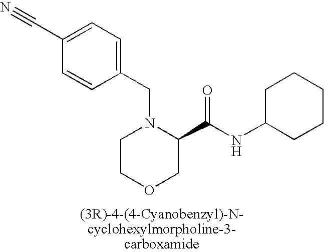 | T | NA | 328.1 |
| 307 | 12.8 | 100 | 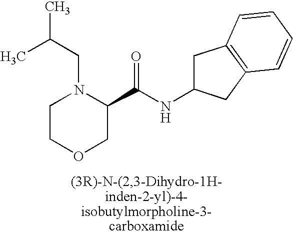 | T | NA | 303.1 |
| 308 | 5.71 | 97.7 | 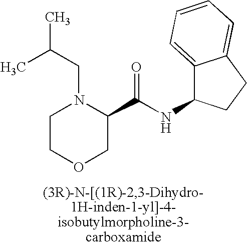 | T | NA | 303.1 |
| 309 | 32 | 86.1 | 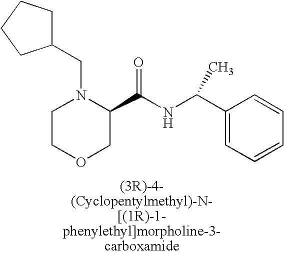 | T | NA | 317.2 |
| 310 | 29.5 | 92 | 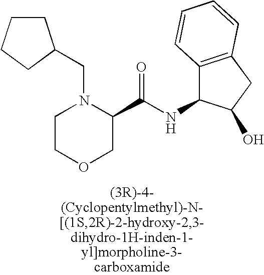 | T | NA | 345.2 |
| 311 | 6.52 | 98.5 | 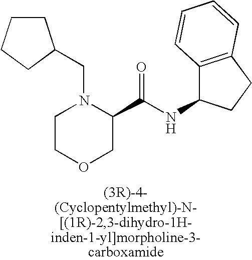 | T | NA | 359.2 |
| 312 | 14 | 95.3 | 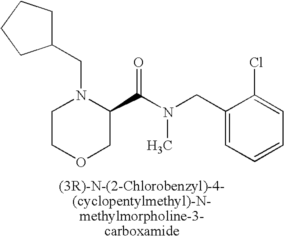 | T | NA | 351.1 |
| 313 | 4.89 | 97 | 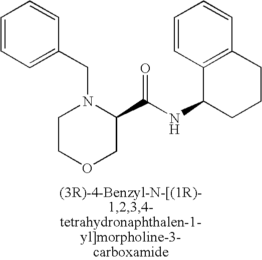 | T | NA | 351.1 |
| 314 | 8.96 | 94.8 | 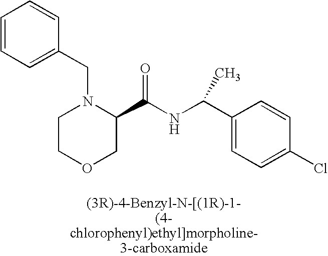 | T | NA | 361.1 |
| 315 | 6.63 | 96.1 | 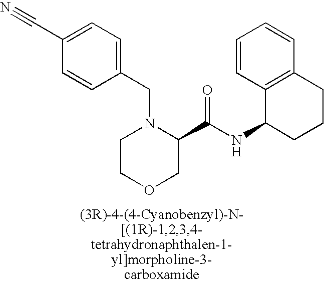 | T | NA | 376.1 |
| 316 | 10.9 | 91 | 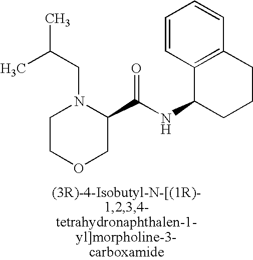 | T | NA | 317.2 |
| 317 | 8.72 | 91.1 | 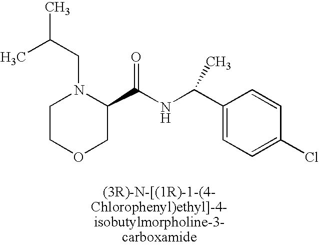 | T | NA | 325.1/ 327.1 |
| 318 | 13 | 84.8 | 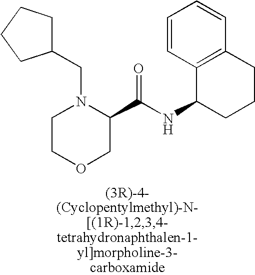 | T | NA | 343.1 |
| 319 | 1.7 | 100 | 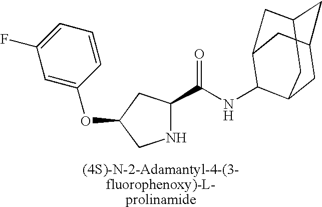 | S | NA | 359.2 |
| 320 | 5.5 | 100 | 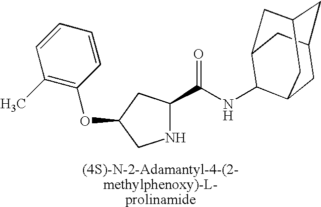 | S | NA | 355.3 |
| 321 | 1 | 100 | 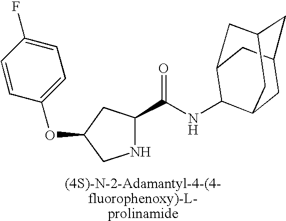 | S | NA | 359.2 |
| 322 | 45 | 62.51 | 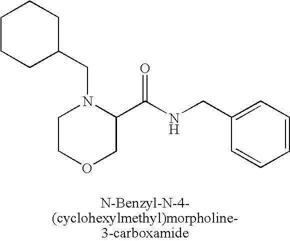 | T | NA | 317.2 |
| 323 | 110 | 69.7 | 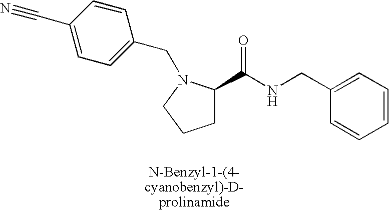 | T | NA | 320.2 |
| 324 | 69 | 69.2 | 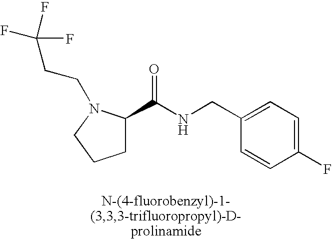 | T | NA | 319.1 |
| 325 | NA | 35.69 | 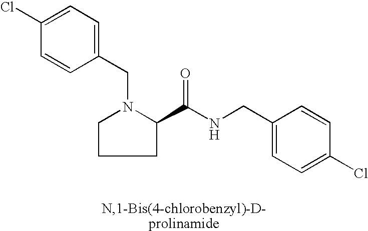 | T | NA | 363.1/ 365.0 |
| 326 | 69 | 69.4 | 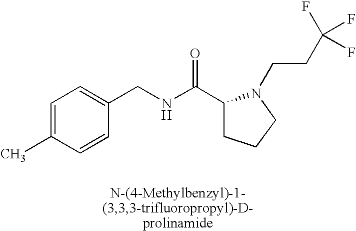 | T | NA | 315.1 |
| 327 | 110 | 68.9 | 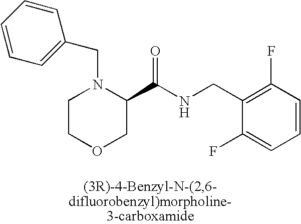 | T | NA | 347.2 |
| 328 | 3.2 | 100 | 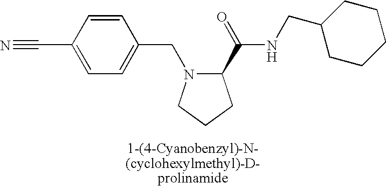 | T | NA | 326.2 |
| 329 | 1 | 100 | 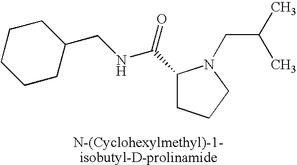 | T | NA | 367.2 |
| 330 | 30 | 83.5 | 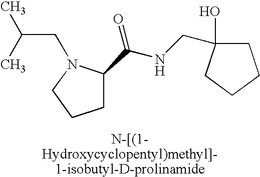 | T | NA | 269.2 |
| 331 | 26 | 86.6 | 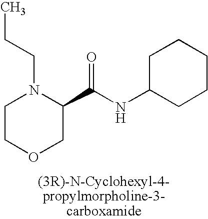 | T | NA | 255.2 |
| 332 | 2.2 | 100 | 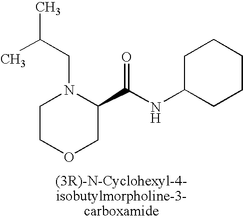 | T | NA | 269.2 |
| 333 | 4 | 100 | 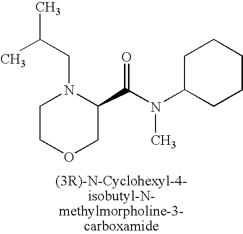 | T | NA | 283.1 |
| 334 | 2.4 | 100 | 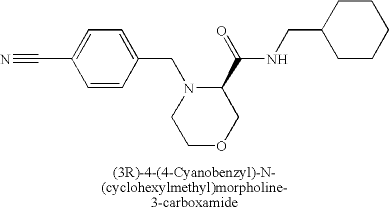 | T | NA | 342.1 |
| 335 | 6.9 | 100 | 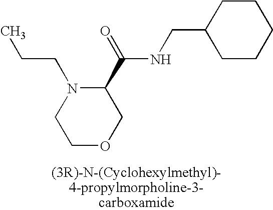 | T | NA | 269.2 |
| 336 | 1 | 100 | 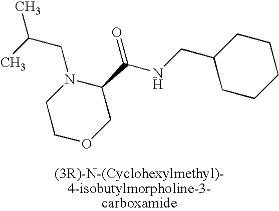 | T | NA | 283.1 |
| 337 | 13 | 97.8 | 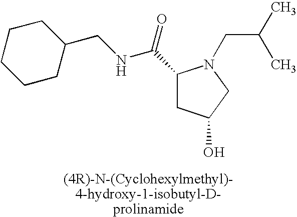 | T | NA | 283.1 |
| 338 | 1 | 100 | 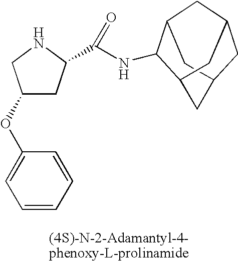 | S | NA | 341.2 |
| 339 | 1 | 977 | 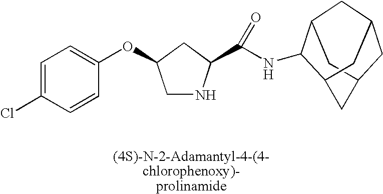 | S | NA | 375.1 |
| 340 | 5.2 | 100 | 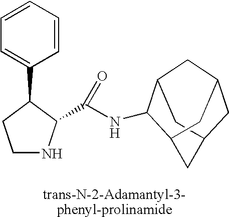 | S | NA | 325.1 |
| 341 | 0.7 | 100 | 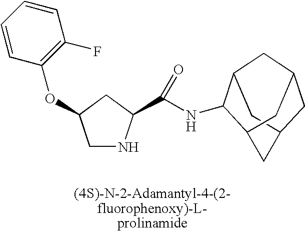 | S | NA | 359 |
| 342 | 9.9 | 97.5 | 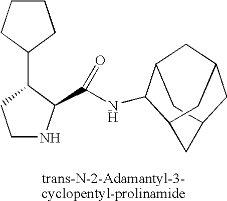 | S | NA | 317.2 |
| 343 | 6.3 | 98.6 | 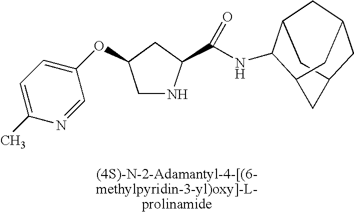 | S | NA | 356.2 |
| 344 | 11 | 90.7 | 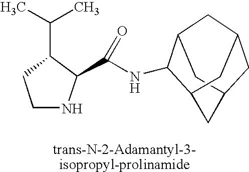 | S | NA | 291.1 |
| 345 | 27 | 76.5 | 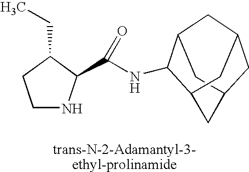 | S | NA | 277.1 |
| 346 | 18 | 90.2 | 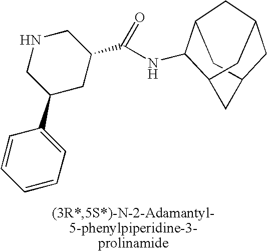 | S | NA | 339.1 |
| 347 | 3.3 | 96.6 | 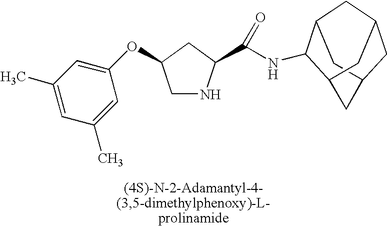 | S | NA | 369.1 |
| 348 | 2.7 | 100 | 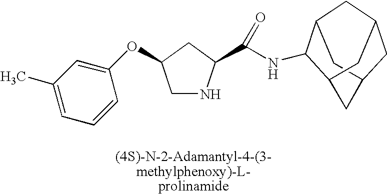 | S | NA | 355.1 |
| 349 | 1.8 | 100 | 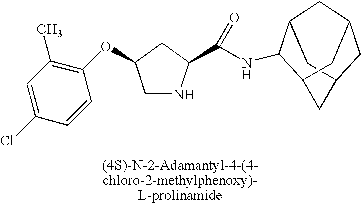 | S | NA | 389 |
| 350 | 2.2 | 100 | 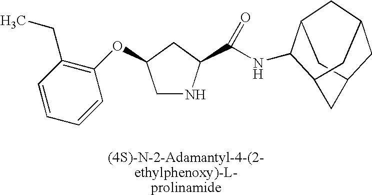 | S | NA | 369.1 |
| 351 | 1 | 100 | 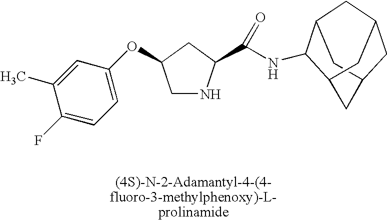 | S | NA | 373.1 |
| 352 | 1 | 100 | 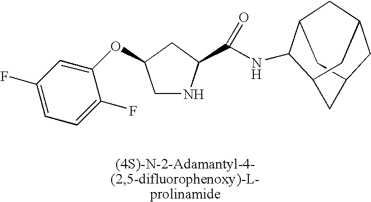 | S | NA | 377 |
| 353 | 4.9 | 100 | 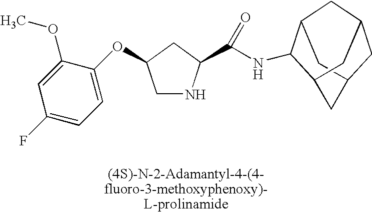 | S | NA | 389.2 |
| 354 | 2.1 | 100 | 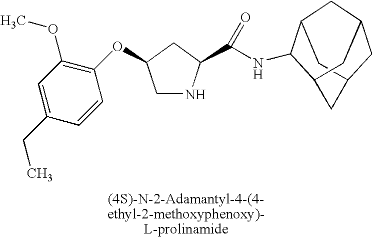 | S | NA | 399.1 |
| 355 | 1 | 100 | 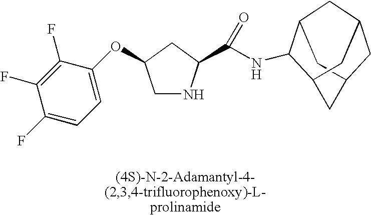 | S | NA | 395 |
| 356 | 1.4 | 100 | 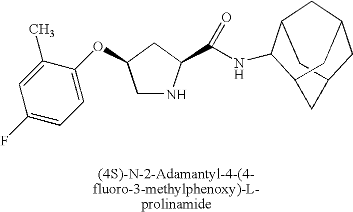 | S | NA | 373.1 |
| 357 | 2.1 | 100 | 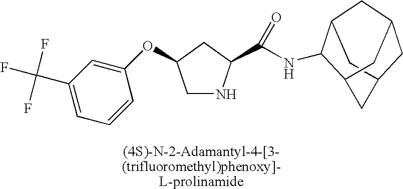 | S | NA | 409.1 |
| 358 | 4.5 | 100 | 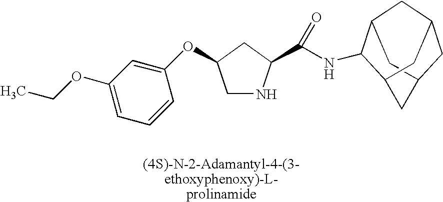 | S | NA | 385.1 |
| 359 | 44 | 80.7 | 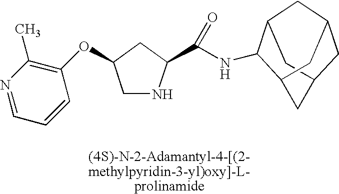 | S | NA | 356.2 |
| 360 | 1.4 | 100 | 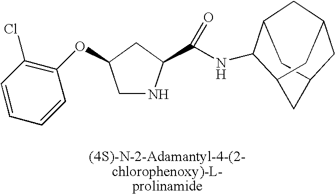 | S | NA | 375.1 |
| 361 | 1.9 | 100 | 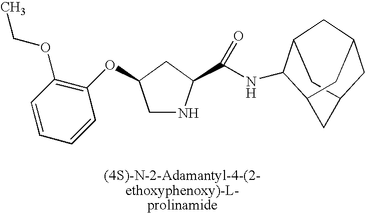 | S | NA | 385.1 |
| 362 | 1.1 | 100 | 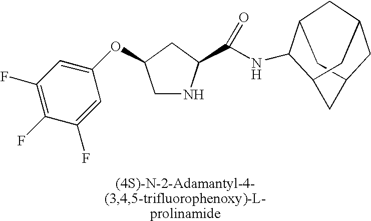 | S | NA | 395 |
| 363 | 1 | 100 | 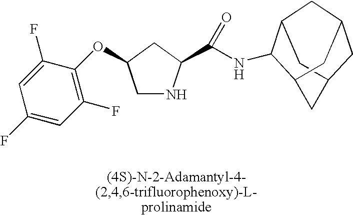 | S | NA | 395 |
| 364 | 4.6 | 100 | 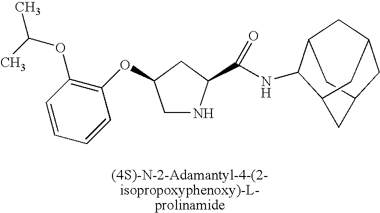 | S | NA | 399.1 |
| 365 | 1 | 100 | 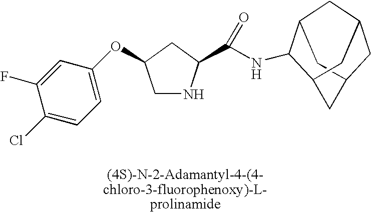 | S | NA | 393.1 |
| 366 | 15 | 94.9 | 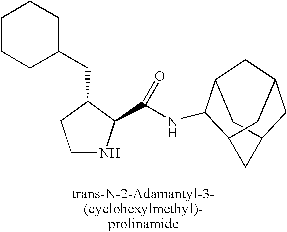 | S | NA | 345.2 |
| 367 | 7.5 | 93.5 | 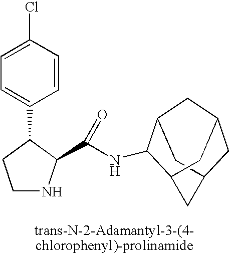 | S | NA | 359 |
| 368 | 6.8 | 96.4 | 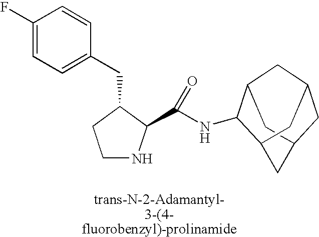 | S | NA | 357.1 |
| 369 | 6.6 | 100 | 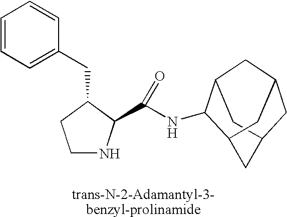 | S | NA | 339.1 |
| 370 | 10 | 88.7 | 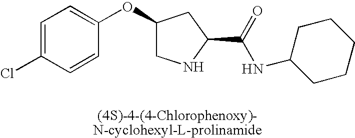 | S | NA | 323 |
| 371 | NA | 86.2 | 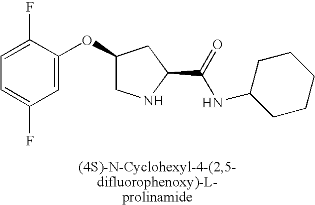 | S | NA | 325.1 |
| 372 | NA | 97.4 | 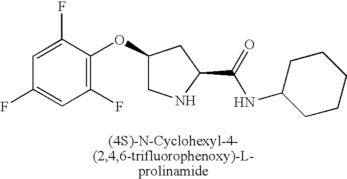 | S | NA | 343.2 |
| 373 | C | 91.8 | 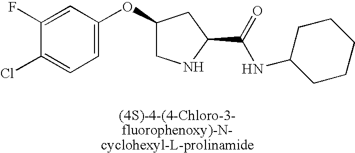 | S | NA | 341 |

Various embodiments of the present invention have been described above but a person in the art realizes further minor alterations that would fall into the scope of the present on. The breadth and scope of the present invention should not be limited by any of the described exemplary embodiments, but should be defined only in accordance with the ing claims and their equivalents.

1. A compound of formula (I): 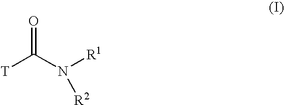
wherein;
R¹ is independently selected from the group consisting of (C₁-C₆)alkyl, —(CR⁴R⁵)ₜ(C₃-C₁₂)cycloalkyl, —(CR⁴R⁵)ₜ(C₆-C₁₂)aryl, and —(CR⁴R⁵)ₜ(4 to 10)-membered heterocyclyl;
k is independently selected from 1 or 2;
j is independently selected from the group consisting of 0, 1, and 2;
t, u, p, q and v are each independently selected from the group consisting of 0, 1, 2, 3, 4, and 5;
T is a (4 to 10)-membered heterocyclyl containing at least one nitrogen atom, wherein said nitrogen atom is optionally substituted by at least one R³ group;
R² is selected from H or (C₁-C₆)alkyl;
each R³ group is independently selected from the group consisting of —CF₃, —CHF₂, —CH₂F, trifluoromethoxy, (C₁-C₆)alkoxy, (C₁-C₆)alkyl, (C₂-C₆)alkenyl, (C₂-C₆)alkynyl, —(C═O)—R⁴, —(C═O)—O—R⁴, —(CR⁴R⁵)ₜ(C₆-C₁₂)aryl, —(CR⁴R⁵)ₜ(C₃-C₁₂)cycloalkyl, —(CR⁴R⁵)ₜ(4 to 10)-membered heterocyclyl, —(CR⁴R⁵)ₜ—(C═O)(CR⁴R⁵)ₜ(C₆-C₁₂)aryl, and —(CR⁴R⁵)ₜ—(C═O)(CR⁴R⁵)ₜ(4 to 10)-membered heterocyclyl;
each R⁴ and R⁵ group is independently selected from H or (C₁-C₆)alkyl;
any nitrogen atom of any (4 to 10)-membered heterocyclyl of the foregoing R³ group is optionally substituted with a substituent independently selected from the group consisting of (C₁-C₆)alkyl, —(SO)ₖ—R⁴, —(C═O)—O—R⁴, and —(C═O)—R⁴;
each carbon atom of T, R¹, R² and R³ is optionally substituted by 1 to 4 R⁶ groups;
each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(C═O)—R⁷, —(C═O)—O—R⁷, —O—R⁷, —O—(C═O)—R⁷, —O—(C═O)—NR⁷R⁸, —NR⁸—((C═O)—R⁹), —(C═O)NR⁸R⁹, —NR⁸R⁹, —NR⁸—(OR⁹), —NR⁸—((C═O)—O—R⁹), —S(O)ₖ—NR⁸R⁹, —S(O)ₖ—R⁸, —O—S(O)ₖ—R⁸, —NR⁸—S(O)ₖ—R⁹, —(CR¹⁰R¹¹)ᵥ(C₆-C₁₂)aryl, —(CR¹⁰R¹¹)ᵥ(C₃-C₁₂)cycloalkyl, —(CR¹⁰R¹¹)ᵥ(4 to 10)-membered heterocyclyl, —(CR¹⁰R¹¹)_{q}(C═O)(CR¹⁰R¹¹)ᵥ(C₆-C₁₂)aryl, —(CR¹⁰R¹¹)_{q}(C═O)(CR¹⁰R¹¹)ᵥ(C₃-C₁₂)cycloalkyl, —(CR¹⁰R¹¹)_{q}(C═O)(CR¹⁰R¹¹)ᵥ(4 to 10)-membered heterocyclyl, —(CR¹⁰R¹¹)ᵥO(CR¹⁰R¹¹)_{q}(C₆-C₁₂)aryl, —(CR¹⁰R¹¹)ᵥO(CR¹⁰R¹¹)_{q}(C₃-C₁₀)cycloalkyl, —(CR¹⁰R¹¹)ᵥO(CR¹⁰R¹¹)_{q}(4 to 10)-membered heterocyclyl, —(CR¹⁰R¹¹)_{q}S(O)ⱼ(CR¹⁰R¹¹)ᵥ(C₆-C₁₂)aryl, —(CR¹⁰R¹¹)_{q}S(O)ⱼ(CR¹⁰R¹¹)ᵥ(C₃-C₁₂)cycloalkyl, and —(CR¹⁰R¹¹)_{q}S(O)ⱼ (CR¹⁰R¹¹)ᵥ(4 to 10)-membered heterocyclyl;
any 1 or 2 carbon atoms of any (4 to 10)-membered heterocyclyl moiety of the foregoing R⁶ groups are optionally substituted with an oxo group;
any carbon atom of any (C₁-C₆)alkyl, any (C₆-C₁₂)aryl, any (C₃-C₁₀)cycloalkyl, or any (4 to 10)-membered heterocyclyl of the foregoing R⁶ groups are optionally substituted with 1 to 3 substituents independently selected from the group consisting of halo, cyano, nitro, —CF₃, —CFH₂, —CF₂H, trifluoromethoxy, azido, —O—R², —(C═O)—R², —(C═O)—O—R², —O—(C═O)—R¹³, —NR¹³—(C═O)R¹⁴, —(C═O)NR¹⁴R¹⁵, —NR¹⁴R¹⁵, —NR¹⁴(OR¹⁵), (C₁-C₆)alkyl, (C₂-C₆)alkenyl, (C₂-C₆)alkynyl, —(CR¹⁶R¹⁷)ᵤ(C₆-C₁₂)aryl, —(CR¹⁶R¹⁷) (C₃-C₁₂)cycloalkyl, and —(CR¹⁶R¹⁷) (4 to 10)-membered heterocyclyl;
each R⁷, R⁸, R⁹, R¹⁰, R¹¹, R¹², R¹³, R¹⁴, R¹⁵, R¹⁶ and R¹⁷ group is independently selected from the group consisting of H, (C₁-C₆)alkyl, —(C═O)NH(R¹⁸), —(CR¹⁸R¹⁹)ₚ(C₆-C₁₂)aryl, —(CR¹⁸R¹⁹)ₚ(C₃-C₁₂)cycloalkyl, and —(CR¹⁸R¹⁹)ₚ(4 to 10)-membered heterocyclyl;
any 1 or 2 carbon atoms of the (4 to 10)-membered heterocyclyl of said each R⁷, R⁸, R⁹, R¹⁰, R¹¹, R¹², R¹³, R¹⁴, R¹⁵, R¹⁶ and R¹⁷ group is optionally substituted with an oxo group;
any carbon atoms of any (C₁-C₆)alkyl, any (C₆-C₁₂)aryl, any (C₃-C₁₂)cycloalkyl or any (4 to 10)-membered heterocyclyl of the foregoing R⁷, R⁸, R⁹, R¹⁰, R¹¹, R¹², R¹³, R¹⁴, R¹⁵, R¹⁶ and R¹⁷ groups are optionally substituted with 1 to 3 substituents independently selected from the group consisting of halo, cyano, nitro, —NR²⁰R²¹, —CF₃, —CHF₂, —CH₂F, hydroxy, trifluoromethoxy, (C₁-C₆)alkyl, (C₂-C₆)alkenyl, (C₂-C₆)alkynyl, and (C₁-C₆)alkoxy;
each R¹⁶, R¹⁹, R²⁰, and R²¹ group is independently selected from H or (C₁-C₆)alkyl;
and wherein any of the above mentioned substituents comprising a —CH₃ (methyl), —CH₂ (methylene), or —CH (methine) group which is not attached to a halo, —SO or —SO₂ group, or to a N, O or S atom optionally bears on said group a substituent independently selected from hydroxy, halo, —(C₁-C₆)alkyl, —(C₁-C₆)alkoxy, —NH₂, —NH((C₁-C₆)(alkyl)) and —N((C₁-C₆)(alkyl))₂;
or a pharmaceutically acceptable salt or solvate thereof.

2. The compound according to claim 1, wherein T is a (5 to 7)-membered heterocyclyl containing at least one nitrogen atom.

3. The compound according to claim 2, wherein R² is H or methyl.

4. The compound according to claim 3, wherein R¹ is independently selected from the group consisting of adamantyl, benzyl, cyclohexyl, 2,3-dihydro-1H-inden-2-yl, —CH₂-pyridinyl, naphthalenyl, —CH₂CH₂-morpholinyl, azabicyclo(2.2.1.)heptyl, bicyclo(2.2.1.)heptyl, cycloheptyl, —CH₂-cyclopentyl, pentacyclo(4.2.0.0^{2,5}0.^{3,8}.0^{4,7})octyl, tetrahydronaphthalenyl, and naphthyridinyl;
wherein each carbon atom is optionally substituted by 1 to 4 R⁶ groups, each R⁶ group is independently selected from the group consisting of halo, cyano, —CF₃, trifluoromethoxy, hydroxy, (C₁-C₆)alkoxy, (C₁-C₆)alkyl, —O—R⁷, —(C═O)—R⁷, —(C═O)—O—R⁷, —O—(C═O)—NR⁷R⁸, —NR⁸R⁹, —NR⁸—((C═O)—R⁹), —NR⁸—((C═O)—O—R⁹), —NR⁸—(S(O)ₖ—R⁹), and —(C═O)—NR⁸R⁹.

5. The compound according to claim 2, wherein T independently selected from the group consisting of 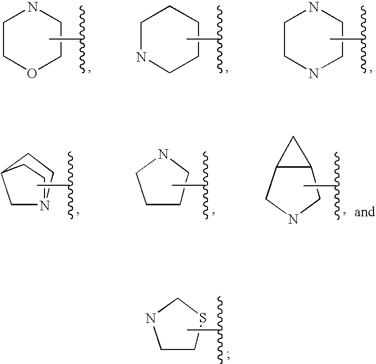
wherein said nitrogen atom is optionally substituted by at least one R³ group, wherein each said R³ group is independently selected from the group consisting of (C₁-C₆)alkyl, —(CR⁴R⁵)ₜ(C₆-C₁₂)aryl, —(CR⁴R⁵)ₜ(C₃-C₁₂)cycloalkyl, —CF₃, (C₁-C₆)alkoxy, —(C═O)—O—R⁴, and —(CR⁴R⁵)ₜ(4 to 10)-membered heterocyclyl.

6. A compound of formula (II): 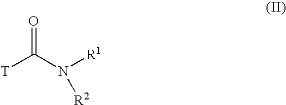
wherein;
R¹ is independently selected from the group consisting of —(CR⁴R⁵)ₜ(C₃-C₁₂)cycloalkyl, —(CR⁴R⁵)ₜ(C₆-C₁₂)aryl, and —(CR⁴R⁵)ₜ(4 to 10)-membered heterocyclyl;
k is independently selected from 1 or 2;
j is independently selected from the group consisting of 0, 1, and 2;
t, u, p, q and v are each independently selected from the group consisting of 0, 1, 2, 3, 4, and 5;
T is a (5 to 7)-membered heterocyclyl containing at least one nitrogen atom, wherein said nitrogen atom is optionally substituted by at least one R³ group;
R² is selected from H or methyl;
each R³ is independently selected from the group consisting of (C₁-C₆)alkyl, —(CR⁴R⁵)ₜ(C₆-C₁₂)aryl, —(CR⁴R⁵)ₜ(C₃-C₁₂)cycloalkyl, —(CR⁴R⁵)ₜ(4 to 10)-membered heterocyclyl, —CF₃, (C₁-C₆)alkoxy, and —(C═O)—O—R⁴;
each R⁴ and R⁵ group is independently selected from H or (C₁-C₆)alkyl;
any nitrogen atom of any (4 to 10)-membered heterocyclyl of the foregoing R³ group is optionally substituted with a substituent independently selected from the group consisting of (C₁-C₆)alkyl, —(SO)ₖ—R⁴, —(C═O)—O—R⁴, —(C═O)—R⁴;
each carbon atom of T, R¹, R² and R³ is optionally substituted by 1 to 3 R⁶ groups;
each R⁵ group is independently selected from the group consisting of halo, cyano, —CF₃, trifluoromethoxy, hydroxy, (C₁-C₆)alkoxy, (C₁-C₆)alkyl, —O—R⁷, —(C═O)—R⁷, —(C═O)—O—R⁷, —O—(C═O)—NR⁷R⁸, —NR⁸R⁹, —NR⁸—((C═O)R⁹), —NR⁸—((C═O)—O—R⁹), —NR⁸—(S(O)ₖ—R⁹), —(C═O)—NR⁸R⁹;
any 1 or 2 carbon atoms of any (4 to 10)-membered heterocyclyl moiety of the foregoing R⁵ groups are optionally substituted with an oxo group;
any carbon atom of any (C₁-C₆)alkyl of the foregoing R⁶ groups are optionally substituted with 1 to 3 substituents independently selected from the group consisting of halo, cyano, —CF₃, —O—R¹⁰, (C₁-C₆)alkyl, NR¹⁰R¹¹, and —(C═O)—NR¹¹R¹²;
each R⁷, R⁸, R⁹, R¹⁰, R¹¹, and R¹² group is independently selected from H, —(C₁-C₆)alkyl;
any carbon atoms of any (C₁-C₆)alkyl of the foregoing R⁷, R⁸, R⁹, R¹⁰, R¹¹, and R¹² groups are optionally substituted with 1 to 3 substituents independently selected from halo, cyano, nitro, —NR¹³R¹⁴, —CF₃, —CHF₂, —CH₂F, trifluoromethoxy, (C₁-C₆)alkyl, (C₂-C₆)alkenyl, (C₂-C₆)alkynyl, hydroxy, and (C₁-C₆)alkoxy;
each R¹³ and R¹⁴ group is independently selected from H or (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from hydroxy, halo,
—(C₁-C₆)alkyl, —(C₁-C₆)alkoxy, —NH₂, —NH((C₁-C₆)(alkyl)) and —N((C₁-C₆)(alkyl))₂;
or a pharmaceutically acceptable salt or solvate thereof.

7. The compound according to claim 6, wherein T independently selected from the group consisting of 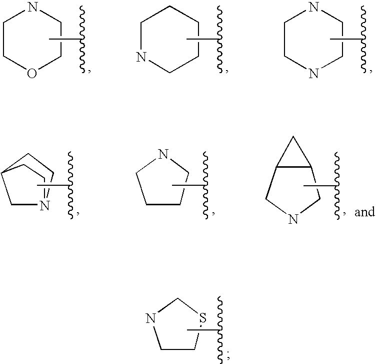
wherein said nitrogen atom is optionally substituted by at least one R³ group, wherein each said R³ group is independently selected from the group consisting of (C₁-C₆)alkyl, —(CR⁴R⁵)ₜ(C₆-C₁₂)aryl, —CF₃, (C₁-C₆)alkoxy, —(C═O)—O—R⁴, —(CR⁴R⁵)ₜ(C₃-C₁₂)cycloalkyl, and —(CR⁴R⁵)ₜ(4 to 10)-membered heterocyclyl.

8. The compound according to claim 6, wherein R² is H or methyl.

9. The compound according to claim 8, wherein R¹ is independently selected from the group consisting of adamantyl, benzyl, cyclohexyl, 2,3-dihydro-1H-inden-2-yl, —CH₂-pyridinyl, naphthalenyl,
—CH₂CH₂-morpholinyl, azabicyclo(2.2.1.)heptyl, bicyclo(2.2.1.)heptyl, cycloheptyl, —CH₂-cyclopentyl, pentacyclo(4.2.0.^{2,5}0.^{3,8}.0^{4,7})octyl, tetrahydronaphthalenyl, and naphthyridinyl;
wherein each carbon atom is optionally substituted by 1 to 4 R⁶ groups, each R⁶ group is independently selected from the group consisting of halo, cyano, —CF₃, trifluoromethoxy, hydroxy, (C₁-C₆)alkoxy, (C₁-C₆)alkyl, —O—R⁷, —(C═O)—R⁷, —(C═O)—O—R⁷, O—(C═O)—NR⁷R⁸, —NR⁸R⁹, —NR⁸—((C═O)—R⁹), —NR⁸—((C═O)—O—R⁹), —NR⁸—(S(O)ₖ—R⁹), and —(C═O)—NR⁸R⁹.

10. A compound of formula (III): 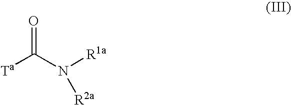
wherein;
R^{1a} is independently selected from the group consisting of adamantyl, bicyclo(2.2.1.)heptyl, and cyclohexyl;
R^{2a} is H;
T^{a} is a (5 or 6)-membered heterocyclyl containing at least one nitrogen atom, independently selected from the group consisting of pyrrolidinyl, morpholinyl, and piperidinyl;
wherein said nitrogen atom is optionally substituted by at least one R^{3a} group;
each R^{3a} is independently selected from the group consisting of methyl, ethyl, propyl, and benzyl;
each carbon atom of R^{1a} and R^{3a} is optionally substituted by 1 to 4 R⁶ groups;
each R^{6a} group is independently selected from the group consisting of —N(CH₃)(CH₃), —NH₂, —N(CH₃)(CH₂C₆H₅), —N(H)(CH₃), pyrrolidinyl, -piperidinyl-((C═O)CH₃), -piperidinyl-(CH₃), cyclohexyl, cyclopentyl, -piperidinyl-(SO₂)CH₃, hydroxy, and cyano.

11. A compound selected from the group consisting of: 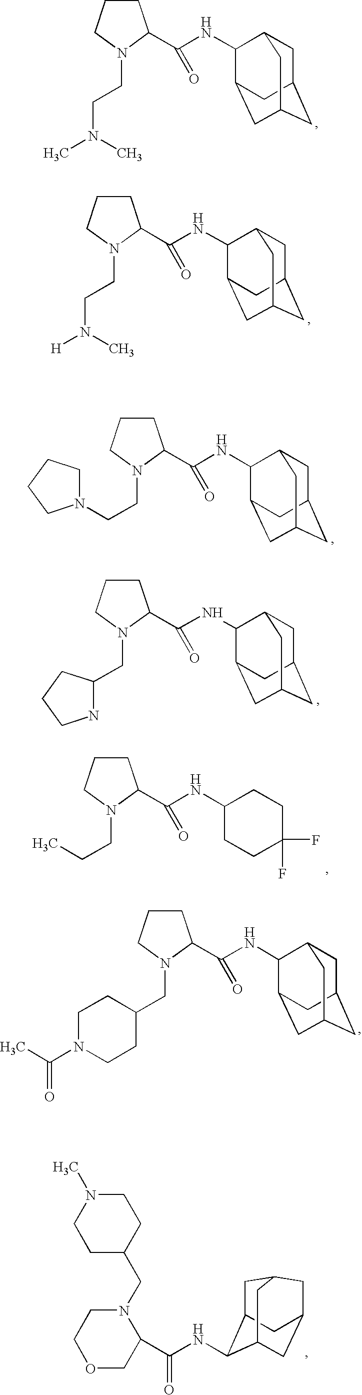 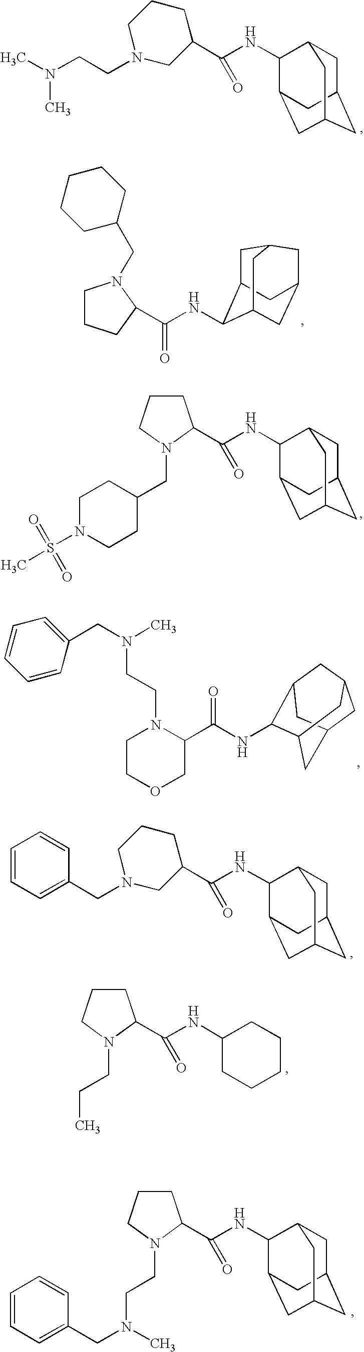 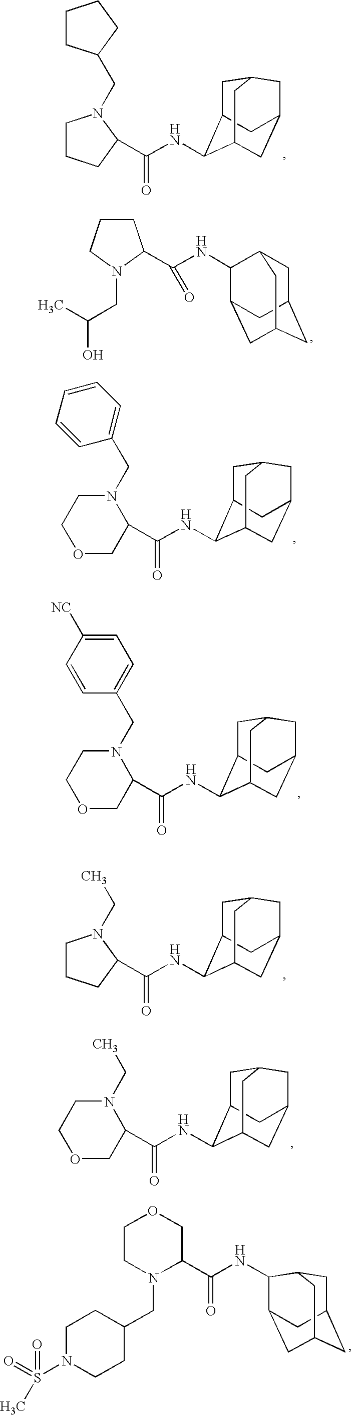 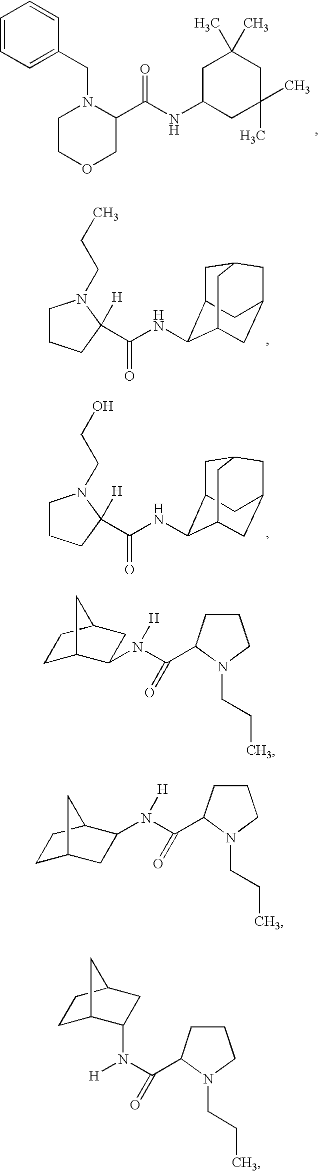 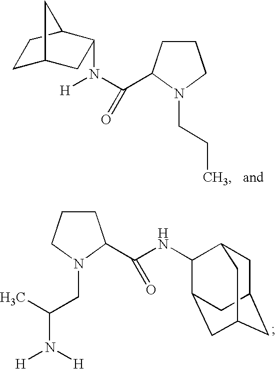
or a pharmaceutically acceptable salt or solvate thereof.

12. A compound selected from the group consisting of: 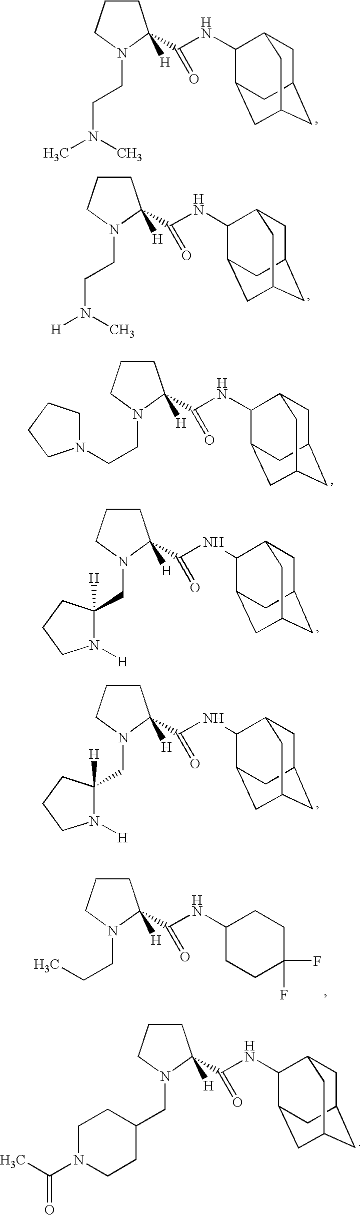 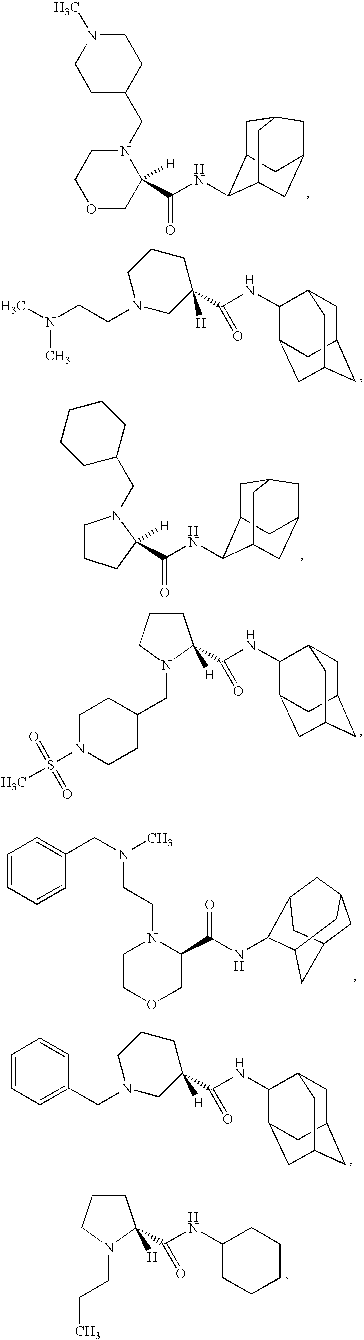 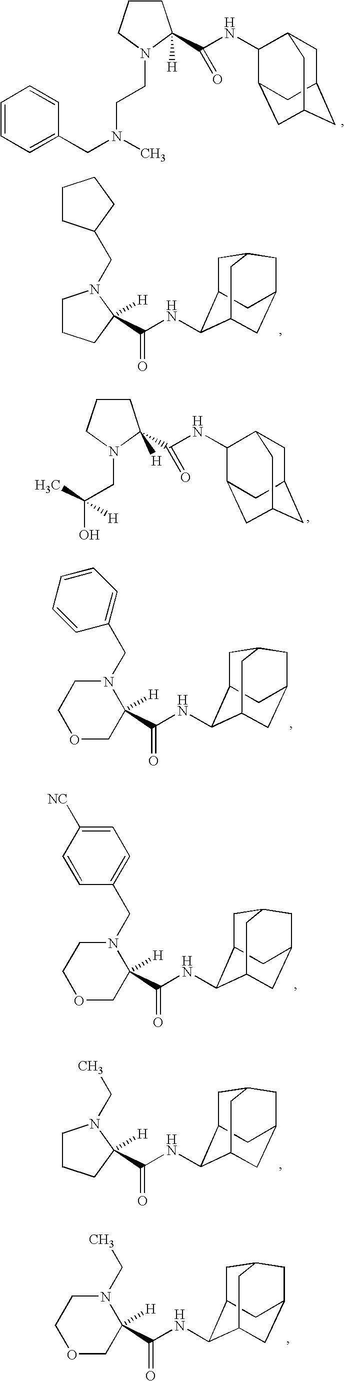 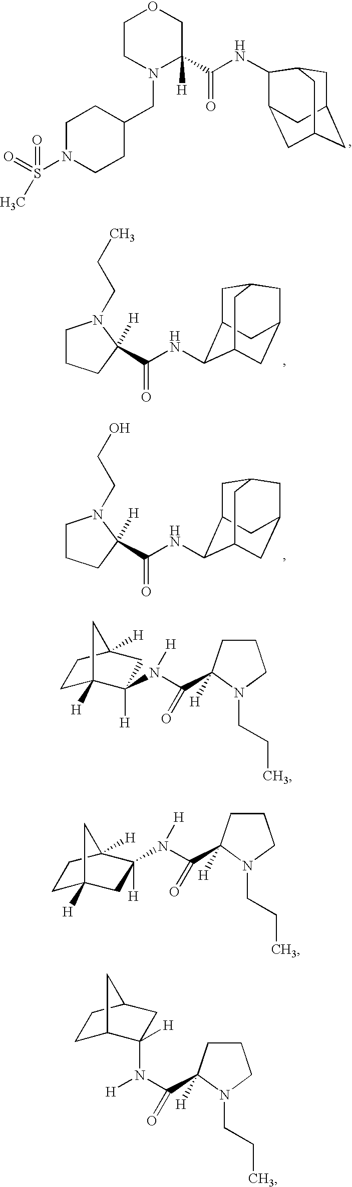 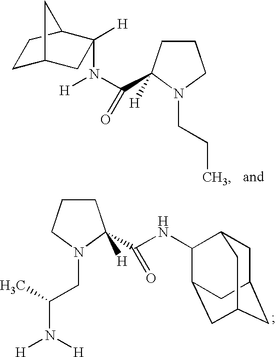
or a pharmaceutically acceptable salt or solvate thereof.

13. A pharmaceutical composition comprising an effective amount of a compound according to claim 1, or a pharmaceutically acceptable salt or solvate thereof, and a pharmaceutically acceptable carrier.

14. A method of treating a condition that is mediated by the modulation of the 11-β-hsd-1 enzyme, the method comprising administering to a mammal an effective amount of a compound according to claim 1, or a pharmaceutically acceptable salt or solvate thereof.

15. A method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, viral diseases, ophthalmic disorders, inflammatory disorders, or diseases in which the liver is a target organ, the method comprising administering to a mammal an effective amount of a compound according to claim 1, or a pharmaceutically acceptable salt or solvate thereof.
